(19) United States
(12) Patent Application Publication
(10) Pub. No.: US 2003/0198640 A1
(43) Pub. Date: Oct. 23, 2003

(54) Methods and compositions for treating inflammatory bowel diseases relating to human tumor necrosis factor-gamma-beta

(75) Inventors: Guo-Liang Yu, Berkeley, CA (US); Jian Ni, Germantown, MD (US); Craig A. Rosen, Laytonsville, MD (US); Jun Zhang, San Diego, CA (US); Ping Wei, Brookeville, MD (US)

Correspondence Address: HUMAN GENOME SCIENCES INC, 9410 KEY WEST AVENUE, ROCKVILLE, MD, 20850

(73) Assignee: Human Genome Sciences, Inc., Rockville, MD

(21) Appl. No.: 10/310,793

(22) Filed: Dec. 6, 2002

Related U.S. Application Data
(63) Continuation-in-part of application No. 10/226,294, filed on Aug. 23, 2002.
(63) Continuation-in-part of application No. 09/899,059, filed on Jul. 6, 2001.
(63) Continuation-in-part of application No. 09/559,290, filed on Apr. 27, 2000, now abandoned.
(63) Continuation-in-part of application No. 09/246,129, filed on Feb. 8, 1999.
(63) Continuation-in-part of application No. 09/131,237, filed on Aug. 7, 1998.
(63) Continuation-in-part of application No. 09/005,020, filed on Jan. 9, 1998, now abandoned.
(63) Continuation-in-part of application No. 08/461,246, filed on Jun. 5, 1995, now abandoned.
(63) Continuation-in-part of application No. PCT/US94/12880, filed on Nov. 7, 1994.
(60) Provisional application No. 60/336,695, filed on Dec. 7, 2001.
(60) Provisional application No. 60/314,381, filed on Aug. 24, 2001.
(60) Provisional application No. 60/278,449, filed on Mar. 26, 2001.
(60) Provisional application No. 60/216,879, filed on Jul. 7, 2000.
(60) Provisional application No. 60/180,908, filed on Feb. 8, 2000.
(60) Provisional application No. 60/134,067, filed on May 13, 1999.
(60) Provisional application No. 60/132,227, filed on May 3, 1999.
(60) Provisional application No. 60/131,963, filed on Apr. 30, 1999.
(60) Provisional application No. 60/074,047, filed on Feb. 9, 1998.

(51) Int. Cl. A61K 39/395

(52) U.S. Cl. 424/145.1

(57) ABSTRACT

The present invention encompasses methods for detection, diagnosis, prevention, treatment, and/or amelioration of inflammatory bowel diseases and disorders using TNF-gamma-β and its receptors DR3 and TR6. In particular the invention encompasses methods of using TNF-gamma-β, DR3 and TR6 polypeptides, as well as antibodies, and antagonists thereto, in the diagnosis, prognosis and treatment of ulcerative colitis and/or Crohn's disease. Methods of screening for antagonists of the TNF-gamma-β polypeptide, together with therapeutic uses of such antagonists are also disclosed.

### CROSS-REFERENCE TO RELATED APPLICATIONS

This application, which claims benefit under 35 U.S.C. §119(e) of U.S. Provisional Application No. 60/336,695, filed Dec. 7, 2001, is a Continuation-In-Part of U.S. patent application Ser. No. 10/226,294, filed Aug. 23, 2002; which in turn claims the benefit of priority under 35 U.S.C. §119(e) based on U.S. Provisional Application No. 60/314,381, filed Aug. 24, 2001, and is a Continuation-In-Part of U.S. patent application Ser. No. 09/899,059, filed Jul. 6, 2001; which in turn claims the benefit of priority under 35 U.S.C. §119(e) based on U.S. Provisional Application Nos. 60/278,449 and 60/216,879, filed Mar. 26, 2001 and Jul. 7, 2000 respectively, and is a Continuation-In-Part of U.S. patent application Ser. No. 09/559,290, filed Apr. 27, 2000; which in turn claims the benefit of priority under 35 U.S.C. §119(e) based on U.S. Provisional Application Nos. 60/180,908, 60/134,067, 60/132,227 and 60/131,963, filed Feb. 8, 2000, May 13, 1999, May 3, 1999 and Apr. 30, 1999 respectively, and is a Continuation-In-Part of U.S. patent application Ser. No. 09/246,129, filed Feb. 8, 1999; which in turn claims the benefit of priority under 35 U.S.C. §119(e) based on U.S. Provisional Application No. 60/074,047, filed Feb. 9, 1998, and is a Continuation-In-Part of U.S. patent application Ser. No. 09/131,237, filed Aug. 7, 1998; which in turn is a Continuation-In-Part of U.S. patent application Ser. No. 09/005,020, filed Jan. 9, 1998, now abandoned; which in turn is a Continuation-In-Part of U.S. patent application Ser. No. 08/461,246, filed Jun. 5, 1995, now abandoned; which in turn is a Continuation-In-Part of PCT/US94/12880 filed Nov. 7, 1994. The contents of each of the above-identified applications and their associated sequence listings are hereby incorporated by reference in their entireties.

### FIELD OF THE INVENTION

The present invention encompasses methods for diagnosis and treatment of inflammatory bowel diseases and disorders using a novel member of the tumor necrosis factor (TNF) family of cytokines. In particular the invention encompasses methods of using TNF-gamma-β, and/or its receptors DR3 and TR6, in the diagnosis, prognosis and treatment of inflammatory bowel diseases and disorders. Furthermore, the invention encompasses methods of using homomultimeric and heteromultimeric polypeptide complexes comprising TNF-gamma-β, and/or its receptors DR3 and TR6, in the diagnosis, prognosis and treatment of inflammatory bowel diseases and disorders. Also encompassed by the invention are methods of using TNF-gamma-β, and/or its receptors DR3 and TR6, and/or homomultimeric or heteromultimeric polypeptide complexes containing TNF-gamma-β, and/or its receptors DR3 and TR6, in the diagnosis, prognosis and treatment of diseases and/or disorders associated with inflammatory bowel diseases and disorders. Also encompassed by the invention are methods of using TNF-gamma-β, and/or its receptors DR3 and TR6, and/or homomultimeric or heteromultimeric polypeptide complexes containing TNF-gamma-β, and/or its receptors DR3 and TR6, in the diagnosis, prognosis and treatment of diseases and/or disorders associated with aberrant interferon gamma secretion and/or activity, including, for example, inflammatory bowel disease. This invention encompasses methods of using polynucleotides, polypeptides encoded by the polynucleotides, antibodies that bind the polypeptides, and antagonists of such polypeptides in the detection, diagnosis, prevention, treatment, and/or amelioration of inflammatory bowel disease. The present invention further encompasses inhibiting the production and function of the polypeptides of the present invention for prevention, treatment, and/or amelioration of inflammatory bowel disease.

### BACKGROUND OF THE INVENTION

TNF Ligand Family

The cytokine known as tumor necrosis factor-α (TNFα; also termed cachectin) is a protein secreted primarily by monocytes and macrophages in response to endotoxin or other stimuli as a soluble homotrimer of 17 kD protein subunits (Smith, R. A. et al., J. Biol. Chem. 262:6951-6954 (1987)). A membrane-bound 26 kD precursor form of TNF has also been described (Kriegler, M. et al., Cell 53:45-53 (1988)).

Accumulating evidence indicates that TNF is a regulatory cytokine with pleiotropic biological activities. These activities include: inhibition of lipoprotein lipase synthesis (“cachectin” activity) (Beutler, B. et al., Nature 316:552 (1985)), activation of polymorphonuclear leukocytes (Klebanoff, S. J. et al., J. Immunol. 136:4220 (1986); Perussia, B., et al., J. Immunol. 138:765 (1987)), inhibition of cell growth or stimulation of cell growth (Vilcek, J. et al., J. Exp. Med. 163:632 (1986); Sugarman, B. J. et al., Science 230:943 (1985); Lachman, L. B. et al., J. Immunol. 138:2913 (1987)), cytotoxic action on certain transformed cell types (Lachman, L. B. et al., supra; Darzynkiewicz, Z. et al., Canc. Res. 44:83 (1984)), antiviral activity (Kohase, M. et al., Cell 45:659 (1986); Wong, G. H. W. et al., Nature 323:819 (1986)), stimulation of bone resorption (Bertolini, D. R. et al., Nature 319:516 (1986); Saklatvala, J., Nature 322:547 (1986)), stimulation of collagenase and prostaglandin E2 production (Dayer, J. -M. et al., J. Exp. Med. 162:2163 (1985)); and immunoregulatory actions, including activation of T cells (Yokota, S. et al., J. Immunol. 140:531 (1988)), B cells (Kehrl, J. H. et al., J. Exp. Med. 166:786 (1987)), monocytes (Philip, R. et al., Nature 323:86 (1986)), thymocytes (Ranges, G. E. et al., J. Exp. Med. 167:1472 (1988)), and stimulation of the cell-surface expression of major histocompatibility complex (MHC) class I and class II molecules (Collins, T. et al., Proc. Natl. Acad. Sci. USA 83:446 (1986); Pujol-Borrel, R. et al., Nature 326:304 (1987)).

TNF is noted for its pro-inflammatory actions which result in tissue injury, such as induction of procoagulant activity on vascular endothelial cells (Pober, J. S. et al., J. Immunol. 136:1680 (1986)), increased adherence of neutrophils and lymphocytes (Pober, J. S. et al., J. Immunol. 138:3319 (1987)), and stimulation of the release of platelet activating factor from macrophages, neutrophils and vascular endothelial cells (Camussi, G. et al., J. Exp. Med. 166:1390 (1987)).

Recent evidence implicates TNF in the pathogenesis of many infections (Cerami, A. et al., Immunol. Today 9:28 (1988)), immune disorders, neoplastic pathology, e.g., in cachexia accompanying some malignancies (Oliff, A. et al., Cell 50:555 (1987)), and in autoimmune pathologies and graft-versus host pathology (Piguet, P. -F. et al., J. Exp. Med. 166:1280 (1987)). The association of TNF with cancer and infectious pathologies is often related to the host's catabolic state. A major problem in cancer patients is weight loss, usually associated with anorexia. The extensive wasting which results is known as “cachexia” (Kern, K. A. et al. J. Parent. Enter. Nutr. 12:286-298 (1988)). Cachexia includes progressive weight loss, anorexia, and persistent erosion of body mass in response to a malignant growth. The cachectic state is thus associated with significant morbidity and is responsible for the majority of cancer mortality. A number of studies have suggested that TNF is an important mediator of the cachexia in cancer, infectious pathology, and in other catabolic states.

TNF is thought to play a central role in the pathophysiological consequences of Gram-negative sepsis and endotoxic shock (Michie, H. R. et al., Br. J. Surg. 76:670-671 (1989); Debets, J. M. H. et al., Second Vienna Shock Forum, p.463-466 (1989); Simpson, S. Q. et al., Crit. Care Clin. 5:27-47 (1989)), including fever, malaise, anorexia, and cachexia. Endotoxin is a potent monocyte/macrophage activator which stimulates production and secretion of TNF (Kornbluth, S. K. et al., J. Immunol. 137:2585-2591 (1986)) and other cytokines. Because TNF could mimic many biological effects of endotoxin, it was concluded to be a central mediator responsible for the clinical manifestations of endotoxin-related illness. TNF and other monocyte-derived cytokines mediate the metabolic and neurohormonal responses to endotoxin (Michie, H. R. et al., N. Eng. J. Med. 318:1481-1486 (1988)). Endotoxin administration to human volunteers produces acute illness with flu-like symptoms including fever, tachycardia, increased metabolic rate and stress hormone release (Revhaug, A. et al., Arch. Surg. 123:162-170 (1988)). Elevated levels of circulating TNF have also been found in patients suffering from Gram-negative sepsis (Waage, A. et al., Lancet 1:355-357 (1987); Hammerle, A. F. et al., Second Vienna Shock Forum p. 715-718 (1989); Debets, J. M. H. et al., Crit. Care Med. 17:489-497 (1989); Calandra, T. et al., J. Infec. Dis. 161:982-987 (1990)).Passive immunotherapy directed at neutralizing TNF may have a beneficial effect in Gram-negative sepsis and endotoxemia, based on the increased TNF production and elevated TNF levels in these pathology states, as discussed above.

Antibodies to a “modulator” material which was characterized as cachectin (later found to be identical to TNF) were disclosed by Cerami et al. (EPO Patent Publication 0,212,489, Mar. 4, 1987). Such antibodies were said to be useful in diagnostic immunoassays and in therapy of shock in bacterial infections. Rubin et al. (EPO Patent Publication 0,218,868, Apr. 22, 1987) disclosed monoclonal antibodies to human TNF, the hybridomas secreting such antibodies, methods of producing such antibodies, and the use of such antibodies in immunoassay of TNF. Yone et al. (EPO Patent Publication 0,288,088, Oct. 26, 1988) disclosed anti-TNF antibodies, including mAbs, and their utility in immunoassay diagnosis of pathologies, in particular Kawasaki's pathology and bacterial infection. The body fluids of patients with Kawasaki's pathology (infantile acute febrile mucocutaneous lymph node syndrome; Kawasaki, T., Allergy 16:178 (1967); Kawasaki, T., Shonica (Pediatrics) 26:935 (1985)) were said to contain elevated TNF levels which were related to progress of the pathology (Yone et al., supra).

Other investigators have described mAbs specific for recombinant human TNF which had neutralizing activity in vitro (Liang, C-M. et al. Biochem. Biophys. Res. Comm. 137:847-854 (1986); Meager, A. et al., Hybridoma 6:305-311 (1987); Fendly et al., Hybridoma 6:359-369 (1987); Bringman, T. S. et al., Hybridoma 6:489-507 (1987); Hirai, M. et al., J. Immunol. Meth. 96:57-62 (1987); Moller, A. et al. (Cytokine 2:162-169 (1990)). Some of these mAbs were used to map epitopes of human TNF and develop enzyme immunoassays (Fendly et al., supra; Hirai et al., supra; Moller et al., supra) and to assist in the purification of recombinant TNF (Bringman et al., supra). However, these studies do not provide a basis for producing TNF neutralizing antibodies that can be used for in vivo diagnostic or therapeutic uses in humans, due to immunogenicity, lack of specificity and/or pharmaceutical suitability.

Neutralizing antisera or mAbs to TNF have been shown in mammals other than man to abrogate adverse physiological changes and prevent death after lethal challenge in experimental endotoxemia and bacteremia. This effect has been demonstrated, e.g., in rodent lethality assays and in primate pathology model systems (Mathison, J. C. et al., J. Clin. Invest. 81:1925-1937 (1988); Beutler, B. et al., Science 229:869-871 (1985); Tracey, K. J. et al, Nature 330:662-664 (1987); Shimamoto, Y. et al., Immunol. Lett. 17:311-318 (1988); Silva, A. T. et al., J. Infect. Dis. 162:421-427 (1990); Opal, S. M. et al., J. Infect. Dis. 161:1148-1152 (1990); Hinshaw, L. B. et al., Circ. Shock 30:279-292 (1990)).

To date, experience with anti-TNF mAb therapy in humans has been limited but shows beneficial therapeutic results, e.g., in arthritis and sepsis. See, e.g., Elliott, M. J. et al., Baillieres Clin. Rheumatol. 9:633-52 (1995); Feldmann M, et al., Ann. N. Y. Acad. Sci. USA 766:272-8 (1995); van der Poll, T. et al., Shock 3:1-12 (1995); Wherry et al., Crit. Care. Med. 21:S436-40 (1993); Tracey K. J., et al., Crit. Care Med. 21:S415-22 (1993).

Sequence analysis of cytokine receptors has defined several subfamilies of membrane proteins (1) the Ig superfamily, (2) the hematopoietin (cytokine receptor superfamily and (3) the tumor necrosis factor (TNF)/nerve growth factor (NGF) receptor superfamily (for review of TNF superfamily see, Gruss and Dower, Blood 85(12):3378-3404 (1995) and Aggarwal and Natarajan, Eur. Cytokine Netw., 7(2):93-124 (1996)). The TNF/NGF receptor superfamily contains at least 10 different proteins. Gruss and Dower, supra. Ligands for these receptors have been identified and belong to at least two cytokine superfamilies. Gruss and Dower, supra.

Inflammatory Bowel Disease

Inflammatory Bowel Disease (IBD) includes a number of chronic inflammatory disorders of the intestines. The two most common Inflammatory Bowel Diseases are Crohn's disease and ulcerative colitis. While both are inflammatory diseases of the bowel, there are several significant differences between Crohn's disease and ulcerative colitis. In ulcerative colitis, inflammation is confined to the inner lining (mucosa and/or submucosa) of the large intestine (colon and/or rectum), while in Crohn's disease inflammation extends beyong the inner lining and penetrates deeper layers of the intestinal wall of any part of the digestive system (esophagus, stomach, small intestine, large intestine, and/or anus). These disorders can cause painful, often life altering symptoms including, for example, diarrhea, cramping and rectal bleeding. Depending on the severity of these symptoms, patients may be unable to work or leave the home due to pain, fatigue, and the need for constant access to bathroom facilities.

IBD is a chronic, lifelong disease, which occurs most frequently in the industrialized world, where it is estimated to affect approximately one million (1,000,000) patients in the U.S., Europe and Japan. Age of onset of IBD falls into two distinct ranges, 15 to 30 years of age and 60 to 80 years of age. The highest mortality is during the first years of disease and in cases where the disease symptoms are longlasting, due to an increased risk of colon cancer. IBD accounts for approximately 700,000 physician vists and 100,000 hospitalizations per annum in the U.S. alone. Approximately 50% of IBD cases in the U.S. are diagnosed as ulcerative colitis and 50% as Crohn's disease. Crohn's disease presently accounts for approximately two thirds of IBD physician visits and hospitalizations, and 50 to 80% of Crohn's disease patients eventually require surgical treatment.

The exact causes of Inflammatory Bowel Disease remain unknown, however, both genetic and environmental factors are believed to be involved in their development. In the United States, Europe and South Africa, there is a two to four-fold increased frequency of IBD occurrence in Jewish populations, with Ashkenazi Jews showing a two-fold increase in IBD occurrence compared to Sephardic, Israeli, or Oriental Jews. IBD also occurs more frequently in non-Jewish Caucasian than in African-American populations, and more frequently in African-American than in Hispanic populations, and more frequently in Hispanic than in Asian populations. Despite a preponderance of evidence showing inheritance of a risk for IBD, molecular genetics has yet to provide convincing evidence of the existence of an “IBD” gene.

A number of environmental factors also influence the risk of diagnosis with IBD. Such factors include smoking, with smokers having a 40% greater risk than non-smokers of being diagnosed with ulcerative colitis, and a two-fold increased risk of being diagnosed with Crohn's disease. Furthermore, oral contraceptive use among women leads to an approximately two-fold increase in the risk of being diagnosed with Crohn's disease, while appendectomy (removal of the appendix) may be protective for ulcerative colitis.

Development of IBD is influenced by environmental and host specific factors, as described above, together with “exogenous biological factors” such as, for example, the constituents of the intestinal flora, the naturally occurring bacteria found in the intestine. It is believed that in genetically predisposed individuals, exogenous factors such as, for example, infectious agents and/or commensal organisms, and host specific characteristics such as, for example, intestinal barrier function and/or blood supply, combine with specific environmental factors such as, for example, smoking, to cause a chronic state of improperly regulated immune function. In this hypothetical model, microorganisms trigger an immune response the intestine and in susceptible individuals this immune response is not turned off when the microorganism is cleared from the body. The chronically “turned on” immune response causes damage to the intestine resulting in the symptoms of IBD.

Intestinal inflammation associated with ulcerative colitis is limited to the large intestine only. Approximately 40 to 50% of patients diagnosed with ulcerative colitis have inflammation restricted to the rectum and sigmoid colon; approximately 15% have inflammation that spreads from the rectum and sigmoid colon through the entire large intestine leading to inflammation of the entire colon (pancolitis); and approximately 30 to 40% of patients have inflammation that extends beyond the rectum and sigmoid colon but does not involve the entire colon. Inflammation associated with ulcerative colitis is limited to the inner lining of the intestine, and while it can be mild, moderate, or severe but is always continuous, with inflammation beginning at the rectum and spreading evenly without skipping any areas.

Unlike ulcerative colitis, Crohn's disease involves inflammation that can affect any part of the gastrointestinal tract. Approximately 30 to 40% of patients diagnosed with Crohn's disease have inflammation restricted to the small intestine; approximately 40 to 55% have inflammation of both small and large intestines; and approximately 15 to 25% have inflammation restricted to the colon. Crohn's disease inflammation most commonly occurs in the region where the large and small intestines meet, the ileocecal region. Whereas in ulcerative colitis the rectum has the most severe inflammation, in Crohn's disease the rectum is often free from inflammation. Inflammation associated with Crohn's disease may penetrate the entire intestinal wall and may be segmental, with areas of healthy bowel interspersed with inflamed areas. One pathologic hallmark of Crohn's disease is the appearance of granulomas, small, firm, persistent nodular inflammatory growths containing immune cells, in the intestine.

Ulcerative colitis and Crohn's disease have features similar to those of many other diseases and there is no key diagnostic test useful in there identification, therefore a combination of clinical, laboratory, histopathological (biopsies), radiographic and therapeutic observations contribute to their diagnosis. Once a diagnosis of IBD is made, it may not be possible to distinguish between Crohn's disease and ulcerative colitis in approximately 10 to 20% of cases, the “indeterminate” cases.

As many as one third of all patients diagnosed as having an IBD may also display one or more of a variety of symptoms outside of the intestines. Such symptoms include, for example, skin diseases, connective tissue diseases such as arthritis, eye diseases, liver disease, gallbladder disease, and diseases of the urinary system such as kidney stones.

It is well established that patients having long-standing ulcerative colitis are at increased risk of developing pre-cancerous colon lesions and colon cancer. The risk of colon cancer in chronic ulcerative colitis patients increases with duration and extent of the disease. Therefore, patients with chronic ulcerative colitis should receive surveillance colonoscopies and biopsies routinely as standard care. Risk factors for developing colorectal cancer in Crohn's disease are a history of colonic or ileocolonic involvement together with a long disease duration. Cancer risks in Cohn's disease patients are similar to those of ulcerative colitis patients and therefore surveillance endoscopies and biopsies are recommended as standard care.

Accordingly, more effective treatments for inflammatory bowel disease would not only improve the health of vast numbers of people worldwide, but would also reduce the economic costs of these afflictions at the individual and societal level.

### SUMMARY OF THE INVENTION

The present invention encompasses the detection, diagnosis, prognosis and/or treatment of inflammatory bowel diseases and disorders, including but not limited to Crohn's disease and ulcerative colitis, using compositions comprising polynucleotides encoding TNF-gamma-β and/or its receptors DR3 and TR6, the polypeptides encoded by these polynucleotides and antibodies that immunospecifically bind these polypeptides. See PCT Publication Nos. WO96/14328, WO00/66608, WO97/33904, WO00/64465, WO98/30694 and WO00/52028, the contents of which are hereby incorporated by reference in their entireties. More specifically, the present invention encompasses isolated TNF-gamma-β, DR3 and TR6 nucleic acid molecules, which encode TNF-gamma-β, DR3 and TR6 polypeptides respectively, as well as with antibodies that bind to these polypeptides. Also encompassed are vectors, host cells, and recombinant and synthetic methods for producing TNF-gamma-β, DR3 and TR6 polynucleotides, polypeptides, and/or antibodies. The invention further encompasses diagnostic and therapeutic methods useful for diagnosing, treating, ameliorating, preventing and/or prognosing inflammatory bowel disease. The invention further encompasses screening methods for identifying agonists and antagonists of polynucleotides and polypeptides of the invention. The invention further encompasses methods and/or compositions for inhibiting or promoting the production and/or function of the polypeptides of the invention. The invention is based in part on the ability of TNF-gamma-β to stimulate interferon-gamma secretion by lamina propria mononuclear cells and thus exacerbate inflammation in patients having inflammatory bowel disease, as demonstrated in Examples 37 and 38, below.

In preferred embodiments of the invention, inflammatory bowel disease is treated by inhibiting the activity of TNF-gamma-β, preferably by inhibiting its binding to DR3. TR6 is a soluble receptor protein that binds TNF-gamma-β. Thus, particular preferred embodiments of the invention include prevention or treatment of inflammatory bowel disease using antagonists of TNF-gamma-β, including, but not limited to the following antagonists of these two proteins: anti-TNF-gamma-β antibodies, antagonistic anti-DR3 antibodies (i.e., antibodies that do not agonistically trigger intracellular signaling), soluble DR3 extracellular domain polypeptides, TR6 polypeptides, and antagonistic peptide fragments of TNF-gamma-β and the DR3 extracellular domain. Antagonists against TNF-gamma-β, such as antibodies and peptides, may bind to TNF-gamma-β homotrimers or the TNF-gamma-β-containing heteromeric proteins described in detail below.

In specific non-limiting embodiments, the antagonists prevent or ameliorate Crohn's disease by inhibiting TNF-gamma-β/DR-mediated activation of T_{H}1 cells.

In accordance with one embodiment, the present invention encompasses the use of one or more TNF-gamma-β, DR3 and/or TR6 polypeptides, as well as biologically active fragments, analogs and derivatives thereof, together with fragments, analogs and derivatives thereof, in the diagnosis, prevention, treatment, and/or amelioration of inflammatory bowel diseases or disorders including, for example, Crohn's disease and ulcerative colitis.

In accordance with a further embodiment, the present invention encompasses the use of one or more multimeric complexes of TNF-gamma-β, DR3 and/or TR6 polypeptides, or biologically active fragments, analogs and derivatives thereof, in the diagnosis, prevention, treatment, and/or amelioration of inflammatory bowel diseases or disorders including, for example, Crohn's disease and ulcerative colitis.

In accordance with a further embodiment encompassed by the present invention, the multimeric polypeptide complex used to detect, diagnose, prognose, treat and/or ameliorate an inflammatory bowel disease, may be a homodimer, a homotrimer, a homotetramer or a higher homomultimeric complex of TNF-gamma-β, DR3 and/or TR6 polypeptides, or fragments, analogs or derivatives thereof.

In accordance with a further embodiment encompassed by the present invention, the multimeric polypeptide complex used to detect, diagnose, prognose, treat and/or ameliorate an inflammatory bowel disease, may be a heterodimer, a heterotrimer, a heterotetramer or a higher heteromultimeric complex of TNF-gamma-β, DR3 and/or TR6 polypeptides, or fragments, analogs or derivatives thereof.

In specific embodiments, the present invention encompasses the use of heteromultimeric complexes, particularly heterotrimeric complexes, comprising TNF-gamma-β, DR3 and/or TR6 polypeptides, wherein said TNF-gamma-β, DR3 and/or TR6 polypeptides may be full-length polypeptides or polypeptide domains as described previously, in the detection, diagnosis, prognosis, treatment and/or amelioration of inflammatory bowel disease. See PCT Publication Nos. WO96/14328, WO00/66608, WO97/33904, WO00/64465, WO98/30694 and WO00/52028.

In further specific embodiments the present invention encompasses the use of heteromultimeric complexes, particularly heterotrimeric complexes, comprising polypeptides at least 80% identical, more preferably at least 85% or 90% identical, and still more preferably 95%, 96%, 97%, 98% or 99% identical to TNF-gamma-β, DR3 and/or TR6, wherein said TNF-gamma-β, DR3 and/or TR6 polypeptides may full length polypeptides or polypeptide domains as described previously, in the detection, diagnosis, prognosis, treatment and/or amelioration of inflammatory bowel disease. See PCT Publication Nos. WO96/14328, WO00/66608, WO97/33904, WO00/64465, WO98/30694 and WO00/52028.

In specific embodiments the present invention encompasses the use of heterotrimeric polypeptide complexes, which contain three full-length TNF-gamma-β, DR3 and/or TR6 polypeptides; three TNF-gamma-β, DR3 and/or TR6 domain-containing polypeptides; one full-length TNF-gamma-β, DR3 and/or TR6 polypeptide together with two TNF-gamma-β, DR3 and/or TR6 domain-containing polypeptides; or two full-length TNF-gamma-β, DR3 and/or TR6 polypeptides together with one TNF-gamma-β, DR3 and/or TR6 domain-containing polypeptide.

In further specific embodiments the present invention encompasses the use of heterotrimeric polypeptide complexes, which contain two full-length TNF-gamma-β, DR3 and/or TR6 polypeptides together with one full-length TNF family member polypeptide; two TNF-gamma-β, DR3 and/or TR6 domain-containing polypeptides together with one full-length TNF family member polypeptide; two full-length TNF-gamma-β, DR3 and/or TR6 polypeptides together with one TNF family member extracellular domain polypeptide; two TNF-gamma-β, DR3 and/or TR6 domain-containing polypeptides together with one TNF family member extracellular domain polypeptide; one full-length TNF-gamma-β, DR3 and/or TR6 polypeptide together with two full-length TNF family member polypeptides; one TNF-gamma-β, DR3 and/or TR6 domain-containing polypeptide together with two full-length TNF family member polypeptides; one full-length TNF-gamma-β, DR3 and/or TR6 polypeptide together with two TNF family member extracellular domain polypeptides; or one TNF-gamma-β, DR3 and/or TR6 domain-containing polypeptide together with two TNF family member extracellular domain polypeptides, wherein a TNF family member polypeptide may be any of the TNF polypeptides disclosed in Table 4 or otherwise known in the art.

In further embodiments the present invention encompasses heteromultimeric complexes, which comprise polypeptides of two (2), or three (3) distinct TNF family member polypeptides in addition to TNF-gamma-β, DR3 and/or TR6, for example, as described herein, wherein said TNF family polypeptides may be full length polypeptides or extracellular polypeptide domains as described herein.

In accordance with another embodiment, the present invention encompasses the use of isolated nucleic acid molecules encoding human TNF-gamma-β, DR3 and/or TR6, including mRNAs, DNAs, cDNAs, genomic DNAs as well as analogs and biologically active and diagnostically or therapeutically useful fragments and derivatives thereof.

The present invention encompasses the use of isolated nucleic acid molecules comprising, or alternatively, consisting of, a polynucleotide encoding a polypeptide that has at least a portion of the amino acid sequence of TNF-gamma-β as described in FIG. 1 (SEQ ID NO:2) or the amino acid sequence of TNF-gamma-β encoded by the cDNA clone HEMCZ56 deposited as ATCC Deposit Number 203055 on Jul. 9, 1998.

The present invention encompasses the use of isolated nucleic acid molecules comprising, or alternatively, consisting of, a polynucleotide encoding a polypeptide that has at least a portion of the amino acid sequence of DR3 as described in FIG. 3 (SEQ ID NO:4) or the amino acid sequence of DR3 encoded by a cDNA contained in ATCC Deposit Number 97757 on Oct. 10, 1996.

The present invention encompasses the use of isolated nucleic acid molecules comprising, or alternatively, consisting of, a polynucleotide encoding a polypeptide that has at least a portion of the amino acid sequence of TR6 as described in FIG. 5 (SEQ ID NO:6) or the amino acid sequence of TR6 encoded by the cDNA clone HPHAE52 deposited as ATCC Deposit Number ATCC 97810 on Nov. 22, 1996.

In one embodiment, the heterotrimeric complex of the present invention comprises TNF-gamma-β polypeptides of SEQ ID NO:2, together with full-length or extracellular portions of other TNF ligand family member polypeptides, as described herein.

In one embodiment, the heterotrimeric complex of the present invention comprises TNF-gamma-β polypeptides of SEQ ID NO:2, together with full-length or extracellular portions of Lymphotoxin-alpha polypeptides of SEQ ID NO:8.

In one embodiment, the heterotrimeric complex of the present invention comprises TNF-gamma-β polypeptides of SEQ ID NO:2, together with full-length or extracellular portions of TNF-alpha polypeptides of SEQ ID NO:10.

In one embodiment, the heterotrimeric complex of the present invention comprises TNF-gamma-β polypeptides of SEQ ID NO:2, together with full-length or extracellular portions of Lymphotoxin-beta polypeptides of SEQ ID NO:12.

In one embodiment, the heterotrimeric complex of the present invention comprises TNF-gamma-β polypeptides of SEQ ID NO:2, together with full-length or extracellular portions of OX40L polypeptides of SEQ ID NO:14.

In one embodiment, the heterotrimeric complex of the present invention comprises TNF-gamma-β polypeptides of SEQ ID NO:2, together with full-length or extracellular portions of CD40L polypeptides of SEQ ID NO:16.

In one embodiment, the heterotrimeric complex of the present invention comprises TNF-gamma-β polypeptides of SEQ ID NO:2, together with full-length or extracellular portions of FasL polypeptides of SEQ ID NO:18.

In one embodiment, the heterotrimeric complex of the present invention comprises TNF-gamma-β polypeptides of SEQ ID NO:2, together with full-length or extracellular portions of CD70 polypeptides of SEQ ID NO:20.

In one embodiment, the heterotrimeric complex of the present invention comprises TNF-gamma-β polypeptides of SEQ ID NO:2, together with full-length or extracellular portions of CD30LG polypeptides of SEQ ID NO:22.

In one embodiment, the heterotrimeric complex of the present invention comprises TNF-gamma-β polypeptides of SEQ ID NO:2, together with full-length or extracellular portions of 4-1BB-L polypeptides of SEQ ID NO:24.

In one embodiment, the heterotrimeric complex of the present invention comprises TNF-gamma-β polypeptides of SEQ ID NO:2, together with full-length or extracellular portions of TRAIL polypeptides of SEQ ID NO:26.

In one embodiment, the heterotrimeric complex of the present invention comprises TNF-gamma-β polypeptides of SEQ ID NO:2, together with full-length or extracellular portions of RANKL polypeptides of SEQ ID NO:28.

In one embodiment, the heterotrimeric complex of the present invention comprises TNF-gamma-β polypeptides of SEQ ID NO:2, together with full-length or extracellular portions of TWEAK polypeptides of SEQ ID NO:30.

In one embodiment, the heterotrimeric complex of the present invention comprises TNF-gamma-β polypeptides of SEQ ID NO:2, together with full-length or extracellular portions of APRIL polypeptides of SEQ ID NO:32.

In one embodiment, the heterotrimeric complex of the present invention comprises TNF-gamma-β polypeptides of SEQ ID NO:2, together with full-length or extracellular portions of APRIL-SV polypeptides of SEQ ID NO:34.

In one embodiment, the heterotrimeric complex of the present invention comprises TNF-gamma-β polypeptides of SEQ ID NO:2, together with full-length or extracellular portions of polypeptides of SEQ ID NO:36.

In one embodiment, the heterotrimeric complex of the present invention comprises TNF-gamma-β polypeptides of SEQ ID NO:2, together with full-length or extracellular portions of BLyS™-SV polypeptides of SEQ ID NO:38.

In one embodiment, the heterotrimeric complex of the present invention comprises TNF-gamma-β polypeptides of SEQ ID NO:2, together with full-length or extracellular portions of LIGHT polypeptides of SEQ ID NO:40.

In one embodiment, the heterotrimeric complex of the present invention comprises TNF-gamma-β polypeptides of SEQ ID NO:2, together with full-length or extracellular portions of VEGI-SV polypeptides of SEQ ID NO:42.

In one embodiment, the heterotrimeric complex of the present invention comprises TNF-gamma-β polypeptides of SEQ ID NO:2, together with full-length or extracellular portions of Endokine alpha polypeptides of SEQ ID NO:44.

In one embodiment, the heterotrimeric complex of the present invention comprises TNF-gamma-β polypeptides of SEQ ID NO:2, together with full-length or extracellular portions of EDA polypeptides of SEQ ID NO:46.

In one embodiment, the heterotrimeric complex of the present invention comprises DR3 polypeptides of SEQ ID NO:4, together with full-length or extracellular portions of other TNF receptor family member polypeptides, as described herein or as otherwise known and appreciated in the art.

In a further embodiment, the heterotrimeric complex of the present invention comprises TR6 polypeptides of SEQ ID NO:6, together with full-length or extracellular portions of other TNF receptor family member polypeptides, as described herein or as otherwise known and appreciated in the art.

In further embodiments the present invention also encompasses heteromultimeric complexes, particularly heterotrimeric complexes, comprising TNF-gamma-β, DR3 and/or TR6 polypeptides, as described herein, fused to one or more heterologous polypeptide sequences.

In further embodiments the present invention also encompasses heteromultimeric complexes, particularly heterotrimeric complexes, comprising polypeptides at least 80% identical, more preferably at least 85% or 90% identical, and still more preferably 95%, 96%, 97%, 98% or 99% identical to TNF-gamma-β, DR3 and/or TR6 polypeptides, as described herein, fused to one or more heterologous polypeptide sequences.

The present invention further encompasses methods for isolating antibodies that bind specifically to heteromultimeric complexes, particularly heterotrimeric complexes, as described above. Such antibodies are useful diagnostically or therapeutically as described below.

The invention further encompasses methods for isolating antibodies that bind specifically to a TNF-gamma-β, DR3 and/or TR6 polypeptide, as described herein having an amino acid sequence as described above. Such antibodies may be useful diagnostically and/or therapeutically as antagonists in the treatment of inflammatory bowel diseases and disorders. The invention also provides a diagnostic method for determining the presence of inflammatory bowel diseases and disorders.

The present invention also encompasses pharmaceutical compositions comprising TNF-gamma-β, DR3 and/or TR6 polypeptides, as described herein, which may be used for instance, to treat, prevent, prognose and/or diagnose inflammatory bowel diseases or disorders and/or conditions associated with such diseases or disorders.

The invention further encompasses compositions comprising heteromultimeric polypeptide complexes, particularly heterotrimeric polypeptide complexes, and/or anti-heteromultimeric complex antibodies, for administration to cells in vitro, to cells ex vivo, and to cells in vivo, or to a multicellular organism. In preferred embodiments, the compositions of the invention comprise TNF-gamma-β-encoding polynucleotides for expression of a heteromultimeric polypeptide complex in a host organism for treatment of disease. In a most preferred embodiment, the compositions of the invention comprise TNF-gamma-β, DR3 and/or TR6-encoding polynucleotides for expression of a heteromultimeric polypeptide complex in a host organism for treatment of an inflammatory bowel disease or disorder and/or conditions associated with an inflammatory bowel disease or disorder. Particularly preferred in this regard is expression in a human patient for treatment of an inflammatory bowel disease or disorder and/or conditions associated with an inflammatory bowel disease or disorder.

The present invention further encompasses methods and compositions for preventing, treating and/or ameliorating diseases or disorders associated with aberrant or inappropriate interferon-gamma expression (e.g., excessive inflammation) in an animal, preferably a mammal, and most preferably a human, comprising, or alternatively consisting of, administering to an animal in which such treatment, prevention or amelioration is desired one or more compositions of the invention (including, for example, antagonists and/or antibodies to TNF-gamma-β polypeptides) in an amount effective to treat prevent or ameliorate the disease or disorder.

The present invention further encompasses methods and compositions for inhibiting interferon-gamma expression comprising, or alternatively consisting of, administering to an animal in which such inhibition is desired, one or more compositions of the invention in an amount effective to inhibit interferon-gamma expression.

The present invention further encompasses methods and compositions for stimulating interferon-gamma expression comprising, or alternatively consisting of, administering to an animal in which such stimulation is desired, one or more compositions of the invention in an amount effective to stimulate interferon-gamma expression.

The present invention also provides a screening method for identifying compounds capable of inhibiting a cellular response induced by TNF-gamma-β, DR3 and/or TR6, which involves contacting cells which express polypeptide compositions of the invention with the candidate compound, assaying a cellular response, and comparing the cellular response to a standard cellular response, the standard being assayed when contact is made in absence of the candidate compound; whereby, an increased cellular response over the standard indicates that the compound is an agonist and a decreased cellular response over the standard indicates that the compound is an antagonist.

In another embodiment, a method for identifying molecules that bind compositions of the invention is provided, as well as a screening assay for agonists and antagonists using such molecules. This assay involves determining the effect of a candidate compound on binding of a composition of the invention to its binding molecule. In particular, the method involves contacting a molecule with a composition of the invention and a candidate compound and determining whether binding to the molecule is increased or decreased due to the presence of the candidate compound. The antagonists may be employed to prevent or treat inflammatory bowel diseases or disorders and conditions associated with such diseases or disorders.

The present invention also provides pharmaceutical compositions, which may be used for instance, to treat, prevent, prognose and/or diagnose inflammatory bowel diseases or disorders and/or conditions associated with such diseases or disorders.

In certain embodiments the present invention encompasses the use of, polypeptides and polypeptide complexes, particularly heterotrimeric complexes, or antagonists thereof, to treat, prevent, prognose and/or diagnose diseases and/or disorders of the gastrointestinal tract, including but not limited to, disorders of the mouth, esophagus, stomach, duodenum, small intestine, large intestine, colon, caecum, rectum and/or anus.

In certain embodiments the present invention encompasses the use of, polypeptides and polypeptide complexes, particularly heterotrimeric complexes, or antagonists thereof, to treat, prevent, prognose and/or diagnose diseases and/or disorders associated with diseases and/or disorders of the gastrointestinal tract, including but not limited to, disorders of the mouth, esophagus, stomach, duodenum, small intestine, large intestine, colon, caecum, rectum and/or anus.

In certain embodiments the present invention encompasses the use of, polypeptides and polypeptide complexes, particularly heterotrimeric complexes, or antagonists thereof, to treat, prevent, prognose and/or diagnose diseases and/or disorders which may lead to and/or cause diseases and/or disorders of the gastrointestinal tract, including but not limited to, disorders of the mouth, esophagus, stomach, duodenum, small intestine, large intestine, colon, caecum, rectum and/or anus.

In a specific embodiment, one or more compositions of the invention, or agonists or antagonists thereof, are administered to treat, prevent, prognose and/or diagnose diseases of the gastrointestinal tract.

In a specific embodiment, one or more compositions of the invention, or agonists or antagonists thereof, are administered to treat, prevent, prognose and/or diagnose inflammatory bowel disease.

In a specific embodiment, one or more compositions of the invention, or agonists or antagonists thereof, are administered to treat, prevent, prognose and/or diagnose ulcerative colitis.

In a specific embodiment, one or more compositions of the invention, or agonists or antagonists thereof, are administered to treat, prevent, prognose and/or diagnose Crohn's disease.

The present invention encompasses methods and products for diagnosing diseases of the gastrointestinal tract by determining the presence of RNA transcribed from the human TNF-gamma-β, DR3 and/or TR6 genes, or DNA corresponding to such RNA in a sample derived from a host.

The present invention also encompasses methods and products for diagnosing inflammatory bowel disease by determining the presence of RNA transcribed from the human TNF-gamma-β, DR3 and/or TR6 genes, or DNA corresponding to such RNA in a sample derived from a host.

The present invention also encompasses methods and products for diagnosing ulcerative colitis by determining the presence of RNA transcribed from the human TNF-gamma-β, DR3 and/or TR6 genes, or DNA corresponding to such RNA in a sample derived from a host.

The present invention also encompasses methods and products for diagnosing Crohn's disease by determining the presence of RNA transcribed from the human TNF-gamma-β, DR3 and/or TR6 genes, or DNA corresponding to such RNA in a sample derived from a host.

The present invention also encompasses methods and products for diagnosing diseases of the gastrointestinal tract by detecting an altered level of TNF-gamma-β, DR3 and/or TR6 polypeptide expression in a sample derived from a host, whereby an elevated level of the polypeptide is indicative of a disease of the gastrointestinal tract.

The present invention also encompasses methods and products for diagnosing inflammatory bowel disease by detecting an altered level of TNF-gamma-β, DR3 and/or TR6 polypeptide expression in a sample derived from a host, whereby an elevated level of the polypeptide is indicative of inflammatory bowel disease.

The present invention also encompasses methods and products for diagnosing ulcerative colitis by detecting an altered level of TNF-gamma-β, DR3 and/or TR6 polypeptide expression in a sample derived from a host, whereby an elevated level of the polypeptide is indicative of ulcerative colitis.

The present invention also encompasses methods and products for diagnosing Crohn's disease by detecting an altered level of TNF-gamma-[, DR3 and/or TR6 polypeptide expression in a sample derived from a host, whereby an elevated level of the polypeptide is indicative of Crohn's disease.

The present invention also encompasses the use of antibodies specific to such TNF-gamma-β, DR3 and/or TR6 polypeptides, as well as biologically active and diagnostically or therapeutically useful fragments, analogs, and derivatives thereof.

The present invention also encompasses the use of TNF-gamma-β, DR3 and/or TR6 antagonists, including antibodies, polypeptides, peptides, polynucleotides (including RNA, DNA, and synthetic polynucleotide derivitives), and small molecules useful in preventing, treating, and/or ameliorating diseases of the gastrointestinal tract.

The present invention also encompasses TTNF-gamma-β, DR3 and/or TR6 antagonists, including antibodies, polypeptides, peptides, polynucleotides (including RNA, DNA, and synthetic polynucleotide derivitives), and small molecules useful in preventing, treating, and/or ameliorating inflammatory bowel disease.

The present invention also encompasses TNF-gamma-β, DR3 and/or TR6 antagonists, including antibodies, polypeptides, peptides, polynucleotides (including RNA, DNA, and synthetic polynucleotide derivitives), and small molecules useful in preventing, treating, and/or ameliorating ulcerative colitis.

The present invention also encompasses TNF-gamma-β, DR3 and/or TR6 antagonists, including antibodies, polypeptides, peptides, polynucleotides (including RNA, DNA, and synthetic polynucleotide derivitives), and small molecules useful in preventing, treating, and/or ameliorating Crohn's disease.

The present invention also encompasses methods for using the, polynucleotides, polypeptides, and antibodies of the present invention to detect, prevent, treat, and/or ameliorate diseases of the gastrointestinal tract.

The present invention also encompasses methods for using the, polynucleotides, polypeptides, and antibodies of the present invention to detect, prevent, treat, and/or ameliorate inflammatory bowel disease.

The present invention also encompasses methods for using the, polynucleotides, polypeptides, and antibodies of the present invention to detect, prevent, treat, and/or ameliorate ulcerative colitis.

The present invention also encompasses methods for using the, polynucleotides, polypeptides, and antibodies of the present invention to detect, prevent, treat, and/or ameliorate Crohn's disease.

The present invention also encompasses methods for utilizing such polypeptides, polynucleotides encoding such polypeptides, and antibodies that bind such polypeptides for in vitro purposes related to scientific research of inflammatory bowel disease.

The present invention also encompasses methods for utilizing such polypeptides, polynucleotides encoding such polypeptides, and antibodies that bind such polypeptides for in vitro purposes related to scientific research of ulcerative colitis.

The present invention also encompasses methods for utilizing such polypeptides, polynucleotides encoding such polypeptides, and antibodies that bind such polypeptides for in vitro purposes related to scientific research of Crohn's disease.

These and other aspects of the present invention should be apparent to those skilled in the art from the teachings herein.

### BRIEF DESCRIPTION OF THE DRAWINGS

The following drawings are illustrative of embodiments of the invention and are not meant to limit the scope of the invention as encompassed by the claims.

FIGS. 1A-1B (cDNA and amino acid sequence) shows the cDNA sequence (SEQ ID NO:1) and the corresponding deduced amino acid sequence (SEQ ID NO:2) for the human TNF-gamma-β Gene disclosed in this application. The standard one-letter abbreviations for amino acids are used. Underlining demarcates residues comprising the predicted transmembrane domain. It is predicted that amino acid residues 1-35 constitute the intracellular domain, amino acid residues 36-61 constitute the transmembrane domain, and amino acid residues 62-251 constitute the extracellular domain. Potential asparagine-linked glycosylation sites are indicated by bold face type (N) and a bolded pound sign (#) above the first nucleotide encoding that residue, and are found at amino acid residues 133-136 and 229-232. Potential Protein Kinase C (PKC) phosphorylation sites are indicated by bold face type (S or T) and an asterisk (*) above the first nucleotide encoding that residue, and are found at amino acid residues 23-25; 32-34; 135-137; and 154-156. Potential Casein Kinase II (CK2) phosphorylation sites are indicated by bold face type (S or T) and an asterisk (*) above the first nucleotide encoding that residue, and are found at amino acid residues 8-11; 187-190; 200-203; 219-222; 234-237; and 239-242. Potential myristylation sites are indicated by a double underline and are found at amino acid residues 6-11; 124-129; and 215-220.

FIG. 2 (Polypeptide Domains, Epitopes, and Motifs) shows an analysis of the amino acid sequence (SEQ ID NO:2). Alpha, beta, turn and coil regions; hydrophilicity and hydrophobicity; amphipathic regions; flexible regions; antigenic index and surface probability are shown, and all were generated using the default settings of the recited computer algorithms. In the “Antigenic Index or Jameson-Wolf” graph, the positive peaks indicate locations of the highly antigenic regions of the protein, i.e., regions from which epitope-bearing peptides of the invention can be obtained. Polypeptides comprising, or alternatively consisting of, domains defined by these graphs are contemplated by the present invention, as are polynucleotides encoding these polypeptides.

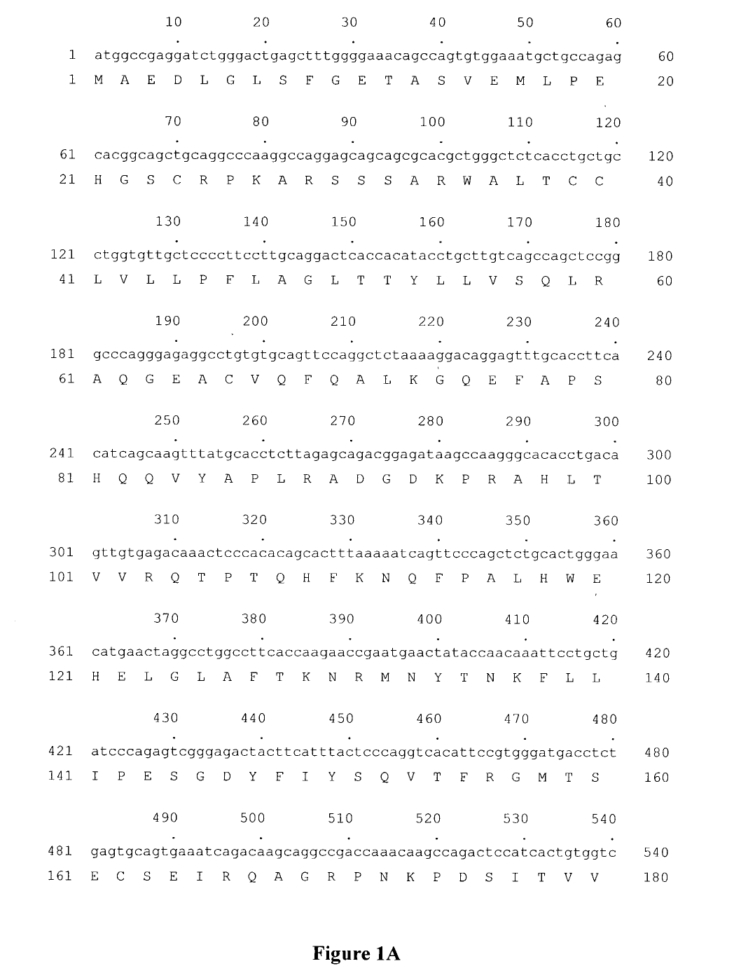

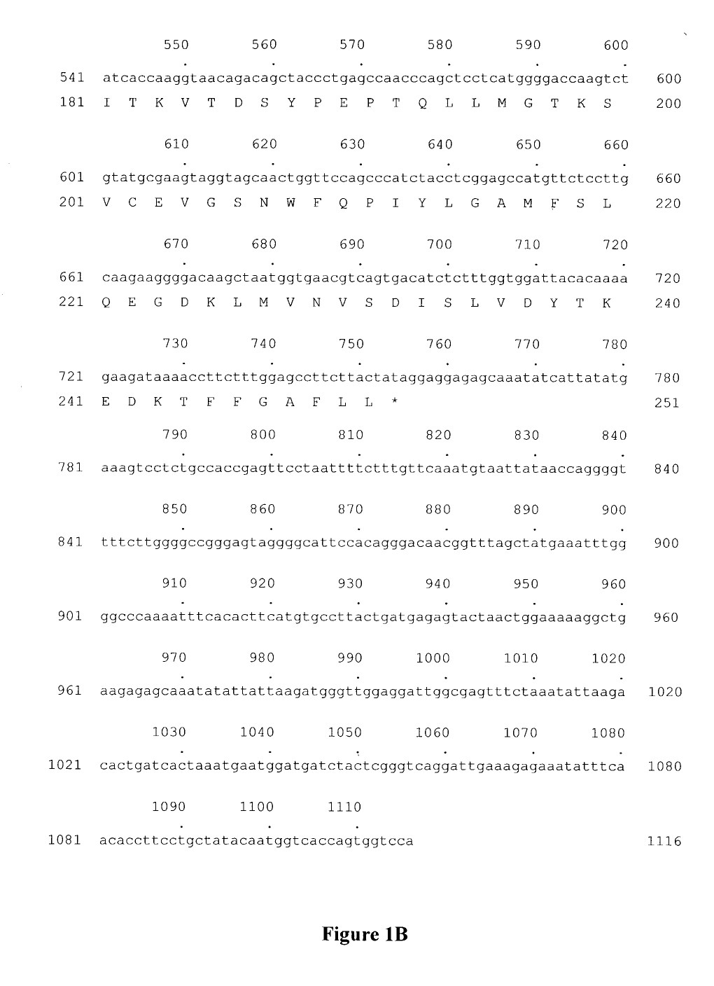

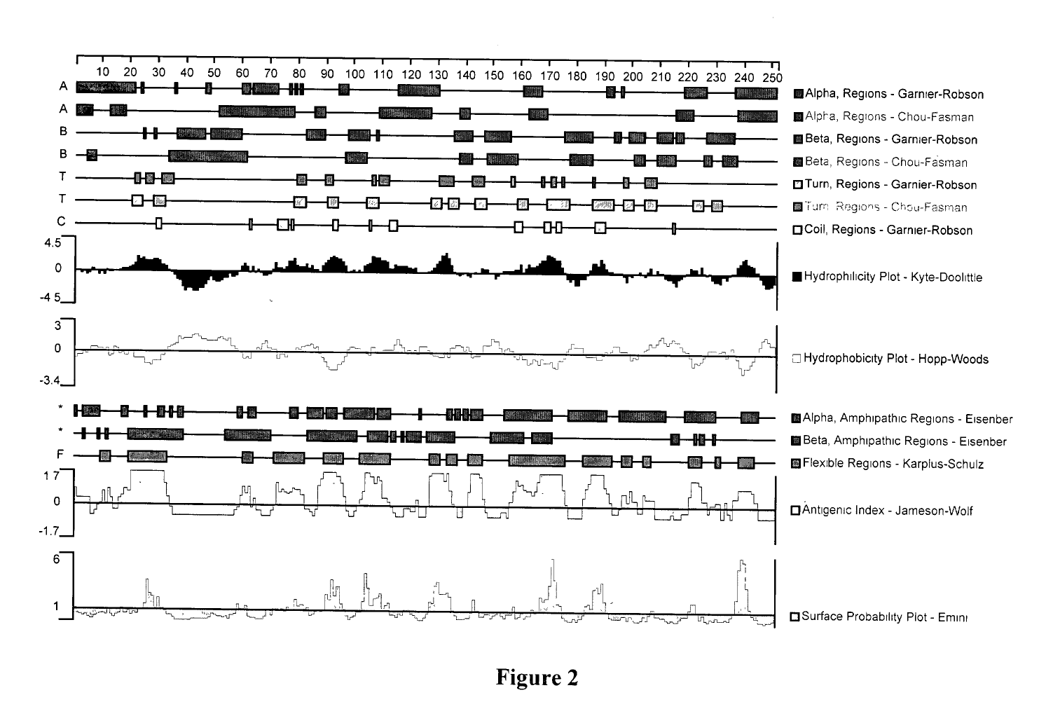

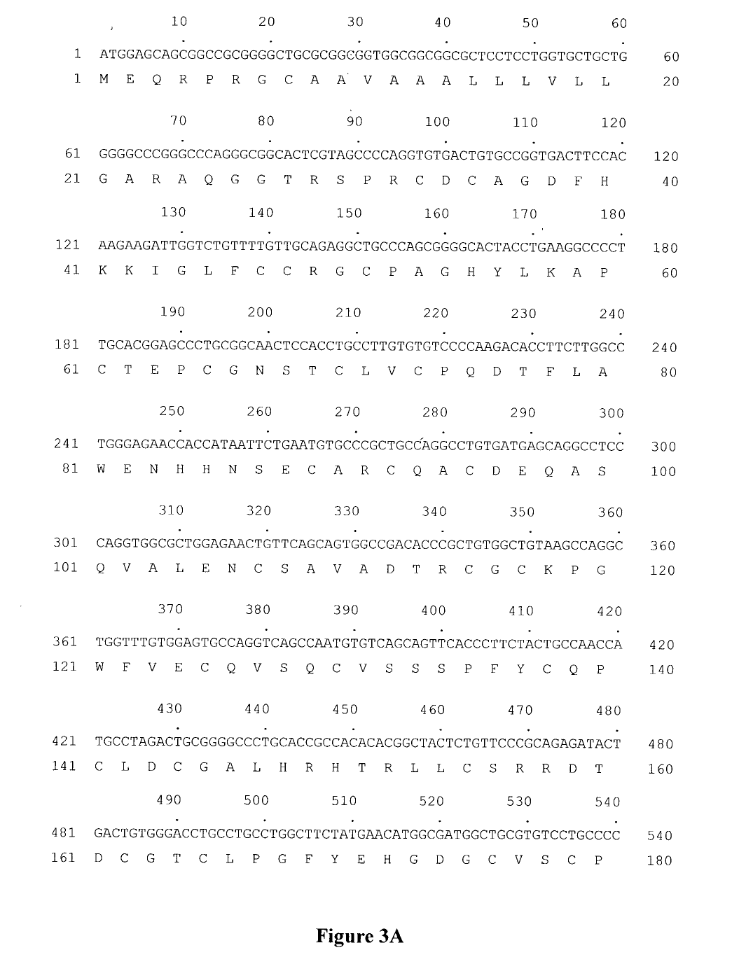

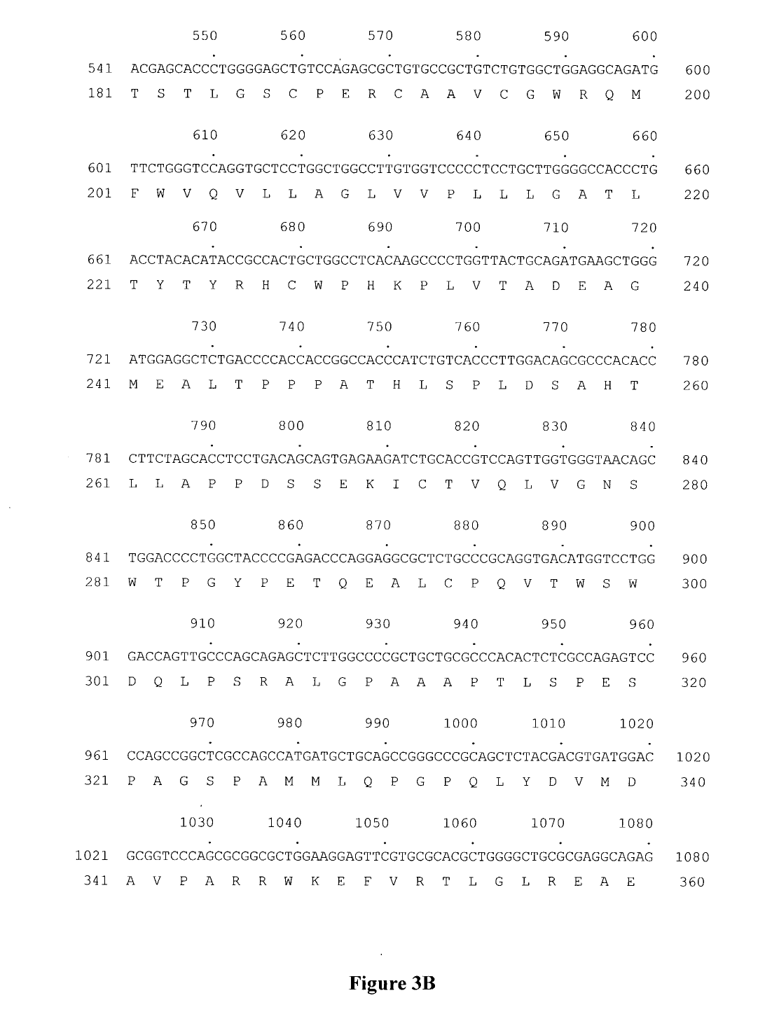

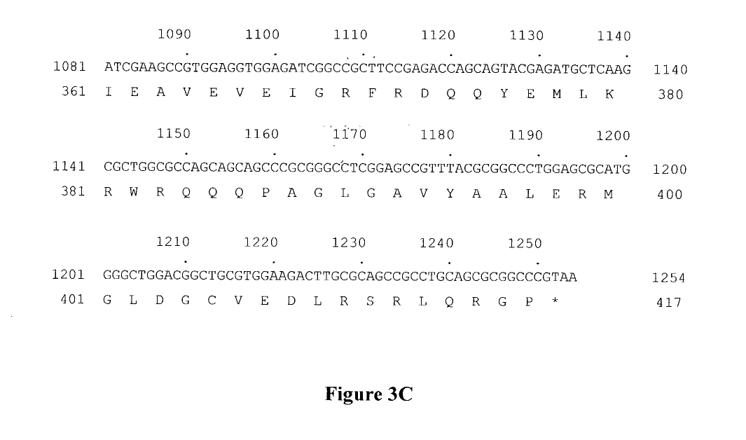

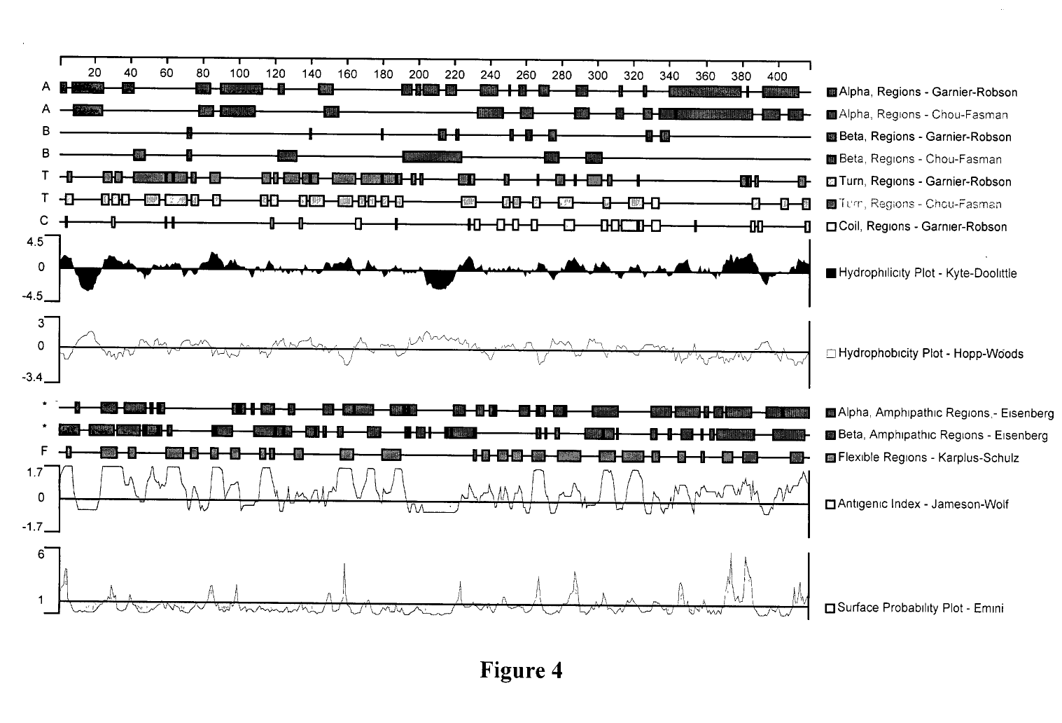

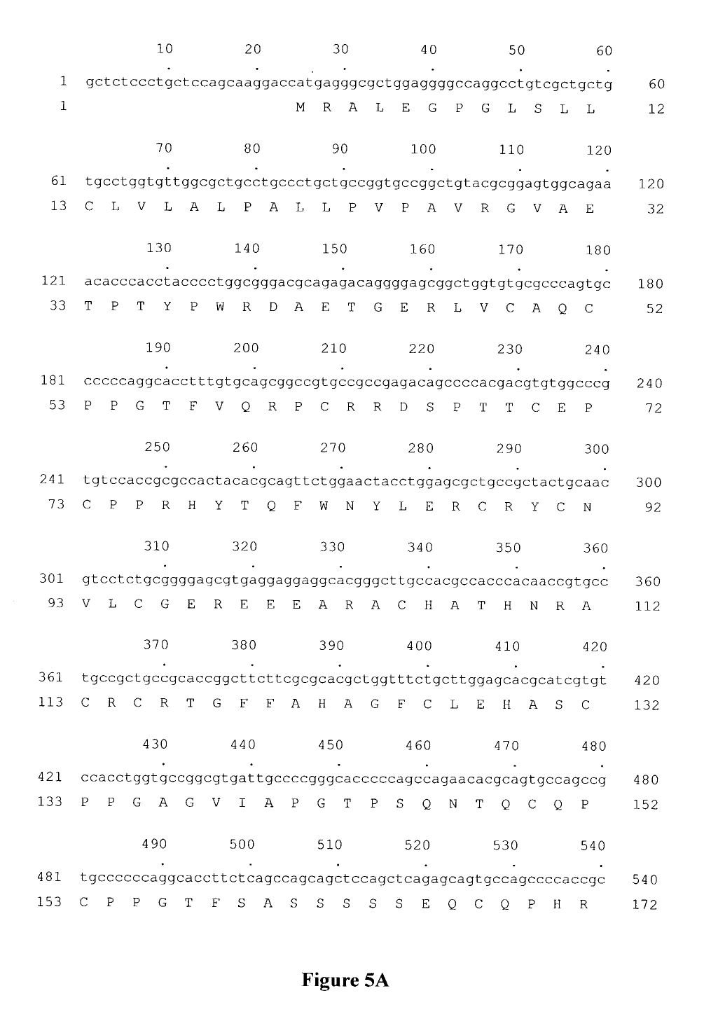

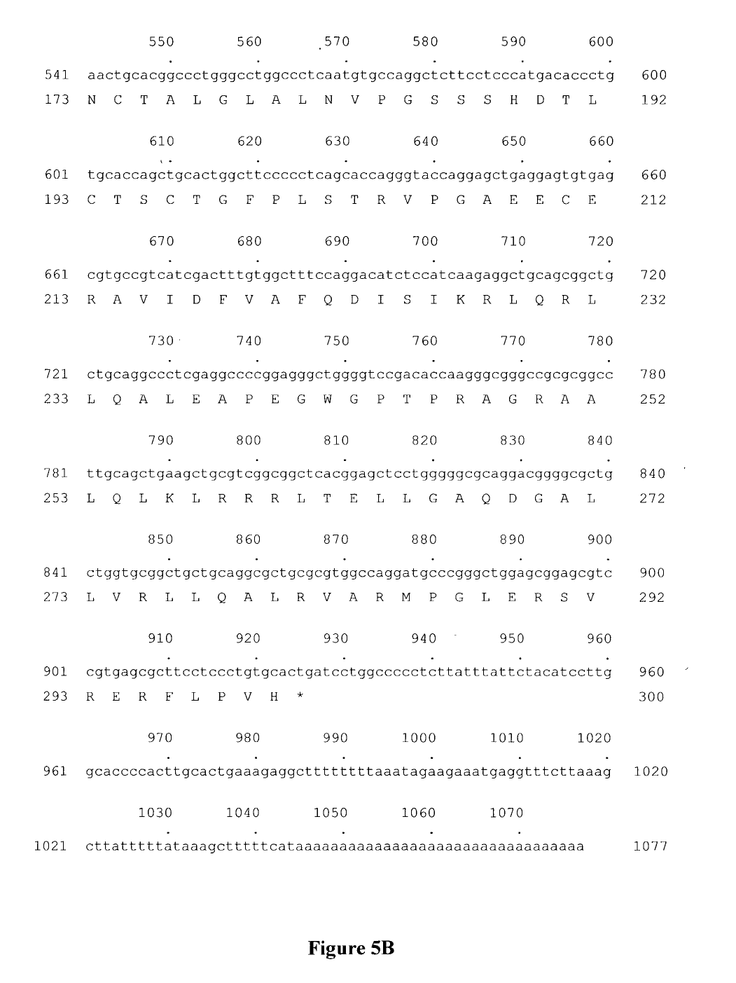

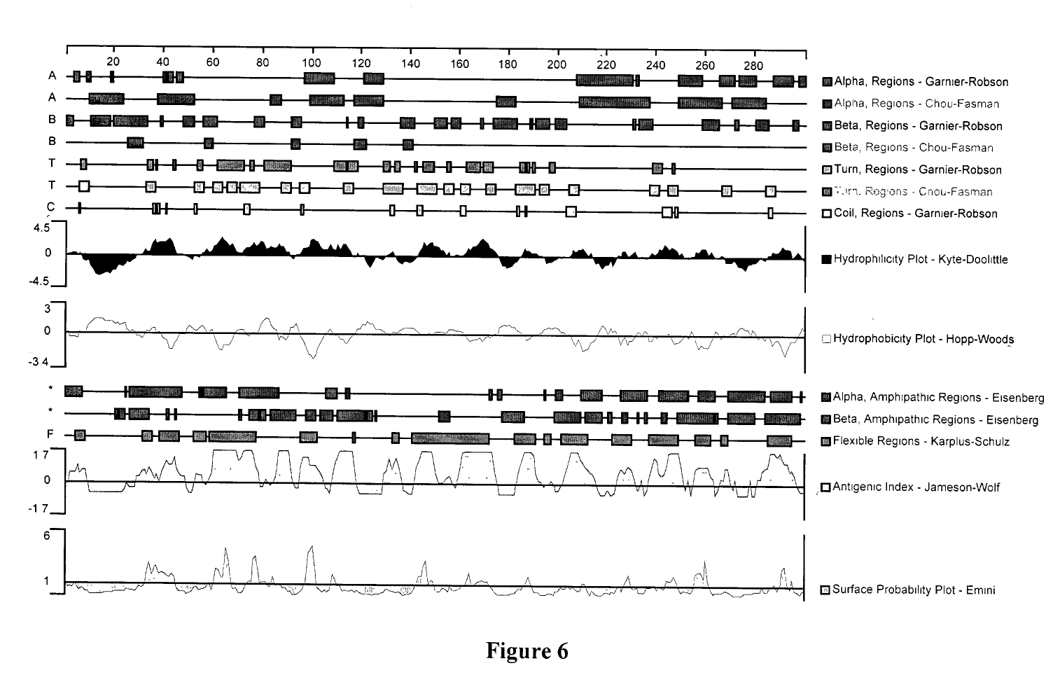

The data presented in FIG. 2 are also represented in tabular form in Table 1. The columns are labeled with the headings “Res,” “Pos,” and Roman Numerals I-XIV. The column headings refer to the following features of the amino acid sequence presented in FIG. 2, and Table 1: “Res”: amino acid residue of SEQ ID NO:2 and FIGS. 1A-1B; “Position”: position of the corresponding residue within SEQ ID NO:2 and FIGS. 1A-1B; I: Alpha, Regions—Garnier-Robson; II: Alpha, Regions—Chou-Fasman; III: Beta, Regions—Garnier-Robson; IV: Beta, Regions—Chou-Fasman; V: Turn, Regions—Garnier-Robson; VI: Turn, Regions—Chou-Fasman; VII: Coil, Regions—Garnier-Robson; VIII: Hydrophilicity Plot—Kyte-Doolittle; IX: Hydrophobicity Plot—Hopp-Woods; X: Alpha, Amphipathic Regions—Eisenberg; XI: Beta, Amphipathic Regions—Eisenberg; XII: Flexible Regions—Karplus-Schulz; XIII: Antigenic Index—Jameson-Wolf; and XIV: Surface Probability Plot—Emini.

Preferred embodiments of the invention in this regard include fragments that comprise, or alternatively consisting of, one or more of the following regions: alpha-helix and alpha-helix forming regions (“alpha-regions”), beta-sheet and beta-sheet forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions. The data representing the structural or functional attributes of the protein set forth in FIG. 2 and/or Table 1, as described above, was generated using PROTEAN™ sequence analysis software set on default parameters (WINDOWS 32 PROTEAN 4.05©; 1994-2000 DNASTAR, Inc.). In a preferred embodiment, the data presented in columns VIII, IX, XIII, and XIV of Table 1 can be used to determine regions of the protein which exhibit a high degree of potential for antigenicity. Regions of high antigenicity are determined from the data presented in columns VIII, IX, XIII, and/or XIV by choosing values, which represent regions of the polypeptide which are likely to be exposed on the surface of the polypeptide in an environment in which antigen recognition may occur in the process of initiation of an immune response. Certain preferred regions in this regard are set out in graphical form in FIG. 2, but may also be represented or identified with numerical data (as shown in Table 1). The DNA*STAR computer algorithm used to generate FIG. 2 (set on the original default parameters) was used to present the data in FIG. 2 in a tabular format (See Table 1). The tabular format of the data in FIG. 2 is used to easily determine specific boundaries of a preferred region.

FIGS. 3A-3C (cDNA and amino acid sequence) shows the cDNA sequence (SEQ ID NO:3) and the corresponding deduced amino acid sequence (SEQ ID NO:4) for the human TNF-gamma-β receptor DR3 gene disclosed in this application. The standard one-letter abbreviations for amino acids are used. Underlining demarcates residues comprising the predicted signal peptide sequence. It is predicted that amino acid residues 1-24 constitute the signal peptide, amino acid residues 25-201 constitute the extracellular domain, amino acid residues 202-224 constitute the transmembrane domain, and amino acid residues 225-417 constitute the intracellular domain, with amino acid residues 342-408 constitute the death domain.

FIG. 4 (Polypeptide Domains, Epitopes, and Motifs) shows an analysis of the amino acid sequence (SEQ ID NO:4). Alpha, beta, turn and coil regions; hydrophilicity and hydrophobicity; amphipathic regions; flexible regions; antigenic index and surface probability are shown, and all were generated using the default settings of the recited computer algorithms. In the “Antigenic Index or Jameson-Wolf” graph, the positive peaks indicate locations of the highly antigenic regions of the protein, i.e., regions from which epitope-bearing peptides of the invention can be obtained. Polypeptides comprising, or alternatively consisting of, domains defined by these graphs are contemplated by the present invention, as are polynucleotides encoding these polypeptides.

The data presented in FIG. 4 are also represented in tabular form in Table 2. The columns are labeled with the headings “Res,” “Pos,” and Roman Numerals I-XIV. The column headings refer to the following features of the amino acid sequence presented in FIG. 4, and Table 2: “Res”: amino acid residue of SEQ ID NO:4 and FIGS. 3A-3C; “Position”: position of the corresponding residue within SEQ ID NO:4 and FIGS. 3A-3C; I: Alpha, Regions—Garnier-Robson; II: Alpha, Regions—Chou-Fasman; III: Beta, Regions—Garnier-Robson; IV: Beta, Regions—Chou-Fasman; V: Turn, Regions—Garnier-Robson; VI: Turn, Regions—Chou-Fasman; VII: Coil, Regions—Garnier-Robson; VIII: Hydrophilicity Plot—Kyte-Doolittle; IX: Hydrophobicity Plot—Hopp-Woods; X: Alpha, Amphipathic Regions—Eisenberg; XI: Beta, Amphipathic Regions—Eisenberg; XII: Flexible Regions—Karplus-Schulz; XIII: Antigenic Index—Jameson-Wolf; and XIV: Surface Probability Plot—Emini.

Preferred embodiments of the invention in this regard include fragments that comprise, or alternatively consisting of, one or more of the following regions: alpha-helix and alpha-helix forming regions (“alpha-regions”), beta-sheet and beta-sheet forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions. The data representing the structural or functional attributes of the protein set forth in FIG. 4 and/or Table 2, as described above, was generated using PROTEAN sequence analysis software set on default parameters (WINDOWS 32 PROTEAN 4.05©; 1994-2000 DNASTAR, Inc.). In a preferred embodiment, the data presented in columns VIII, IX, XIII, and XIV of Table 2 can be used to determine regions of the protein which exhibit a high degree of potential for antigenicity. Regions of high antigenicity are determined from the data presented in columns VIII, IX, XIII, and/or XIV by choosing values, which represent regions of the polypeptide which are likely to be exposed on the surface of the polypeptide in an environment in which antigen recognition may occur in the process of initiation of an immune response. Certain preferred regions in this regard are set out in graphical form in FIG. 4, but may also be represented or identified with numerical data (as shown in Table 2). The DNA*STAR computer algorithm used to generate FIG. 4 (set on the original default parameters) was used to present the data in FIG. 4 in a tabular format (See Table 2). The tabular format of the data in FIG. 4 is used to easily determine specific boundaries of a preferred region.

FIGS. 5A-5B (cDNA and amino acid sequence) shows the cDNA sequence (SEQ ID NO:5) and the corresponding deduced amino acid sequence (SEQ ID NO:6) for the human TNF-gamma-β receptor TR6 gene disclosed in this application. The standard one-letter abbreviations for amino acids are used. Underlining demarcates residues comprising the predicted signal peptide sequence. It is predicted that amino acid residues 1-30 constitute the leader (signal) peptide.

FIG. 6 (Polypeptide Domains, Epitopes, and Motifs) shows an analysis of the amino acid sequence (SEQ ID NO:6). Alpha, beta, turn and coil regions; hydrophilicity and hydrophobicity; amphipathic regions; flexible regions; antigenic index and surface probability are shown, and all were generated using the default settings of the recited computer algorithms. In the “Antigenic Index or Jameson-Wolf” graph, the positive peaks indicate locations of the highly antigenic regions of the protein, i.e., regions from which epitope-bearing peptides of the invention can be obtained. Polypeptides comprising, or alternatively consisting of, domains defined by these graphs are contemplated by the present invention, as are polynucleotides encoding these polypeptides.

The data presented in FIG. 6 are also represented in tabular form in Table 3. The columns are labeled with the headings “Res,” “Pos,” and Roman Numerals I-XIV. The column headings refer to the following features of the amino acid sequence presented in FIG. 6, and Table 3: “Res”: amino acid residue of SEQ ID NO:6 and FIGS. 5A-5B; “Position”: position of the corresponding residue within SEQ ID NO:6 and FIGS. 5A-5B; I: Alpha, Regions—Garnier-Robson; II: Alpha, Regions—Chou-Fasman; III: Beta, Regions—Garnier-Robson; IV: Beta, Regions—Chou-Fasman; V: Turn, Regions—Garnier-Robson; VI: Turn, Regions—Chou-Fasman; VII: Coil, Regions—Garnier-Robson; VIII: Hydrophilicity Plot—Kyte-Doolittle; IX: Hydrophobicity Plot—Hopp-Woods; X: Alpha, Amphipathic Regions—Eisenberg; XI: Beta, Amphipathic Regions—Eisenberg; XII: Flexible Regions—Karplus-Schulz; XIII: Antigenic Index—Jameson-Wolf; and XIV: Surface Probability Plot—Emini.

Preferred embodiments of the invention in this regard include fragments that comprise, or alternatively consisting of, one or more of the following regions: alpha-helix and alpha-helix forming regions (“alpha-regions”), beta-sheet and beta-sheet forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions. The data representing the structural or functional attributes of the protein set forth in FIG. 6 and/or Table 3, as described above, was generated using PROTEAN™ sequence analysis software set on default parameters (WINDOWS 32 PROTEAN 4.05©; 1994-2000 DNASTAR, Inc.). In a preferred embodiment, the data presented in columns VII, IX, XIII, and XIV of Table 3 can be used to determine regions of the protein which exhibit a high degree of potential for antigenicity. Regions of high antigenicity are determined from the data presented in columns VIII, IX, XIII, and/or XIV by choosing values, which represent regions of the polypeptide which are likely to be exposed on the surface of the polypeptide in an environment in which antigen recognition may occur in the process of initiation of an immune response. Certain preferred regions in this regard are set out in graphical form in FIG. 6, but may also be represented or identified with numerical data (as shown in Table 3). The DNA*STAR computer algorithm used to generate FIG. 6 (set on the original default parameters) was used to present the data in FIG. 6 in a tabular format (See Table 3). The tabular format of the data in FIG. 6 is used to easily determine specific boundaries of a preferred region.

### DETAILED DESCRIPTION

The TNF-gamma-β, DR3 and TR6 polynucleotides and proteins as described herein have been described previously. See e.g., PCT Publication Nos. WO96/14328, WO00/66608, WO97/33904, WO00/64465, WO98/30694, and WO00/52028, the contents of which are hereby incorporated by reference in their entireties.

Therapeutic Uses of the Invention

Inflammatory Bowel Disease

While the exact cause or causes of inflammatory bowel disease are unknown, it is known that intestinal mucosal inflammation, as seen for example in inflammatory bowel disease, is characterized by a powerful TH 1 response, well known to be dependent on IL-12 and IL-18. This phenomenon is marked by increased levels of expression of the inflammatory mediators interferon gamma (IFNγ) and Tumor Necrosis Factor alpha (TNFα) by lamina propria T cells.

Data described herein show that TNF-gamma-β acted in synergy with, but independently of, IL-12 and IL-18 to stimulate increased production of IFNγ from CD3 activated peripheral T cells. Agonistic antibodies directed against the TNF-gamma-β receptor DR3 (anti-DR3) also acted to stimulate increased production of IFNγ from peripheral T cells. Furthermore, peripheral T cell activation resulted in upregulated expression of DR3 in a large fraction of the activated T cells. See Examples 37 and 38 below. Data described herein also shows that a large fraction of lamina propria T cells isolated from patients having inflammatory bowel disease expressed DR3 at the cell surface. TNF-gamma-β was shown to increase, while anti-TNF-gamma-β neutralizing antibodies were shown to reduce, IFNγ secretion from isolated lamina propria T cells. Furthermore, the effects of TNF-gamma-β and anti-TNF-gamma-β on IFNγ secrtetion were shown to be greatest on lamina propria T cells isolated from a patient having inflammatory bowel disease. See Examples 37 and 38 below. Hence, stimulation of IFNγ secretion by activated T cells may constitute a mechanism whereby TNF-gamma-β activation of cells expressing DR3 cause increased inflammation in patients having inflammatory bowel disease.

Accordingly, the present invention encompasses the use of polynucleotides, polypeptides, antibodies and/or antagonists of the invention in the detection, prevention, treatment, and amelioration of diseases of the gastrointestinal tract and/or diseases requiring regulation of IFNγ secretion by activated T cells. Specific useful embodiments of the invention include polynucleotides, polypeptides, and antibodies of the invention, together with fragments and variants thereof as well as agonists and antagonists thereto, as described in the section entitled “Compositions of the Invention” below.

The present invention encompasses methods of detection, treatment, amelioration and/or prevention of gastrointestinal diseases, such as those described in the section entitled “Other Gastrointestinal and Digestive Diseases” below. Preferred among the gastrointestinal diseases treatable using embodiments of the invention, are inflammatory bowel diseases such as, for example, Crohn's disease and ulcerative colitis.

Specifically, the present invention encompasses methods of detection, treatment, amelioration and/or prevention of inflammatory bowel diseases, such as Crohn's disease and ulcerative colitis, which result in destruction of the mucosal surface, and/or underlying layers, of the small and/or large intestine. Thus, particular methods of the invention, including treatment using polynucleotides or polypeptides, as well as antagonists or antibodies thereto, could be used to reduce inflammation of the mucosal surface to aid more rapid healing and to prevent or attenuate progression of inflammatory bowel disease. Treatment with particular methods of the invention, including the use of polynucleotides or polypeptides, as well as antagonists or antibodies thereto, is expected to have a significant effect on the production of mucus throughout the gastrointestinal tract and could be used to protect the intestinal mucosa from injurious substances that are ingested or following surgery. Accordingly, particular methods of the invention, including treatment with polynucleotides or polypeptides, as well as antagonists or antibodies thereto, can also be used to promote healing of intestinal or colonic anastomosis and to treat diseases associate with the over expression of TNF-gamma-β.

Furthermore, the present invention encompasses methods of detection, treatment, amelioration and/or prevention of the side effects of gut toxicity that result from radiation, chemotherapy treatments or viral infections. Such methods may have a cytoprotective effect on the small intestine mucosa. Such methods may also stimulate healing of mucositis (mouth ulcers) that result from chemotherapy and viral infections. Furthermore, such methods can also be used to treat gastric and doudenal ulcers and help heal by scar formation of the mucosal lining and regeneration of glandular mucosa and duodenal mucosal lining more rapidly.

In addition, as described in the section entitled “Wound Healing and Epithelial Cell Proliferation” below, the present invention encompasses methods, for example, to reduce IFNγ-mediated inflammation for the purpose of wound healing, and to stimulate hair follicle production and healing of dermal wounds. Such methods can also be used to promote dermal reestablishment subsequent to dermal loss. Additionally, such methods can be used to increase the adherence of skin grafts to a wound bed and to stimulate re-epithelialization from the wound bed. Furthermore, such methods can be used to treat epidermolysis bullosa, a defect in adherence of the epidermis to the underlying dermis which results in frequent, open and painful blisters by accelerating reepithelialization of these lesions.

While the present invention is described in terms of the detection, treatment and/or amelioration of inflammatory bowel disease, it may also be used in the detection, treatment and/or amelioration of additional gastrointestinal disorders as well as other disorders as described herein.

Other Gastrointestinal and Digestive Diseases

TNF-gamma-β has been shown to stimulate secretion of IFNγ by activated T cells derived from the gastrointestinal tract. Accordingly, the present invention encompasses methods of detection, treatment, amelioration and/or prevention of gastrointestinal and digestive diseases.

Gastrointestinal diseases whose detection, treatment, amelioration and/or prevention is encompassed by the present invention include, but are not limited to, gastroenteritis such as cholera morbus, gastrointestinal hemorrhage (such as, for example, hematemesis, melena and peptic ulcer), intestinal diseases (such as, for example, cecal diseases which include appendicitis), colonic diseases (such as, for example, colitis which include ischemic colitis), ulcerative colitis (such as, for example, toxic megacolon), enterocolitis (such as, for example, pseudomembranous entercolitis), proctocolitis, megacolon (such as, for example, Hirschsprung Disease and toxic megacolon), sigmoid diseases (such as, for example, proctocolitis and sigmoid neoplasms), Crohn's disease, diarrhea (such as, for example, infantile diarrhea), dysentery (such as, for example, amebic dysentery and bacillary dysentery), duodenal ulcer (such as, for example, Curling's Ulcer and duodenitis), enteritis (such as, for example, enterocolitis which includes pseudomembranous entercolitis), immunoproliferative small intestinal disease, inflammatory bowel diseases (such as, for example, ulcerative colitis and Crohn's Disease), proctitis (such as, for example, proctocolitis), rectal fistula (such as, for example, rectovaginal fistula), peptic ulcer, Peptic esophagitis, marginal ulcer, peptic ulcer hemorrhage, peptic ulcer perforation, stomach ulcer, Zollinger-Ellison Syndrome, gastritis (such as, for example, atrophic gastritis and hypertrophic gastritis), stomach rupture, stomach ulcer, pancreatic diseases (such as, for example, cystic fibrosis), pancreatic fistula, pancreatic insufficiency, pancreatic neoplasms and pancreatitis, mesenteric lymphadenitis, peritoneal paniculitis, peritonitis, and subphrenic abscess.

Digestive diseases whose detection, treatment, amelioration and/or prevention is encompassed by the present invention include, but are not limited to, biliary tract diseases (such as, for example, bile duct diseases which include cholangitis; gallbladder diseases such as cholecystitis), digestive system abnormialities (such as, for example, Barrett esophagus), digestive system fistula (which includes biliary fistula and esophageal fistula such as tracheoesophageal fistula, gastric fistula, intestinal fistula such, for example, as rectal fistula), digestive system fistula (such as, for example, intestinal fistula such as rectal fistula which includes rectovaginal fistula and pancreatic fistula), and esophageal motility disorders (such as, for example, CREST Syndrome).

Further examples of digestive diseases, whose detection, treatment, amelioration and/or prevention is encompassed by the present invention include, but are not limited to, liver diseases. Liver diseases include, but are not limited to, hepatitis (such as, for example, alcoholic hepatitis), toxic hepatitis, human viral hepatitis (such as, for example, delta infection, hepatitis A, hepatitis B, hepatitis C, chronic active hepatitis and hepatitis E), hepatomegaly, hepatorenal syndrome, liver abscess (such as, for example, amebic liver abscess), liver cirrhosis (such as, for example, alcoholic liver cirrhosis, biliary liver cirrhosis and experimental liver cirrhosis), alcoholic liver diseases (such as, for example, alcoholic hepatitis and alcoholic liver cirrhosis), and parasitic liver diseases (such as, for example, amebic liver abscess).

Stomatognathic diseases whose detection, treatment, amelioration and/or prevention is encompassed by the present invention include, but are not limited to, mouth diseases (such as, for example, Behcet's Syndrome, oral candidiasis, cheilitis, herpes labialis, lip neoplasms, Ludwig's Angina, Melkersson-Rosenthal Syndrome, oral hemorrhage such as gingival hemorrhage, oral manifestations, oral submucous fibrosis, periapical periodontitis, periapical abscess, periapical granuloma, radicular cyst, periodontal diseases (such as, for example, gingivitis, necrotizing ulcerative gingivitis, pericoronitis, periodontitis, and periodontal abscess), sialadenitis, necrotizing sialometaplasia, stomatitis (such as, for example, Stevens-Johnson Syndrome, aphthous stomatitis, denture stomatitis and herpetic stomatitis), tongue diseases (such as, for example, glossitis such as benign migratory glossitis), peritonsillar abscess, pharyngitis, retropharyngeal abscess, and tonsillitis.

Wound Healing and Epithelial Cell Proliferation

The present invention further encompasses methods, utilizing TNF-gamma-β, DR3, and/or TR6 polynucleotides or polypeptides, as well as antibodies and/or agonists of thereto, for therapeutic purposes, for example, to inhibit IFNγ secretion and thereby reduce inflammation for the purpose of wound healing, and to stimulate hair follicle production and healing of dermal wounds. Wounds and injuries whose treatment and/or amelioration is encompassed by the present invention include, but are not limited to, surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, bums resulting from heat exposure or chemicals, and other abnormal wound-healing conditions such as uremia, malnutrition, vitamin deficiencies and complications associted with systemic treatment with steroids, radiation therapy and antineoplastic drugs and antimetabolites. The present invention further encompasses methods of using embodiments of the invention to promote dermal reestablishment subsequent to dermal loss.

The present invention further encompasses methods of using embodiments of the invention to increase the adherence of skin grafts to a wound bed and to stimulate re-epithelialization from the wound bed. Grafts whose treatment and/or amelioration is encompassed by the present invention include, but are not limited to, autografts, artificial skin, allografts, autodermic graft, autoepidermic grafts, avacular grafts, Blair-Brown grafts, bone graft, brephoplastic grafts, cutis graft, delayed graft, dermic graft, epidermic graft, fascia graft, full thickness graft, heterologous graft, xenograft, homologous graft, hyperplastic graft, lamellar graft, mesh graft, mucosal graft, Ollier-Thiersch graft, omenpal graft, patch graft, pedicle graft, penetrating graft, split skin graft, and thick split graft. The present invention further encompasses methods of using embodiments of the invention to promote skin strength and to improve the appearance of aged skin.

The present invention further encompasses methods of using embodiments of the invention to produce changes in hepatocyte proliferation, and epithelial cell proliferation in the lung, breast, pancreas, stomach, small intestine, and large intestine. The present invention further encompasses methods of using embodiments of the invention to promote proliferation of epithelial cells such as sebocytes, hair follicles, hepatocytes, type II pneumocytes, mucin-producing goblet cells, and other epithelial cells and their progenitors contained within the skin, lung, liver, and gastrointestinal tract. The present invention further encompasses methods of using embodiments of the invention to promote proliferation of endothelial cells, keratinocytes, and basal keratinocytes.

The present invention further encompasses methods of using embodiments of the invention to reduce the side effects of gut toxicity that result from radiation, chemotherapy treatments or viral infections. The present invention further encompasses methods of using embodiments of the invention to have a cytoprotective effect on the small intestine mucosa. The present invention further encompasses methods of using embodiments of the invention to stimulate healing of mucositis (mouth ulcers) that result from chemotherapy and viral infections.

The present invention further encompasses methods of using embodiments of the invention to stimulate full regeneration of skin in full and partial thickness skin defects, including burns, (i.e., repopulation of hair follicles, sweat glands, and sebaceous glands), treatment of other skin defects such as psoriasis. The present invention further encompasses methods of using embodiments of the invention to treat epidermolysis bullosa, a defect in adherence of the epidermis to the underlying dermis which results in frequent, open and painful blisters by accelerating reepithelialization of these lesions. The present invention further encompasses methods of using embodiments of the invention to treat gastric and doudenal ulcers and help heal by scar formation of the mucosal lining and regeneration of glandular mucosa and duodenal mucosal lining more rapidly. Inflamamatory bowel diseases, such as Crohn's disease and ulcerative colitis, are diseases which result in destruction of the mucosal surface of the small or large intestine, respectively. Thus, the present invention further encompasses methods of using embodiments of the invention to promote the resurfacing of the mucosal surface to aid more rapid healing and to prevent progression of inflammatory bowel disease. The present invention further encompasses methods of using embodiments of the invention to regulate the production of mucus throughout the gastrointestinal tract and to thereby protect the intestinal mucosa from injurious substances that are ingested or following surgery. The present invention further encompasses methods of using embodiments of the invention to treat diseases associated with the aberrant expression of TNF-gamma-β, DR3, and/or TR6.

Moreover, the present invention further encompasses methods of using embodiments of the invention to prevent and heal damage to the lungs due to various pathological states. The present invention encompasses methods of using embodiments of the invention to inhibit IFNγ secretion and therby reduce inflammation and promote the repair of alveoli and brochiolar epithelium to prevent or treat acute or chronic lung damage. For example, emphysema, which results in the progressive loss of aveoli, and inhalation injuries, i.e., resulting from smoke inhalation and burns, that cause necrosis of the bronchiolar epithelium and alveoli could be effectively treated using embodiments of the present invention.

The present invention further encompasses methods of using embodiments of the invention to inhibit IFNγ secretion therby reducing inflammation and, thus, to alleviate or treat liver diseases and pathologies such as fulminant liver failure caused by cirrhosis, liver damage caused by viral hepatitis and toxic substances (i.e., acetaminophen, carbon tetrachloride and other hepatotoxins known in the art).

In addition, the present invention further encompasses methods of using embodiments of the invention to treat or prevent the onset of diabetes mellitus. In patients with newly diagnosed Types I and II diabetes, where some islet cell function remains, embodiments of the present invention could be used to maintain the islet function so as to alleviate, delay or prevent permanent manifestation of the disease. Also, embodiments of the present invention could be used as an auxiliary in islet cell transplantation to improve or promote islet cell function.

Compositions of the Invention

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention.

Polynucleotides of the invention encompass a nucleic acid sequence contained in SEQ ID NO:1, or cDNA clone HEMCZ56 as contained within ATCC Deposit No: 203055; SEQ ID NO:3, or a cDNA clone as contained within ATCC Deposit Number 97757; or SEQ ID NO:5, or cDNA clone HPHAE52 as contained within ATCC Deposit No: ATCC 97810. For example, a polynucleotide can contain the nucleotide sequence of a full length cDNA sequence, including the 5′ and 3′ untranslated sequences, the coding region, with or without a natural or artificial signal sequence, the protein coding region, as well as fragments, epitopes, domains, and variants of the nucleic acid sequence. Moreover, as used herein, polypeptides of the invention encompass molecules having an amino acid sequence encoded by a polynucleotide of the invention as broadly defined (obviously excluding poly-Phenylalanine or poly-Lysine peptide sequences which result from translation of a polyA tail of a sequence corresponding to a cDNA).

Polynucleotides of the present invention also include those polynucleotides capable of hybridizing, under stringent hybridization conditions, to sequences contained in SEQ ID NOs:1, 3, or 5, or the complements thereof (e.g., the complement of any one, two, three, four, or more of the polynucleotide fragments described herein) and/or sequences of the cDNA contained in the deposited clones (e.g., the complement of any one, two, three, four, or more of the polynucleotide fragments described herein). “Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65 degree C.

Also encompassed by polynucleotides of the present invention are nucleic acid molecules that hybridize to the polynucleotides of the present invention at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37 degree C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone generated using oligo dT as a primer).

The polynucleotides of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In specific embodiments, the polynucleotides of the invention are at least 15, at least 30, at least 50, at least 100, at least 125, at least 500, or at least 1000 continuous nucleotides in length. In a further embodiment, polynucleotides of the invention comprise a portion of the coding sequences, as disclosed herein.

The polypeptides of the present invention can be composed of amino acids joined to each other by peptide bonds or modified peptide bonds, i.e., peptide isosteres, and may contain amino acids other than the 20 gene-encoded amino acids. The polypeptides may be modified by either natural processes such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, including the secreted polypeptide, can be substantially purified using techniques described herein or otherwise known in the art, such as, for example, by the one-step method described in Smith and Johnson, Gene 67:31-40 (1988). Polypeptides of the invention also can be purified from natural, synthetic or recombinant sources using techniques described herein or otherwise known in the art, such as, for example, antibodies of the invention raised against the polypeptides of the present invention in methods which are well known in the art.

By a polypeptide demonstrating a “functional activity” is meant, a polypeptide capable of displaying one or more known functional activities associated with a full-length (complete) protein of the invention. Such functional activities include, but are not limited to, biological activity, antigenicity [ability to bind (or compete with a polypeptide for binding) to an anti-polypeptide antibody], immunogenicity (ability to generate antibody which binds to a specific polypeptide of the invention), ability to form multimers with polypeptides of the invention, and ability to bind to a receptor or ligand for a polypeptide.

“A polypeptide having functional activity” refers to polypeptides exhibiting activity similar, but not necessarily identical to, an activity of a polypeptide of the present invention, including mature forms, as measured in a particular assay, such as, for example, a biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention).

The functional activity of the polypeptides, and fragments, variants derivatives, and analogs thereof, can be assayed by various methods.

For example, in one embodiment where one is assaying for the ability to bind or compete with full-length polypeptide of the present invention for binding to an antibody to the full length polypeptide, various immunoassays known in the art can be used, including but not limited to, competitive and non-competitive assay systems using techniques such as radioimmunoassays, ELISA (enzyme linked immunosorbent assay), fluorescence-activated cell sorting (FACS), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In another embodiment, where a ligand is identified, or the ability of a polypeptide fragment, variant or derivative of the invention to multimerize is being evaluated, binding can be assayed, e.g., by means well-known in the art, such as, for example, reducing and non-reducing gel chromatography, protein affinity chromatography, and affinity blotting. See generally, Phizicky, E., et al., Microbiol. Rev. 59:94-123 (1995). In another embodiment, physiological correlates polypeptide of the present invention binding to its substrates (signal transduction) can be assayed.

In addition, assays described herein (see Examples) and otherwise known in the art may routinely be applied to measure the ability of polypeptides of the present invention and fragments, variants derivatives and analogs thereof to elicit polypeptide related biological activity (either in vitro or in vivo). Other methods will be known to the skilled artisan and are within the scope of the invention.

Polynucleotides

One embodiment of the present invention encompasses isolated nucleic acids (polynucleotides) which encode for the mature polypeptide having the deduced amino acid sequence of FIGS. 1A-1C (SEQ ID NO:2) or for the mature polypeptide encoded by the cDNA of the clone HEMCZ56 deposited as ATCC Deposit No. 203055 on Jul. 9, 1998. The ATCC number referred to above is directed to a biological deposit with the ATCC, 10801 University Boulevard, Manassas, Va. 20110-2209. The strain referred to is being maintained under terms of the Budapest Treaty and will be made available to a patent office signatory to the Budapest Treaty.

A polynucleotide encoding TNF-gamma-β of the present invention was isolated as clone HEMCZ56 from a human cDNA library. The polynucleotide contains an open reading frame encoding a protein of 251 amino acid residues. It is predicted that amino acid residues 1-35 constitute the intracellular domain, amino acid residues 36-61 constitute the transmembrane domain, and amino acid residues 62-251 constitute the extracellular domain.

One embodiment of the present invention encompasses isolated nucleic acids (polynucleotides) which encode for the mature polypeptide having the deduced amino acid sequence of FIGS. 3A-3C (SEQ ID NO:4) or for the mature polypeptide encoded by a cDNA deposited as ATCC Deposit No. 97757 on Oct. 10, 1996. The ATCC number referred to above is directed to a biological deposit with the ATCC, 10801 University Boulevard, Manassas, Va. 20110-2209. The strain referred to is being maintained under terms of the Budapest Treaty and will be made available to a patent office signatory to the Budapest Treaty.

A polynucleotide encoding DR3 of the present invention was isolated as a clone from a human vascular endothelial cell cDNA library. The polynucleotide contains an open reading frame encoding a protein of 417 amino acid residues. It is predicted that amino acid residues 1-24 constitute the signal peptide, amino acid residues 25-201 constitute the extracellular domain, amino acid residues 202-224 constitute the transmembrane domain, and amino acid residues 225-417 constitute the intracellular domain, with amino acid residues 342-408 constitute the death domain.

A further embodiment of the present invention encompasses isolated nucleic acids (polynucleotides) which encode for the mature polypeptide having the deduced amino acid sequence of FIGS. 5A-5B (SEQ ID NO:6) or for the mature polypeptide encoded by the cDNA clone HPHAE52 deposited as ATCC Deposit No. ATCC 97810 on Nov. 22, 1996. The ATCC number referred to above is directed to a biological deposit with the ATCC, 10801 University Boulevard, Manassas, Va. 20110-2209. The strain referred to is being maintained under terms of the Budapest Treaty and will be made available to a patent office signatory to the Budapest Treaty.

A polynucleotide encoding TR6 of the present invention was isolated as clone HPHAE52 from a human cDNA library. The polynucleotide contains an open reading frame encoding a protein of 300 amino acid residues. It is predicted that amino acid residues 1-30 constitute the signal peptide.

The polynucleotides of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the mature polypeptides may be identical to the coding sequence shown in FIGS. 1A-1B (SEQ ID NO:1), 3A-3C (SEQ ID NO:3), or 5A-5B (SEQ ID NO:5), or that of any of the deposited clone(s) or may be a different coding sequence which coding sequence, as a result of the redundancy or degeneracy of the genetic code, encodes the same mature polypeptides as the DNA of FIGS. 1A-1C (SEQ ID NO:1), 3A-3C (SEQ ID NO:3), or 5A-5B (SEQ ID NO:5), or any deposited cDNA.

The polynucleotides which encode for the mature polypeptide of FIGS. 1A-1C (SEQ ID NO:2), 3A-3C (SEQ ID NO:4), or 5A-5B (SEQ ID NO:6), or for the mature polypeptide encoded by any of the deposited cDNAs may include: only the coding sequence for a mature polypeptide; the coding sequence for a mature polypeptide and additional coding sequence such as a leader or secretory sequence or a proprotein sequence; the coding sequence for a mature polypeptide (and optionally additional coding sequence) and non-coding sequence, such as introns or non-coding sequence 5′ and/or 3′ of the coding sequence for the mature polypeptides.

Thus, the term “polynucleotide encoding a polypeptide” encompasses a polynucleotide which includes only coding sequence for the polypeptide as well as a polynucleotide which includes additional coding and/or non-coding sequence.

Polypeptides

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, including the secreted polypeptide, can be substantially purified using techniques described herein or otherwise known in the art, such as, for example, by the one-step method described in Smith and Johnson, Gene 67:31-40 (1988). Polypeptides of the invention also can be purified from natural, synthetic or recombinant sources using techniques described herein or otherwise known in the art, such as, for example, antibodies of the invention raised against the secreted protein.

The present invention provides a polynucleotide comprising, or alternatively consisting of, a polynucleotide having a nucleic acid sequence selected from those of SEQ ID NOs:1, 3, and 5, and/or cDNAs contained in ATCC deposits 203055, 97757, and 97810. The present invention also provides a polypeptide comprising, or alternatively, consisting of, a polypeptide having an amino acid sequence selected from those of SEQ ID NOs:2, 4, and 6, and/or those encoded by the cDNAs contained in ATCC deposits 203055, 97757, and 97810. Polynucleotides encoding a polypeptide comprising, or alternatively consisting of a polypeptide having an amino acid sequence selected from those of SEQ ID NOs:2, 4, and 6, and/or those encoded by the cDNAs contained in ATCC deposits 203055, 97757, and 97810, are also encompassed by the invention.

Signal Sequences

The present invention also encompasses mature forms of the polypeptides having the amino acid sequences of SEQ ID NOs:2, 4, and 6, and/or the amino acid sequences encoded by the cDNA clones in ATCC deposits 203055, 97757, and 97810. Polynucleotides encoding the mature forms (such as, for example, the polynucleotide sequence in SEQ ID NO:1 and/or the polynucleotide sequence contained in the cDNA of a deposited clone) are also encompassed by the invention. According to the signal hypothesis, proteins secreted by mammalian cells have a signal or secretary leader sequence which is cleaved from the mature protein once export of the growing protein chain across the rough endoplasmic reticulum has been initiated. Most mammalian cells and even insect cells cleave secreted proteins with the same specificity. However, in some cases, cleavage of a secreted protein is not entirely uniform, which results in two or more mature species of the protein. Further, it has long been known that cleavage specificity of a secreted protein is ultimately determined by the primary structure of the complete protein, that is, it is inherent in the amino acid sequence of the polypeptide.

Methods for predicting whether a protein has a signal sequence, as well as the cleavage point for that sequence, are available. For instance, the method of McGeoch, Virus Res. 3:271-286 (1985), uses the information from a short N-terminal charged region and a subsequent uncharged region of the complete (uncleaved) protein. The method of von Heinje, Nucleic Acids Res. 14:4683-4690 (1986) uses the information from the residues surrounding the cleavage site, typically residues −13 to +2, where +1 indicates the amino terminus of the secreted protein. The accuracy of predicting the cleavage points of known mammalian secretory proteins for each of these methods is in the range of 75-80%. (von Heinje, supra.) However, the two methods do not always produce the same predicted cleavage point(s) for a given protein.

In the present case, the deduced amino acid sequence of the TNF-gamma-β polypeptide was analyzed and the signal peptide is predicted to comprise the first 35 amino acids of the polypeptide sequence shown in SEQ ID NO:2 (i.e. amino acid residues MAEDLGLSFGETASVEMLPEHGSCRPKARSSSARW). See, FIGS. 1A-1B. Accordingly, the signal cleavage site is predicted to occur between amino acid residues 35 and 36 in SEQ ID NO:2. Hence, the mature form of the protein is predicted to comprise amino acid residues 36-251 of SEQ ID NO:2. See, FIGS. 1A-1B.

In the present case, the deduced amino acid sequence of the DR3 polypeptide was analyzed and the signal peptide is predicted to comprise the first 24 amino acids of the polypeptide sequence shown in SEQ ID NO:4 (i.e. amino acid residues MEQPPRGCAAVAAALLLVLLGARA). See, FIGS. 3A-3C. Accordingly, the signal cleavage site is predicted to occur between amino acid residues 24 and 25 in SEQ ID NO:4. Hence, the mature form of the protein is predicted to comprise amino acid residues 25-417 of SEQ ID NO:4. See, FIGS. 3A-3C.

In the present case, the deduced amino acid sequence of the TR6 polypeptide was analyzed and the signal peptide is predicted to comprise the first 30 amino acids of the polypeptide sequence shown in SEQ ID NO:6 (i.e. amino acid residues MRALEGPGLSLLCLVLALPALLPVPAVRGV). See, FIGS. 5A-5B. Accordingly, the signal cleavage site is predicted to occur between amino acid residues 30 and 31 in SEQ ID NO:6. Hence, the mature form of the protein is predicted to comprise amino acid residues 31-300 of SEQ ID NO:6. See, FIGS. 5A-5B.

As one of ordinary skill would appreciate, however, cleavage sites sometimes vary from organism to organism and cannot be predicted with absolute certainty. Accordingly, the present invention provides secreted polypeptides having a sequence shown in SEQ ID NOs:2, 4, and 6, which have an N-terminus beginning within 5 residues (i.e., + or −5 residues) of the predicted cleavage point described above. Similarly, it is also recognized that in some cases, cleavage of the signal sequence from a secreted protein is not entirely uniform, resulting in more than one secreted species. These polypeptides, and the polynucleotides encoding such polypeptides, are contemplated by the present invention.

Moreover, the signal sequence identified by the above analysis may not necessarily predict the naturally occurring signal sequence. Nonetheless, the present invention provides the mature protein, produced by expression of the polynucleotide sequence of SEQ ID NOs:1, 3, and 5, and/or the polynucleotide sequence contained in the cDNAs of the deposited clones, in a mammalian cell (e.g., COS cells, as described below). These polypeptides, and the polynucleotides encoding such polypeptides, are contemplated by the present invention.

Polynucleotide and Polypeptide Variants

The present invention encompasses variants of the polynucleotide sequences disclosed in SEQ ID NOs:1, 3, and 5, (FIGS. 1A-1B, 3A-3C, and 5A-5B), the complementary strands thereto, and/or the cDNA sequences contained in the deposited clones.

The present invention also encompasses variants of the polypeptide sequences disclosed in SEQ ID NOs:2, 4, and 6, (FIGS. 1A-1B, 3A-3C, and 5A-5B) and/or encoded by the deposited clones.

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

The present invention also encompasses nucleic acid molecules which comprise, or alternatively consist of, a nucleotide sequence which is at least 80%, 85%, 90%, 95%, 96%, 97%, 98% or 99% identical to, for example, a nucleotide coding sequence in SEQ ID NOs:1, 3, and 5, or the complementary strands thereto, a nucleotide coding sequence contained in the deposited cDNA clones or the complementary strands thereto, a nucleotide sequence encoding a polypeptide of SEQ ID NOs:2, 4, and 6, a nucleotide sequence encoding a polypeptide encoded by a cDNA contained in a deposited clone, and/or polynucleotide fragments of any of these nucleic acid molecules (e.g., those fragments described herein). Polynucleotides which hybridize to these nucleic acid molecules under stringent hybridization conditions or lower stringency conditions are also encompassed by the invention, as are polypeptides encoded by these polynucleotides.

The present invention also encompasses polypeptides which comprise, or alternatively consist of, an amino acid sequence which is at least 80%, 85%, 90%, 95%, 96%, 97%, 98%, 99% identical to, for example, the polypeptide sequence shown in SEQ ID NO:2, the polypeptide sequence encoded by the cDNA contained in the deposited clone, and/or polypeptide fragments of any of these polypeptides (e.g., those fragments described herein).

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding the polypeptide. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown in one of SEQ ID NOs:1, 3, and/or 5, the ORF (open reading frame), or any fragment specified as described herein.

As a practical matter, whether any particular nucleic acid molecule or polypeptide is at least 80%, 85%, 90%, 95%, 96%, 97%, 98% or 99% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245(1990)). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to, for instance, an amino acid sequence shown in one of SEQ ID NOs:2, 4, and/or 6, or to an amino acid sequence encoded by a cDNA contained in a deposited clone, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245(1990)). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons, e.g., to optimize codon expression for a particular host (change codons in the human mRNA to those preferred by a bacterial host such as E. coli).

Naturally occurring variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level and are included in the present invention. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

Moreover, ample evidence demonstrates that variants often retain a biological activity similar to that of the naturally occurring protein. For example, Gayle and coworkers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” Id. In fact, only 23 unique amino acid sequences, out of more than 3,500 nucleotide sequences examined, produced a protein that significantly differed in activity from wild-type. Id.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include (i) substitutions with one or more of the non-conserved amino acid residues, where the substituted amino acid residues may or may not be one encoded by the genetic code, or

(ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as, for example, an IgG Fc fusion region peptide, or leader or secretory sequence, or a sequence facilitating purification or (v) fusion of the polypeptide with another compound, such as albumin (including, but not limited to, recombinant albumin (see, e.g., U.S. Pat. No. 5,876,969, issued Mar. 2, 1999, EP Patent 0 413 622, and U.S. Pat. No. 5,766,883, issued Jun. 16, 1998, herein incorporated by reference in their entirety)). Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

A further embodiment of the invention relates to a polypeptide which comprises the amino acid sequence of the present invention having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a peptide or polypeptide to have an amino acid sequence which comprises the amino acid sequence of the present invention, which contains at least one, but not more than 10, 9, 8, 7, 6, 5, 4, 3, 2 or 1 amino acid substitutions. In specific embodiments, the number of additions, substitutions, and/or deletions in the amino acid sequence of the present invention or fragments thereof (e.g., the mature form and/or other fragments described herein), is 1-5,5-10, 5-25, 5-50, 10-50 or 50-150, conservative amino acid substitutions are preferable.

Polynucleotide and Polypeptide Fragments

The present invention is also encompasses polynucleotide fragments of the polynucleotides of the invention.

In the present invention, a “polynucleotide fragment” refers to a short polynucleotide having a nucleic acid sequence which: is a portion of that contained in a deposited clone, or encoding the polypeptide encoded by the cDNA in the deposited clone; is a portion of that shown in one of SEQ ID NOs:1, 3, or 5, or the complementary strands thereto, or is a portion of a polynucleotide sequence encoding the polypeptide of one of SEQ ID NOs:2, 4, or 6. The nucleotide fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A fragment “at least 20 nt in length,” for example, is intended to include 20 or more contiguous bases from a cDNA sequence contained in a deposited clone or a nucleotide sequence shown in SEQ ID NOs:1, 3, or 5. In this context “about” includes the particularly recited value, a value larger or smaller by several (5, 4, 3, 2, or 1) nucleotides, at either terminus or at both termini. These nucleotide fragments have uses that include, but are not limited to, as diagnostic probes and primers as discussed herein. Of course, larger fragments (e.g., 50, 150, 500, 600, 1000, or 1114 nucleotides) are preferred.

Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, or 1051-1116 of SEQ ID NO:1, or the complementary strand thereto, or the cDNA contained in a deposited clone. Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, or 1201-1254 of SEQ ID NO:3, or the complementary strand thereto, or the cDNA contained in a deposited clone. Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, or 1051-1077 of SEQ ID NO:5, or the complementary strand thereto, or the cDNA contained in a deposited clone. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Polynucleotides which hybridize to these nucleic acid molecules under stringent hybridization conditions or lower stringency conditions are also encompassed by the invention, as are polypeptides encoded by these polynucleotides.

In the present invention, a “polypeptide fragment” refers to an amino acid sequence which is a portion of that contained in one of SEQ ID NOs:2, 4, or 6, (FIGS. 1A-1B, 3A-3C, or 5A-5B) or encoded by a cDNA contained in a deposited clone. Protein (polypeptide) fragments may be “free-standing,” or comprised within a larger polypeptide of which the fragment forms a part or region, most preferably as a single continuous region. Representative examples of polypeptide fragments of the invention, include, for example, fragments comprising, or alternatively consisting of, from about amino acid number 1-20, 21-40, 41-60, 61-80, 81-100, 102-120, 121-140, 141-160, 161-180, 181-200, 201-220, or 221-251 of SEQ ID NO:2. Moreover, polypeptide fragments of SEQ ID NO:2 can be about 20, 30, 40, 50, 60, 70, 80, 90, 100, 110, 120, 130, or 140 amino acids in length. Representative examples of polypeptide fragments of the invention, include, for example, fragments comprising, or alternatively consisting of, from about amino acid number 1-20, 21-40, 41-60, 61-80, 81-100, 102-120, 121-140, 141-160, 161-180, 181-200, 201-220, 221-240, 241-260, 261-280, 281-300, 301-320, 321-340, 341-360, 361-380, or 381-417 of SEQ ID NO:4. Moreover, polypeptide fragments of SEQ ID NO:4 can be about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 amino acids in length. Representative examples of polypeptide fragments of the invention, include, for example, fragments comprising, or alternatively consisting of, from about amino acid number 1-20, 21-40, 41-60, 61-80, 81-100, 102-120, 121-140, 141-160, 161-180, 181-200, 201-220, 221-240, 241-260, 261-280, or 281-300 of SEQ ID NO:6. Moreover, polypeptide fragments of SEQ ID NO:6 can be about 20, 30, 40, 50, 60, 70, 80, 90, 100, 110, 120, 130, 140, 150, 160, 170, 180, 190, 200, 210, 220, 230, 240, 250, 260, 270, 280, 290, or 300 amino acids in length. In this context “about” includes the particularly recited ranges or values, and ranges or values larger or smaller by several (5, 4, 3, 2, or 1) amino acids, at either extreme or at both extremes. Polynucleotides encoding these polypeptides are also encompassed by the invention.

A preferred embodiment of the present invention includes antibodies that bind the above-identified fragments.

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids, ranging from 1-220, can be deleted from the amino terminus of either the secreted polypeptide or the mature form of a polypeptide having an amino acid sequence shown in SEQ ID NO:2. Similarly, for example, any number of amino acids, ranging from 1-220, can be deleted from the carboxy terminus of the secreted protein or mature form of a polypeptide having an amino acid sequence shown in SEQ ID NO:2. Furthermore, any combination of the above amino and carboxy terminus deletions are preferred. Polynucleotides encoding these polypeptide fragments and antibodies that bind these polypeptide fragments are encompassed by the invention.

Accordingly, the present invention further provides polypeptides having one or more residues deleted from the amino terminus of the amino acid sequence of SEQ ID NO:2 (FIGS. 1A-1B), and polynucleotides encoding such polypeptides. For example, the present invention provides polypeptides comprising, or alternatively consisting of, the amino acid sequence of residues n¹-251 of SEQ ID NO:2, where n¹ is an integer in the range of 2-246. More in particular, in certain embodiments, the invention provides polynucleotides encoding polypeptides comprising, or alternatively consisting of, an amino acid sequence selected from the group consisting of residues of A-2 to L-251; E-3 to L-251; D-4 to L-251; L-5 to L-251; G-6 to L-251; L-7 to L-251;S-8 to L-251; F-9 to L-251; G-10 to L-251; E-11 to L-251; T-12 to L-251; A-13 to L-251; S-14 toL-251; V-15 to L-251; E-16 to L-251; M-17 toL-251; L-18 to L-251; P-19 to L-251; E-20 toL-251; H-21 to L-251; G-22 to L-251; S-23 toL-251; C-24 to L-251; R-25 to L-251; P-26 toL-251; K-27 to L-251; A-28 to L-251; R-29 toL-251; S-30 to L-251; S-31 to L-251; S-32 toL-251; A-33 to L-251; R-34 to L-251; W-35 toL-251; A-36 to L-251; L-37 to L-251; T-38 toL-251; C-39 to L-251; C-40 to L-251; L-41 toL-251; V-42 to L-251; L-43 to L-251; L-44 toL-251; P-45 to L-251; F-46 to L-251; L-47 toL-251; A-48 to L-251; G-49 to L-251; L-50 toL-251; T-51 to L-251; T-52 to L-251; Y-53 toL-251; L-54 to L-251; L-55 to L-251; V-56 toL-251; S-57 to L-251; Q-58 to L-251; L-59 toL-251; R-60 to L-251; A-61 to L-251; Q-62 toL-251; G-63 to L-251; E-64 to L-251; A-65 toL-251; C-66 to L-251; V-67 to L-251; Q-68 toL-251; F-69 to L-251; Q-70 to L-251; A-71 toL-251; L-72 to L-251; K-73 to L-251; G-74 toL-251; Q-75 to L-251; E-76 to L-251; F-77 toL-251; A-78 to L-251; P-79 to L-251; S-80 toL-251; H-81 to L-251; Q-82 to L-251; Q-83 toL-251; V-84 to L-251; Y-85 to L-251; A-86 toL-251; P-87 to L-251; L-88 to L-251; R-89 toL-251; A-90 to L-251; D-91 to L-251; G-92 toL-251; D-93 to L-251; K-94 to L-251; P-95 toL-251; R-96 to L-251; A-97 to L-251; H-98 toL-251; L-99 to L-251; T-100 to L-251; V-101 toL-251; V-102 to L-251; R-103 to L-251; Q-104 to L-251; T-105 to L-251; P-106 to L-251; T-107 to L-251; Q-108 to L-251; H-109 to L-251;F-110 to L-251; K-111 to L-251; N-112 toL-251; Q-113 to L-251; F-114 to L-251; P-115 toL-251; A-116 to L-251; L-117 to L-251; H-118 toL-251; W-119 to L-251; E-120 to L-251; H-121 to L-251; E-122 to L-251; L-123 to L-251; G-124 to L-251; L-125 to L-251; A-126 to L-251; F-127 to L-251; T-128 to L-251; K-129 to L-251;N-130 to L-251; R-131 to L-251; M-132 toL-251; N-133 to L-251; Y-134 to L-251; T-135 to L-251; N-136 to L-251; K-137 to L-251;F-138 to L-251; L-139 to L-251; L-140 to L-251;I-141 to L-251; P-142 to L-251; E-143 to L-251;S-144 to L-251; G-145 to L-251; D-146 to L-251;Y-147 to L-251; F-148 to L-251; 1-149 to L-251;Y-150 to L-251; S-151 to L-251; Q-152 toL-251; V-153 to L-251; T-154 to L-251; F-155 toL-251; R-156 to L-251; G-157 to L-251; M-158 to L-251; T-159 to L-251; S-160 to L-251; E-161 to L-251; C-162 to L-251; S-163 to L-251; E-164 to L-251; I-165 to L-251; R-166 to L-251; Q-167 to L-251; A-168 to L-251; G-169 to L-251;R-170 to L-251; P-171 to L-251; N-172 to L-251;K-173 to L-251; P-174 to L-251; D-175 toL-251; S-176 to L-251; I-177 to L-251; T-178 toL-251; V-179 to L-251; V-180 to L-251; 1-181 toL-251; T-182 to L-251; K-183 to L-251; V-184 to L-251; T-185 to L-251; D-186 to L-251; S-187 to L-251; Y-188 to L-251; P-189 to L-251; E-190 to L-251; P-191 to L-251; T-192 to L-251; Q-193 to L-251; L-194 to L-251; L-195 to L-251;M-196 to L-251; G-197 to L-251; T-198 toL-251; K-199 to L-251; S-200 to L-251; V-201 to L-251; C-202 to L-251; E-203 to L-251; V-204 to L-251; G-205 to L-251; S-206 to L-251;N-207 to L-251; W-208 to L-251; F-209 toL-251; Q-210 to L-251; P-211 to L-251; 1-212 toL-251; Y-213 to L-251; L-214 to L-251; G-215 toL-251; A-216 to L-251; M-217 to L-251; F-218 to L-251; S-219 to L-251; L-220 to L-251; Q-221 to L-251; E-222 to L-251, G-223 to L-251; D-224 to L-251; K-225 to L-251; L-226 to L-251; M-227 to L-251; V-228 to L-251; N-229 toL-251; V-230 to L-251; S-231 to L-251; D-232 toL-251; 1-233 to L-251; S-234 to L-251; L-235 toL-251; V-236 to L-251; D-237 to L-251; Y-238 to L-251; T-239 to L-251; K-240 to L-251; E-241 to L-251; D-242 to L-251; K-243 to L-251;T-244 to L-251; F-245 to L-251; F-246 to L-251;of SEQ ID NO:2. Polynucleotides encoding the above polypeptide fragments and antibodies that bind the above polypeptide fragments are also encompassed by the invention.

The present invention also encompasses nucleic acid molecules comprising, or alternatively, consisting of, a polynucleotide sequence at least 80%, 85%, 90%, 92%, 95%, 96%, 97%, 98% or 99% identical to the polynucleotide sequence encoding polypeptides as described above. The present invention also encompasses the above polynucleotide sequences fused to a heterologous polynucleotide sequence. Polypeptides encoded by these nucleic acids and/or polynucleotide sequences are also encompassed by the invention, as are polypeptides comprising, or alternatively consisting of, an amino acid sequence at least 80%, 85%, 90%, 92%, 95%, 96%, 97%, 98% or 99% identical to the amino acid sequence described above, and polynucleotides that encode such polypeptides.

Moreover, the present invention further provides polypeptides having one or more residues deleted from the carboxy terminus of the amino acid sequence of the polypeptide shown in SEQ ID NO:2 (FIGS. 1A-1B). For example, the present invention provides polypeptides comprising, or alternatively consisting of, the amino acid sequence of residues 1-m¹ of the amino acid sequence in SEQ ID NO:2, where ml is any integer in the range 6-250. More in particular, in certain embodiments, the invention provides polynucleotides encoding polypeptides comprising, or alternatively consisting of, an amino acid sequence selected from the group consisting of residues M-1 to L-250; M-1 to F-249; M-1 toA-248; M-1 to G-247; M-1 to F-246; M-1 toF-245; M-1 to T-244; M-1 to K-243; M-1 toD-242; M-1 to E-241; M-1 to K-240; M-1 toT-239; M-1 to Y-238; M-1 to D-237; M-1 toV-236; M-1 to L-235; M-1 to S-234; M-1 toL-233; M-1 to D-232; M-1 to S-231; M-1 toV-230; M-1 to N-229; M-1 to V-228; M-1 toM-227; M-1 to L-226; M-1 to K-225; M-1 toD-224; M-1 to G-223; M-1 to E-222; M-1 toQ-221; M-1 to L-220; M-1 to S-219; M-1 toF-218; M-1 to M-217; M-1 to A-216; M-1 toG-215; M-1 to L-214; M-1 to Y-213; M-1 toI-212; M-I to P-211; M-I to Q-210; M-I toF-209; M-1 to W-208; M-1 to N-207; M-1 toS-206; M-1 to G-205; M-1 to V-204; M-1 toE-203; M-1 to C-202; M-1 to V-201; M-1 toS-200; M-1 to K-199; M-1 to T-198; M-1 toG-197; M-1 to M-196; M-1 to L-195; M-1 toL-194; M-1 to Q-193; M-1 to T-192; M-1 toP-191; M-1 to E-190; M-1 to P-189; M-1 toY-188; M-1 to S-187; M-1 to D-186; M-1 toT-185; M-1 to V-184; M-1 to K-183; M-1 toT-182; M-1 to 1-181; M-1 to V-180; M-1 toV-179; M-1 to T-178; M-1 to 1-177; M-1 toS-176; M-1 to D-175; M-1 to P-174; M-1 toK-173; M-1 to N-172; M-1 to P-171; M-1 toR-170; M-1 to G-169; M-1 to A-168; M-1 toQ-167; M-1 to R-166; M-1 to 1-165; M-1 toE-164; M-1 to S-163; M-1 to C-162; M-1 toE-161; M-1 to S-160; M-1 to T-159; M-1 toM-158; M-1 to G-157; M-1 to R-156; M-1 toF-155; M-1 to T-154; M-1 to V-153; M-1 toQ-152; M-1 to S-151; M-1 to Y-150; M-1 toL-149; M-1 to F-148; M-1 to Y-147; M-1 toD-146; M-1to G-145; M-1 to S-144; M-1 toE-143; M-1 to P-142; M-1 to 1-141; M-1 toL-140; M-1 to L-139; M-1 to F-138; M-1 toK-137; M-1 to N-136; M-1 to T-135; M-1 toY-134; M-1 to N-133; M-1 to M-132; M-1 toR-131; M-1 to N-130; M-1 to K-129; M-1 toT-128; M-1 to F-127; M-1 to A-126; M-1 toL-125; M-1 to G-124; M-1 to L-123; M-1 toE-122; M-1 to H-121; M-1 to E-120; M-1 toW-119; M-1 to H-118; M-1 to L-117; M-1 toA-116; M-1 to P-115; M-1 to F-I 14; M-1 toQ-113; M-1 to N-112; M-1 to K-111; M-1 toF-110; M-1 to H-109; M-1 to Q-108; M-1 toT-107; M-1 to P-106; M-1 to T-105; M-1 toQ-104; M-1 to R-103; M-1 to V-102; M-1 toV-101; M-1 to T-100; M-1 to L-99; M-1 to H-98;M-1 to A-97; M-1 to R-96; M-1 to P-95; M-1 toK-94; M-1 to D-93; M-1 to G-92; M-1 to D-91;M-1 to A-90; M-1 to R-89; M-1 to L-88; M-1 toP-87; M-1 to A-86; M-1 to Y-85; M-1 to V-84;M-1 to Q-83; M-1 to Q-82; M-1 to H-81; M-1 toS-80; M-1 to P-79; M-1 to A-78; M-1 to F-77;M-1 to E-76; M-1 to Q-75; M-1 to G-74; M-1 toK-73; M-1 to L-72; M-1 to A-71; M-1 to Q-70;M-1 to F-69; M-1 to Q-68; M-1 to V-67; M-1 toC-66; M-1 to A-65; M-1 to E-64; M-1 to G-63;M-1 to Q-62; M-1 to A-61; M-1 to R-60; M-1 toL-59; M-1 to Q-58; M-1 to S-57; M-1 to V-56;M-1 to L-55; M-1 to L-54; M-1 to Y-53; M-1 toT-52; M-1 to T-51; M-1 to L-50; M-1 to G-49;M-1 to A-48; M-1 to L-47; M-1 to F-46; M-1 toP-45; M-1 to L-44; M-1 to L-43; M-1 to V-42;M-1 to L-41; M-1 to C-40; M-1 to C-39; M-1 toT-38; M-1 to L-37; M-1 to A-36; M-1 to W-35;M-1 to R-34; M-1 to A-33; M-1 to S-32; M-1 toS-31; M-1 to S-30; M-1 to R-29; M-1 to A-28;M-1 to K-27; M-1 to P-26; M-1 to R-25; M-1 toC-24; M-1 to S-23; M-1 to G-22; M-1 to H-21;M-1 to E-20; M-1 to P-19; M-1 to L-18; M-1 toM-17; M-1 to E-16; M-1 to V-15; M-1 to S-14;M-1 to A-13; M-1 to T-12; M-1 to E-11; M-1 toG-10; M-1 to F-9; M-1 to S-8; M-1 to L-7; M-1 to G-6; of SEQ ID NO:2. Polynucleotides encoding the above polypeptide fragments and antibodies that bind the above polypeptide fragments are also encompassed by the invention.

The present invention encompasses nucleic acid molecules comprising, or alternatively, consisting of, a polynucleotide sequence at least 80%, 85%, 90%, 92%, 95%, 96%, 97%, 98% or 99% identical to the polynucleotide sequence encoding the polypeptides described above. The present invention also encompasses the above polynucleotide sequences fused to a heterologous polynucleotide sequence. Polypeptides encoded by these nucleic acids and/or polynucleotide sequences are also encompassed by the invention, as are polypeptides comprising, or alternatively consisting of, an amino acid sequence at least 80%, 85%, 90%, 92%, 95%, 96%, 97%, 98% or 99% identical to the amino acid sequence described above, and polynucleotides that encode such polypeptides.

The invention also provides polypeptides having one or more amino acids deleted from both the amino and the carboxyl termini of the polypeptide of SEQ ID NO:2 (FIGS. 1A-1B). For example, amino terminal and carboxyl terminal deletions of the polypeptide sequence may be described generally, for example, as having residues n¹-m¹ of SEQ ID NO:2 where n¹ is an integer in the range of 1-237 and m¹ is an integer in the range of 16-251. For example, and more in particular, the invention provides polynucleotides encoding polypeptides comprising, or alternatively consisting of, an amino acid sequence selected from the group consisting of residues of M-1 to V-15; A-2 to E-16; E-3 to M-17; D-4 toL-18; L-5 to P-19; G-6 to E-20; L-7 to H-21; S-8 to G-22; F-9 to S-23; G-10 to C-24; E-11 toR-25; T-12 to P-26; A-13 to K-27; S-14 to A-28;V-15 to R-29; E-16 to S-30; M-17 to S-31; L-18 to S-32; P-19 to A-33; E-20 to R-34; H-21 toW-35; G-22 to A-36; S-23 to L-37; C-24 to T-38;R-25 to C-39; P-26 to C-40; K-27 to L-41; A-28 to V-42; R-29 to L-43; S-30 to L-44; S-31 toP-45; S-32 to F-46; A-33 to L-47; R-34 to A-48;W-35 to G-49; A-36 to L-50; L-37 to T-51; T-38 to T-52; C-39 to Y-53; C-40 to L-54; L-41 toL-55; V-42 to V-56; L-43 to S-57; L-44 to Q-58;P-45 to L-59; F-46 to R-60; L-47 to A-61; A-48 to Q-62; G-49 to G-63; L-50 to E-64; T-51 toA-65; T-52 to C-66; Y-53 to V-67; L-54 to Q-68;L-55 to F-69; V-56 to Q-70; S-57 to A-71; Q-58 to L-72; L-59 to K-73; R-60 to G-74; A-61 toQ-75; Q-62 to E-76; G-63 to F-77; E-64 to A-78;A-65 to P-79; C-66 to S-80; V-67 to H-81; Q-68 to Q-82; F-69 to Q-83; Q-70 to V-84; A-71 toY-85; L-72 to A-86; K-73 to P-87; G-74 to L-88;Q-75 to R-89; E-76 to A-90; F-77 to D-91; A-78 to G-92; P-79 to D-93; S-80 to K-94; H-81 toP-95; Q-82 to R-96; Q-83 to A-97; V-84 to H-98;Y-85 to L-99; A-86 to T-100; P-87 to V-101; L-88 to V-102; R-89 to R-103; A-90 to Q-104;D-91 to T-105; G-92 to P-106; D-93 to T-107;K-94 to Q-108; P-95 to H-109; R-96 to F-110; A-97 to K-111; H-98 to N-112; L-99 to Q-113;T-100 to F-114; V-101 to P-115; V-102 toA-116; R-103 to L-117; Q-104 to H-118; T-105 to W-119; P-106 to E-120; T-107 to H-121;Q-108 to E-122; H-109 to L-123; F-110 toG-124; K-Ill to L-125; N-112 to A-126; Q-113 to F-127; F-114 to T-128; P-115 to K-129;A-116 to N-130; L-117 to R-131; H-118 toM-132; W-119 to N-133; E-120 to Y-134; H-121 to T-135; E-122 to N-136; L-123 to K-137;G-124 to F-138; L-125 to L-139; A-126 to L-140;F-127 to 1-141; T-128 to P-142; K-129 to E-143;N-130 to S-144; R-131 to G-145; M-132 toD-146; N-133 to Y-147; Y-134 to F-148; T-135 to 1-149; N-136 to Y-150; K-137 to S-151; F-138 to Q-152; L-139 to V-153; L-140 to T-154; I-141 to F-155; P-142 to R-156; E-143 to G-157; S-144 to M-158; G-145 to T-159; D-146 to S-160;Y-147 to E-161; F-148 to C-162; 1-149 to S-163;Y-150 to E-164; S-151 to 1-165; Q-152 to R-166;V-153 to Q-167; T-154 to A-168; F-155 toG-169; R-156 to R-170; G-157 to P-171; M-158 to N-172; T-159 to K-173; S-160 to P-174;E-161 to D-175; C-162 to S-176; S-163 to I-177;E-164 to T-178; 1-165 to V-179; R-166 to V-180;Q-167 to 1-181; A-168 to T-182; G-169 toK-183; R-170 to V-184; P-171 to T-185; N-172 to D-186; K-173 to S-187; P-174 to Y-188;D-175 to P-189; S-176 to E-190; 1-177 to P-191;T-178 to T-192; V-179 to Q-193; V-180 toL-194; 1-181 to L-195; T-182 to M-196; K-183 toG-197; V-184 to T-198; T-185 to K-199; D-186 to S-200; S-187 to V-201; Y-188 to C-202;P-189 to E-203; E-190 to V-204; P-191 to G-205;T-192 to S-206; Q-193 to N-207; L-194 toW-208; L-195 to F-209; M-196 to Q-210; G-197 to P-211; T-198 to 1-212; K-199 to Y-213; S-200 to L-214; V-201 to G-215; C-202 to A-216;E-203 to M-217; V-204 to F-218; G-205 toS-219; S-206 to L-220; N-207 to Q-221; W-208 to E-222; F-209 to G-223; Q-210 to D-224;P-211 to K-225; 1-212 to L-226; Y-213 toM-227; L-214 to V-228; G-215 to N-229; A-216 to V-230; M-217 to S-231; F-218 to D-232;S-219 to 1-233; L-220 to S-234; Q-221 to L-235;E-222 to V-236; G-223 to D-237; D-224 toY-238; K-225 to T-239; L-226 to K-240; M-227 to E-241; V-228 to D-242; N-229 to K-243;V-230 to T-244; S-231 to F-245; D-232 to F-246;I-233 to G-247; S-234 to A-248; L-235 to F-249;V-236 to L-250; D-237 to L-251; of SEQ ID NO:2 Polynucleotides encoding the above polypeptide fragments and antibodies that bind the above polypeptide fragments are also encompassed by the invention.

The present invention encompasses nucleic acid molecules comprising, or alternatively, consisting of, a polynucleotide sequence at least 80%, 85%, 90%, 92%, 95%, 96%, 97%, 98% or 99% identical to the polynucleotide sequence encoding the polypeptides described above. The present invention also encompasses the above polynucleotide sequences fused to a heterologous polynucleotide sequence. Polypeptides encoded by these nucleic acids and/or polynucleotide sequences are also encompassed by the invention, as are polypeptides comprising, or alternatively consisting of, an amino acid sequence at least 80%, 85%, 90%, 92%, 95%, 96%, 97%, 98% or 99% identical to the amino acid sequence described above, and polynucleotides that encode such polypeptides.

Also preferred are polypeptide and polynucleotide fragments characterized by structural or functional domains (See, FIG. 2 and Table 1), such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. See FIG. 2 and Table 1. Polypeptide fragments of SEQ ID NO:2 falling within conserved domains, hydrophillic, and antigenic domains are specifically contemplated by the present invention. Moreover, polynucleotides encoding these domains and antibodies that bind to these domains are also contemplated.

TABLE 1

| Res: | Pos: | I | II | III | IV | V | VI | VII | VIII | IX | X | XI | XII | XIII | XIV |
|---|---|---|---|---|---|---|---|---|---|---|---|---|---|---|---|
| Met | 1 | A | A | . | . | . | . | . | −0.06 | −0.60 | * | . | . | 0.75 | 1.08 |
| Ala | 2 | A | A | . | . | . | . | . | −0.01 | −0.34 | . | . | . | 0.30 | 0.70 |
| Glu | 3 | A | A | . | . | . | . | . | −0.43 | −0.34 | . | . | . | 0.30 | 0.54 |
| Asp | 4 | A | A | . | . | . | . | . | −0.34 | −0.09 | * | * | . | 0.30 | 0.45 |
| Leu | 5 | A | A | . | B | . | . | . | −0.66 | −0.31 | * | . | . | 0.30 | 0.60 |
| Gly | 6 | A | A | . | B | . | . | . | −0.40 | −0.03 | * | . | . | 0.30 | 0.30 |
| Leu | 7 | A | . | . | B | . | . | . | 0.19 | 0.40 | * | . | . | −0.60 | 0.18 |
| Ser | 8 | A | . | . | . | . | . | . | −0.12 | 0.40 | * | . | . | −0.40 | 0.37 |
| Phe | 9 | A | . | . | . | . | . | . | −0.71 | 0.20 | * | * | . | −0.10 | 0.54 |
| Gly | 10 | A | . | . | . | . | . | . | −0.20 | 0.27 | . | . | F | 0.05 | 0.66 |
| Glu | 11 | A | . | . | . | . | . | . | −0.71 | −0.03 | . | . | F | 0.65 | 0.66 |
| Thr | 12 | A | . | . | . | . | . | . | 0.10 | 0.23 | . | * | F | 0.05 | 0.57 |
| Ala | 13 | A | A | . | . | . | . | . | −0.20 | −0.56 | . | . | F | 0.75 | 1.00 |
| Ser | 14 | A | A | . | . | . | . | . | −0.31 | −0.37 | . | . | . | 0.30 | 0.57 |
| Val | 15 | A | A | . | . | . | . | . | −0.18 | 0.31 | . | . | . | −0.30 | 0.33 |
| Glu | 16 | A | A | . | . | . | . | . | −0.18 | 0.26 | . | . | . | −0.30 | 0.50 |
| Met | 17 | A | A | . | . | . | . | . | 0.10 | −0.24 | . | . | . | 0.30 | 0.64 |
| Leu | 18 | A | A | . | . | . | . | . | 0.34 | −0.13 | * | . | . | 0.45 | 1.18 |
| Pro | 19 | A | . | . | . | . | . | . | 0.34 | −0.34 | * | . | . | 0.81 | 0.67 |
| Glu | 20 | A | . | . | . | . | . | . | 0.53 | 0.04 | . | * | F | 0.67 | 0.91 |
| His | 21 | A | . | . | . | . | T | . | 0.64 | 0.00 | . | * | F | 1.78 | 0.59 |
| Gly | 22 | . | . | . | . | T | T | . | 1.03 | −0.69 | . | * | F | 2.79 | 0.75 |
| Ser | 23 | . | . | . | . | T | T | . | 1.89 | −0.69 | . | * | F | 3.10 | 0.67 |
| Cys | 24 | A | . | . | . | . | T | . | 1.51 | −0.69 | . | * | F | 2.39 | 0.99 |
| Arg | 25 | . | . | B | . | . | . | . | 1.62 | −0.69 | . | * | F | 2.03 | 1.01 |
| Pro | 26 | . | . | . | . | T | . | . | 1.36 | −1.11 | * | * | F | 2.42 | 1.47 |
| Lys | 27 | . | . | . | . | T | . | . | 1.40 | −1.11 | . | * | F | 2.41 | 3.68 |
| Ala | 28 | . | . | . | . | T | . | . | 1.40 | −1.30 | . | * | F | 2.40 | 2.52 |
| Arg | 29 | . | . | B | . | . | T | . | 1.48 | −0.91 | . | * | F | 2.50 | 2.18 |
| Ser | 30 | . | . | . | . | . | T | C | 1.48 | −0.84 | . | * | F | 3.00 | 1.10 |
| Ser | 31 | . | . | . | . | . | T | C | 1.40 | −0.84 | * | * | F | 2.70 | 2.14 |
| Ser | 32 | . | . | . | . | T | T | . | 0.77 | −0.43 | * | * | F | 2.30 | 1.15 |
| Ala | 33 | . | . | . | . | T | . | . | 0.54 | 0.07 | . | * | F | 1.05 | 0.87 |
| Arg | 34 | . | . | . | B | T | . | . | 0.12 | 0.37 | . | * | . | 0.40 | 0.53 |
| Trp | 35 | . | . | . | B | T | . | . | −0.24 | 0.47 | * | * | . | −0.20 | 0.57 |
| Ala | 36 | A | . | . | B | . | . | . | −0.61 | 0.66 | . | * | . | −0.60 | 0.30 |
| Leu | 37 | . | . | B | B | . | . | . | −1.12 | 0.73 | . | * | . | −0.60 | 0.08 |
| Thr | 38 | . | . | B | B | . | . | . | −1.39 | 1.41 | * | * | . | −0.60 | 0.07 |
| Cys | 39 | . | . | B | B | . | . | . | −2.31 | 1.14 | * | * | . | −0.60 | 0.05 |
| Cys | 40 | . | . | B | B | . | . | . | −2.83 | 1.33 | . | . | . | −0.60 | 0.05 |
| Leu | 41 | . | . | B | B | . | . | . | −2.46 | 1.33 | . | . | . | −0.60 | 0.03 |
| Val | 42 | . | . | B | B | . | . | . | −2.34 | 1.27 | . | . | . | −0.60 | 0.08 |
| Leu | 43 | . | . | B | B | . | . | . | −2.84 | 1.49 | . | . | . | −0.60 | 0.13 |
| Leu | 44 | . | . | B | B | . | . | . | −2.77 | 1.60 | . | . | . | −0.60 | 0.13 |
| Pro | 45 | . | . | B | B | . | . | . | −2.44 | 1.41 | . | . | . | −0.60 | 0.17 |
| Phe | 46 | . | . | B | B | . | . | . | −2.44 | 1.20 | . | . | . | −0.60 | 0.21 |
| Leu | 47 | A | . | . | B | . | . | . | −1.90 | 1.20 | . | . | . | −0.60 | 0.21 |
| Ala | 48 | A | . | . | B | . | . | . | −1.40 | 1.00 | . | . | . | −0.60 | 0.19 |
| Gly | 49 | . | . | B | B | . | . | . | −0.83 | 1.06 | . | . | . | −0.60 | 0.32 |
| Leu | 50 | . | . | B | B | . | . | . | −1.43 | 1.03 | . | . | . | −0.60 | 0.62 |
| Thr | 51 | . | . | B | B | . | . | . | −1.54 | 1.03 | . | . | . | −0.60 | 0.50 |
| Thr | 52 | . | A | B | B | . | . | . | −1.59 | 1.21 | . | . | . | −0.60 | 0.42 |
| Tyr | 53 | . | A | B | B | . | . | . | −1.30 | 1.43 | . | . | . | −0.60 | 0.38 |
| Leu | 54 | . | A | B | B | . | . | . | −0.96 | 1.13 | . | . | . | −0.60 | 0.35 |
| Leu | 55 | . | A | B | B | . | . | . | −0.96 | 1.04 | . | * | . | −0.60 | 0.42 |
| Val | 56 | . | A | B | B | . | . | . | −0.53 | 1.24 | . | * | . | −0.60 | 0.22 |
| Ser | 57 | . | A | B | B | . | . | . | −0.81 | 0.49 | . | * | . | −0.60 | 0.53 |
| Gln | 58 | . | A | B | B | . | . | . | −0.57 | 0.30 | . | * | . | −0.30 | 0.64 |
| Leu | 59 | . | A | B | B | . | . | . | −0.10 | 0.01 | * | * | . | −0.15 | 1.50 |
| Arg | 60 | A | A | . | B | . | . | . | 0.71 | −0.20 | * | * | . | 0.45 | 1.11 |
| Ala | 61 | A | A | . | B | . | . | . | 0.98 | −0.59 | . | * | F | 0.90 | 1.11 |
| Gln | 62 | A | A | . | . | . | . | . | 0.61 | −0.49 | . | * | F | 0.60 | 1.36 |
| Gly | 63 | . | A | . | . | . | . | C | −0.24 | −0.60 | * | * | F | 0.95 | 0.37 |
| Glu | 64 | A | A | . | . | . | . | . | 0.57 | 0.04 | * | * | F | −0.15 | 0.27 |
| Ala | 65 | A | A | . | . | . | . | . | −0.24 | −0.06 | * | * | . | 0.30 | 0.27 |
| Cys | 66 | A | A | . | . | . | . | . | 0.34 | 0.33 | . | * | . | −0.30 | 0.24 |
| Val | 67 | A | A | . | . | . | . | . | −0.24 | 0.30 | . | * | . | −0.30 | 0.24 |
| Gln | 68 | A | A | . | . | . | . | . | −0.71 | 0.80 | . | * | . | −0.60 | 0.24 |
| Phe | 69 | A | A | . | . | . | . | . | −0.67 | 0.99 | . | * | . | −0.60 | 0.37 |
| Gln | 70 | A | A | . | . | . | . | . | −0.42 | 0.41 | . | * | . | −0.60 | 0.99 |
| Ala | 71 | A | A | . | . | . | . | . | 0.24 | 0.20 | . | . | . | −0.30 | 0.57 |
| Leu | 72 | A | A | . | . | . | . | . | 1.10 | 0.20 | . | . | F | 0.00 | 1.13 |
| Lys | 73 | . | A | . | . | . | . | C | 0.40 | −0.59 | . | . | F | 1.10 | 1.13 |
| Gly | 74 | . | A | . | . | . | . | C | 0.51 | −0.20 | . | . | F | 0.65 | 0.97 |
| Gln | 75 | . | A | . | . | . | . | C | 0.30 | −0.20 | . | . | F | 0.80 | 1.19 |
| Glu | 76 | . | A | . | . | . | . | C | 0.59 | −0.46 | . | . | F | 0.65 | 0.92 |
| Phe | 77 | A | A | . | . | . | . | . | 1.37 | −0.07 | . | . | F | 0.60 | 1.25 |
| Ala | 78 | . | A | . | . | . | . | C | 1.32 | 0.00 | * | . | F | 0.65 | 0.98 |
| Pro | 79 | A | . | . | . | . | T | . | 1.67 | 0.00 | * | . | F | 0.85 | 0.98 |
| Ser | 80 | . | . | . | . | T | T | . | 0.81 | 0.40 | * | . | F | 0.80 | 1.96 |
| His | 81 | A | . | . | . | T | T | . | 0.57 | 0.26 | . | . | F | 0.80 | 1.44 |
| Gln | 82 | . | . | . | . | T | T | . | 0.68 | 0.51 | . | . | F | 0.50 | 1.46 |
| Gln | 83 | . | . | B | . | . | . | . | 1.06 | 0.59 | . | . | . | −0.25 | 1.10 |
| Val | 84 | . | . | B | . | . | . | . | 0.46 | 0.63 | * | * | . | −0.25 | 1.25 |
| Tyr | 85 | . | . | B | . | . | . | . | 0.87 | 0.81 | * | * | . | −0.40 | 0.59 |
| Ala | 86 | . | A | B | . | . | . | . | 0.31 | 0.41 | * | * | . | −0.60 | 0.67 |
| Pro | 87 | . | A | B | . | . | . | . | 0.31 | 0.51 | * | * | . | −0.26 | 0.92 |
| Leu | 88 | . | A | B | . | . | . | . | −0.03 | −0.13 | * | * | . | 0.98 | 0.98 |
| Arg | 89 | . | A | B | . | . | . | . | 0.82 | −0.46 | * | * | . | 1.32 | 0.96 |
| Ala | 90 | . | . | . | . | T | . | . | 1.11 | −0.96 | . | * | F | 2.86 | 1.03 |
| Asp | 91 | . | . | . | . | T | T | . | 1.49 | −1.39 | * | * | F | 3.40 | 2.50 |
| Gly | 92 | . | . | . | . | T | T | . | 1.81 | −1.64 | * | * | F | 3.06 | 1.98 |
| Asp | 93 | . | . | . | . | . | T | C | 2.03 | −1.64 | * | * | F | 2.52 | 3.83 |
| Lys | 94 | . | . | . | . | . | T | C | 1.89 | −1.64 | * | * | F | 2.18 | 2.32 |
| Pro | 95 | A | . | . | . | . | . | . | 1.67 | −1.14 | . | * | F | 1.44 | 3.19 |
| Arg | 96 | A | . | . | . | . | . | . | 1.36 | −0.89 | . | * | F | 1.10 | 1.57 |
| Ala | 97 | A | . | . | B | . | . | . | 0.84 | −0.40 | * | * | . | 0.45 | 1.14 |
| His | 98 | . | . | B | B | . | . | . | −0.01 | 0.24 | * | * | . | −0.30 | 0.55 |
| Leu | 99 | . | . | B | B | . | . | . | 0.06 | 0.46 | * | * | . | −0.60 | 0.21 |
| Thr | 100 | . | . | B | B | . | . | . | 0.27 | 0.46 | * | * | . | −0.60 | 0.40 |
| Val | 101 | . | . | B | B | . | . | . | −0.16 | 0.36 | * | * | . | −0.30 | 0.51 |
| Val | 102 | . | . | B | B | . | . | . | 0.22 | 0.34 | * | . | . | −0.06 | 0.89 |
| Arg | 103 | . | . | B | B | . | . | . | −0.06 | 0.09 | * | . | F | 0.33 | 0.96 |
| Gln | 104 | . | . | B | B | . | . | . | 0.76 | 0.09 | * | . | F | 0.72 | 1.86 |
| Thr | 105 | . | . | B | . | . | T | . | 1.03 | −0.16 | * | . | F | 1.96 | 4.34 |
| Pro | 106 | . | . | . | . | . | T | C | 1.19 | −0.30 | * | * | F | 2.40 | 3.01 |
| Thr | 107 | . | . | . | . | T | T | . | 2.09 | 0.49 | * | * | F | 1.46 | 1.51 |
| Gln | 108 | . | . | B | . | . | T | . | 1.98 | 0.09 | . | * | F | 1.12 | 2.09 |
| His | 109 | . | A | . | . | T | . | . | 1.98 | 0.00 | * | * | F | 1.48 | 2.17 |
| Phe | 110 | . | A | . | . | T | . | . | 1.59 | −0.03 | * | * | F | 1.24 | 2.61 |
| Lys | 111 | . | A | . | . | T | . | . | 1.59 | 0.27 | * | * | F | 0.40 | 1.30 |
| Asn | 112 | . | A | . | . | T | . | . | 1.31 | 0.30 | * | * | F | 0.40 | 1.48 |
| Gln | 113 | . | A | . | . | . | . | C | 0.50 | 0.30 | * | . | F | 0.20 | 1.73 |
| Phe | 114 | . | A | . | . | . | . | C | 0.50 | 0.20 | . | * | . | −0.10 | 0.71 |
| Pro | 115 | . | A | . | . | . | . | C | 0.91 | 0.70 | . | * | . | −0.40 | 0.60 |
| Ala | 116 | A | A | . | . | . | . | . | 0.87 | 1.21 | . | . | . | −0.60 | 0.37 |
| Leu | 117 | A | A | . | . | . | . | . | 0.83 | 0.81 | . | . | . | −0.60 | 0.73 |
| His | 118 | A | A | . | . | . | . | . | 0.83 | 0.53 | . | * | . | −0.60 | 0.64 |
| Trp | 119 | A | A | . | . | . | . | . | 0.72 | 0.10 | . | . | . | −0.15 | 1.10 |
| Glu | 120 | A | A | . | . | . | . | . | 0.59 | 0.29 | . | * | . | −0.15 | 1.10 |
| His | 121 | A | A | . | . | . | . | . | 0.37 | 0.03 | . | * | . | −0.30 | 0.80 |
| Glu | 122 | A | A | . | . | . | . | . | 0.59 | 0.21 | . | * | . | −0.30 | 0.63 |
| Leu | 123 | A | A | . | . | . | . | . | −0.08 | −0.20 | . | * | . | 0.30 | 0.37 |
| Gly | 124 | A | A | . | . | . | . | . | −0.10 | 0.59 | * | * | . | −0.60 | 0.23 |
| Leu | 125 | A | A | . | . | . | . | . | −0.06 | 0.57 | . | . | . | −0.60 | 0.19 |
| Ala | 126 | A | A | . | . | . | . | . | −0.02 | 0.57 | . | . | . | −0.60 | 0.47 |
| Phe | 127 | A | A | . | . | . | . | . | 0.09 | 0.29 | . | * | . | −0.02 | 0.77 |
| Thr | 128 | A | . | . | . | . | T | . | 0.30 | −0.14 | . | * | F | 1.56 | 1.82 |
| Lys | 129 | A | . | . | . | . | T | . | 0.64 | −0.21 | . | * | F | 1.84 | 1.79 |
| Asn | 130 | A | . | . | . | . | T | . | 1.21 | −0.31 | . | * | F | 2.12 | 3.32 |
| Arg | 131 | . | . | . | . | T | T | . | 1.49 | −0.34 | . | * | F | 2.80 | 3.60 |
| Met | 132 | . | . | . | . | T | . | . | 2.19 | −0.34 | . | * | . | 2.17 | 2.60 |
| Asn | 133 | . | . | . | . | T | . | . | 2.54 | 0.06 | . | * | . | 1.29 | 2.60 |
| Tyr | 134 | . | . | . | . | T | T | . | 1.80 | −0.34 | * | * | F | 1.96 | 2.65 |
| Thr | 135 | . | . | . | . | T | T | . | 0.99 | 0.44 | * | * | F | 0.78 | 2.32 |
| Asn | 136 | . | . | B | . | . | T | . | 0.07 | 0.51 | . | * | F | 0.10 | 1.19 |
| Lys | 137 | . | . | B | . | . | T | . | −0.22 | 0.80 | * | . | F | −0.05 | 0.63 |
| Phe | 138 | . | A | B | B | . | . | . | −0.43 | 0.73 | * | . | . | −0.60 | 0.30 |
| Leu | 139 | . | A | B | B | . | . | . | −0.19 | 0.67 | . | . | . | −0.60 | 0.29 |
| Leu | 140 | . | A | B | B | . | . | . | −0.18 | 0.27 | * | . | . | −0.02 | 0.25 |
| Ile | 141 | . | A | B | B | . | . | . | −0.52 | 0.66 | * | . | . | −0.04 | 0.39 |
| Pro | 142 | . | . | B | B | . | . | . | −0.57 | 0.30 | . | . | F | 0.69 | 0.47 |
| Glu | 143 | . | . | . | . | T | . | . | −0.11 | −0.39 | * | . | F | 2.17 | 0.95 |
| Ser | 144 | . | . | . | . | T | T | . | 0.00 | −0.31 | * | . | F | 2.80 | 2.13 |
| Gly | 145 | . | . | . | . | T | T | . | −0.08 | −0.21 | * | . | F | 2.52 | 1.19 |
| Asp | 146 | . | . | . | . | T | T | . | 0.57 | 0.04 | * | . | F | 1.49 | 0.48 |
| Tyr | 147 | . | . | B | . | . | T | . | 0.48 | 0.80 | . | . | . | 0.36 | 0.56 |
| Phe | 148 | . | . | B | B | . | . | . | 0.48 | 0.80 | . | . | . | −0.32 | 0.76 |
| Ile | 149 | . | . | B | B | . | . | . | −0.08 | 0.77 | . | . | . | −0.60 | 0.79 |
| Tyr | 150 | . | . | B | B | . | . | . | −0.04 | 1.41 | . | * | . | −0.60 | 0.38 |
| Ser | 151 | . | . | B | B | . | . | . | −0.74 | 1.14 | . | * | . | −0.60 | 0.63 |
| Gln | 152 | . | . | B | B | . | . | . | −0.39 | 1.14 | . | * | . | −0.60 | 0.77 |
| Val | 153 | . | . | B | B | . | . | . | −0.03 | 0.46 | . | * | . | −0.60 | 0.97 |
| Thr | 154 | . | . | B | B | . | . | . | 0.26 | 0.13 | . | * | . | −0.30 | 0.71 |
| Phe | 155 | . | . | B | B | . | . | . | 0.19 | 0.36 | * | * | . | −0.30 | 0.41 |
| Arg | 156 | . | . | B | B | . | . | . | 0.19 | 0.44 | * | * | . | −0.60 | 0.79 |
| Gly | 157 | . | . | . | B | T | . | . | 0.19 | 0.19 | * | * | F | 0.25 | 0.74 |
| Met | 158 | . | . | . | B | . | . | C | 0.38 | −0.30 | * | * | F | 0.80 | 1.47 |
| Thr | 159 | . | . | . | . | . | T | C | 0.39 | −0.51 | * | * | F | 1.35 | 0.40 |
| Ser | 160 | . | . | . | . | . | T | C | 1.09 | −0.13 | * | * | F | 1.05 | 0.55 |
| Glu | 161 | A | . | . | . | . | T | . | 0.09 | −0.56 | * | * | F | 1.15 | 0.96 |
| Cys | 162 | A | . | . | . | . | T | . | 0.54 | −0.49 | * | . | F | 0.85 | 0.46 |
| Ser | 163 | A | A | . | . | . | . | . | 1.14 | −0.97 | * | . | F | 0.75 | 0.68 |
| Glu | 164 | A | A | . | . | . | . | . | 0.87 | −0.96 | * | . | F | 0.75 | 0.68 |
| Ile | 165 | A | A | . | . | . | . | . | 0.82 | −0.46 | * | * | F | 0.60 | 1.28 |
| Arg | 166 | A | A | . | . | . | . | . | 0.93 | −0.60 | * | * | F | 0.75 | 0.94 |
| Gln | 167 | A | A | . | . | . | . | . | 1.39 | −0.99 | * | * | F | 1.24 | 1.07 |
| Ala | 168 | . | A | . | . | T | . | . | 1.69 | −0.56 | * | * | F | 1.98 | 2.35 |
| Gly | 169 | . | A | . | . | . | . | C | 1.73 | −0.84 | * | * | F | 2.12 | 1.93 |
| Arg | 170 | . | . | . | . | . | T | C | 2.41 | −0.84 | * | * | F | 2.86 | 2.23 |
| Pro | 171 | . | . | . | . | T | T | . | 2.30 | −0.81 | * | * | F | 3.40 | 3.42 |
| Asn | 172 | . | . | . | . | T | T | . | 2.00 | −1.31 | . | . | F | 3.06 | 5.77 |
| Lys | 173 | . | . | . | . | . | T | C | 1.70 | −1.36 | . | . | F | 2.52 | 3.94 |
| Pro | 174 | . | . | . | . | . | T | C | 1.73 | −0.67 | . | . | F | 2.18 | 1.79 |
| Asp | 175 | . | . | . | . | T | T | . | 0.77 | −0.61 | . | . | F | 2.04 | 1.60 |
| Ser | 176 | . | . | B | . | . | T | . | 0.12 | −0.37 | . | . | F | 0.85 | 0.60 |
| Ile | 177 | . | . | B | . | . | T | . | −0.77 | 0.27 | . | . | . | 0.10 | 0.29 |
| Thr | 178 | . | . | B | B | . | . | . | −1.12 | 0.53 | * | . | . | −0.60 | 0.12 |
| Val | 179 | . | . | B | B | . | . | . | −0.87 | 1.01 | * | . | . | −0.60 | 0.13 |
| Val | 180 | . | . | B | B | . | . | . | −1.72 | 0.63 | * | . | . | −0.60 | 0.37 |
| Ile | 181 | . | . | B | B | . | . | . | −1.73 | 0.59 | * | . | . | −0.60 | 0.19 |
| Thr | 182 | . | . | B | B | . | . | . | −0.84 | 0.59 | * | . | . | −0.60 | 0.37 |
| Lys | 183 | . | . | B | B | . | . | . | −0.83 | −0.06 | * | . | F | 0.45 | 0.83 |
| Val | 184 | . | . | B | B | . | . | . | −0.22 | −0.31 | * | . | F | 0.90 | 1.59 |
| Thr | 185 | . | . | B | B | . | . | . | 0.42 | −0.24 | * | . | F | 1.20 | 1.72 |
| Asp | 186 | . | . | . | . | T | T | . | 1.31 | −0.30 | * | . | F | 2.30 | 1.33 |
| Ser | 187 | . | . | . | . | . | T | C | 1.41 | −0.30 | * | . | F | 2.40 | 3.11 |
| Tyr | 188 | . | . | . | . | . | T | C | 1.06 | −0.51 | * | . | F | 3.00 | 3.33 |
| Pro | 189 | . | . | . | . | . | T | C | 1.91 | −0.51 | * | . | F | 2.70 | 2.88 |
| Glu | 190 | . | . | . | . | . | T | C | 1.41 | −0.11 | * | . | F | 2.10 | 3.72 |
| Pro | 191 | A | . | . | . | . | T | . | 0.60 | 0.19 | * | . | F | 1.00 | 1.96 |
| Thr | 192 | A | . | . | . | . | T | . | 0.30 | 0.11 | . | . | F | 0.70 | 1.04 |
| Gln | 193 | A | . | . | . | . | T | . | 0.20 | 0.30 | . | . | F | 0.25 | 0.60 |
| Leu | 194 | . | . | B | . | . | . | . | 0.10 | 0.73 | . | . | . | −0.40 | 0.38 |
| Leu | 195 | . | . | B | . | . | . | . | 0.14 | 0.79 | . | . | . | −0.40 | 0.38 |
| Met | 196 | A | . | . | . | . | . | . | 0.06 | 0.30 | * | . | . | −0.10 | 0.44 |
| Gly | 197 | . | . | . | . | T | T | . | −0.49 | 0.29 | * | . | F | 0.65 | 0.72 |
| Thr | 198 | . | . | . | . | T | T | . | −1.16 | 0.24 | * | . | F | 0.65 | 0.64 |
| Lys | 199 | . | . | B | . | . | T | . | −0.34 | 0.13 | * | . | F | 0.25 | 0.35 |
| Ser | 200 | . | . | B | . | . | T | . | −0.39 | −0.49 | * | . | F | 0.85 | 0.61 |
| Val | 201 | . | . | B | B | . | . | . | −0.13 | −0.27 | * | . | . | 0.30 | 0.31 |
| Cys | 202 | . | . | B | B | . | . | . | −0.09 | −0.33 | * | . | . | 0.30 | 0.16 |
| Glu | 203 | . | . | B | B | . | . | . | 0.22 | 0.06 | * | . | . | −0.30 | 0.16 |
| Val | 204 | . | . | B | B | . | . | . | −0.11 | 0.07 | * | . | . | −0.30 | 0.34 |
| Gly | 205 | . | . | . | . | T | T | . | −0.51 | 0.34 | * | . | F | 0.65 | 0.66 |
| Ser | 206 | . | . | . | . | T | T | . | 0.34 | 0.56 | * | . | F | 0.35 | 0.33 |
| Asn | 207 | . | . | . | . | T | T | . | 0.80 | 0.96 | * | . | F | 0.35 | 0.77 |
| Trp | 208 | . | . | . | . | T | T | . | −0.09 | 0.74 | * | . | . | 0.35 | 1.20 |
| Phe | 209 | . | . | B | B | . | . | . | 0.52 | 1.00 | * | . | . | −0.60 | 0.63 |
| Gln | 210 | . | . | B | B | . | . | . | 0.06 | 1.37 | * | . | . | −0.60 | 0.61 |
| Pro | 211 | . | . | B | B | . | . | . | 0.01 | 1.66 | * | . | . | −0.60 | 0.48 |
| Ile | 212 | . | . | B | B | . | . | . | −0.58 | 1.17 | * | . | . | −0.60 | 0.55 |
| Tyr | 213 | . | . | B | B | . | . | . | −0.89 | 0.89 | . | . | . | −0.60 | 0.32 |
| Leu | 214 | . | . | B | B | . | . | . | −0.89 | 1.10 | . | . | . | −0.60 | 0.21 |
| Gly | 215 | . | . | . | B | . | . | C | −1.19 | 1.46 | . | * | . | −0.40 | 0.25 |
| Ala | 216 | . | A | B | . | . | . | . | −1.79 | 1.16 | . | * | . | −0.60 | 0.22 |
| Met | 217 | . | A | B | . | . | . | . | −0.90 | 1.09 | . | * | . | −0.60 | 0.22 |
| Phe | 218 | . | A | B | . | . | . | . | −0.66 | 0.80 | . | . | . | −0.60 | 0.38 |
| Ser | 219 | A | A | . | . | . | . | . | −0.19 | 0.37 | . | . | . | −0.30 | 0.65 |
| Leu | 220 | A | A | . | . | . | . | . | 0.16 | 0.30 | * | . | . | −0.30 | 0.65 |
| Gln | 221 | A | A | . | . | . | . | . | 0.79 | −0.31 | * | . | F | 0.60 | 1.26 |
| Glu | 222 | A | . | . | . | . | T | . | 0.58 | −1.10 | * | . | F | 1.30 | 1.87 |
| Gly | 223 | A | . | . | . | . | T | . | 0.68 | −0.80 | * | * | F | 1.30 | 1.87 |
| Asp | 224 | A | . | . | . | . | T | . | 0.12 | −0.87 | * | . | F | 1.30 | 1.07 |
| Lys | 225 | A | . | . | . | . | T | . | 0.93 | −0.63 | * | * | F | 1.15 | 0.46 |
| Leu | 226 | A | . | . | B | . | . | . | 0.08 | −0.23 | * | * | . | 0.30 | 0.75 |
| Met | 227 | . | . | B | B | . | . | . | −0.22 | −0.01 | * | . | . | 0.30 | 0.33 |
| Val | 228 | . | . | B | B | . | . | . | 0.12 | 0.37 | * | . | . | −0.30 | 0.22 |
| Asn | 229 | . | . | B | . | . | T | . | −0.77 | 0.37 | * | . | . | 0.10 | 0.45 |
| Val | 230 | . | . | B | . | . | T | . | −1.11 | 0.37 | * | * | . | 0.10 | 0.32 |
| Ser | 231 | . | . | B | . | . | T | . | −1.11 | 0.14 | . | . | F | 0.25 | 0.58 |
| Asp | 232 | . | . | B | . | . | T | . | −1.37 | 0.19 | . | . | F | 0.25 | 0.29 |
| Ile | 233 | . | . | B | B | . | . | . | −0.51 | 0.43 | . | . | . | −0.60 | 0.29 |
| Ser | 234 | . | . | B | B | . | . | . | −0.76 | −0.21 | . | . | . | 0.30 | 0.37 |
| Leu | 235 | . | . | B | B | . | . | . | −0.21 | 0.16 | . | . | . | −0.30 | 0.34 |
| Val | 236 | . | . | B | B | . | . | . | 0.13 | 0.64 | . | . | . | −0.60 | 0.71 |
| Asp | 237 | A | . | . | B | . | . | . | 0.13 | −0.04 | . | . | . | 0.45 | 1.06 |
| Tyr | 238 | A | A | . | . | . | . | . | 1.02 | −0.43 | . | . | . | 0.45 | 2.23 |
| Thr | 239 | A | A | . | . | . | . | . | 1.37 | −1.11 | . | . | F | 0.90 | 5.01 |
| Lys | 240 | A | A | . | . | . | . | . | 1.87 | −1.76 | * | . | F | 0.90 | 6.00 |
| Glu | 241 | A | A | . | . | . | . | . | 2.02 | −1.27 | * | . | F | 0.90 | 5.52 |
| Asp | 242 | A | A | . | . | . | . | . | 1.32 | −1.24 | * | . | F | 0.90 | 3.31 |
| Lys | 243 | A | A | . | . | . | . | . | 1.22 | −0.94 | * | . | F | 0.90 | 1.43 |
| Thr | 244 | A | A | . | . | . | . | . | 0.94 | −0.51 | * | . | F | 0.75 | 0.82 |
| Phe | 245 | A | A | . | . | . | . | . | 0.20 | −0.01 | * | . | . | 0.30 | 0.50 |
| Phe | 246 | A | A | . | . | . | . | . | −0.61 | 0.77 | . | . | . | −0.60 | 0.21 |
| Gly | 247 | A | A | . | . | . | . | . | −1.42 | 1.46 | . | . | . | −0.60 | 0.12 |
| Ala | 248 | A | A | . | . | . | . | . | −1.86 | 1.66 | . | . | . | −0.60 | 0.12 |
| Phe | 249 | A | A | . | . | . | . | . | −1.93 | 1.30 | . | . | . | −0.60 | 0.17 |
| Leu | 250 | A | A | . | . | . | . | . | −1.62 | 0.94 | . | . | . | −0.60 | 0.22 |
| Leu | 251 | A | A | . | . | . | . | . | −1.31 | 0.94 | . | . | . | −0.60 | 0.28 |

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids, ranging from 1-411, can be deleted from the amino terminus of either the secreted polypeptide or the mature form of a polypeptide having an amino acid sequence shown in SEQ ID NO:4. Similarly, for example, any number of amino acids, ranging from 1-411, can be deleted from the carboxy terminus of the secreted protein or mature form of a polypeptide having an amino acid sequence shown in SEQ ID NO:4. Furthermore, any combination of the above amino and carboxy terminus deletions are preferred. Polynucleotides encoding these polypeptide fragments and antibodies that bind these polypeptide fragments are encompassed by the invention.

Accordingly, the present invention further provides polypeptides having one or more residues deleted from the amino terminus of the amino acid sequence of SEQ ID NO:4 (FIGS. 3A-3C), and polynucleotides encoding such polypeptides. For example, the present invention provides polypeptides comprising, or alternatively consisting of, the amino acid sequence of residues n¹-417 of SEQ ID NO:4, where n¹ is an integer in the range of 2-412. More in particular, in certain embodiments, the invention provides polynucleotides encoding polypeptides comprising, or alternatively consisting of, an amino acid sequence selected from the group consisting of residues of E-2 to P-417; Q-3 to P-417; R-4 toP-417; P-5 to P-417; R-6 to P-417; G-7 to P-417;C-8 to P-417; A-9 to P-417; A-10 to P-417; V-1 Ito P-417; A-12 to P-417; A-13 to P-417; A-14 toP-417; L-15 to P-417; L-16 to P-417; L-17 toP-417; V-18 to P-417; L-19 to P-417; L-20 toP-417; G-21 to P-417; A-22 to P-417; R-23 toP-417; A-24 to P-417; Q-25 to P-417; G-26 toP-417; G-27 to P-417; T-28 to P-417; R-29 toP-417; S-30 to P-417; P-31 to P-417; R-32 toP-417; C-33 to P-417; D-34 to P-417; C-35 toP-417; A-36 to P-417; G-37 to P-417; D-38 toP-417; F-39 to P-417; H-40 to P-417; K-41 toP-417; K-42 to P-417; 1-43 to P-417; G-44 toP-417; L-45 to P-417; F-46 to P-417; C-47 toP-417; C-48 to P-417; R-49 to P-417; G-50 toP-417; C-51 to P-417; P-52 to P-417; A-53 toP-417; G-54 to P-417; H-55 to P-417; Y-56 toP-417; L-57 to P-417; K-58 to P-417; A-59 toP-417; P-60 to P-417; C-61 to P-417; T-62 toP-417; E-63 to P-417; P-64 to P-417; C-65 toP-417; G-66 to P-417; N-67 to P-417; S-68 toP-417; T-69 to P-417; C-70 to P-417; L-71 toP-417; V-72 to P-417; C-73 to P-417; P-74 toP-417; Q-75 to P-417; D-76 to P-417; T-77 toP-417; F-78 to P-417; L-79 to P-417; A-80 toP-417; W-81 to P-417; E-82 to P-417; N-83 toP-417; H-84 to P-417; H-85 to P-417; N-86 toP-417; S-87 to P-417; E-88 to P-417; C-89 toP-417; A-90 to P-417; R-91 to P-417; C-92 toP-417; Q-93 to P-417; A-94 to P-417; C-95 toP-417; D-96 to P-417; E-97 to P-417; Q-98 toP-417; A-99 to P-417; S-100 to P-417; Q-101 toP-417; V-102 to P-417; A-103 to P-417; L-104 toP-417; E-105 to P-417; N-106 to P-417; C-107 to P-417; S-108 to P-417; A-109 to P-417; V-110 to P-417; A-Ill to P-417; D-112 to P-417; T-113 to P-417; R-114 to P-417; C-115 to P-417;G-116 to P-417; C-117 to P-417; K-118 toP-417; P-119 to P-417; G-120 to P-417; W-121 to P-417; F-122 to P-417; V-123 to P-417; E-124 to P-417; C-125 to P-417; Q-126 to P-417;V-127 to P-417; S-128 to P-417; Q-129 toP-417; C-130 to P-417; V-131 to P-417; S-132 toP-417; S-133 to P-417; S-134 to P-417; P-135 toP-417; F-136 to P-417; Y-137 to P-417; C-138 toP-417; Q-139 to P-417; P-140 to P-417; C-141 to P-417; L-142 to P-417; D-143 to P-417; C-144 to P-417; G-145 to P-417; A-146 to P-417; L-147 to P-417; H-148 to P-417; R-149 to P-417;H-150 to P-417; T-151 to P-417; R-152 to P-417;L-153 to P-417; L-154 to P-417; C-155 to P-417;S-156 to P-417; R-157 to P-417; R-158 to P-417;D-159 to P-417; T-160 to P-417; D-161 to P-417;C-162 to P-417; G-163 to P-417; T-164 to P-417;C-165 to P-417; L-166 to P-417; P-167 to P-417;G-168 to P-417; F-169 to P-417; Y-170 to P-417;E-171 to P-417; H-172 to P-417; G-173 to P-417;D-174 to P-417; G-175 to P-417; C-176 toP-417; V-177 to P-417; S-178 to P-417; C-179 toP-417; P-180 to P-417; T-181 to P-417; S-182 toP-417; T-183 to P-417; L-184 to P-417; G-185 toP-417; S-186 to P-417; C-187 to P-417; P-188 toP-417; E-189 to P-417; R-190 to P-417; C-191 toP-417; A-192 to P-417; A-193 to P-417; V-194 to P-417; C-195 to P-417; G-196 to P-417;W-197 to P-417; R-198 to P-417; Q-199 toP-417; M-200 to P-417; F-201 to P-417; W-202 to P-417; V-203 to P-417; Q-204 to P-417;V-205 to P-417; L-206 to P-417; L-207 to P-417;A-208 to P-417; G-209 to P-417; L-210 to P-417;V-211 to P-417; V-212 to P-417; P-213 to P-417;L-214 to P-417; L-215 to P-417; L-216 to P-417;G-217 to P-417; A-218 to P-417; T-219 to P-417;L-220 to P-417; T-221 to P-417; Y-222 to P-417;T-223 to P-417; Y-224 to P-417; R-225 to P-417;H-226 to P-417; C-227 to P-417; W-228 toP-417; P-229 to P-417; H-230 to P-417; K-23Ito P-417; P-232 to P-417; L-233 to P-417; V-234 to P-417; T-235 to P-417; A-236 to P-417; D-237 to P-417; E-238 to P-417; A-239 to P-417; G-240 to P-417; M-241 to P-417; E-242 to P-417;A-243 to P-417; L-244 to P-417; T-245 to P-417;P-246 to P-417; P-247 to P-417; P-248 to P-417;A-249 to P-417; T-250 to P-417; H-251 to P-417;L-252 to P-417; S-253 to P-417; P-254 to P-417;L-255 to P-417; D-256 to P-417; S-257 to P-417;A-258 to P-417; H-259 to P-417; T-260 to P-417;L-261 to P-417; L-262 to P-417; A-263 to P-417;P-264 to P-417; P-265 to P-417; D-266 to P-417;S-267 to P-417; S-268 to P-417; E-269 to P-417;K-270 to P-417; 1-271 to P-417; C-272 to P-417;T-273 to P-417; V-274 to P-417; Q-275 toP-417; L-276 to P-417; V-277 to P-417; G-278 toP-417; N-279 to P-417; S-280 to P-417; W-281 to P-417; T-282 to P-417; P-283 to P-417; G-284 to P-417; Y-285 to P-417; P-286 to P-417; E-287 to P-417; T-288 to P-417; Q-289 to P-417; E-290 to P-417; A-291 to P-417; L-292 to P-417; C-293 to P-417; P-294 to P-417; Q-295 to P-417;V-296 to P-417; T-297 to P-417; W-298 toP-417; S-299 to P-417; W-300 to P-417; D-301 to P-417; Q-302 to P-417; L-303 to P-417; P-304 to P-417; S-305 to P-417; R-306 to P-417; A-307 to P-417; L-308 to P-417; G-309 to P-417; P-310 to P-417; A-311 to P-417; A-312 to P-417;A-313 to P-417; P-314 to P-417; T-315 to P-417;L-316 to P-417; S-317 to P-417; P-318 to P-417;E-319 to P-417; S-320 to P-417; P-321 to P-417;A-322 to P-417; G-323 to P-417; S-324 to P-417;P-325 to P-417; A-326 to P-417; M-327 toP-417; M-328 to P-417; L-329 to P-417; Q-330 to P-417; P-331 to P-417; G-332 to P-417; P-333 to P-417; Q-334 to P-417; L-335 to P-417;Y-336 to P-417; D-337 to P-417; V-338 toP-417; M-339 to P-417; D-340 to P-417; A-341 to P-417; V-342 to P-417; P-343 to P-417; A-344 to P-417; R-345 to P-417; R-346 to P-417;W-347 to P-417; K-348 to P-417; E-349 toP-417; F-350 to P-417; V-351 to P-417; R-352 toP-417; T-353 to P-417; L-354 to P-417; G-355 toP-417; L-356 to P-417; R-357 to P-417; E-358 toP-417; A-359 to P-417; E-360 to P-417; 1-361 toP-417; E-362 to P-417; A-363 to P-417; V-364 toP-417; E-365 to P-417; V-366 to P-417; E-367 toP-417; 1-368 to P-417; G-369 to P-417; R-370 toP-417; F-371 to P-417; R-372 to P-417; D-373 toP-417; Q-374 to P-417; Q-375 to P-417; Y-376 to P-417; E-377 to P-417; M-378 to P-417;L-379 to P-417; K-380 to P-417; R-381 to P-417;W-382 to P-417; R-383 to P-417; Q-384 toP-417; Q-385 to P-417; Q-386 to P-417; P-387 to P-417; A-388 to P-417; G-389 to P-417; L-390 to P-417; G-391 to P-417; A-392 to P-417;V-393 to P-417; Y-394 to P-417; A-395 toP-417; A-396 to P-417; L-397 to P-417; E-398 toP-417; R-399 to P-417; M-400 to P-417; G-401 to P-417; L-402 to P-417; D-403 to P-417; G-404 to P-417; C-405 to P-417; V-406 to P-417; E-407 to P-417; D-408 to P-417; L-409 to P-417; R-410 to P-417; S-411 to P-417; R-412 to P-417; of SEQ ID NO:4. Polynucleotides encoding the above polypeptide fragments and antibodies that bind the above polypeptide fragments are also encompassed by the invention.

The present invention also encompasses nucleic acid molecules comprising, or alternatively, consisting of, a polynucleotide sequence at least 80%, 85%, 90%, 92%, 95%, 96%, 97%, 98% or 99% identical to the polynucleotide sequence encoding polypeptides as described above. The present invention also encompasses the above polynucleotide sequences fused to a heterologous polynucleotide sequence. Polypeptides encoded by these nucleic acids and/or polynucleotide sequences are also encompassed by the invention, as are polypeptides comprising, or alternatively consisting of, an amino acid sequence at least 80%, 85%, 90%, 92%, 95%, 96%, 97%, 98% or 99% identical to the amino acid sequence described above, and polynucleotides that encode such polypeptides.

Moreover, the present invention further provides polypeptides having one or more residues deleted from the carboxy terminus of the amino acid sequence of the polypeptide shown in SEQ ID NO:4 (FIGS. 3A-3C). For example, the present invention provides polypeptides comprising, or alternatively consisting of, the amino acid sequence of residues 1-m¹ of the amino acid sequence in SEQ ID NO:4, where ml is any integer in the range 6-416. More in particular, in certain embodiments, the invention provides polynucleotides encoding polypeptides comprising, or alternatively consisting of, an amino acid sequence selected from the group consisting of residues M-1 to G-416; M-1 to R-415; M-1 toQ-414; M-1 to L-413; M-1 to R-412; M-1 toS-411; M-1 to R-410; M-1 to L-409; M-1 toD-408; M-1 to E-407; M-1 to V-406; M-1 toC-405; M-1 to G-404; M-1 to D-403; M-1 toL-402; M-1 to G-401; M-1 to M-400; M-1 toR-399; M-1 to E-398; M-1 to L-397; M-1 toA-396; M-1 to A-395; M-1 to Y-394; M-1 toV-393; M-1 to A-392; M-1 to G-391; M-1 toL-390; M-1 to G-389; M-1 to A-388; M-1 toP-387; M-1 to Q-386; M-1 to Q-385; M-1 toQ-384; M-1 to R-383; M-1 to W-382; M-1 toR-381; M-1 to K-380; M-1 to L-379; M-1 toM-378; M-1 to E-377; M-1 to Y-376; M-1 toQ-375; M-1 to Q-374; M-1 to D-373; M-1 toR-372; M-1 to F-371; M-1 to R-370; M-1 toG-369; M-1 to 1-368; M-1 to E-367; M-1 toV-366; M-1 to E-365; M-1 to V-364; M-1 toA-363; M-1 to E-362; M-1 to 1-361; M-1 toE-360; M-1 to A-359; M-1 to E-358; M-1 toR-357; M-1 to L-356; M-1 to G-355; M-1 toL-354; M-1 to T-353; M-1 to R-352; M-1 toV-351; M-1 to F-350; M-1 to E-349; M-1 toK-348; M-1 to W-347; M-1 to R-346; M-1 toR-345; M-1 to A-344; M-1 to P-343; M-1 toV-342; M-1 to A-341; M-1 to D-340; M-1 toM-339; M-1 to V-338; M-1 to D-337; M-1 toY-336; M-1 to L-335; M-1 to Q-334; M-1 toP-333; M-1 to G-332; M-1 to P-331; M-1 toQ-330; M-1 to L-329; M-1 to M-328; M-1 toM-327; M-1 to A-326; M-1 to P-325; M-1 toS-324; M-1 to G-323; M-1 to A-322; M-1 toP-321; M-1 to S-320; M-1 to E-319; M-1 toP-318; M-1 to S-317; M-1 to L-316; M-1 toT-315; M-1 to P-314; M-1 to A-313; M-1 toA-312; M-I to A-311; M-1 to P-310; M-1 toG-309; M-1 to L-308; M-1 to A-307; M-1 toR-306; M-1 to S-305; M-1 to P-304; M-1 toL-303; M-1 to Q-302; M-1 to D-301; M-1 toW-300; M-1 to S-299; M-1 to W-298; M-1 toT-297; M-1 to V-296; M-1 to Q-295; M-1 toP-294; M-1 to C-293; M-1 to L-292; M-1 toA-291; M-1 to E-290; M-1 to Q-289; M-1 toT-288; M-1 to E-287; M-1 to P-286; M-1 toY-285; M-1 to G-284; M-1 to P-283; M-1 toT-282; M-1 to W-281; M-1 to S-280; M-1 toN-279; M-1 to G-278; M-1 to V-277; M-1 toL-276; M-1 to Q-275; M-1 to V-274; M-1 toT-273; M-1 to C-272; M-1 to 1-271; M-1 toK-270; M-1 to E-269; M-1 to S-268; M-1 toS-267; M-1 to D-266; M-1 to P-265; M-1 toP-264; M-1 to A-263; M-1 to L-262; M-1 toL-261; M-1 to T-260; M-1 to H-259; M-1 toA-258; M-1 to S-257; M-1 to D-256; M-1 toL-255; M-1 to P-254; M-1 to S-253; M-1 toL-252; M-1 to H-251; M-1 to T-250; M-1 toA-249; M-1 to P-248; M-1 to P-247; M-1 toP-246; M-1 to T-245; M-1 to L-244; M-1 toA-243; M-1 to E-242; M-1 to M-241; M-1 toG-240; M-1 to A-239; M-1 to E-238; M-1 toD-237; M-1 to A-236; M-1 to T-235; M-1 toV-234; M-1 to L-233; M-1 to P-232; M-1 toK-231; M-1 to H-230; M-1 to P-229; M-1 toW-228; M-1 to C-227; M-1 to H-226; M-1 toR-225; M-1 to Y-224; M-1 to T-223; M-1 toY-222; M-1 to T-221; M-1 to L-220; M-1 toT-219; M-1 to A-218; M-1 to G-217; M-1 toL-216; M-1 to L-215; M-1 to L-214; M-1 toP-213; M-1 to V-212; M-1 to V-211; M-1 toL-210; M-1 to G-209; M-1 to A-208; M-1 toL-207; M-1 to L-206; M-1 to V-205; M-1 toQ-204; M-1 to V-203; M-1 to W-202; M-1 toF-201; M-1 to M-200; M-1 to Q-199; M-1 toR-198; M-1 to W-197; M-1 to G-196; M-1 toC-195; M-1 to V-194; M-1 to A-193; M-1 toA-192; M-1 to C-191; M-1 to R-190; M-1 toE-189; M-1 to P-188; M-1 to C-187; M-1 toS-186; M-1 to G-185; M-1 to L-184; M-1 toT-183; M-1 to S-182; M-1 to T-181; M-1 toP-180; M-1 to C-179; M-1 to S-178; M-1 toV-177; M-1 to C-176; M-1 to G-175; M-1 toD-174; M-1 to G-173; M-1 to H-172; M-1 toE-171; M-1 to Y-170; M-1 to F-169; M-1 toG-168; M-1 to P-167; M-1 to L-166; M-1 toC-165; M-1 to T-164; M-1 to G-163; M-1 toC-162; M-1 to D-161; M-1 to T-160; M-1 toD-159; M-1 to R-158; M-1 to R-157; M-1 toS-156; M-1 to C-155; M-1 to L-154; M-1 toL-153; M-1 to R-152; M-1 to T-151; M-1 toH-150; M-1 to R-149; M-1 to H-148; M-1 toL-147; M-1 to A-146; M-1 to G-145; M-1 toC-144; M-1 to D-143; M-1 to L-142; M-1 toC-141; M-1 to P-140; M-1 to Q-139; M-1 toC-138; M-1 to Y-137; M-1 to F-136; M-1 toP-135; M-1 to S-134; M-1 to S-133; M-1 toS-132; M-1 to V-131; M-1 to C-130; M-1 toQ-129; M-1 to S-128; M-1 to V-127; M-1 toQ-126; M-1 to C-125; M-1 to E-124; M-1 toV-123; M-1 to F-122; M-1 to W-121; M-1 toG-120; M-1 to P-119; M-1 to K-118; M-1 toC-117; M-1 to G-116; M-1 to C-115; M-1 toR-114; M-1 to T-113; M-1 to D-112; M-1 toA-111; M-1 to V-110; M-1 to A-109; M-1 toS-108; M-1 to C-107; M-1 to N-106; M-1 toE-105; M-1 to L-104; M-1 to A-103; M-1 toV-102; M-1 to Q-101; M-1 to S-100; M-1 toA-99; M-1 to Q-98; M-1 to E-97; M-1 to D-96;M-1 to C-95; M-1 to A-94; M-I to Q-93; M-1 toC-92; M-1 to R-91; M-1 to A-90; M-I to C-89;M-1 to E-88; M-1 to S-87; M-1 to N-86; M-1 toH-85; M-1 to H-84; M-1 to N-83; M-1 to E-82;M-1 to W-81; M-1 to A-80; M-1 to L-79; M-1 toF-78; M-1 to T-77; M-1 to D-76; M-1 to Q-75;M-1 to P-74; M-I to C-73; M-I to V-72; M-1 toL-71; M-1 to C-70; M-1 to T-69; M-1 to S-68;M-1 to N-67; M-1 to G-66; M-1 to C-65; M-1 toP-64; M-1 to E-63; M-1 to T-62; M-1 to C-61;M-1 to P-60; M-1 to A-59; M-1 to K-58; M-1 toL-57; M-1 to Y-56; M-1 to H-55; M-1 to G-54;M-1 to A-53; M-1 to P-52; M-1 to C-51; M-1 toG-50; M-1 to R-49; M-1 to C-48; M-1 to C-47;M-1 to F-46; M-1 to L-45; M-1 to G-44; M-1 toI-43; M-1 to K-42; M-1 to K-41; M-1 to H-40;M-1 to F-39; M-1 to D-38; M-1 to G-37; M-1 toA-36; M-1 to C-35; M-1 to D-34; M-1 to C-33;M-1 to R-32; M-1 to P-31; M-1 to S-30; M-1 toR-29; M-1 to T-28; M-1 to G-27; M-1 to G-26;M-1 to Q-25; M-1 to A-24; M-1 to R-23; M-1 toA-22; M-1 to G-21; M-1 to L-20; M-1 to L-19;M-1 to V-18; M-1 to L-17; M-1 to L-16; M-1 toL-15; M-1 to A-14; M-1 to A-13; M-1 to A-12;M-1 to V-11; M-1 to A-10; M-1 to A-9; M-1 toC-8; M-1 to G-7; M-1 to R-6; of SEQ ID NO:4. Polynucleotides encoding the above polypeptide fragments and antibodies that bind the above polypeptide fragments are also encompassed by the invention.

The present invention encompasses nucleic acid molecules comprising, or alternatively, consisting of, a polynucleotide sequence at least 80%, 85%, 90%, 92%, 95%, 96%, 97%, 98% or 99% identical to the polynucleotide sequence encoding the polypeptides described above. The present invention also encompasses the above polynucleotide sequences fused to a heterologous polynucleotide sequence. Polypeptides encoded by these nucleic acids and/or polynucleotide sequences are also encompassed by the invention, as are polypeptides comprising, or alternatively consisting of, an amino acid sequence at least 80%, 85%, 90%, 92%, 95%, 96%, 97%, 98% or 99% identical to the amino acid sequence described above, and polynucleotides that encode such polypeptides.

The invention also provides polypeptides having one or more amino acids deleted from both the amino and the carboxyl termini of the polypeptide of SEQ ID NO:4 (FIGS. 3A-3C). For example, amino terminal and carboxyl terminal deletions of the polypeptide sequence may be described generally, for example, as having residues n¹-m¹ of SEQ ID NO:2 where n¹ is an integer in the range of 1-403 and m¹ is an integer in the range of 15-417. For example, and more in particular, the invention provides polynucleotides encoding polypeptides comprising, or alternatively consisting of, an amino acid sequence selected from the group consisting of residues of M-1 to L-15; E-2 to L-16; Q-3 to L-17; R-4 toV-18; P-5 to L-19; R-6 to L-20; G-7 to G-21; C-8 to A-22; A-9 to R-23; A-10 to A-24; V-11 toQ-25; A-12 to G-26; A-13 to G-27; A-14 to T-28;L-15 to R-29; L-16 to S-30; L-17 to P-31; V-18 to R-32; L-19 to C-33; L-20 to D-34; G-21 toC-35; A-22 to A-36; R-23 to G-37; A-24 to D-38;Q-25 to F-39; G-26 to H-40; G-27 to K-41; T-28 to K-42; R-29 to 1-43; S-30 to G-44; P-31 toL-45; R-32 to F-46; C-33 to C-47; D-34 to C-48;C-35 to R-49; A-36 to G-50; G-37 to C-51; D-38 to P-52; F-39 to A-53; H-40 to G-54; K-41 toH-55; K-42 to Y-56; 1-43 to L-57; G-44 to K-58;L-45 to A-59; F-46 to P-60; C-47 to C-61; C-48 to T-62; R-49 to E-63; G-50 to P-64; C-51 toC-65; P-52 to G-66; A-53 to N-67; G-54 to S-68;H-55 to T-69; Y-56 to C-70; L-57 to L-71; K-58 to V-72; A-59 to C-73; P-60 to P-74; C-61 toQ-75; T-62 to D-76; E-63 to T-77; P-64 to F-78;C-65 to L-79; G-66 to A-80; N-67 to W-81; S-68 to E-82; T-69 to N-83; C-70 to H-84; L-71 toH-85; V-72 to N-86; C-73 to S-87; P-74 to E-88;Q-75 to C-89; D-76 to A-90; T-77 to R-91; F-78 to C-92; L-79 to Q-93; A-80 to A-94; W-81 toC-95; E-82 to D-96; N-83 to E-97; H-84 to Q-98;H-85 to A-99; N-86 to S-100; S-87 to Q-101; E-88 to V-102; C-89 to A-103; A-90 to L-104;R-91 to E-105; C-92 to N-106; Q-93 to C-107;A-94 to S-108; C-95 to A-109; D-96 to V-110; E-97 to A-111; Q-98 to D-112; A-99 to T-113;S-100 to R-114; Q-101 to C-115; V-102 toG-116; A-103 to C-117; L-104 to K-118; E-105 to P-119; N-106 to G-120; C-107 to W-121;S-108 to F-122; A-109 to V-123; V-110 toE-124; A-111 to C-125; D-112 to Q-126; T-113 to V-127; R-114 to S-128; C-115 to Q-129;G-116 to C-130; C-117 to V-131; K-118 toS-132; P-119 to S-133; G-120 to S-134; W-121 to P-135; F-122 to F-136; V-123 to Y-137; E-124 to C-138; C-125 to Q-139; Q-126 to P-140;V-127 to C-141; S-128 to L-142; Q-129 toD-143; C-130 to C-144; V-131 to G-145; S-132 to A-146; S-133 to L-147; S-134 to H-148; P-135 to R-149; F-136 to H-150; Y-137 to T-151;C-138 to R-152; Q-139 to L-153; P-140 toL-154; C-141 to C-155; L-142 to S-156; D-143 to R-157; C-144 to R-158; G-145 to D-159;A-146 to T-160; L-147 to D-161; H-148 toC-162; R-149 to G-163; H-150 to T-164; T-151 to C-165; R-152 to L-166; L-153 to P-167; L-154 to G-168; C-155 to F-169; S-156 to Y-170;R-157 to E-171; R-158 to H-172; D-159 toG-173; T-160 to D-174; D-161 to G-175; C-162 to C-176; G-163 to V-177; T-164 to S-178;C-165 to C-179; L-166 to P-180; P-167 to T-181;G-168 to S-182; F-169 to T-183; Y-170 to L-184;E-171 to G-185; H-172 to S-186; G-173 toC-187; D-174 to P-188; G-175 to E-189; C-176 to R-190; V-177 to C-191; S-178 to A-192;C-179 to A-193; P-180 to V-194; T-181 toC-195; S-182 to G-196; T-183 to W-197; L-184 to R-198; G-185 to Q-199; S-186 to M-200;C-187 to F-201; P-188 to W-202; E-189 toV-203; R-190 to Q-204; C-191 to V-205; A-192 to L-206; A-193 to L-207; V-194 to A-208;C-195 to G-209; G-196 to L-210; W-197 toV-211; R-198 to V-212; Q-199 to P-213; M-200 to L-214; F-201 to L-215; W-202 to L-216;V-203 to G-217; Q-204 to A-218; V-205 toT-219; L-206 to L-220; L-207 to T-221; A-208 toY-222; G-209 to T-223; L-210 to Y-224; V-21Ito R-225; V-212 to H-226; P-213 to C-227;L-214 to W-228; L-215 to P-229; L-216 toH-230; G-217 to K-231; A-218 to P-232; T-219 to L-233; L-220 to V-234; T-221 to T-235; Y-222 to A-236; T-223 to D-237; Y-224 to E-238;R-225 to A-239; H-226 to G-240; C-227 toM-241; W-228 to E-242; P-229 to A-243; H-230 to L-244; K-231 to T-245; P-232 to P-246; L-233 to P-247; V-234 to P-248; T-235 to A-249;A-236 to T-250; D-237 to H-251; E-238 toL-252; A-239 to S-253; G-240 to P-254; M-241 to L-255; E-242 to D-256; A-243 to S-257; L-244 to A-258; T-245 to H-259; P-246 to T-260; P-247 to L-261; P-248 to L-262; A-249 to A-263; T-250 to P-264; H-251 to P-265; L-252 to D-266; S-253 to S-267; P-254 to S-268; L-255 to E-269; D-256 to K-270; S-257 to I-271; A-258 to C-272;H-259 to T-273; T-260 to V-274; L-261 toQ-275; L-262 to L-276; A-263 to V-277; P-264 to G-278; P-265 to N-279; D-266 to S-280;S-267 to W-281; S-268 to T-282; E-269 toP-283; K-270 to G-284; 1-271 to Y-285; C-272 to P-286; T-273 to E-287; V-274 to T-288;Q-275 to Q-289; L-276 to E-290; V-277 toA-291; G-278 to L-292; N-279 to C-293; S-280 to P-294; W-281 to Q-295; T-282 to V-296;P-283 to T-297; G-284 to W-298; Y-285 toS-299; P-286 to W-300; E-287 to D-301; T-288 to Q-302; Q-289 to L-303; E-290 to P-304;A-291 to S-305; L-292 to R-306; C-293 toA-307; P-294 to L-308; Q-295 to G-309; V-296 to P-310; T-297 to A-311; W-298 to A-312;S-299 to A-313; W-300 to P-314; D-301 toT-315; Q-302 to L-316; L-303 to S-317; P-304 toP-318; S-305 to E-319; R-306 to S-320; A-307 toP-321; L-308 to A-322; G-309 to G-323; P-310 to S-324; A-311 to P-325; A-312 to A-326;A-313 to M-327; P-314 to M-328; T-315 toL-329; L-316 to Q-330; S-317 to P-331; P-318 toG-332; E-319 to P-333; S-320 to Q-334; P-321 to L-335; A-322 to Y-336; G-323 to D-337;S-324 to V-338; P-325 to M-339; A-326 toD-340; M-327 to A-341; M-328 to V-342; L-329 to P-343; Q-330 to A-344; P-331 to R-345;G-332 to R-346; P-333 to W-347; Q-334 toK-348; L-335 to E-349; Y-336 to F-350; D-337 to V-351; V-338 to R-352; M-339 to T-353;D-340 to L-354; A-341 to G-355; V-342 toL-356; P-343 to R-357; A-344 to E-358; R-345 toA-359; R-346 to E-360; W-347 to 1-361; K-348 to E-362; E-349 to A-363; F-350 to V-364;V-351 to E-365; R-352 to V-366; T-353 toE-367; L-354 to 1-368; G-355 to G-369; L-356 toR-370; R-357 to F-371; E-358 to R-372; A-359 to D-373; E-360 to Q-374; 1-361 to Q-375; E-362 to Y-376; A-363 to E-377; V-364 to M-378;E-365 to L-379; V-366 to K-380; E-367 toR-381; 1-368 to W-382; G-369 to R-383; R-370 to Q-384; F-371 to Q-385; R-372 to Q-386;D-373 to P-387; Q-374 to A-388; Q-375 toG-389; Y-376 to L-390; E-377 to G-391; M-378 to A-392; L-379 to V-393; K-380 to Y-394;R-381 to A-395; W-382 to A-396; R-383 toL-397; Q-384 to E-398; Q-385 to R-399; Q-386 to M-400; P-387 to G-401; A-388 to L-402;G-389 to D-403; L-390 to G-404; G-391 toC-405; A-392 to V-406; V-393 to E-407; Y-394 to D-408; A-395 to L-409; A-396 to R-410;L-397 to S-411; E-398 to R-412; R-399 to L-413;M-400 to Q-414; G-401 to R-415; L-402 toG-416; or D-403 to P-417 of SEQ ID NO:4. Polynucleotides encoding the above polypeptide fragments and antibodies that bind the above polypeptide fragments are also encompassed by the invention.

The present invention encompasses nucleic acid molecules comprising, or alternatively, consisting of, a polynucleotide sequence at least 80%, 85%, 90%, 92%, 95%, 96%, 97%, 98% or 99% identical to the polynucleotide sequence encoding the polypeptides described above. The present invention also encompasses the above polynucleotide sequences fused to a heterologous polynucleotide sequence. Polypeptides encoded by these nucleic acids and/or polynucleotide sequences are also encompassed by the invention, as are polypeptides comprising, or alternatively consisting of, an amino acid sequence at least 80%, 85%, 90%, 92%, 95%, 96%, 97%, 98% or 99% identical to the amino acid sequence described above, and polynucleotides that encode such polypeptides.

Also preferred are polypeptide and polynucleotide fragments characterized by structural or functional domains (See, FIG. 4 and Table 2), such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. See FIG. 4 and Table 2. Polypeptide fragments of SEQ ID NO:4 falling within conserved domains, hydrophillic, and antigenic domains are specifically contemplated by the present invention. Moreover, polynucleotides encoding these domains and antibodies that bind to these domains are also contemplated.

TABLE 2

| Res: | Pos: | I | II | III | IV | V | VI | VII | VIII | IX | X | XI | XII | XIII | XIV |
|---|---|---|---|---|---|---|---|---|---|---|---|---|---|---|---|
| Met | 1 | A | . | . | . | . | . | . | 1.24 | −0.70 | . | * | . | 1.29 | 2.18 |
| Glu | 2 | A | . | . | . | . | . | . | 1.74 | −0.70 | . | * | . | 1.63 | 2.63 |
| Gln | 3 | A | . | . | . | . | . | . | 1.79 | −1.13 | . | * | . | 1.97 | 4.04 |
| Arg | 4 | . | . | . | . | . | T | C | 1.51 | −1.13 | . | * | . | 2.71 | 4.04 |
| Pro | 5 | . | . | . | . | T | T | . | 1.31 | −1.17 | . | * | F | 3.40 | 1.25 |
| Arg | 6 | . | . | . | . | T | T | . | 1.32 | −0.67 | . | * | F | 2.91 | 0.73 |
| Gly | 7 | A | . | . | . | . | T | . | 0.47 | −0.57 | . | * | . | 2.02 | 0.38 |
| Cys | 8 | A | A | . | . | . | . | . | −0.12 | 0.07 | . | * | . | 0.38 | 0.18 |
| Ala | 9 | A | A | . | . | . | . | . | −0.82 | 0.14 | . | * | . | 0.04 | 0.09 |
| Ala | 10 | A | A | . | . | . | . | . | −1.20 | 0.64 | * | * | . | −0.60 | 0.09 |
| Val | 11 | A | A | . | . | . | . | . | −2.12 | 0.71 | * | * | . | −0.60 | 0.18 |
| Ala | 12 | A | A | . | . | . | . | . | −2.59 | 0.83 | . | . | . | −0.60 | 0.15 |
| Ala | 13 | A | A | . | . | . | . | . | −2.73 | 1.01 | . | . | . | −0.60 | 0.12 |
| Ala | 14 | A | A | . | . | . | . | . | −3.00 | 1.20 | . | . | . | −0.60 | 0.13 |
| Leu | 15 | A | A | . | . | . | . | . | −3.22 | 1.20 | . | . | . | −0.60 | 0.10 |
| Leu | 16 | A | A | . | . | . | . | . | −3.18 | 1.39 | . | . | . | −0.60 | 0.08 |
| Leu | 17 | A | A | . | . | . | . | . | −2.93 | 1.57 | . | . | . | −0.60 | 0.06 |
| Val | 18 | A | A | . | . | . | . | . | −2.93 | 1.50 | . | * | . | −0.60 | 0.08 |
| Leu | 19 | A | A | . | . | . | . | . | −2.23 | 1.31 | . | * | . | −0.60 | 0.10 |
| Leu | 20 | A | A | . | . | . | . | . | −2.01 | 0.63 | . | * | . | −0.60 | 0.23 |
| Gly | 21 | A | A | . | . | . | . | . | −1.20 | 0.44 | . | * | . | −0.60 | 0.31 |
| Ala | 22 | A | A | . | . | . | . | . | −0.73 | 0.20 | . | * | . | 0.04 | 0.65 |
| Arg | 23 | A | A | . | . | . | . | . | −0.22 | −0.06 | . | * | . | 0.98 | 0.78 |
| Ala | 24 | A | . | . | . | . | T | . | 0.28 | −0.31 | * | * | F | 1.87 | 0.78 |
| Gln | 25 | . | . | . | . | T | T | . | 1.20 | −0.26 | * | * | F | 2.76 | 1.11 |
| Gly | 26 | . | . | . | . | T | T | . | 1.24 | −0.76 | * | * | F | 3.40 | 1.11 |
| Gly | 27 | . | . | . | . | T | T | . | 1.62 | −0.37 | * | * | F | 2.76 | 1.47 |
| Thr | 28 | . | . | . | . | T | . | . | 1.62 | −0.44 | * | * | F | 2.53 | 1.32 |
| Arg | 29 | . | . | . | . | T | . | . | 1.54 | −0.84 | * | * | F | 2.80 | 2.60 |
| Ser | 30 | . | . | . | . | . | T | C | 1.54 | −0.70 | * | * | F | 2.77 | 1.41 |
| Pro | 31 | . | . | . | . | T | T | . | 1.22 | −1.13 | * | . | F | 2.94 | 1.63 |
| Arg | 32 | . | . | . | . | T | T | . | 0.98 | −1.04 | * | . | F | 3.10 | 0.45 |
| Cys | 33 | . | . | . | . | T | T | . | 0.94 | −0.54 | . | * | . | 2.64 | 0.34 |
| Asp | 34 | . | . | . | . | T | . | . | 0.83 | −0.50 | . | * | . | 1.83 | 0.22 |
| Cys | 35 | A | . | . | . | . | T | . | 0.43 | −0.93 | . | * | . | 1.62 | 0.18 |
| Ala | 36 | A | . | . | . | . | T | . | 0.61 | −0.14 | . | * | . | 1.01 | 0.30 |
| Gly | 37 | A | . | . | . | . | T | . | 0.54 | −0.21 | * | * | . | 0.70 | 0.24 |
| Asp | 38 | A | . | . | . | . | T | . | 1.26 | −0.21 | * | * | . | 0.70 | 0.90 |
| Phe | 39 | A | . | . | . | . | . | . | 0.37 | −0.79 | * | * | F | 1.10 | 1.79 |
| His | 40 | A | . | . | . | . | . | . | 0.69 | −0.60 | * | * | F | 1.10 | 1.26 |
| Lys | 41 | A | . | . | . | . | . | . | 0.47 | −0.60 | * | * | F | 0.95 | 0.75 |
| Lys | 42 | . | . | . | B | T | . | . | 0.11 | 0.09 | * | * | F | 0.25 | 0.71 |
| Ile | 43 | . | . | . | B | T | . | . | −0.56 | 0.09 | * | * | . | 0.10 | 0.45 |
| Gly | 44 | . | . | . | B | T | . | . | −0.52 | 0.16 | * | * | . | 0.10 | 0.12 |
| Leu | 45 | . | . | . | B | T | . | . | −0.38 | 0.73 | * | * | . | −0.20 | 0.03 |
| Phe | 46 | . | . | . | B | T | . | . | −0.77 | 0.73 | * | . | . | −0.20 | 0.09 |
| Cys | 47 | . | . | . | B | T | . | . | −1.48 | 0.47 | * | * | . | −0.20 | 0.09 |
| Cys | 48 | . | . | . | . | T | T | . | −0.80 | 0.61 | * | . | . | 0.42 | 0.06 |
| Arg | 49 | . | . | . | . | T | T | . | −1.04 | 0.36 | . | * | . | 0.94 | 0.11 |
| Gly | 50 | . | . | . | . | T | T | . | −0.58 | 0.07 | . | * | . | 1.16 | 0.20 |
| Cys | 51 | . | . | . | . | T | T | . | 0.09 | −0.07 | * | * | . | 1.98 | 0.37 |
| Pro | 52 | . | . | . | . | T | T | . | 0.51 | −0.14 | * | * | . | 2.20 | 0.26 |
| Ala | 53 | . | . | . | . | T | T | . | 0.37 | 0.61 | . | * | . | 1.08 | 0.41 |
| Gly | 54 | . | . | . | . | T | T | . | 0.30 | 0.87 | . | * | . | 0.86 | 0.62 |
| His | 55 | . | . | . | . | T | T | . | 0.06 | 0.30 | * | . | . | 0.94 | 0.81 |
| Tyr | 56 | . | . | . | . | T | . | . | 0.51 | 0.37 | . | * | . | 0.52 | 0.81 |
| Leu | 57 | . | . | . | . | T | . | . | 0.06 | 0.30 | * | * | . | 0.76 | 1.26 |
| Lys | 58 | . | . | . | . | T | . | . | 0.33 | 0.44 | * | . | . | 0.62 | 0.50 |
| Ala | 59 | . | . | . | . | . | T | C | 0.68 | 0.43 | . | . | . | 0.93 | 0.46 |
| Pro | 60 | . | . | . | . | T | T | . | 0.50 | −0.33 | . | . | F | 2.49 | 0.96 |
| Cys | 61 | . | . | . | . | T | T | . | 0.08 | −0.59 | . | * | F | 3.10 | 0.74 |
| Thr | 62 | . | . | . | . | T | T | . | 0.54 | −0.01 | . | * | F | 2.49 | 0.39 |
| Glu | 63 | . | . | . | . | . | T | C | 0.50 | −0.09 | . | . | F | 2.11 | 0.25 |
| Pro | 64 | . | . | . | . | T | T | . | 0.79 | −0.11 | . | . | F | 2.13 | 0.76 |
| Cys | 65 | . | . | . | . | T | T | . | 0.69 | −0.30 | . | . | F | 1.95 | 0.70 |
| Gly | 66 | . | . | . | . | T | T | . | 0.69 | −0.30 | . | . | F | 1.77 | 0.58 |
| Asn | 67 | . | . | . | . | T | T | . | 0.19 | 0.27 | . | . | F | 1.30 | 0.20 |
| Ser | 68 | . | . | . | . | T | T | . | −0.67 | 0.53 | . | . | F | 0.87 | 0.31 |
| Thr | 69 | . | . | . | . | T | T | . | −1.12 | 0.60 | . | . | F | 0.74 | 0.23 |
| Cys | 70 | . | . | . | . | T | T | . | −0.67 | 0.74 | . | . | . | 0.46 | 0.08 |
| Leu | 71 | . | . | B | B | . | . | . | −0.32 | 0.77 | . | . | . | −0.47 | 0.09 |
| Val | 72 | . | . | B | B | . | . | . | −0.32 | 0.79 | . | . | . | −0.60 | 0.11 |
| Cys | 73 | . | . | B | B | . | . | . | −0.33 | 0.30 | . | . | . | −0.30 | 0.34 |
| Pro | 74 | . | . | . | . | T | T | . | −0.72 | 0.21 | . | . | F | 0.65 | 0.59 |
| Gln | 75 | . | . | . | . | T | T | . | −0.87 | 0.31 | . | . | F | 0.65 | 0.69 |
| Asp | 76 | A | . | . | . | . | T | . | −0.64 | 0.36 | . | . | F | 0.40 | 1.06 |
| Thr | 77 | A | . | . | . | . | T | . | −0.08 | 0.29 | . | . | F | 0.25 | 0.69 |
| Phe | 78 | A | A | . | . | . | . | . | 0.59 | 0.77 | . | . | . | −0.60 | 0.42 |
| Leu | 79 | A | A | . | . | . | . | . | 0.80 | 0.37 | . | . | . | −0.30 | 0.43 |
| Ala | 80 | A | A | . | . | . | . | . | 0.77 | 0.77 | . | . | . | −0.60 | 0.48 |
| Trp | 81 | A | A | . | . | . | . | . | 0.73 | 0.79 | . | . | . | −0.60 | 0.76 |
| Glu | 82 | A | A | . | . | . | . | . | 1.04 | 0.50 | . | . | . | −0.45 | 1.26 |
| Asn | 83 | A | A | . | . | . | . | . | 1.44 | 0.21 | . | . | . | −0.15 | 2.00 |
| His | 84 | . | A | . | . | T | . | . | 2.26 | 0.10 | . | . | . | 0.56 | 2.55 |
| His | 85 | . | A | . | . | T | . | . | 2.18 | −0.81 | . | . | F | 1.92 | 2.55 |
| Asn | 86 | . | . | . | . | T | T | . | 1.88 | −0.24 | . | * | F | 2.18 | 0.85 |
| Ser | 87 | . | . | . | . | T | T | . | 1.99 | −0.14 | . | . | F | 2.49 | 0.63 |
| Glu | 88 | . | . | . | . | T | T | . | 1.32 | −0.64 | . | * | F | 3.10 | 0.91 |
| Cys | 89 | . | . | . | . | T | T | . | 1.36 | −0.57 | . | * | . | 2.64 | 0.30 |
| Ala | 90 | A | A | . | . | . | . | . | 0.80 | −0.57 | . | * | . | 1.53 | 0.39 |
| Arg | 91 | A | A | . | . | . | . | . | 0.13 | −0.46 | . | * | . | 0.92 | 0.23 |
| Cys | 92 | A | A | . | . | . | . | . | 0.43 | 0.11 | . | * | . | 0.01 | 0.23 |
| Gln | 93 | A | A | . | . | . | . | . | 0.43 | −0.46 | . | * | . | 0.30 | 0.38 |
| Ala | 94 | A | A | . | . | . | . | . | 1.10 | −0.96 | . | * | . | 0.60 | 0.33 |
| Cys | 95 | A | A | . | . | . | . | . | 1.10 | −0.56 | . | * | . | 0.75 | 1.08 |
| Asp | 96 | A | A | . | . | . | . | . | 0.69 | −0.63 | . | * | F | 0.75 | 0.63 |
| Glu | 97 | A | A | . | . | . | . | . | 1.36 | −0.64 | * | . | F | 0.75 | 0.83 |
| Gln | 98 | A | A | . | . | . | . | . | 0.50 | −0.74 | * | . | F | 0.90 | 2.69 |
| Ala | 99 | A | A | . | . | . | . | . | 0.50 | −0.67 | . | . | F | 0.90 | 1.20 |
| Ser | 100 | A | A | . | . | . | . | . | 0.36 | −0.17 | * | . | F | 0.45 | 0.70 |
| Gln | 101 | A | A | . | . | . | . | . | 0.36 | 0.51 | . | . | . | −0.60 | 0.33 |
| Val | 102 | A | A | . | . | . | . | . | 0.36 | 0.11 | * | . | . | −0.30 | 0.57 |
| Ala | 103 | A | A | . | . | . | . | . | −0.31 | 0.01 | * | . | . | −0.30 | 0.68 |
| Leu | 104 | A | A | . | . | . | . | . | −0.02 | 0.20 | . | . | . | −0.30 | 0.21 |
| Glu | 105 | A | A | . | . | . | . | . | −0.31 | 0.19 | . | . | . | −0.30 | 0.38 |
| Asn | 106 | A | A | . | . | . | . | . | −1.17 | 0.04 | . | . | . | −0.30 | 0.38 |
| Cys | 107 | A | A | . | . | . | . | . | −0.90 | 0.19 | * | . | . | −0.30 | 0.34 |
| Ser | 108 | A | A | . | . | . | . | . | −0.31 | 0.00 | * | . | . | −0.30 | 0.20 |
| Ala | 109 | A | . | . | . | . | . | . | 0.19 | 0.00 | . | * | . | −0.10 | 0.21 |
| Val | 110 | A | . | . | . | . | . | . | 0.30 | 0.09 | . | * | . | −0.10 | 0.56 |
| Ala | 111 | A | . | . | . | . | . | . | −0.37 | −0.49 | . | * | . | 0.78 | 0.82 |
| Asp | 112 | A | . | . | . | . | T | . | −0.04 | −0.30 | . | * | F | 1.41 | 0.43 |
| Thr | 113 | . | . | . | . | T | T | . | −0.41 | −0.37 | * | * | F | 2.09 | 0.58 |
| Arg | 114 | . | . | . | . | T | T | . | 0.22 | −0.44 | * | * | F | 2.37 | 0.31 |
| Cys | 115 | . | . | . | . | T | T | . | 0.87 | −0.94 | * | * | . | 2.80 | 0.37 |
| Gly | 116 | . | . | . | . | T | . | . | 1.11 | −0.51 | * | * | . | 2.32 | 0.39 |
| Cys | 117 | . | . | . | . | T | . | . | 0.82 | −0.57 | * | * | . | 2.04 | 0.20 |
| Lys | 118 | . | . | . | . | . | T | C | 0.43 | 0.34 | * | * | F | 1.01 | 0.39 |
| Pro | 119 | . | . | . | . | T | T | . | −0.53 | 0.56 | * | * | F | 0.63 | 0.34 |
| Gly | 120 | . | . | . | . | T | T | . | 0.13 | 0.77 | . | . | . | 0.20 | 0.47 |
| Trp | 121 | . | . | . | . | T | T | . | −0.19 | 0.20 | . | * | . | 0.50 | 0.41 |
| Phe | 122 | A | . | . | B | . | . | . | 0.48 | 0.77 | . | * | . | −0.60 | 0.14 |
| Val | 123 | A | . | . | B | . | . | . | −0.42 | 0.74 | . | * | . | −0.60 | 0.25 |
| Glu | 124 | A | . | . | B | . | . | . | −0.51 | 0.96 | . | * | . | −0.60 | 0.18 |
| Cys | 125 | . | . | . | B | T | . | . | −0.17 | 0.43 | . | . | . | −0.20 | 0.27 |
| Gln | 126 | . | . | . | B | T | . | . | −0.54 | 0.04 | . | * | . | 0.10 | 0.63 |
| Val | 127 | . | . | . | B | T | . | . | −0.70 | −0.03 | . | * | . | 0.70 | 0.20 |
| Ser | 128 | . | . | . | B | T | . | . | −0.14 | 0.61 | * | * | . | −0.20 | 0.27 |
| Gln | 129 | . | . | . | B | T | . | . | −0.44 | 0.43 | * | * | . | −0.20 | 0.21 |
| Cys | 130 | . | . | . | B | T | . | . | −0.08 | 0.41 | * | . | . | −0.20 | 0.38 |
| Val | 131 | . | . | . | B | T | . | . | −0.29 | 0.16 | . | . | F | 0.25 | 0.38 |
| Ser | 132 | . | . | . | . | T | . | . | −0.13 | 0.20 | . | . | F | 0.45 | 0.34 |
| Ser | 133 | . | . | . | . | T | . | . | −0.08 | 0.59 | . | . | F | 0.15 | 0.55 |
| Ser | 134 | . | . | . | . | . | T | C | −0.74 | 0.77 | . | . | F | 0.30 | 1.16 |
| Pro | 135 | . | . | . | . | T | T | . | −0.08 | 0.70 | . | . | F | 0.35 | 0.46 |
| Phe | 136 | . | . | . | . | T | T | . | 0.57 | 0.71 | . | . | . | 0.20 | 0.60 |
| Tyr | 137 | . | . | . | . | T | T | . | 0.20 | 0.76 | . | . | . | 0.20 | 0.69 |
| Cys | 138 | . | . | . | . | T | . | . | −0.31 | 0.94 | . | * | . | 0.00 | 0.24 |
| Gln | 139 | . | . | B | . | . | T | . | −0.01 | 1.20 | . | * | . | −0.20 | 0.23 |
| Pro | 140 | . | . | . | . | T | T | . | −0.47 | 0.41 | . | * | . | 0.20 | 0.24 |
| Cys | 141 | . | . | . | . | T | T | . | −0.11 | 0.23 | . | * | . | 0.50 | 0.24 |
| Leu | 142 | . | . | . | . | T | T | . | −0.46 | 0.09 | . | . | . | 0.50 | 0.14 |
| Asp | 143 | . | . | . | . | T | T | . | −0.60 | 0.19 | . | * | . | 0.50 | 0.09 |
| Cys | 144 | A | . | . | . | . | T | . | −0.63 | 0.44 | . | * | . | −0.20 | 0.14 |
| Gly | 145 | A | . | . | . | . | T | . | −0.31 | 0.37 | . | . | . | 0.10 | 0.23 |
| Ala | 146 | A | . | . | . | . | T | . | 0.32 | −0.31 | . | . | . | 0.70 | 0.27 |
| Leu | 147 | A | A | . | . | . | . | . | 0.82 | 0.19 | * | * | . | −0.30 | 0.69 |
| His | 148 | A | A | . | . | . | . | . | 0.93 | 0.10 | * | * | . | −0.30 | 1.00 |
| Arg | 149 | A | A | . | . | . | . | . | 0.79 | −0.33 | * | . | . | 0.45 | 1.94 |
| His | 150 | A | A | . | . | . | . | . | 0.32 | −0.14 | * | . | . | 0.45 | 1.94 |
| Thr | 151 | A | A | . | . | . | . | . | 0.24 | −0.14 | * | . | . | 0.45 | 1.17 |
| Arg | 152 | . | A | . | . | T | . | . | 0.76 | −0.07 | * | . | . | 0.70 | 0.32 |
| Leu | 153 | . | A | . | . | T | . | . | 0.90 | 0.31 | . | . | . | 0.44 | 0.32 |
| Leu | 154 | . | A | . | . | T | . | . | 0.90 | −0.19 | . | . | . | 1.38 | 0.43 |
| Cys | 155 | . | . | . | . | T | T | . | 0.93 | −0.67 | . | * | . | 2.42 | 0.43 |
| Ser | 156 | . | . | . | . | T | T | . | 0.93 | −0.67 | . | * | . | 2.76 | 0.87 |
| Arg | 157 | . | . | . | . | T | T | . | 0.82 | −0.87 | . | * | F | 3.40 | 1.52 |
| Arg | 158 | . | . | . | . | T | T | . | 0.97 | −1.56 | * | . | F | 3.06 | 4.74 |
| Asp | 159 | . | . | . | . | T | T | . | 1.43 | −1.56 | * | . | F | 2.81 | 1.90 |
| Thr | 160 | . | . | . | . | T | T | . | 1.79 | −1.51 | * | . | F | 2.41 | 0.96 |
| Asp | 161 | . | . | . | . | T | T | . | 1.42 | −1.03 | * | . | F | 2.16 | 0.71 |
| Cys | 162 | . | . | . | . | T | T | . | 0.50 | −0.46 | * | . | F | 1.61 | 0.23 |
| Gly | 163 | . | . | . | . | T | . | . | 0.18 | 0.23 | * | . | F | 0.90 | 0.13 |
| Thr | 164 | . | . | . | . | T | . | . | −0.17 | 0.17 | . | . | . | 0.66 | 0.12 |
| Cys | 165 | . | . | . | . | . | . | C | −0.56 | 0.60 | * | . | . | 0.07 | 0.22 |
| Leu | 166 | . | . | . | . | . | T | C | −0.80 | 0.81 | * | . | . | 0.18 | 0.19 |
| Pro | 167 | . | . | . | . | . | T | C | −0.13 | 1.14 | * | . | . | 0.09 | 0.21 |
| Gly | 168 | . | . | . | . | T | T | . | 0.18 | 0.66 | * | . | . | 0.45 | 0.68 |
| Phe | 169 | . | . | . | . | T | T | . | 0.14 | 0.59 | * | . | . | 0.85 | 1.12 |
| Tyr | 170 | . | . | . | . | T | . | . | 0.81 | 0.33 | * | . | . | 1.05 | 0.72 |
| Glu | 171 | . | . | . | . | T | . | . | 1.28 | −0.10 | * | . | . | 2.05 | 1.21 |
| His | 172 | . | . | . | . | T | T | . | 0.82 | −0.10 | * | * | . | 2.50 | 1.38 |
| Gly | 173 | . | . | . | . | T | T | . | 0.31 | −0.31 | * | * | . | 2.10 | 0.47 |
| Asp | 174 | . | . | . | . | T | T | . | 0.71 | −0.43 | * | * | . | 1.85 | 0.20 |
| Gly | 175 | . | . | . | . | T | T | . | 0.29 | −0.04 | . | * | . | 1.60 | 0.20 |
| Cys | 176 | . | . | . | . | T | . | . | 0.08 | 0.03 | . | * | . | 0.55 | 0.11 |
| Val | 177 | . | . | . | . | T | . | . | −0.20 | 0.03 | . | * | . | 0.30 | 0.10 |
| Ser | 178 | . | . | . | . | T | . | . | −0.16 | 0.51 | . | * | . | 0.00 | 0.15 |
| Cys | 179 | . | . | B | . | . | T | . | −0.47 | 0.47 | . | . | . | −0.20 | 0.37 |
| Pro | 180 | . | . | . | . | T | T | . | −0.93 | 0.39 | . | . | F | 0.65 | 0.71 |
| Thr | 181 | . | . | . | . | T | T | . | −0.61 | 0.43 | . | . | F | 0.35 | 0.44 |
| Ser | 182 | . | . | . | . | T | T | . | −0.06 | 0.47 | . | . | F | 0.35 | 0.81 |
| Thr | 183 | . | . | . | . | T | . | . | −0.42 | 0.29 | . | . | F | 0.45 | 070 |
| Leu | 184 | . | . | . | . | T | . | . | 0.03 | 0.43 | . | . | F | 0.46 | 0.26 |
| Gly | 185 | . | . | . | . | T | . | . | 0.24 | 0.37 | * | . | F | 1.07 | 0.30 |
| Ser | 186 | . | . | . | . | T | . | . | 0.67 | −0.01 | * | . | F | 1.98 | 0.36 |
| Cys | 187 | . | . | . | . | . | T | C | 0.30 | −0.50 | * | . | F | 2.29 | 0.85 |
| Pro | 188 | . | . | . | . | T | T | . | 0.02 | −0.61 | * | . | F | 3.10 | 0.46 |
| Glu | 189 | . | . | . | . | T | T | . | 0.24 | −0.54 | * | . | F | 2.79 | 0.35 |
| Arg | 190 | A | . | . | . | . | T | . | −0.27 | −0.43 | * | . | . | 1.63 | 0.66 |
| Cys | 191 | A | . | . | B | . | . | . | −0.63 | −0.36 | * | . | . | 0.92 | 0.32 |
| Ala | 192 | A | . | . | B | . | . | . | −0.31 | −0.21 | . | . | . | 0.61 | 0.10 |
| Ala | 193 | A | . | . | B | . | . | . | −0.39 | 0.21 | * | * | . | −0.30 | 0.05 |
| Val | 194 | A | . | . | B | . | . | . | −0.28 | 1.13 | . | . | . | −0.60 | 0.10 |
| Cys | 195 | A | . | . | B | . | . | . | −0.39 | 0.56 | * | * | . | −0.60 | 0.19 |
| Gly | 196 | . | . | . | B | T | . | . | −0.32 | 0.46 | * | . | . | −0.20 | 0.32 |
| Trp | 197 | . | . | . | B | T | . | . | −0.43 | 0.57 | * | . | . | −0.20 | 0.43 |
| Arg | 198 | A | . | . | B | . | . | . | −0.13 | 0.71 | * | . | . | −0.60 | 0.69 |
| Gln | 199 | A | . | . | B | . | . | . | −0.13 | 1.06 | . | * | . | −0.60 | 0.74 |
| Met | 200 | A | . | . | B | . | . | . | 0.53 | 1.27 | . | * | . | −0.60 | 0.52 |
| Phe | 201 | . | . | . | B | T | . | . | 0.02 | 0.76 | . | * | . | −0.20 | 0.46 |
| Trp | 202 | A | . | . | B | . | . | . | −0.50 | 1.40 | . | * | . | −0.60 | 0.20 |
| Val | 203 | A | . | . | B | . | . | . | −1.42 | 1.69 | . | * | . | −0.60 | 0.16 |
| Gln | 204 | A | . | . | B | . | . | . | −2.01 | 1.76 | . | . | . | −0.60 | 0.16 |
| Val | 205 | A | . | . | B | . | . | . | −1.76 | 1.47 | . | . | . | −0.60 | 0.15 |
| Leu | 206 | A | . | . | B | . | . | . | −1.87 | 0.99 | . | * | . | −0.60 | 0.20 |
| Leu | 207 | A | . | . | B | . | . | . | −2.43 | 1.03 | . | . | . | −0.60 | 0.10 |
| Ala | 208 | A | . | . | B | . | . | . | −2.43 | 1.27 | . | . | . | −0.60 | 0.10 |
| Gly | 209 | A | . | . | B | . | . | . | −2.64 | 1.27 | . | . | . | −0.60 | 0.09 |
| Leu | 210 | A | . | . | B | . | . | . | −2.60 | 1.01 | . | . | . | −0.60 | 0.16 |
| Val | 211 | . | . | B | B | . | . | . | −2.60 | 1.01 | . | . | . | −0.60 | 0.13 |
| Val | 212 | . | . | B | B | . | . | . | −2.60 | 1.20 | . | . | . | −0.60 | 0.11 |
| Pro | 213 | . | . | B | B | . | . | . | −2.36 | 1.46 | . | . | . | −0.60 | 0.11 |
| Leu | 214 | . | . | B | B | . | . | . | −2.60 | 1.20 | . | . | . | −0.60 | 0.15 |
| Leu | 215 | A | . | . | B | . | . | . | −2.10 | 1.06 | . | * | . | −0.60 | 0.20 |
| Leu | 216 | A | . | . | B | . | . | . | −2.06 | 0.90 | . | . | . | −0.60 | 0.19 |
| Gly | 217 | A | . | . | B | . | . | . | −1.51 | 1.16 | . | * | . | −0.60 | 0.19 |
| Ala | 218 | A | . | . | B | . | . | . | −1.54 | 0.96 | . | . | . | −0.60 | 0.32 |
| Thr | 219 | A | . | . | B | . | . | . | −1.04 | 1.03 | . | * | . | −0.60 | 0.62 |
| Leu | 220 | A | . | . | B | . | . | . | −0.48 | 0.83 | * | * | . | −0.60 | 0.90 |
| Thr | 221 | . | . | B | B | . | . | . | 0.44 | 1.16 | * | * | . | −0.45 | 1.40 |
| Tyr | 222 | . | . | . | B | T | . | . | 0.76 | 0.66 | * | * | . | −0.05 | 1.89 |
| Thr | 223 | . | . | . | B | T | . | . | 0.68 | 0.67 | * | * | . | −0.05 | 3.12 |
| Tyr | 224 | . | . | . | . | T | T | . | 0.70 | 0.56 | * | * | . | 0.35 | 1.16 |
| Arg | 225 | . | . | . | . | T | T | . | 1.30 | 0.99 | * | * | . | 0.20 | 0.78 |
| His | 226 | . | . | . | . | T | T | . | 1.58 | 0.66 | . | * | . | 0.20 | 0.83 |
| Cys | 227 | . | . | . | . | T | T | . | 1.87 | 0.67 | . | * | . | 0.20 | 0.72 |
| Trp | 228 | . | . | . | . | . | T | C | 1.97 | −0.09 | . | * | . | 0.90 | 0.74 |
| Pro | 229 | . | . | . | . | T | T | . | 1.40 | 0.34 | . | * | . | 0.50 | 0.84 |
| His | 230 | . | . | . | . | T | T | . | 0.43 | 0.53 | . | * | . | 0.35 | 1.29 |
| Lys | 231 | . | . | . | . | . | T | C | 0.16 | 0.60 | . | . | F | 0.15 | 0.91 |
| Pro | 232 | . | . | . | . | . | . | C | 0.23 | 0.17 | . | * | F | 0.25 | 0.85 |
| Leu | 233 | . | A | . | . | . | . | C | 0.52 | 0.24 | * | . | . | −0.10 | 0.63 |
| Val | 234 | A | A | . | . | . | . | . | 0.73 | −0.26 | * | . | . | 0.30 | 0.53 |
| Thr | 235 | A | A | . | . | . | . | . | 0.18 | −0.26 | * | . | . | 0.30 | 0.59 |
| Ala | 236 | A | A | . | . | . | . | . | −0.21 | −0.19 | * | . | F | 0.45 | 0.72 |
| Asp | 237 | A | A | . | . | . | . | . | −0.60 | −0.44 | . | . | F | 0.45 | 0.97 |
| Glu | 238 | A | A | . | . | . | . | . | 0.21 | −0.47 | . | . | F | 0.45 | 0.66 |
| Ala | 239 | A | A | . | . | . | . | . | 0.48 | −0.96 | . | . | F | 0.90 | 1.14 |
| Gly | 240 | A | A | . | . | . | . | . | −0.02 | −0.96 | * | . | . | 0.60 | 0.69 |
| Met | 241 | A | A | . | . | . | . | . | 0.26 | −0.27 | * | . | . | 0.30 | 0.33 |
| Glu | 242 | A | A | . | . | . | . | . | 0.04 | 0.21 | . | . | . | −0.30 | 0.47 |
| Ala | 243 | A | A | . | . | . | . | . | −0.17 | 0.14 | * | . | . | −0.30 | 0.73 |
| Leu | 244 | . | A | . | . | . | . | C | 0.21 | 0.14 | . | . | . | 0.05 | 1.14 |
| Thr | 245 | . | A | . | . | . | . | C | −0.03 | −0.04 | . | . | F | 0.80 | 1.02 |
| Pro | 246 | . | A | . | . | . | . | C | 0.26 | 0.46 | . | . | F | −0.10 | 1.02 |
| Pro | 247 | . | . | . | . | . | T | C | 0.22 | 0.44 | . | . | F | 0.30 | 1.78 |
| Pro | 248 | . | . | . | . | T | T | . | 0.00 | 0.26 | . | . | F | 0.80 | 1.68 |
| Ala | 249 | . | . | . | . | T | T | . | 0.51 | 0.46 | . | . | F | 0.35 | 0.90 |
| Thr | 250 | A | . | . | . | . | T | . | 0.61 | 0.41 | . | . | . | −0.20 | 0.78 |
| His | 251 | . | . | B | . | . | . | . | 0.01 | 0.41 | . | . | . | −0.40 | 0.78 |
| Leu | 252 | . | . | B | . | . | . | . | 0.22 | 0.67 | . | . | . | −0.40 | 0.63 |
| Ser | 253 | . | . | . | . | . | T | C | 0.13 | 0.17 | . | . | F | 0.45 | 0.73 |
| Pro | 254 | . | . | . | . | . | T | C | 0.13 | 0.07 | . | . | F | 0.45 | 0.72 |
| Leu | 255 | . | . | . | . | . | T | C | 0.41 | 0.07 | . | . | F | 0.45 | 0.89 |
| Asp | 256 | A | . | . | . | . | T | . | 0.13 | −0.11 | . | . | F | 0.85 | 0.90 |
| Ser | 257 | A | A | . | . | . | . | . | 0.13 | −0.01 | * | . | F | 0.45 | 0.84 |
| Ala | 258 | A | A | . | . | . | . | . | −0.38 | 0.24 | * | . | . | −0.30 | 0.84 |
| His | 259 | A | A | . | . | . | . | . | −0.76 | 0.24 | * | . | . | −0.30 | 0.41 |
| Thr | 260 | . | A | B | . | . | . | . | −0.16 | 0.74 | * | . | . | −0.60 | 0.31 |
| Leu | 261 | . | A | B | . | . | . | . | −0.37 | 0.79 | * | . | . | −0.60 | 0.48 |
| Leu | 262 | . | A | B | . | . | . | . | −0.07 | 0.71 | . | . | . | −0.26 | 0.54 |
| Ala | 263 | . | A | . | . | . | . | C | 0.22 | 0.21 | . | . | . | 0.58 | 0.63 |
| Pro | 264 | . | . | . | . | . | T | C | −0.04 | 0.11 | . | . | F | 1.62 | 1.02 |
| Pro | 265 | . | . | . | . | . | T | C | 0.27 | −0.19 | . | . | F | 2.56 | 1.66 |
| Asp | 266 | . | . | . | . | T | T | . | 1.12 | −0.87 | * | * | F | 3.40 | 2.85 |
| Ser | 267 | A | . | . | . | . | T | . | 1.04 | −1.37 | . | * | F | 2.66 | 3.68 |
| Ser | 268 | A | . | . | . | . | . | . | 0.97 | −1.11 | * | * | F | 2.12 | 1.67 |
| Glu | 269 | A | . | . | . | . | . | . | 0.87 | −0.97 | * | . | F | 1.63 | 0.54 |
| Lys | 270 | A | . | . | B | . | . | . | 0.22 | −0.49 | * | . | F | 0.79 | 0.58 |
| Ile | 271 | A | . | . | B | . | . | . | 0.22 | −0.23 | . | * | . | 0.30 | 0.32 |
| Cys | 272 | A | . | . | B | . | . | . | −0.29 | −0.21 | . | . | . | 0.30 | 0.32 |
| Thr | 273 | . | . | B | B | . | . | . | −0.84 | 0.47 | . | . | . | −0.60 | 0.13 |
| Val | 274 | . | . | B | B | . | . | . | −1.19 | 1.11 | . | . | . | −0.60 | 0.14 |
| Gln | 275 | . | . | B | B | . | . | . | −1.23 | 0.86 | . | . | . | −0.60 | 0.26 |
| Leu | 276 | . | . | B | B | . | . | . | −0.64 | 0.69 | * | . | . | −0.60 | 0.29 |
| Val | 277 | . | . | . | B | T | . | . | −0.27 | 0.59 | * | * | . | −0.20 | 0.52 |
| Gly | 278 | . | . | . | . | T | T | . | −0.27 | 0.86 | * | * | F | 0.35 | 0.31 |
| Asn | 279 | . | . | . | . | T | T | . | 0.38 | 0.94 | * | . | F | 0.35 | 0.55 |
| Ser | 280 | . | . | . | . | T | T | . | 0.03 | 0.69 | * | . | F | 0.50 | 1.15 |
| Trp | 281 | . | . | . | . | . | T | C | 0.60 | 0.47 | . | . | F | 0.30 | 1.15 |
| Thr | 282 | . | . | . | . | . | T | C | 1.24 | 0.80 | * | . | F | 0.30 | 1.12 |
| Pro | 283 | . | . | . | . | . | T | C | 1.59 | 0.83 | . | . | F | 0.30 | 1.29 |
| Gly | 284 | . | . | . | . | . | T | C | 1.28 | 0.44 | . | . | F | 0.30 | 2.12 |
| Tyr | 285 | . | . | . | . | . | T | C | 1.58 | 0.01 | . | . | F | 0.60 | 2.12 |
| Pro | 286 | . | . | . | . | . | . | C | 1.87 | −0.07 | . | . | F | 1.00 | 2.38 |
| Glu | 287 | . | A | . | . | T | . | . | 1.59 | −0.50 | . | . | F | 1.00 | 4.16 |
| Thr | 288 | A | A | . | . | . | . | . | 0.99 | −0.43 | . | . | F | 0.60 | 2.68 |
| Gln | 289 | A | A | . | . | . | . | . | 0.67 | −0.50 | . | . | F | 0.60 | 1.43 |
| Glu | 290 | A | A | . | . | . | . | . | 0.70 | −0.36 | . | . | F | 0.45 | 0.44 |
| Ala | 291 | A | A | . | . | . | . | . | 0.91 | 0.07 | . | . | . | −0.30 | 0.47 |
| Leu | 292 | A | A | . | . | . | . | . | 0.06 | −0.01 | . | . | . | 0.30 | 0.47 |
| Cys | 293 | A | A | . | B | . | . | . | 0.06 | 0.23 | . | * | . | −0.30 | 0.20 |
| Pro | 294 | . | A | . | B | T | . | . | −0.23 | 0.71 | . | * | . | −0.20 | 0.29 |
| Gln | 295 | . | . | . | B | T | . | . | −0.53 | 1.13 | . | * | . | −0.20 | 0.37 |
| Val | 296 | . | . | . | B | T | . | . | −0.23 | 0.83 | . | * | . | −0.20 | 0.93 |
| Thr | 297 | . | . | . | B | T | . | . | 0.58 | 1.17 | * | * | . | −0.20 | 0.63 |
| Trp | 298 | . | . | . | B | T | . | . | 1.24 | 0.74 | * | * | . | −0.20 | 0.61 |
| Ser | 299 | . | . | . | B | T | . | . | 0.64 | 0.74 | * | * | . | −0.05 | 1.42 |
| Trp | 300 | . | . | . | B | T | . | . | 0.43 | 0.79 | * | * | . | 0.10 | 0.81 |
| Asp | 301 | . | . | . | B | T | . | . | 0.99 | 0.73 | * | * | F | 0.70 | 1.19 |
| Gln | 302 | . | . | . | . | . | . | C | 1.41 | 0.20 | * | * | F | 1.30 | 1.19 |
| Leu | 303 | . | . | . | . | . | T | C | 1.11 | −0.19 | * | . | F | 2.40 | 2.22 |
| Pro | 304 | . | . | . | . | . | T | C | 0.60 | −0.60 | * | * | F | 3.00 | 1.34 |
| Ser | 305 | . | . | . | . | T | T | . | 0.54 | 0.09 | * | * | F | 1.85 | 0.64 |
| Arg | 306 | . | . | . | . | T | T | . | 0.33 | 0.11 | * | * | F | 1.55 | 0.77 |
| Ala | 307 | . | . | . | . | T | . | . | −0.26 | −0.14 | * | * | F | 1.65 | 0.77 |
| Leu | 308 | . | . | . | . | . | . | C | −0.03 | −0.07 | * | * | F | 1.15 | 0.58 |
| Gly | 309 | . | . | . | . | . | . | C | −0.41 | 0.04 | * | . | F | 0.25 | 0.30 |
| Pro | 310 | . | A | . | . | . | . | C | −0.32 | 0.54 | * | . | . | −0.40 | 0.30 |
| Ala | 311 | . | A | . | . | . | . | C | −0.74 | 0.47 | * | * | . | −0.40 | 0.56 |
| Ala | 312 | A | A | . | . | . | . | . | −0.97 | 0.27 | . | . | . | −0.30 | 0.82 |
| Ala | 313 | . | A | . | . | . | . | C | −0.46 | 0.53 | . | . | . | −0.40 | 0.43 |
| Pro | 314 | . | . | . | . | . | . | C | −0.32 | 0.49 | . | . | F | −0.05 | 0.58 |
| Thr | 315 | . | . | . | . | . | . | C | −0.11 | 0.41 | . | . | F | −0.05 | 0.88 |
| Leu | 316 | . | . | . | . | . | . | C | 0.18 | −0.09 | . | . | F | 1.00 | 1.51 |
| Ser | 317 | . | . | . | . | . | T | C | 0.56 | −0.20 | . | . | F | 1.20 | 1.31 |
| Pro | 318 | . | . | . | . | . | T | C | 0.56 | −0.20 | . | . | F | 1.45 | 1.41 |
| Glu | 319 | . | . | . | . | . | T | C | 0.42 | −0.19 | . | . | F | 1.70 | 1.72 |
| Ser | 320 | . | . | . | . | . | T | C | 0.43 | −0.44 | . | . | F | 1.95 | 1.27 |
| Pro | 321 | . | . | . | . | . | T | C | 1.03 | −0.44 | . | . | F | 2.20 | 1.10 |
| Ala | 322 | . | . | . | . | T | T | . | 0.74 | −0.44 | . | . | F | 2.50 | 0.98 |
| Gly | 323 | . | . | . | . | . | T | C | 0.36 | 0.06 | . | . | F | 1.45 | 0.74 |
| Ser | 324 | . | . | . | . | . | T | C | −0.24 | 0.29 | . | . | F | 1.20 | 0.47 |
| Pro | 325 | A | A | . | . | . | . | . | −0.76 | 0.47 | . | . | F | 0.05 | 0.47 |
| Ala | 326 | A | A | . | . | . | . | . | −0.54 | 0.66 | . | . | . | −0.35 | 0.39 |
| Met | 327 | . | A | B | . | . | . | . | −0.17 | 0.63 | . | . | . | −0.60 | 0.50 |
| Met | 328 | . | A | B | . | . | . | . | −0.17 | 0.67 | . | . | . | −0.60 | 0.50 |
| Leu | 329 | . | A | B | . | . | . | . | −0.08 | 0.67 | . | . | . | −0.60 | 0.49 |
| Gln | 330 | . | . | . | . | . | T | C | 0.13 | 0.60 | * | * | . | 0.00 | 0.77 |
| Pro | 331 | . | . | . | . | . | T | C | −0.09 | 0.39 | * | * | F | 0.60 | 1.34 |
| Gly | 332 | . | . | . | . | . | T | C | 0.27 | 0.46 | * | * | F | 0.30 | 1.34 |
| Pro | 333 | . | . | . | . | . | T | C | 0.87 | 0.53 | * | . | F | 0.30 | 1.21 |
| Gln | 334 | . | A | . | . | . | . | C | 0.82 | 0.13 | * | . | F | 0.20 | 1.31 |
| Leu | 335 | . | A | B | . | . | . | . | 0.22 | 0.34 | * | . | . | −0.30 | 0.98 |
| Tyr | 336 | . | A | B | . | . | . | . | 0.43 | 0.53 | * | . | . | −0.60 | 0.63 |
| Asp | 337 | . | A | B | . | . | . | . | 0.19 | 0.10 | * | . | . | −0.30 | 0.61 |
| Val | 338 | . | A | B | . | . | . | . | −0.46 | 0.20 | * | . | . | −0.30 | 0.74 |
| Met | 339 | . | A | B | . | . | . | . | −0.67 | 0.16 | * | . | . | −0.30 | 0.35 |
| Asp | 340 | A | A | . | . | . | . | . | −0.44 | −0.17 | * | * | . | 0.30 | 0.33 |
| Ala | 341 | A | A | . | . | . | . | . | −0.09 | 0.33 | . | . | . | −0.30 | 0.44 |
| Val | 342 | A | A | . | . | . | . | . | 0.02 | −0.31 | . | . | . | 0.30 | 0.88 |
| Pro | 343 | A | . | . | . | . | . | . | 0.59 | −0.93 | . | . | . | 0.95 | 1.03 |
| Ala | 344 | A | A | . | . | . | . | . | 1.23 | −0.01 | * | . | . | 0.45 | 1.07 |
| Arg | 345 | A | A | . | . | . | . | . | 1.23 | −0.51 | * | . | F | 0.90 | 2.89 |
| Arg | 346 | A | A | . | . | . | . | . | 1.12 | −1.16 | * | . | F | 0.90 | 3.24 |
| Trp | 347 | A | A | . | . | . | . | . | 1.12 | −0.80 | * | * | F | 0.90 | 2.77 |
| Lys | 348 | A | A | . | . | . | . | . | 1.44 | −0.66 | * | * | F | 0.90 | 1.05 |
| Glu | 349 | A | A | . | . | . | . | . | 1.72 | −0.66 | * | * | . | 0.75 | 1.05 |
| Phe | 350 | A | A | . | . | . | . | . | 0.80 | −0.17 | * | * | . | 0.45 | 1.44 |
| Val | 351 | A | A | . | . | . | . | . | 0.34 | −0.40 | * | * | . | 0.30 | 0.59 |
| Arg | 352 | A | A | . | . | . | . | . | −0.18 | 0.03 | * | * | . | −0.30 | 0.34 |
| Thr | 353 | A | A | . | . | . | . | . | −0.11 | 0.71 | * | . | . | −0.60 | 0.32 |
| Leu | 354 | A | A | . | . | . | . | C | −0.11 | −0.07 | * | . | . | 0.50 | 0.85 |
| Gly | 355 | A | A | . | . | . | . | . | 0.00 | −0.71 | * | . | . | 0.60 | 0.76 |
| Leu | 356 | A | A | . | . | . | . | . | 0.86 | −0.21 | * | . | . | 0.30 | 0.53 |
| Arg | 357 | A | A | . | . | . | . | . | −0.14 | −0.70 | . | . | . | 0.75 | 1.11 |
| Glu | 358 | A | A | . | . | . | . | . | 0.17 | −0.70 | . | * | F | 0.75 | 0.79 |
| Ala | 359 | A | A | . | . | . | . | . | 0.39 | −1.13 | . | . | F | 0.90 | 1.65 |
| Glu | 360 | A | A | . | . | . | . | . | −0.12 | −1.31 | * | . | . | 0.60 | 0.85 |
| Ile | 361 | A | A | . | . | . | . | . | 0.69 | −0.67 | * | . | . | 0.60 | 0.37 |
| Glu | 362 | A | A | . | . | . | . | . | −0.28 | −0.67 | . | . | . | 0.60 | 0.63 |
| Ala | 363 | A | A | . | . | . | . | . | −0.28 | −0.53 | . | * | . | 0.60 | 0.27 |
| Val | 364 | A | A | . | . | . | . | . | −0.58 | −0.53 | . | * | . | 0.60 | 0.66 |
| Glu | 365 | A | A | . | . | . | . | . | −0.92 | −0.53 | * | . | . | 0.60 | 0.27 |
| Val | 366 | A | A | . | . | . | . | . | 0.08 | −0.10 | * | . | . | 0.30 | 0.26 |
| Glu | 367 | A | A | . | . | . | . | . | −0.62 | −0.60 | * | * | . | 0.60 | 0.69 |
| Ile | 368 | A | A | . | . | . | . | . | 0.08 | −0.46 | * | * | . | 0.30 | 0.35 |
| Gly | 369 | A | A | . | . | . | . | . | 0.93 | −0.46 | * | * | . | 0.30 | 0.91 |
| Arg | 370 | A | A | . | . | . | . | . | 0.93 | −1.10 | . | * | F | 0.75 | 0.88 |
| Phe | 371 | A | A | . | . | . | . | . | 1.79 | −0.70 | . | * | F | 0.90 | 2.18 |
| Arg | 372 | A | A | . | . | . | . | . | 1.54 | −0.99 | * | * | F | 0.90 | 3.81 |
| Asp | 373 | A | A | . | . | . | . | . | 2.43 | −0.66 | * | * | F | 0.90 | 3.05 |
| Gln | 374 | A | A | . | . | . | . | . | 2.18 | −0.66 | * | * | F | 0.90 | 6.10 |
| Gln | 375 | A | A | . | . | . | . | . | 1.26 | −0.83 | * | * | F | 0.90 | 3.08 |
| Tyr | 376 | A | A | . | . | . | . | . | 2.00 | −0.14 | * | * | . | 0.45 | 1.52 |
| Glu | 377 | A | A | . | . | . | . | . | 2.00 | −0.14 | * | * | . | 0.45 | 1.76 |
| Met | 378 | A | A | . | . | . | . | . | 1.71 | −0.54 | * | * | . | 0.75 | 1.99 |
| Leu | 379 | A | A | . | . | . | . | . | 1.82 | −0.03 | * | * | . | 0.45 | 1.33 |
| Lys | 380 | . | A | . | . | T | . | . | 1.82 | −0.79 | * | * | . | 1.15 | 1.51 |
| Arg | 381 | . | A | . | . | T | . | . | 2.07 | −0.39 | * | * | F | 1.00 | 2.64 |
| Trp | 382 | . | A | . | . | T | . | . | 2.07 | −0.60 | * | * | F | 1.30 | 5.55 |
| Arg | 383 | A | A | . | . | . | . | . | 2.46 | −0.89 | * | * | F | 0.90 | 4.80 |
| Gln | 384 | . | A | . | . | T | . | . | 2.68 | −0.46 | * | * | F | 1.00 | 3.79 |
| Gln | 385 | . | A | . | . | . | . | C | 2.29 | 0.04 | * | * | F | 0.20 | 3.64 |
| Gln | 386 | . | . | . | . | . | T | C | 1.37 | −0.44 | . | * | F | 1.20 | 1.84 |
| Pro | 387 | . | . | . | . | . | T | C | 1.31 | 0.24 | . | * | F | 0.45 | 0.88 |
| Ala | 388 | . | . | . | . | T | T | . | 0.61 | 0.27 | . | . | F | 0.65 | 0.50 |
| Gly | 389 | . | . | . | . | . | T | C | −0.24 | 0.37 | . | . | . | 0.30 | 0.29 |
| Leu | 390 | . | . | . | . | . | . | C | −0.49 | 0.61 | . | . | . | −0.20 | 0.14 |
| Gly | 391 | . | . | . | . | . | . | C | −1.08 | 0.94 | . | . | . | −0.20 | 0.22 |
| Ala | 392 | A | A | . | . | . | . | . | −1.46 | 0.94 | . | . | . | −0.60 | 0.22 |
| Val | 393 | A | A | . | . | . | . | . | −1.68 | 1.01 | . | . | . | −0.60 | 0.27 |
| Tyr | 394 | A | A | . | . | . | . | . | −1.33 | 1.01 | * | . | . | −0.60 | 0.23 |
| Ala | 395 | A | A | . | . | . | . | . | −0.41 | 0.59 | * | . | . | −0.60 | 0.39 |
| Ala | 396 | A | A | . | . | . | . | . | −0.67 | 0.09 | * | . | . | −0.15 | 1.03 |
| Leu | 397 | A | A | . | . | . | . | . | −0.42 | 0.06 | * | . | . | −0.30 | 0.65 |
| Glu | 398 | A | A | . | . | . | . | . | −0.38 | −0.27 | * | * | . | 0.30 | 0.63 |
| Arg | 399 | A | A | . | . | . | . | . | −0.13 | −0.09 | * | * | . | 0.30 | 0.52 |
| Met | 400 | A | A | . | . | . | . | . | 0.11 | −0.59 | * | * | . | 0.75 | 1.05 |
| Gly | 401 | A | . | . | . | . | . | . | 0.03 | −0.84 | * | * | . | 0.80 | 0.60 |
| Leu | 402 | A | . | . | . | . | T | . | −0.01 | −0.27 | * | * | . | 0.70 | 0.16 |
| Asp | 403 | A | . | . | . | . | T | . | −0.01 | 0.37 | . | * | . | 0.10 | 0.12 |
| Gly | 404 | A | . | . | . | . | T | . | −0.12 | −0.24 | * | * | . | 0.70 | 0.22 |
| Cys | 405 | A | . | . | . | . | T | . | −0.33 | −0.67 | * | * | . | 1.00 | 0.44 |
| Val | 406 | A | A | . | . | . | . | . | 0.12 | −0.67 | * | * | . | 0.60 | 0.22 |
| Glu | 407 | A | A | . | . | . | . | . | 0.63 | −0.67 | * | * | . | 0.60 | 0.43 |
| Asp | 408 | A | A | . | . | . | . | . | 0.74 | −0.71 | * | * | F | 0.90 | 1.07 |
| Leu | 409 | A | A | . | . | . | . | . | 0.28 | −1.29 | * | * | F | 0.90 | 2.81 |
| Arg | 410 | A | A | . | . | . | . | . | 0.94 | −1.24 | * | * | F | 0.90 | 1.34 |
| Ser | 411 | A | A | . | . | . | . | . | 1.91 | −0.84 | * | * | F | 0.90 | 1.39 |
| Arg | 412 | . | A | . | . | T | . | . | 1.57 | −0.84 | * | * | F | 1.30 | 3.30 |
| Leu | 413 | . | A | . | . | T | . | . | 1.36 | −1.10 | * | * | F | 1.30 | 1.67 |
| Gln | 414 | . | . | . | . | T | T | . | 1.78 | −0.67 | * | * | F | 1.70 | 1.92 |
| Arg | 415 | . | . | . | . | T | T | . | 1.28 | −0.63 | * | * | . | 1.55 | 1.25 |
| Gly | 416 | . | . | . | . | . | T | C | 1.19 | −0.20 | * | . | . | 1.05 | 1.94 |
| Pro | 417 | . | . | . | . | . | T | C | 0.69 | −0.46 | * | . | . | 1.05 | 1.44 |

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids, ranging from 1-295, can be deleted from the amino terminus of either the secreted polypeptide or the mature form of a polypeptide having an amino acid sequence shown in SEQ ID NO:6. Similarly, for example, any number of amino acids, ranging from 1-295, can be deleted from the carboxy terminus of the secreted protein or mature form of a polypeptide having an amino acid sequence shown in SEQ ID NO:6. Furthermore, any combination of the above amino and carboxy terminus deletions are preferred. Polynucleotides encoding these polypeptide fragments and antibodies that bind these polypeptide fragments are encompassed by the invention.

Accordingly, the present invention further provides polypeptides having one or more residues deleted from the amino terminus of the amino acid sequence of SEQ ID NO:6 (FIGS. 5A-5B), and polynucleotides encoding such polypeptides. For example, the present invention provides polypeptides comprising, or alternatively consisting of, the amino acid sequence of residues n¹-300 of SEQ ID NO:6, where n¹ is an integer in the range of 2-295. More in particular, in certain embodiments, the invention provides polynucleotides encoding polypeptides comprising, or alternatively consisting of, an amino acid sequence selected from the group consisting of residues of R-2 to H-300; A-3 to H-300; L-4 to H-300; E-5 to H-300; G-6 to H-300; P-7 to H-300; G-8 toH-300; L-9 to H-300; S-10 to H-300; L-11 to H-300; L-12 to H-300; C-13 to H-300; L-14 to H-300; V-15 to H-300;L-16 to H-300; A-17 to H-300; L-18 to H-300; P-19 to H-300; A-20 to H-300; L-21 to H-300; L-22 to H-300; P-23 toH-300; V-24 to H-300; P-25 to H-300; A-26 to H-300; V-27 to H-300; R-28 to H-300; G-29 to H-300; V-30 to H-300;A-31 to H-300; E-32 to H-300; T-33 to H-300; P-34 to H-300; T-35 to H-300; Y-36 to H-300; P-37 to H-300; W-38 toH-300; R-39 to H-300; D-40 to H-300; A-41 to H-300; E-42 to H-300; T-43 to H-300; G-44 to H-300; E-45 to H-300;R-46 to H-300; L-47 to H-300; V-48 to H-300; C-49 to H-300; A-50 to H-300; Q-51 to H-300; C-52 to H-300; P-53 toH-300; P-54 to H-300; G-55 to H-300; T-56 to H-300; F-57 to H-300; V-58 to H-300; Q-59 to)H-300; R-60 to H-300;P-61 to H-300; C-62 to H-300; R-63 to H-300; R-64 to H-300; D-65 to H-300; S-66 to H-300; P-67 to H-300; T-68 to 1H-300; T-69 to H-300; C-70 to H-300; G-71 to H-300; P-72 to H-300; C-73 to H-300; P-74 to H-300; P-75 to H-300;R-76 to H-300; H-77 to H-300; Y-78 to H-300; T-79 to H-300; Q-80 to H1-300; F-81 to H-300; W-82 to H-300; N-83 toH-300; Y-84 to H-300; L-85 to H-300; E-86 to H-300; R-87 to H-300; C-88 to H-300; R-89 to H-300; Y-90 to H-300;C-91 to H-300; N-92 to H-300; V-93 to H-300; L-94 to H-300; C-95 to H-300; G-96 to H-300; E-97 to H-300; R-98 to 1H-300; E-99 to H-300; E-100 to H-300; E-101 to H-300; A-102 to H-300; R-103 to H1-300; A-104 to H-300; C-105 to H-300; H-106 to H-300; A-107 to H-300; T-108 to H-300; H-109 to H-300; N-10 to H-300; R-111 to H-300; A-112 toH-300; C-113 to H-300; R-114 to H-300; C-115 to H-300; R-116 to H-300; T-117 to H-300; G-118 to H-300; F-119 to 1H-300; F-120 to H-300; A-121 to H-300; H-122 to H-300; A-123 to H-300; G-124 to H-300; F-125 to H-300; C-126 to 1H-300; L-127 to H-300; E-128 to H-300; H-129 to H-300; A-130 to H-300; S-131 to H-300; C-132 to H-300; P-133 to 1H-300; P-134 to H1-300; G-135 to H-300; A-136 to H-300; G-137 to H-300; V-138 to H-300; I-139 to H-300; A-140 to 1H-300; P-141 to H-300; G-142 to H-300; T-143 to H-300; P-144 to H-300; S-145 to H-300; Q-146-to H-300; N-147 to 1H-300; T-148 to H-300; Q-149 to H-300; C-150 to H-300; Q-151 to H-300; P-152 to H-300; C-153 to H-300; P-154 to 1H-300; P-155 to H-300; G-156 to H-300; T-157 to H-300; F-158 to H-300; S-159 to H-300; A-160 to H-300; S-161 to 1H-300; S-162 to H-300; S-163 to H-300; S-164 to H-300; S-165 to H-300; E-166 to H-300; Q-167 to H-300; C-168 to 1H-300; Q-169 to H-300; P-170 to H-300; H-171 to H-300; R-172 to H-300; N-173 to H-300; C-174 to H-300; T-175 to 1H-300; A-176 to H-300; L-177 to H-300; G-178 to H-300; L-179 to H-300; A-180 to H-300; L-181 to H-300; N-182 to 1H-300; V-183 to H-300; P-184 to H-300; G-185 to H-300; S-186 to H-300; S-187 to H-300; S-188 to H-300; H-189 toH-300; D-190 to H-300; T-191 to H-300; L-192 to H-300; C-193 to H-300; T-194 to H-300; S-195 to H-300; C-196 to 1H-300; T-197 to 1′-300; G-198 to H-300; F-199 to H-300; P-200 to H-300; L-201 to H-300; S-202 to H-300; T-203 toH-300; R-204 to H-300; V-205 to H-300; P-206 to H-300; G-207 to H-300; A-208 to H-300; E-209 to H-300; E-210 to 1H-300; C-211 to H-300; E-212 to H-300; R-213 to H-300; A-214 to H-300; V-215 to H-300; I-216 to H-300; D-217 to 1H-300; F-218 to H-300; V-219 to H-300; A-220 to H-300; F-221 to H-300; Q-222 to H-300; D-223 to H-300; 1-224 to 1H-300; S-225 to H-300; 1-226 to H-300; K-227 to H-300; R-228 to H-300; L-229 to H-300; Q-230 to H-300; R-231 to 1H-300; L-232 to H-300; L-233 to H-300; Q-234 to H-300; A-235 to H-300; L-236 to H-300; E-237 to H-300; A-238 toH-300; P-239 to H-300; E-240 to H-300; G-241 to H-300; W-242 to H-300; G-243 to H-300; P-244 to H-300; T-245 toH-300; P-246 to H-300; R-247 to H-300; A-248 to H-300; G-249 to H-300; R-250 to H-300; A-251 to H-300; A-252 toH-300; L-253 to H-300; Q-254 to H-300; L-255 to H-300; K-256 to H-300; L-257 to H-300; R-258 to H-300; R-259 toH-300; R-260 to H-300; L-261 to H-300; T-262 to H-300; E-263 to H-300; L-264 to H-300; L-265 to H-300; G-266 toH-300; A-267 to H-300; Q-268 to H-300; D-269 to H-300; G-270 to H-300; A-271 to H-300; L-272 to H-300; L-273 toH-300; V-274 to H-300; R-275 to H-300; L-276 to H-300; L-277 to H-300; Q-278 to H-300; A-279 to H-300; L-280 toH-300; R-281 to H-300; V-282 to H-300; A-283 to H-300; R-284 to H-300; M-285 to H-300; P-286 to H-300; G-287 to H-300; L-288 to H-300; E-289 to H-300; R-290 to H-300; S-291 to H-300; V-292 to H-300; R-293 to H-300; E-294 to H-300; R-295 to H-300; of SEQ ID NO:6. Polynucleotides encoding the above polypeptide fragments and antibodies that bind the above polypeptide fragments are also encompassed by the invention.

The present invention also encompasses nucleic acid molecules comprising, or alternatively, consisting of, a polynucleotide sequence at least 80%, 85%, 90%, 92%, 95%, 96%, 97%, 98% or 99% identical to the polynucleotide sequence encoding polypeptides as described above. The present invention also encompasses the above polynucleotide sequences fused to a heterologous polynucleotide sequence. Polypeptides encoded by these nucleic acids and/or polynucleotide sequences are also encompassed by the invention, as are polypeptides comprising, or alternatively consisting of, an amino acid sequence at least 80%, 85%, 90%, 92%, 95%, 96%, 97%, 98% or 99% identical to the amino acid sequence described above, and polynucleotides that encode such polypeptides.

Moreover, the present invention further provides polypeptides having one or more residues deleted from the carboxy terminus of the amino acid sequence of the polypeptide shown in SEQ ID NO:6 (FIGS. 5A-5B). For example, the present invention provides polypeptides comprising, or alternatively consisting of, the amino acid sequence of residues 1-m¹ of the amino acid sequence in SEQ ID NO:6, where m¹ is any integer in the range 6-299. More in particular, in certain embodiments, the invention provides polynucleotides encoding polypeptides comprising, or alternatively consisting of, an amino acid sequence selected from the group consisting of residues M-1 to V-299; M-1 to P-298; M-1 to L-297; M-1 to F-296; M-1 to R-295; M-1 toE-294; M-1 to R-293; M-1 to V-292; M-1 to S-291; M-1 to R-290; M-1 toE-289; M-1 to L-288; M-1 to G-287; M-1 toP-286; M-1 to M-285; M-1 to R-284; M-1 to A-283; M-1 to V-282; M-1 to R-281; M-1 to L-280; M-1 to A-279; M-1 to Q-278; M-1 to L-277; M-1 to L-276; M-1 to R-275; M-1 to V-274; M-1 to L-273; M-1 to L-272; M-1 to A-271; M-1 to G-270; M-1 to D-269; M-1 to Q-268; M-1 to A-267; M-1 to G-266; M-1 to L-265; M-1 to L-264; M-1 to E-263;M-1 to T-262; M-1 to L-261; M-1 to R-260; M-1 to R-259; M-1 to R-258; M-1 to L-257; M-1 to K-256; M-1 to L-255;M-1 to Q-254; M-1 to L-253; M-1 to A-252; M-1 to A-251; M-1 to R-250; M-1 to G-249; M-1 to A-248; M-1 toR-247; M-1 to P-246; M-1 to T-245; M-1 to P-244; M-1 to G-243; M-1 to W-242; M-1 to G-241; M-1 to E-240; M-1 to P-239; M-1 to A-238; M-1 to E-237; M-1 to L-236; M-1 to A-235; M-1 to Q-234; M-1 to L-233; M-1 to L-232; M-1 to R-231; M-1 to Q-230; M-1 to L-229; M-1 to R-228; M-1 to K-227; M-1 to 1-226; M-1 to S-225; M-1 to 1-224; M-1 to D-223; M-1 to Q-222; M-1 to F-221; M-1 to A-220; M-1 to V-219; M-1 to F-218; M-1 to D-217; M-1 to 1-216; M-1 to V-215; M-1 to A-214; M-1 to R-213; M-1 to E-212; M-1 to C-211; M-1 to E-210; M-1 to E-209; M-1 to A-208;M-1 to G-207; M-1 to P-206; M-1 to V-205; M-1 to R-204; M-1 to T-203; M-1 to S-202; M-I to L-201; M-I to P-200;M-1 to F-199; M-1 to G-198; M-I to T-197; M-I to C-196; M-1 to S-195; M-1 to T-194; M-1 to C-193; M-1 to L-192;M-1 to T-191; M-1 to D-190; M-1 to H-189; M-1 to S-188; M-1 to S-187; M-1 to S-186; M-1 to G-185; M-1 to P-184;M-1 to V-183; M-1 to N-182; M-1 to L-181; M-1 to A-180; M-1 to L-179; M-1 to G-178; M-1 to L-177; M-1 toA-176; M-1 to T-175; M-1 to C-174; M-1 to N-173; M-1 to R-172; M-1 to H-171; M-1 to P-170; M-1 to Q-169; M-1 to C-168; M-1 to Q-167; M-1 to E-166; M-1 to S-165; M-1 to S-164; M-1 to S-163; M-1 to S-162; M-1 to S-161; M-Ito A-160; M-1 to S-159; M-1 to F-158; M-1 to T-157; M-1 to G-156; M-1 to P-155; M-1 to P-154; M-1 to C-153; M-Ito P-152; M-1 to Q-151; M-1 to C-150; M-1 to Q-149; M-1 to T-148; M-1 to N-147; M-1 to Q-146; M-1 to S-145;M-1 to P-144; M-1 to T-143; M-1 to G-142; M-1 to P-141; M-1 to A-140; M-1 to 1-139; M-1 to V-138; M-1 to G-137;M-1 to A-136; M-1 to G-135; M-1 to P-134; M-1 to P-133; M-1 to C-132; M-1 to S-131; M-1 to A-130; M-1 toH-129; M-1 to E-128; M-1 to L-127; M-1 to C-126; M-1 to F-125; M-1 to G-124; M-1 to A-123; M-1 to H-122; M-1 to A-121; M-1 to F-120; M-1 to F-119; M-1 to G-118; M-1 to T-117; M-1 to R-116; M-1 to C-115; M-1 to R-114; M-1 to C-113; M-1 to A-112;M-1 to R-11; M-1 to N-110; M-1 to H-109; M-1 toT-108;M-1 toA-107;M-1 to H-106;M-1 to C-105; M-1 to A-104; M-1 to R-103; M-1 to A-102; M-1 to E-101; M-1 to E-100; M-1 to E-99; M-1 to R-98;M-1 to E-97; M-1 to G-96; M-1 to C-95; M-1 to L-94; M-1 to V-93; M-1 to N-92; M-1 to C-91; M-1 to Y-90; M-1 toR-89; M-1 to C-88; M-1 to R-87; M-1 to E-86; M-1 to L-85; M-1 to Y-84; M-1 to N-83; M-1 to W-82; M-1 to F-81;M-1 to Q-80; M-1 to T-79; M-1 to Y-78; M-1 to H-77; M-1 to R-76; M-1 to P-75; M-1 to P-74; M-1 to C-73; M-1 toP-72; M-1 to G-71; M-1 to C-70; M-1 to T-69; M-1 to T-68; M-1 to P-67; M-1 to S-66; M-1 to D-65; M-1 to R-64;M-1 to R-63; M-1 to C-62; M-1 to P-61; M-1 to R-60; M-1 to Q-59; M-1 to V-58; M-1 to F-57; M-1 to T-56; M-1 toG-55; M-1 to P-54; M-1 to P-53; M-1 to C-52; M-1 to Q-51; M-1 to A-50; M-1 to C-49; M-1 to V-48; M-1 to L-47;M-1 to R-46; M-1 to E-45; M-1 to G-44; M-1 to T-43; M-1 to E-42; M-1 to A-41; M-1 to D-40; M-1 to R-39; M-1 toW-38; M-1 to P-37; M-1 to Y-36; M-1 to T-35; M-1 to P-34; M-1 to T-33; M-1 to E-32; M-1 to A-31; M-1 to V-30;M-1 to G-29; M-1 to R-28; M-1 to V-27; M-1 to A-26; M-1 to P-25; M-1 to V-24; M-1 to P-23; M-1 to L-22; M-1 toL-21; M-1 to A-20; M-1 to P-19; M-1 to L-18; M-1 to A-17; M-1 to L-16; M-1 to V-15; M-1 to L-14; M-1 to C-13;M-1 to L-12; M-1 to L-11; M-1 to S-10; M-1 to L-9; M-1 to G-8; M-1 to P-7; M-1 to G-6; of SEQ ID NO:6. Polynucleotides encoding the above polypeptide fragments and antibodies that bind the above polypeptide fragments are also encompassed by the invention.

The present invention encompasses nucleic acid molecules comprising, or alternatively, consisting of, a polynucleotide sequence at least 80%, 85%, 90%, 92%, 95%, 96%, 97%, 98% or 99% identical to the polynucleotide sequence encoding the polypeptides described above. The present invention also encompasses the above polynucleotide sequences fused to a heterologous polynucleotide sequence. Polypeptides encoded by these nucleic acids and/or polynucleotide sequences are also encompassed by the invention, as are polypeptides comprising, or alternatively consisting of, an amino acid sequence at least 80%, 85%, 90%, 92%, 95%, 96%, 97%, 98% or 99% identical to the amino acid sequence described above, and polynucleotides that encode such polypeptides.

The invention also provides polypeptides having one or more amino acids deleted from both the amino and the carboxyl termini of the polypeptide of SEQ ID NO:6 (FIGS. 5A-5B). For example, amino terminal and carboxyl terminal deletions of the polypeptide sequence may be described generally, for example, as having residues n¹-m¹ of SEQ ID NO:6 where n¹ is an integer in the range of 1-286 and m¹ is an integer in the range of 15-300. For example, and more in particular, the invention provides polynucleotides encoding polypeptides comprising, or alternatively consisting of, an amino acid sequence selected from the group consisting of residues of M-1 to V-15; R-2 to L-16;A-3 to A-17; L-4 to L-18; E-5 to P-19; G-6 to A-20; P-7 to L-21; G-8 to L-22; L-9 toP-23; S-10 to V-24; L-11 to P-25;L-12 to A-26; C-13 to V-27; L-14 to R-28; V-15 to G-29; L-16 to V-30; A-17 to A-31; L-18 to E-32; P-19 to T-33;A-20 to P-34; L-21 to T-35; L-22 to Y-36; P-23 to P-37; V-24 to W-38; P-25 to R-39; A-26 to D-40; V-27 to A-41;R-28 to E-42; G-29 to T-43; V-30 to G-44; A-31 to E-45; E-32 to R-46; T-33 to L-47; P-34 to V-48; T-35 to C-49;Y-36 to A-50; P-37 to Q-51; W-38 to C-52; R-39 to P-53; D-40 to P-54; A-41 to G-55; E-42 to T-56; T-43 to F-57;G-44 to V-58; E-45 to Q-59; R-46 to R-60; L-47 to P-61; V-48 to C-62; C-49 to R-63; A-50 to R-64; Q-51 to D-65;C-52 to S-66; P-53 to P-67; P-54 to T-68; G-55 to T-69; T-56 to C-70; F-57 to G-71; V-58 to P-72; Q-59 to C-73;R-60 to P-74; P-61 to P-75; C-62 to R-76; R-63 to H-77; R-64 to Y-78; D-65 to T-79; S-66 to Q-80; P-67 to F-81;T-68 to W-82; T-69 to N-83; C-70 to Y-84; G-71 to L-85; P-72 to E-86; C-73 to R-87; P-74 to C-88; P-75 to R-89;R-76 to Y-90; H-77 to C-91; Y-78 to N-92; T-79 to V-93; Q-80 to L-94; F-81 to C-95; W-82 to G-96; N-83 to E-97;Y-84 to R-98; L-85 to E-99; E-86 to E-100; R-87 to E-101; C-88 to A-102; R-89 to R-103; Y-90 to A-104; C-91 toC-105; N-92 to H-106; V-93 to A-107; L-94 to T-108; C-95 to H-109; G-96 to N-110; E-97 to R-111; R-98 to A-112;E-99 to C-113; E-100 to R-114; E-101 to C-115; A-102 to R-116; R-103 to T-117; A-104 to G-118; C-105 to F-119;H-106 to F-120; A-107 to A-121; T-108 to H-122; H-109 to A-123; N-110 to G-124; R-1 II to F-125; A-112 to C-126;C-113 to L-127; R-114 to E-128; C-115 to H-129; R-116 to A-130; T-117 to S-131; G-118 to C-132; F-I 19 to P-133;F-120 to P-134; A-121 to G-135; H-122 to A-136; A-123 to G-137; G-124 to V-138; F-125 to 1-139; C-126 to A-140;L-127 to P-141; E-128 to G-142; H-129 to T-143; A-130 to P-144; S-131 to S-145; C-132 to Q-146; P-133 to N-147;P-134 to T-148; G-135 to Q-149; A-136 to C-150; G-137 to Q-151; V-138 to P-152; 1-139 to C-153; A-140 to P-154;P-141 to P-155; G-142 to G-156; T-143 to T-157; P-144 to F-158; S-145 to S-159; Q-146 to A-160; N-147 to S-161;T-148 to S-162; Q-149 to S-163; C-150 to S-164; Q-151 to S-165; P-152 to E-166; C-153 to Q-167; P-154 to C-168;P-155 to Q-169; G-156 to P-170; T-157 to H-171; F-158 to R-172; S-159 to N-173; A-160 to C-174; S-161 to T-175;S-162 to A-176; S-163 to L-177; S-164 to G-178; S-165 to L-179; E-166 to A-180; Q-167 to L-181; C-168 to N-182;Q-169 to V-183; P-170 to P-184; H-171 to G-185; R-172 to S-186; N-173 to S-187; C-174 to S-188; T-175 to H-189;A-176 to D-190; L-177 to T-191; G-178 to L-192; L-179 to C-193; A-180 to T-194; L-181 to S-195; N-182 to C-196;V-183 to T-197; P-184 to G-198; G-185 to F-199; S-186 to P-200; S-187 to L-201; S-188 to S-202; H-189 to T-203;D-190 to R-204; T-191 to V-205; L-192 to P-206; C-193 to G-207; T-194 to A-208; S-195 to E-209; C-196 to E-210;T-197 to C-211; G-198 to E-212; F-199 to R-213; P-200 to A-214; L-201 to V-215; S-202 to 1-216; T-203 to D-217;R-204 to F-218; V-205 to V-219; P-206 to A-220; G-207 to F-221; A-208 to Q-222; E-209 to D-223; E-210 to 1-224;C-211 to S-225; E-212 to 1-226; R-213 to K-227; A-214 to R-228; V-215 to L-229; 1-216 to Q-230; D-217 to R-231;F-218 to L-232; V-219 to L-233; A-220 to Q-234; F-221 to A-235; Q-222 to L-236; D-223 to E-237; 1-224 to A-238;S-225 to P-239; 1-226 to E-240; K-227 to G-241; R-228 to W-242; L-229 to G-243; Q-230 to P-244; R-231 to T-245;L-232 to P-246; L-233 to R-247; Q-234 to A-248; A-235 to G-249; L-236 to R-250; E-237 to A-251; A-238 to A-252;P-239 to L-253; E-240 to Q-254; G-241 to L-255; W-242 to K-256; G-243 to L-257; P-244 to R-258; T-245 to R-259;P-246 to R-260; R-247 to L-261; A-248 to T-262; G-249 to E-263; R-250 to L-264; A-251 to L-265; A-252 to G-266;L-253 to A-267; Q-254 to Q-268; L-255 to D-269; K-256 to G-270; L-257 to A-271; R-258 to L-272; R-259 to L-273;R-260 to V-274; L-261 to R-275; T-262 to L-276; E-263 to L-277; L-264 to Q-278; L-265 to A-279; G-266 to L-280;A-267 to R-281; Q-268 to V-282; D-269 to A-283; G-270 to R-284; A-271 to M-285; L-272 to P-286; L-273 to G-287;V-274 to L-288; R-275 to E-289; L-276 to R-290; L-277 to S-291; Q-278 to V-292; A-279 to R-293; L-280 to E-294;R-281 to R-295; V-282 to F-296; A-283 to L-297; R-284 to P-298; M-285 to V-299; or P-286 to H-300 of SEQ ID NO:6. Polynucleotides encoding the above polypeptide fragments and antibodies that bind the above polypeptide fragments are also encompassed by the invention.

The present invention encompasses nucleic acid molecules comprising, or alternatively, consisting of, a polynucleotide sequence at least 80%, 85%, 90%, 92%, 95%, 96%, 97%, 98% or 99% identical to the polynucleotide sequence encoding the polypeptides described above. The present invention also encompasses the above polynucleotide sequences fused to a heterologous polynucleotide sequence. Polypeptides encoded by these nucleic acids and/or polynucleotide sequences are also encompassed by the invention, as are polypeptides comprising, or alternatively consisting of, an amino acid sequence at least 80%, 85%, 90%, 92%, 95%, 96%, 97%, 98% or 99% identical to the amino acid sequence described above, and polynucleotides that encode such polypeptides.

Also preferred are polypeptide and polynucleotide fragments characterized by structural or functional domains (See, FIG. 6 and Table 3), such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. See FIG. 6 and Table 3. Polypeptide fragments of SEQ ID NO:6 falling within conserved domains, hydrophillic, and antigenic domains are specifically contemplated by the present invention. Moreover, polynucleotides encoding these domains and antibodies that bind to these domains are also contemplated.

TABLE 3

| Res: | Pos: | I | II | III | IV | V | VI | VII | VIII | IX | X | XI | XII | XIII | XIV |
|---|---|---|---|---|---|---|---|---|---|---|---|---|---|---|---|
| Met | 1 | . | . | B | . | . | . | . | 0.06 | 0.09 | * | . | . | −0.10 | 0.60 |
| Arg | 2 | . | . | B | . | . | . | . | 0.10 | −0.34 | * | . | . | 0.50 | 0.82 |
| Ala | 3 | . | . | B | . | . | . | . | 0.28 | −0.34 | * | . | . | 0.50 | 0.63 |
| Leu | 4 | A | . | . | . | . | . | . | 0.32 | −0.34 | * | . | . | 0.50 | 0.99 |
| Glu | 5 | A | . | . | . | . | . | . | −0.10 | −0.53 | * | . | F | 0.95 | 0.50 |
| Gly | 6 | . | . | . | . | . | T | C | 0.20 | 0.16 | * | . | F | 0.45 | 0.41 |
| Pro | 7 | . | . | . | . | T | T | . | −0.72 | 0.04 | * | . | F | 0.65 | 0.66 |
| Gly | 8 | . | . | . | . | T | T | . | −0.94 | 0.04 | . | . | F | 0.65 | 0.32 |
| Leu | 9 | A | . | . | . | . | T | . | −0.80 | 0.73 | . | . | . | −0.20 | 0.26 |
| Ser | 10 | A | A | . | . | . | . | . | −1.61 | 0.87 | . | . | . | −0.60 | 0.09 |
| Leu | 11 | . | A | B | . | . | . | . | −2.12 | 1.13 | . | . | . | −0.60 | 0.08 |
| Leu | 12 | . | A | B | . | . | . | . | −2.72 | 1.34 | . | . | . | −0.60 | 0.07 |
| Cys | 13 | . | A | B | . | . | . | . | −2.97 | 1.34 | . | . | . | −0.60 | 0.04 |
| Leu | 14 | . | A | B | . | . | . | . | −2.97 | 1.46 | . | . | . | −0.60 | 0.05 |
| Val | 15 | . | A | B | . | . | . | . | −2.88 | 1.46 | . | . | . | −0.60 | 0.05 |
| Leu | 16 | . | A | B | . | . | . | . | −2.66 | 1.20 | . | . | . | −0.60 | 0.15 |
| Ala | 17 | . | A | B | . | . | . | . | −2.66 | 1.13 | . | . | . | −0.60 | 0.18 |
| Leu | 18 | . | A | B | . | . | . | . | −2.80 | 1.13 | . | . | . | −0.60 | 0.20 |
| Pro | 19 | A | A | . | . | . | . | . | −2.20 | 1.17 | . | . | . | −0.60 | 0.20 |
| Ala | 20 | . | A | B | . | . | . | . | −2.20 | 0.91 | . | . | . | −0.60 | 0.31 |
| Leu | 21 | . | A | B | . | . | . | . | −1.60 | 1.06 | . | * | . | −0.60 | 0.28 |
| Leu | 22 | . | A | B | . | . | . | . | −1.60 | 0.80 | . | . | . | −0.60 | 0.28 |
| Pro | 23 | . | A | B | . | . | . | . | −1.64 | 0.87 | . | * | . | −0.60 | 0.28 |
| Val | 24 | . | . | B | . | . | . | . | −1.32 | 1.01 | . | * | . | −0.40 | 0.25 |
| Pro | 25 | . | . | B | . | . | . | . | −1.08 | 0.33 | * | . | . | −0.10 | 0.60 |
| Ala | 26 | . | . | B | B | . | . | . | −1.12 | 0.07 | . | . | . | −0.30 | 0.38 |
| Val | 27 | . | . | B | B | . | . | . | −0.90 | 0.29 | * | * | . | −0.30 | 0.38 |
| Arg | 28 | . | . | B | B | . | . | . | −0.69 | 0.14 | * | * | . | −0.30 | 0.25 |
| Gly | 29 | . | . | B | B | . | . | . | −0.14 | −0.29 | * | * | . | 0.30 | 0.43 |
| Val | 30 | . | . | B | B | . | . | . | −0.14 | −0.30 | * | * | . | 0.30 | 0.83 |
| Ala | 31 | . | . | B | B | . | . | . | 0.13 | −0.51 | * | * | . | 0.60 | 0.66 |
| Glu | 32 | . | . | B | . | . | . | . | 0.74 | −0.03 | * | * | F | 0.65 | 0.96 |
| Thr | 33 | . | . | B | . | . | T | . | 0.42 | 0.30 | * | * | F | 0.40 | 2.02 |
| Pro | 34 | . | . | . | . | T | T | . | 0.48 | 0.09 | * | * | F | 0.80 | 3.10 |
| Thr | 35 | . | . | . | . | T | T | . | 1.44 | 0.50 | * | . | F | 0.50 | 1.88 |
| Tyr | 36 | . | . | . | . | . | T | C | 2.03 | 0.50 | * | . | . | 0.15 | 2.55 |
| Pro | 37 | . | . | . | . | T | . | . | 1.44 | 0.01 | * | . | . | 0.45 | 2.76 |
| Trp | 38 | . | A | . | . | . | . | C | 1.76 | 0.09 | * | . | . | 0.05 | 1.93 |
| Arg | 39 | . | A | B | . | . | . | . | 1.66 | −0.40 | * | . | F | 0.60 | 2.13 |
| Asp | 40 | A | A | . | . | . | . | . | 1.62 | −0.67 | * | . | F | 0.90 | 1.99 |
| Ala | 41 | . | A | . | . | . | . | C | 1.87 | −0.67 | * | . | F | 1.10 | 1.87 |
| Glu | 42 | A | A | . | . | . | . | . | 2.19 | −1.59 | * | * | F | 0.90 | 1.66 |
| Thr | 43 | A | A | . | . | . | . | . | 1.67 | −1.59 | * | . | F | 0.90 | 1.94 |
| Gly | 44 | . | A | . | . | T | . | . | 0.70 | −0.90 | * | . | F | 1.30 | 1.59 |
| Glu | 45 | A | A | . | . | . | . | . | 0.03 | −0.76 | * | * | F | 0.75 | 0.68 |
| Arg | 46 | A | A | . | . | . | . | . | 0.03 | −0.19 | * | . | F | 0.45 | 0.25 |
| Leu | 47 | A | A | . | . | . | . | . | 0.03 | −0.17 | * | . | . | 0.30 | 0.26 |
| Val | 48 | . | A | B | . | . | . | . | −0.32 | −0.20 | . | . | . | 0.30 | 0.26 |
| Cys | 49 | . | A | B | . | . | . | . | −0.19 | 0.37 | . | . | . | −0.30 | 0.07 |
| Ala | 50 | . | A | B | . | . | . | . | −0.40 | 0.80 | . | . | . | −0.60 | 0.13 |
| Gln | 51 | . | A | B | . | . | . | . | −0.86 | 0.54 | . | . | . | −0.60 | 0.28 |
| Cys | 52 | . | A | B | . | . | . | . | −0.36 | 0.33 | . | . | . | −0.30 | 0.51 |
| Pro | 53 | . | . | . | . | . | T | C | −0.20 | 0.24 | . | . | F | 0.45 | 0.73 |
| Pro | 54 | . | . | . | . | T | T | . | −0.39 | 0.53 | . | . | F | 0.35 | 0.36 |
| Gly | 55 | . | . | . | . | T | T | . | 0.20 | 0.77 | * | . | F | 0.35 | 0.50 |
| Thr | 56 | . | . | B | . | . | T | . | 0.31 | 0.60 | . | . | F | −0.05 | 0.56 |
| Phe | 57 | . | . | B | B | . | . | . | 0.77 | 0.17 | * | . | F | −0.15 | 0.71 |
| Val | 58 | . | . | B | B | . | . | . | 0.31 | 0.17 | * | . | . | 0.19 | 1.12 |
| Gln | 59 | . | . | B | B | . | . | . | 0.63 | 0.31 | * | . | F | 0.53 | 0.41 |
| Arg | 60 | . | . | B | . | . | T | . | 1.09 | −0.17 | * | . | F | 1.87 | 0.94 |
| Pro | 61 | . | . | B | . | . | T | . | 1.40 | −0.96 | * | . | F | 2.66 | 2.47 |
| Cys | 62 | . | . | . | . | T | T | . | 1.80 | −1.60 | * | . | F | 3.40 | 2.38 |
| Arg | 63 | . | . | . | . | T | T | . | 2.44 | −1.61 | * | . | F | 3.06 | 1.63 |
| Arg | 64 | . | . | . | . | T | . | . | 2.13 | −1.19 | * | . | F | 2.77 | 1.63 |
| Asp | 65 | . | . | . | . | T | . | . | 1.71 | −1.13 | * | . | F | 2.68 | 4.39 |
| Ser | 66 | . | . | . | . | T | T | . | 1.26 | −1.21 | . | . | F | 2.79 | 3.24 |
| Pro | 67 | . | . | . | . | T | T | . | 1.58 | −0.64 | . | . | F | 2.55 | 0.89 |
| Thr | 68 | . | . | . | . | T | T | . | 1.26 | −0.21 | . | . | F | 2.50 | 0.52 |
| Thr | 69 | . | . | . | . | T | T | . | 0.48 | 0.21 | . | . | F | 1.65 | 0.61 |
| Cys | 70 | . | . | . | . | T | . | . | 0.27 | 0.40 | . | . | F | 1.14 | 0.21 |
| Gly | 71 | . | . | . | . | T | T | . | 0.36 | 0.40 | * | * | F | 1.33 | 0.22 |
| Pro | 72 | . | . | . | . | T | T | . | 0.68 | 0.34 | * | . | F | 1.62 | 0.24 |
| Cys | 73 | . | . | . | . | . | T | C | 0.96 | −0.14 | * | . | F | 2.01 | 0.88 |
| Pro | 74 | . | . | . | . | . | T | C | 1.02 | −0.21 | * | . | F | 2.40 | 1.21 |
| Pro | 75 | . | . | . | . | T | T | . | 1.38 | 0.11 | * | * | F | 1.76 | 1.23 |
| Arg | 76 | . | . | . | . | T | T | . | 1.72 | 0.17 | * | * | F | 1.52 | 3.30 |
| His | 77 | . | . | B | . | . | T | . | 1.23 | 0.00 | * | * | F | 0.88 | 3.70 |
| Tyr | 78 | . | . | B | . | . | T | . | 1.61 | 0.36 | * | * | . | 0.49 | 2.07 |
| Thr | 79 | . | . | B | . | . | . | . | 1.82 | 0.84 | * | . | . | −0.25 | 1.11 |
| Gln | 80 | . | . | B | . | . | . | . | 1.79 | 1.24 | * | * | . | −0.25 | 1.31 |
| Phe | 81 | . | . | . | . | T | . | . | 0.87 | 1.50 | * | * | . | 0.15 | 1.31 |
| Trp | 82 | . | . | . | . | T | . | . | 0.90 | 1.43 | * | . | . | 0.00 | 0.75 |
| Asn | 83 | . | A | . | . | T | . | . | 1.26 | 0.94 | * | . | . | −0.20 | 0.75 |
| Tyr | 84 | . | A | . | . | T | . | . | 0.90 | 0.54 | * | * | . | −0.05 | 1.70 |
| Leu | 85 | . | A | . | . | T | . | . | 1.01 | 0.33 | * | * | . | 0.38 | 0.87 |
| Glu | 86 | . | A | . | . | T | . | . | 1.47 | −0.59 | * | * | . | 1.71 | 1.05 |
| Arg | 87 | . | A | . | . | T | . | . | 1.09 | −0.23 | . | * | . | 1.69 | 1.05 |
| Cys | 88 | . | . | . | . | T | T | . | 1.09 | −0.41 | . | * | . | 2.22 | 0.69 |
| Arg | 89 | . | . | . | . | T | T | . | 0.48 | −0.70 | . | * | . | 2.80 | 0.64 |
| Tyr | 90 | . | . | . | . | T | T | . | 0.48 | −0.06 | . | * | . | 2.22 | 0.24 |
| Cys | 91 | . | . | . | . | T | T | . | −0.19 | 0.63 | . | * | . | 1.04 | 0.37 |
| Asn | 92 | . | . | B | B | . | . | . | −0.64 | 0.63 | . | * | . | −0.04 | 0.10 |
| Val | 93 | . | . | B | B | . | . | . | 0.02 | 1.06 | . | * | . | −0.32 | 0.06 |
| Leu | 94 | . | . | B | B | . | . | . | 0.02 | 0.30 | . | . | . | −0.30 | 0.21 |
| Cys | 95 | . | . | B | . | . | T | . | 0.27 | −0.27 | . | . | . | 0.70 | 0.25 |
| Gly | 96 | . | . | . | . | . | T | C | 0.93 | −0.67 | . | . | F | 1.35 | 0.59 |
| Glu | 97 | A | . | . | . | . | T | . | 0.93 | −1.31 | . | . | F | 1.30 | 1.24 |
| Arg | 98 | A | . | . | . | . | T | . | 1.20 | −2.00 | . | * | F | 1.30 | 4.00 |
| Glu | 99 | A | A | . | . | . | . | . | 2.12 | −2.07 | . | * | F | 0.90 | 4.08 |
| Glu | 100 | A | A | . | . | . | . | . | 2.20 | −2.50 | . | * | F | 0.90 | 4.61 |
| Glu | 101 | A | A | . | . | . | . | . | 1.88 | −2.00 | . | * | F | 0.90 | 2.38 |
| Ala | 102 | A | A | . | . | . | . | . | 1.84 | −1.43 | . | . | F | 0.75 | 0.74 |
| Arg | 103 | A | A | . | . | . | . | . | 1.14 | −0.93 | . | . | . | 0.60 | 0.58 |
| Ala | 104 | A | A | . | . | . | . | . | 0.83 | −0.43 | . | * | . | 0.30 | 0.34 |
| Cys | 105 | A | A | . | . | . | . | . | 0.80 | 0.06 | . | * | . | −0.30 | 0.48 |
| His | 106 | A | A | . | . | . | . | . | 0.80 | 0.06 | * | * | . | −0.30 | 0.34 |
| Ala | 107 | A | A | . | . | . | . | . | 1.50 | 0.46 | * | * | . | −0.60 | 0.53 |
| Thr | 108 | A | A | . | . | . | . | . | 0.80 | −0.04 | * | * | . | 0.45 | 1.95 |
| His | 109 | . | A | . | . | T | . | . | 0.72 | −0.11 | * | . | . | 1.13 | 1.45 |
| Asn | 110 | . | A | . | . | T | . | . | 1.50 | −0.04 | * | . | . | 1.26 | 0.77 |
| Arg | 111 | . | A | . | . | T | . | . | 0.87 | −0.54 | . | * | . | 1.99 | 1.04 |
| Ala | 112 | . | A | . | . | T | . | . | 1.57 | −0.46 | . | * | . | 1.82 | 0.41 |
| Cys | 113 | . | . | . | . | T | T | . | 1.57 | −0.96 | . | * | . | 2.80 | 0.50 |
| Arg | 114 | . | . | B | . | . | T | . | 1.26 | −0.87 | * | * | . | 2.12 | 0.37 |
| Cys | 115 | . | . | . | . | T | T | . | 0.56 | −0.44 | * | * | . | 1.94 | 0.36 |
| Arg | 116 | . | . | . | . | T | T | . | −0.26 | −0.16 | . | * | . | 1.66 | 0.58 |
| Thr | 117 | . | A | . | B | T | . | . | −0.26 | 0.06 | . | * | F | 0.53 | 0.26 |
| Gly | 118 | . | A | . | B | T | . | . | 0.38 | 0.56 | . | * | . | −0.20 | 0.49 |
| Phe | 119 | . | A | B | B | . | . | . | −0.32 | 0.49 | . | * | . | −0.60 | 0.34 |
| Phe | 120 | . | A | B | B | . | . | . | 0.00 | 0.99 | . | * | . | −0.60 | 0.24 |
| Ala | 121 | A | A | . | B | . | . | . | −0.81 | 0.93 | . | * | . | −0.60 | 0.24 |
| His | 122 | A | A | . | . | . | . | . | −1.17 | 1.29 | . | . | . | −0.60 | 0.24 |
| Ala | 123 | A | A | . | . | . | . | . | −1.63 | 1.07 | . | * | . | −0.60 | 0.15 |
| Gly | 124 | A | A | . | . | . | . | . | −0.93 | 0.97 | . | * | . | −0.60 | 0.12 |
| Phe | 125 | A | A | . | . | . | . | . | −0.27 | 0.47 | . | . | . | −0.60 | 0.15 |
| Cys | 126 | A | A | . | . | . | . | . | −0.27 | 0.47 | . | * | . | −0.60 | 0.20 |
| Leu | 127 | A | A | . | . | . | . | . | −0.53 | 0.47 | . | . | . | −0.60 | 0.21 |
| Glu | 128 | A | A | . | . | . | . | . | −0.61 | 0.43 | . | . | . | −0.60 | 0.32 |
| His | 129 | . | . | . | . | T | T | . | −0.48 | 0.21 | . | . | . | 0.50 | 0.32 |
| Ala | 130 | . | . | . | . | T | T | . | 0.01 | 0.07 | . | . | . | 0.63 | 0.61 |
| Ser | 131 | . | . | . | . | T | T | . | 0.33 | −0.19 | . | . | . | 1.36 | 0.54 |
| Cys | 132 | . | . | . | . | . | T | C | 0.56 | 0.24 | . | . | . | 0.69 | 0.39 |
| Pro | 133 | . | . | . | . | . | T | C | 0.21 | 0.24 | . | . | F | 0.97 | 0.39 |
| Pro | 134 | . | . | . | . | T | T | . | −0.61 | 0.17 | . | . | F | 1.30 | 0.29 |
| Gly | 135 | . | . | . | . | T | T | . | −0.91 | 0.43 | . | . | F | 0.87 | 0.40 |
| Ala | 136 | . | . | B | . | . | T | . | −1.20 | 0.54 | . | . | . | 0.19 | 0.18 |
| Gly | 137 | . | . | B | B | . | . | . | −0.74 | 0.61 | . | . | . | −0.34 | 0.12 |
| Val | 138 | . | . | B | B | . | . | . | −0.88 | 0.61 | . | . | . | −0.47 | 0.19 |
| Ile | 139 | . | . | B | B | . | . | . | −0.98 | 0.61 | . | . | . | −0.60 | 0.18 |
| Ala | 140 | . | . | B | B | . | . | . | −0.84 | 0.60 | . | . | . | −0.60 | 0.27 |
| Pro | 141 | . | . | B | . | . | . | . | −0.56 | 0.60 | . | . | F | −0.25 | 0.55 |
| Gly | 142 | . | . | . | . | T | . | . | −0.21 | 0.34 | . | . | F | 0.88 | 1.06 |
| Thr | 143 | . | . | . | . | . | T | C | 0.64 | 0.06 | . | . | F | 1.16 | 1.82 |
| Pro | 144 | . | . | . | . | . | T | C | 1.22 | −0.04 | . | . | F | 2.04 | 1.89 |
| Ser | 145 | . | . | . | . | T | T | . | 1.81 | 0.01 | . | . | F | 1.92 | 2.76 |
| Gln | 146 | . | . | . | . | T | T | . | 1.36 | −0.01 | . | . | F | 2.80 | 3.31 |
| Asn | 147 | . | . | . | . | T | T | . | 1.70 | 0.07 | . | . | F | 1.92 | 1.15 |
| Thr | 148 | . | . | . | . | T | T | . | 1.80 | 0.04 | . | . | F | 1.64 | 1.48 |
| Gln | 149 | . | . | . | . | T | T | . | 1.34 | 0.09 | . | . | F | 1.36 | 1.32 |
| Cys | 150 | . | . | B | . | . | T | . | 1.43 | 0.26 | . | . | F | 0.53 | 0.44 |
| Gln | 151 | . | . | B | . | . | . | . | 1.22 | 0.29 | . | . | F | 0.05 | 0.47 |
| Pro | 152 | . | . | B | . | . | . | . | 0.88 | 0.23 | . | * | F | 0.05 | 0.42 |
| Cys | 153 | . | . | B | . | . | . | . | 0.88 | 0.26 | . | * | F | 0.05 | 0.78 |
| Pro | 154 | . | . | B | . | . | T | . | 0.18 | 0.17 | . | * | F | 0.25 | 0.65 |
| Pro | 155 | . | . | . | . | T | T | . | 0.54 | 0.56 | . | * | F | 0.35 | 0.36 |
| Gly | 156 | . | . | . | . | T | T | . | −0.04 | 0.51 | . | * | F | 0.35 | 0.91 |
| Thr | 157 | . | . | B | . | . | T | . | −0.13 | 0.44 | | . | F | −0.05 | 0.59 |
| Phe | 158 | . | . | B | . | . | . | . | 0.23 | 0.40 | . | . | F | −0.25 | 0.51 |
| Ser | 159 | . | . | B | . | . | . | . | 0.14 | 0.36 | . | . | F | 0.39 | 0.70 |
| Ala | 160 | . | . | B | . | . | . | . | 0.06 | 0.31 | . | . | F | 0.73 | 0.65 |
| Ser | 161 | . | . | . | . | . | T | C | 0.10 | 0.21 | . | . | F | 1.62 | 1.00 |
| Ser | 162 | . | . | . | . | . | T | C | 0.41 | −0.19 | . | . | F | 2.56 | 1.00 |
| Ser | 163 | . | . | . | . | T | T | . | 1.11 | −0.57 | . | . | F | 3.40 | 1.72 |
| Ser | 164 | . | . | . | . | T | T | . | 0.74 | −0.67 | . | . | F | 3.06 | 2.22 |
| Ser | 165 | . | . | . | . | T | . | . | 1.33 | −0.49 | . | . | F | 2.07 | 0.89 |
| Glu | 166 | . | . | . | . | T | . | . | 1.42 | −0.47 | . | . | F | 1.88 | 1.15 |
| Gln | 167 | . | . | . | . | T | . | . | 1.69 | −0.43 | . | . | F | 1.82 | 1.32 |
| Cys | 168 | . | . | . | . | T | . | . | 2.10 | −0.31 | . | . | F | 1.76 | 1.34 |
| Gln | 169 | . | . | B | . | . | . | . | 2.40 | −0.70 | . | . | F | 1.94 | 1.52 |
| Pro | 170 | . | . | . | . | T | . | . | 2.03 | −0.30 | . | . | F | 2.32 | 1.41 |
| His | 171 | . | . | . | . | T | T | . | 1.72 | −0.13 | . | . | F | 2.80 | 1.41 |
| Arg | 172 | . | . | . | . | T | T | . | 1.13 | −0.21 | . | . | F | 2.52 | 1.18 |
| Asn | 173 | . | . | . | . | T | T | . | 0.99 | −0.11 | * | . | . | 1.94 | 0.77 |
| Cys | 174 | . | . | B | . | . | T | . | 0.64 | 0.14 | . | . | . | 0.66 | 0.47 |
| Thr | 175 | . | A | B | . | . | . | . | 0.04 | 0.07 | . | . | . | −0.02 | 0.24 |
| Ala | 176 | . | A | B | . | . | . | . | −0.51 | 0.76 | * | . | . | −0.60 | 0.12 |
| Leu | 177 | . | A | B | . | . | . | . | −1.43 | 0.86 | * | . | . | −0.60 | 0.23 |
| Gly | 178 | . | A | B | . | . | . | . | −1.43 | 0.97 | . | * | . | −0.60 | 0.13 |
| Leu | 179 | . | A | B | . | . | . | . | −1.62 | 0.89 | . | * | . | −0.60 | 0.21 |
| Ala | 180 | . | A | B | . | . | . | . | −1.52 | 1.03 | . | * | . | −0.60 | 0.19 |
| Leu | 181 | . | A | B | . | . | . | . | −1.28 | 0.77 | . | * | . | −0.60 | 0.29 |
| Asn | 182 | . | A | B | . | . | . | . | −0.77 | 0.77 | . | * | . | −0.60 | 0.35 |
| Val | 183 | . | . | B | . | . | T | . | −0.72 | 0.47 | . | * | F | −0.05 | 0.46 |
| Pro | 184 | . | . | . | . | . | T | C | −0.21 | 0.36 | . | * | F | 0.73 | 0.75 |
| Gly | 185 | . | . | . | . | T | T | . | 0.34 | 0.06 | . | * | F | 1.21 | 0.63 |
| Ser | 186 | . | . | . | . | T | T | . | 1.16 | 0.16 | . | * | F | 1.64 | 1.15 |
| Ser | 187 | . | . | . | . | . | T | C | 0.84 | −0.49 | . | . | F | 2.32 | 1.24 |
| Ser | 188 | . | . | . | . | T | T | . | 0.89 | −0.43 | . | . | F | 2.80 | 1.81 |
| His | 189 | . | . | B | . | . | T | . | 0.43 | −0.17 | . | . | F | 2.12 | 1.11 |
| Asp | 190 | . | . | . | . | T | T | . | 0.47 | 0.01 | . | . | F | 1.49 | 0.45 |
| Thr | 191 | . | . | B | . | . | . | . | 0.47 | 0.11 | . | . | F | 0.61 | 0.48 |
| Leu | 192 | . | . | B | . | . | . | . | 0.10 | 0.11 | . | . | . | 0.18 | 0.47 |
| Cys | 193 | . | . | B | . | . | T | . | 0.09 | 0.19 | . | . | . | 0.10 | 0.15 |
| Thr | 194 | . | . | B | . | . | T | . | −0.22 | 0.67 | . | . | . | −0.20 | 0.15 |
| Ser | 195 | . | . | B | . | . | T | . | −0.92 | 0.61 | * | . | F | −0.05 | 0.18 |
| Cys | 196 | . | . | B | . | . | T | . | −0.82 | 0.71 | . | . | F | −0.05 | 0.29 |
| Thr | 197 | . | . | . | . | T | . | . | −0.82 | 0.57 | . | . | F | 0.15 | 0.31 |
| Gly | 198 | . | . | . | . | T | . | . | −0.46 | 0.77 | . | . | . | 0.00 | 0.19 |
| Phe | 199 | . | . | B | . | . | . | . | −0.46 | 0.77 | . | * | . | −0.40 | 0.48 |
| Pro | 200 | . | . | B | . | . | . | . | −0.04 | 0.69 | * | * | . | −0.40 | 0.48 |
| Leu | 201 | . | . | B | . | . | . | . | −0.23 | 0.20 | * | * | . | −0.10 | 0.96 |
| Ser | 202 | . | . | B | . | . | . | . | −0.13 | 0.41 | * | * | F | 0.02 | 0.82 |
| Thr | 203 | . | . | B | . | . | . | . | −0.13 | 0.06 | . | * | F | 0.59 | 0.82 |
| Arg | 204 | . | . | . | . | . | . | C | −0.02 | 0.06 | . | * | F | 1.06 | 0.99 |
| Val | 205 | . | . | . | . | . | T | C | 0.19 | −0.13 | . | * | F | 2.13 | 0.74 |
| Pro | 206 | . | . | . | . | . | T | C | 1.00 | −0.51 | . | * | F | 2.70 | 0.89 |
| Gly | 207 | . | . | . | . | . | T | C | 0.63 | −1.00 | . | * | F | 2.43 | 0.79 |
| Ala | 208 | A | . | . | . | . | T | . | 0.94 | −0.43 | . | * | F | 1.66 | 0.57 |
| Glu | 209 | A | A | . | . | . | . | . | 0.94 | −1.07 | . | * | F | 1.29 | 0.64 |
| Glu | 210 | A | A | . | . | . | . | . | 1.21 | −1.50 | * | . | F | 1.17 | 1.26 |
| Cys | 211 | A | A | . | . | . | . | . | 0.57 | −1.43 | * | . | F | 0.90 | 1.26 |
| Glu | 212 | A | A | . | . | . | . | . | 0.02 | −1.29 | * | * | F | 0.75 | 0.54 |
| Arg | 213 | A | A | . | . | . | . | . | 0.61 | −0.60 | * | * | . | 0.60 | 0.22 |
| Ala | 214 | A | A | . | . | . | . | . | −0.09 | −0.60 | * | * | . | 0.60 | 0.68 |
| Val | 215 | A | A | . | . | . | . | . | −0.94 | −0.39 | * | * | . | 0.30 | 0.34 |
| Ile | 216 | A | A | . | . | . | . | . | −0.87 | 0.26 | * | * | . | −0.30 | 0.13 |
| Asp | 217 | A | A | . | . | . | . | . | −1.57 | 0.76 | * | * | . | −0.60 | 0.13 |
| Phe | 218 | A | A | . | . | . | . | . | −1.68 | 1.04 | * | * | . | −0.60 | 0.15 |
| Val | 219 | A | A | . | . | . | . | . | −1.09 | 0.80 | . | . | . | −0.60 | 0.37 |
| Ala | 220 | A | A | . | . | . | . | . | −1.12 | 0.11 | . | . | . | −0.30 | 0.37 |
| Phe | 221 | A | A | . | . | . | . | . | −0.53 | 0.80 | . | * | . | −0.60 | 0.30 |
| Gln | 222 | A | A | . | . | . | . | . | −1.42 | 0.40 | . | * | . | −0.60 | 0.54 |
| Asp | 223 | A | A | . | . | . | . | . | −0.68 | 0.44 | . | . | F | −0.45 | 0.38 |
| Ile | 224 | A | A | . | . | . | . | . | 0.29 | −0.06 | . | . | F | 0.45 | 0.87 |
| Ser | 225 | A | A | . | . | . | . | . | 0.07 | −0.84 | . | . | F | 0.75 | 0.99 |
| Ile | 226 | A | A | . | . | . | . | . | 0.77 | −0.56 | * | . | F | 0.75 | 0.49 |
| Lys | 227 | A | A | . | . | . | . | . | 0.88 | −0.16 | * | * | F | 0.60 | 1.20 |
| Arg | 228 | A | A | . | . | . | . | . | 0.07 | −0.84 | * | * | F | 0.90 | 1.76 |
| Leu | 229 | A | A | . | . | . | . | . | 0.14 | −0.54 | * | . | F | 0.90 | 2.07 |
| Gln | 230 | A | A | . | . | . | . | . | 0.44 | −0.54 | * | . | F | 0.75 | 0.85 |
| Arg | 231 | . | A | B | . | . | . | . | 0.74 | −0.14 | * | . | . | 0.30 | 0.76 |
| Leu | 232 | A | A | . | . | . | . | . | −0.11 | 0.36 | * | . | . | −0.30 | 0.93 |
| Leu | 233 | . | A | B | . | . | . | . | −0.22 | 0.36 | * | * | . | −0.30 | 0.44 |
| Gln | 234 | . | A | B | . | . | . | . | 0.00 | −0.04 | * | . | . | 0.30 | 0.39 |
| Ala | 235 | . | A | B | . | . | . | . | −0.21 | 0.46 | * | . | . | −0.60 | 0.48 |
| Leu | 236 | . | A | B | . | . | . | . | −0.32 | 0.20 | * | * | . | −0.30 | 0.89 |
| Glu | 237 | . | A | B | . | . | . | . | 0.14 | −0.49 | . | . | . | 0.30 | 0.89 |
| Ala | 238 | . | . | B | . | . | T | . | 0.67 | −0.46 | . | . | F | 0.85 | 0.88 |
| Pro | 239 | . | . | . | . | T | T | . | 0.32 | −0.04 | . | . | F | 1.40 | 1.12 |
| Glu | 240 | . | . | . | . | T | T | . | 0.70 | −0.30 | . | . | F | 1.25 | 0.64 |
| Gly | 241 | . | . | . | . | T | T | . | 1.20 | 0.13 | . | . | F | 0.65 | 0.98 |
| Trp | 242 | . | . | . | . | T | . | . | 0.99 | 0.11 | * | . | F | 0.45 | 0.91 |
| Gly | 243 | . | . | . | . | . | . | C | 1.69 | 0.11 | * | * | F | 0.59 | 0.81 |
| Pro | 244 | . | . | . | . | . | . | C | 1.31 | 0.11 | * | * | F | 1.08 | 1.61 |
| Thr | 245 | . | . | . | . | . | T | C | 0.97 | 0.19 | * | . | F | 1.62 | 1.55 |
| Pro | 246 | . | . | . | . | . | T | C | 1.42 | −0.30 | * | . | F | 2.56 | 1.55 |
| Arg | 247 | . | . | . | . | T | T | . | 1.12 | −0.73 | * | . | F | 3.40 | 1.96 |
| Ala | 248 | . | . | . | . | . | T | C | 0.88 | −0.66 | * | . | F | 2.86 | 1.37 |
| Gly | 249 | A | A | . | . | . | . | . | 0.28 | −0.64 | * | * | F | 1.77 | 0.90 |
| Arg | 250 | A | A | . | . | . | . | . | 0.59 | −0.39 | * | * | . | 0.98 | 0.38 |
| Ala | 251 | A | A | . | . | . | . | . | −0.01 | 0.01 | * | * | . | 0.04 | 0.65 |
| Ala | 252 | A | A | . | . | . | . | . | −0.08 | 0.20 | * | * | . | −0.30 | 0.54 |
| Leu | 253 | A | A | . | . | . | . | . | −0.30 | −0.23 | * | * | . | 0.30 | 0.55 |
| Gln | 254 | A | A | . | . | . | . | . | 0.16 | 0.46 | . | * | . | −0.60 | 0.45 |
| Leu | 255 | A | A | . | . | . | . | . | 0.16 | −0.04 | . | * | . | 0.30 | 0.87 |
| Lys | 256 | A | A | . | . | . | . | . | 0.86 | −0.54 | . | * | . | 0.75 | 2.07 |
| Leu | 257 | A | A | . | . | . | . | . | 0.63 | −1.23 | . | * | F | 0.90 | 2.34 |
| Arg | 258 | A | A | . | . | . | . | . | 1.13 | −0.94 | * | * | F | 0.90 | 2.34 |
| Arg | 259 | . | A | B | . | . | . | . | 1.13 | −1.14 | * | * | F | 0.90 | 1.69 |
| Arg | 260 | . | A | B | . | . | . | . | 1.13 | −1.14 | * | * | F | 0.90 | 3.55 |
| Leu | 261 | . | A | B | . | . | . | . | 0.28 | −1.14 | * | * | F | 0.90 | 1.49 |
| Thr | 262 | . | A | B | . | . | . | . | 0.74 | −0.46 | * | * | F | 0.45 | 0.63 |
| Glu | 263 | . | A | B | . | . | . | . | 0.04 | −0.03 | * | * | . | 0.30 | 0.32 |
| Leu | 264 | . | A | B | . | . | . | . | −0.07 | 0.47 | * | . | . | −0.60 | 0.39 |
| Leu | 265 | . | A | B | . | . | . | . | −0.18 | 0.19 | . | * | . | −0.30 | 0.47 |
| Gly | 266 | A | A | . | . | . | . | . | 0.29 | −0.30 | . | . | . | 0.30 | 0.45 |
| Ala | 267 | A | . | . | . | . | T | . | 0.01 | 0.13 | . | . | F | 0.25 | 0.54 |
| Gln | 268 | A | . | . | . | . | T | . | −0.80 | −0.06 | . | . | F | 0.85 | 0.66 |
| Asp | 269 | A | . | . | . | . | T | . | −0.80 | −0.06 | . | . | F | 0.85 | 0.55 |
| Gly | 270 | A | . | . | . | . | T | . | −0.84 | 0.20 | * | * | . | 0.10 | 0.45 |
| Ala | 271 | A | A | . | . | . | . | . | −0.39 | 0.34 | * | * | . | −0.30 | 0.19 |
| Leu | 272 | . | A | B | . | . | . | . | −0.61 | −0.06 | * | * | . | 0.30 | 0.23 |
| Leu | 273 | . | A | B | . | . | . | . | −1.42 | 0.63 | * | * | . | −0.60 | 0.19 |
| Val | 274 | A | A | . | . | . | . | . | −1.42 | 0.89 | * | * | . | −0.60 | 0.15 |
| Arg | 275 | A | A | . | . | . | . | . | −1.67 | 0.79 | * | * | . | −0.60 | 0.32 |
| Leu | 276 | A | A | . | . | . | . | . | −1.89 | 0.60 | * | * | . | −0.60 | 0.40 |
| Leu | 277 | A | A | . | . | . | . | . | −0.97 | 0.60 | * | * | . | −0.60 | 0.44 |
| Gln | 278 | A | A | . | . | . | . | . | −1.01 | −0.04 | * | * | . | 0.30 | 0.44 |
| Ala | 279 | A | A | . | . | . | . | . | −0.74 | 0.60 | * | * | . | −0.60 | 0.40 |
| Leu | 280 | A | A | . | . | . | . | . | −0.74 | 0.41 | * | * | . | −0.60 | 0.49 |
| Arg | 281 | . | A | B | . | . | . | . | −0.53 | −0.27 | * | . | . | 0.30 | 0.55 |
| Val | 282 | . | A | B | . | . | . | . | 0.07 | −0.06 | * | . | . | 0.30 | 0.54 |
| Ala | 283 | . | A | B | . | . | . | . | −0.28 | −0.13 | * | . | . | 0.72 | 1.01 |
| Arg | 284 | . | A | B | . | . | . | . | −0.50 | −0.39 | * | . | . | 0.84 | 0.51 |
| Met | 285 | . | . | B | . | . | T | . | 0.31 | 0.30 | . | * | . | 0.91 | 0.57 |
| Pro | 286 | . | . | . | . | . | T | C | 0.31 | −0.34 | . | * | F | 2.13 | 0.97 |
| Gly | 287 | . | . | . | . | . | T | C | 0.87 | −0.84 | * | * | F | 2.70 | 0.97 |
| Leu | 288 | A | . | . | . | . | T | . | 0.60 | −0.46 | * | * | F | 2.08 | 1.32 |
| Glu | 289 | A | . | . | . | . | . | . | 0.60 | −0.43 | * | * | F | 1.46 | 0.63 |
| Arg | 290 | A | . | . | . | . | . | . | 1.20 | −0.86 | * | * | F | 1.64 | 1.25 |
| Ser | 291 | A | . | . | . | . | . | . | 1.52 | −1.29 | * | * | F | 1.37 | 2.62 |
| Val | 292 | A | . | . | . | . | . | . | 1.17 | −1.97 | * | * | F | 1.10 | 2.97 |
| Arg | 293 | A | . | . | . | . | . | . | 1.17 | −1.19 | * | * | F | 1.10 | 1.31 |
| Glu | 294 | A | . | . | . | . | . | . | 0.96 | −0.50 | * | * | F | 0.65 | 0.81 |
| Arg | 295 | A | . | . | . | . | . | . | −0.01 | −0.46 | * | * | F | 0.80 | 1.68 |
| Phe | 296 | . | . | B | . | . | . | . | 0.26 | −0.46 | . | * | . | 0.50 | 0.64 |
| Leu | 297 | . | . | B | . | . | . | . | 0.72 | 0.04 | . | * | . | −0.10 | 0.50 |
| Pro | 298 | A | . | . | . | . | . | . | 0.22 | 0.47 | . | * | . | −0.40 | 0.33 |
| Val | 299 | A | . | . | . | . | . | . | −0.17 | 0.90 | * | . | . | −0.40 | 0.48 |
| His | 300 | A | . | . | . | . | . | . | −0.67 | 0.54 | . | . | . | −0.40 | 0.75 |

Other preferred polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity. Polynucleotides encoding these polypeptide fragments are also encompassed by the invention.

Preferably, the polynucleotide fragments of the invention encode a polypeptide which demonstrates a functional activity. By a polypeptide demonstrating a “functional activity” is meant, a polypeptide capable of displaying one or more known functional activities associated with a full-length (complete) polypeptide of invention protein. Such functional activities include, but are not limited to, biological activity, antigenicity [ability to bind (or compete with a polypeptide of the invention for binding) to an antibody to the polypeptide of the invention], immunogenicity (ability to generate antibody which binds to a polypeptide of the invention), ability to form multimers with polypeptides of the invention, and ability to bind to a receptor or ligand for a polypeptide of the invention.

The functional activity of polypeptides of the invention, and fragments, variants derivatives, and analogs thereof, can be assayed by various methods.

For example, in one embodiment where one is assaying for the ability to bind or compete with full-length polypeptide of the invention for binding to an antibody of the polypeptide of the invention, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In another embodiment, where a ligand for a polypeptide of the invention identified, or the ability of a polypeptide fragment, variant or derivative of the invention to multimerize is being evaluated, binding can be assayed, e.g., by means well-known in the art, such as, for example, reducing and non-reducing gel chromatography, protein affinity chromatography, and affinity blotting. See generally, Phizicky, E., et al., 1995, Microbiol. Rev. 59:94-123. In another embodiment, physiological correlates of binding of a polypeptide of the invention to its substrates (signal transduction) can be assayed.

In addition, assays described herein (see Examples) and otherwise known in the art may routinely be applied to measure the ability of polypeptides of the invention and fragments, variants derivatives and analogs thereof to elicit related biological activity related to that of the polypeptide of the invention (either in vitro or in vivo). Other methods will be known to the skilled artisan and are within the scope of the invention.

Fusion Proteins

The present invention also includes polynucleotides, wherein the coding sequence for the mature polypeptides may be fused in the same reading frame to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and may have the leader sequence cleaved by the host cell to form the mature form of the polypeptide. The polynucleotides may also encode for a proprotein which is the mature protein plus additional 5′ amino acid residues. A mature protein having a prosequence is a proprotein and is an inactive form of the protein. Once the prosequence is cleaved an active mature protein remains. Thus, for example, the polynucleotides of the present invention may encode for a mature protein, or for a protein having a prosequence or for a protein having both a prosequence and a presequence (leader sequence).

The polynucleotides of the present invention may also have the coding sequence fused in frame to a marker sequence which allows for purification of the polypeptide of the present invention. The marker sequence may be a hexa-histidine tag supplied by a pQE-9 vector to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I., et al., Cell, 37:767 (1984)).

Any polypeptide of the present invention can be used to generate fusion proteins. For example, the polypeptide of the present invention, when fused to a second protein, can be used as an antigenic tag. Antibodies raised against the polypeptide of the present invention can be used to indirectly detect the second protein by binding to the polypeptide. Moreover, because secreted proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Also, peptide moieties may be added to the polypeptide to facilitate purification. Such regions may be removed prior to final preparation of the polypeptide. The addition of peptide moieties to facilitate handling of polypeptides is familiar and routine techniques in the art.

Moreover, polypeptides of the present invention, including fragments, and specifically epitopes, can be combined with parts of the constant domain of immunoglobulins (IgA, IgE, IgG, IgM) or portions thereof (CH1, CH2, CH3, and any combination thereof, including both entire domains and portions thereof), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).) Polynucleotides comprising or alternatively consisting of nucleic acids that encode these fusion proteins are also encompassed by the invention.

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the polypeptides of the present invention can be fused to marker sequences, such as a peptide, which facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984).)

Thus, any of these above fusions can be engineered using the polynucleotides or the polypeptides of the present invention.

Diagnostics

This invention is also related to the use of a embodiments of the present invention as a diagnostic. For example, some diseases result from inherited defective genes. For example, TNF-gamma-β overexpressed may lead to increased inflammation in the bowel of patients having an inflammatory bowel disease. A mutation in a TNF-gamma-β gene of the present invention at the DNA level may be detected by a variety of techniques. Nucleic acids used for diagnosis (genomic DNA, mRNA, etc.) may be obtained from a patient's cells, other than from the colon, such as from blood, urine, saliva, tissue biopsy and autopsy material. The genomic DNA may be used directly for detection or may be amplified enzymatically by using PCR (Saiki, et al., Nature, 324:163-166 (1986)) prior to analysis. RNA or cDNA may also be used for the same purpose. As an example, PCR primers complementary to the nucleic acid of the instant invention can be used to identify and analyze mutations in a TNF-gamma-β polynucleotide of the present invention. For example, deletions and insertions can be detected by a change in size of the amplified product in comparison to the normal genotype. Furthermore, for example, point mutations can be identified by hybridizing amplified DNA to radiolabeled TNF-gamma-β RNA or, alternatively, radiolabeled antisense DNA sequences.

Another well-established method for screening for mutations in particular segments of DNA after PCR amplification is single-strand conformation polymorphism (SSCP) analysis. PCR products are prepared for SSCP by ten cycles of reamplification to incorporate ³²P-dCTP, digested with an appropriate restriction enzyme to generate 200-300 bp fragments, and denatured by heating to 85° C. for 5 min. and then plunged into ice. Electrophoresis is then carried out in a nondenaturing gel (5% glycerol, 5% acrylamide) (Glavac, D. and Dean, M., Human Mutation, 2:404-414 (1993)).

Sequence differences between the reference gene and “mutants” may be revealed by the direct DNA sequencing method. In addition, cloned DNA segments may be used as probes to detect specific DNA segments. The sensitivity of this method is greatly enhanced when combined with PCR. For example, a sequencing primer is used with double-stranded PCR product or a single-stranded template molecule generated by a modified PCR. The sequence determination is performed by conventional procedures with radiolabeled nucleotides or by automatic sequencing procedures with fluorescent-tags.

Genetic testing based on DNA sequence differences may be achieved by detection of alteration in electrophoretic mobility of DNA fragments and gels with or without denaturing agents. Small sequence deletions and insertions can be visualized by high-resolution gel electrophoresis. DNA fragments of different sequences may be distinguished on denaturing formamide gradient gels in which the mobilities of different DNA fragments are retarded in the gel at different positions according to their specific melting or partial melting temperatures (see, e.g., Myers, et al., Science, 230:1242 (1985)). In addition, sequence alterations, in particular small deletions, may be detected as changes in the migration pattern of DNA.

Sequence changes at specific locations may also be revealed by nuclease protection assays, such as RNase and S1 protection or the chemical cleavage method (e.g., Cotton, et al., PNAS, USA, 85:4397-4401 (1985)).

Thus, the detection of the specific DNA sequence may be achieved by methods such as hybridization, RNase protection, chemical cleavage, direct DNA sequencing, or the use of restriction enzymes (e.g., Restriction Fragment Length Polymorphisms (RFLP)) and Southern blotting.

The sequences of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with gene associated with disease.

Briefly, sequences can be mapped to chromosomes by preparing PCR primers (preferably 15-25 bp) from the cDNA. Computer analysis of the 3′ untranslated region is used to rapidly select primers that do not span more than one exon in the genomic DNA, thus complicating the amplification process. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in, situ hybridization, prescreening with labeled flow-sorted chromosomes and preselection by hybridization to construct chromosome specific-cDNA libraries.

Fluorescence in situ hybridization (FISH) of a cDNA clone to a metaphase chromosomal spread can be used to provide a precise chromosomal location in one step. This technique can be used with cDNA as short as 50 or 60 bases. For a review of this technique, see Verma et al., Human Chromosomes: a Manual of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between gene and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb).

Demonstration of Therapeutic or Prophylactic Activity

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

Therapeutic/Prophylactic Administration and Composition

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Diagnosis and Imaging

Labeled antibodies, and derivatives and analogs thereof, which specifically bind to a polypeptide of interest can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen et al., J. Cell. Biol. 101:976-985 (1985); Jalkanen et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (14C), sulfur (³⁵S), tritium (3H), indium (112In), and technetium (99Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: a) administering (for example, parenterally, subcutaneously, or intraperitoneally) to a subject an effective amount of a labeled molecule which specifically binds to the polypeptide of interest; b) waiting for a time interval following the administering for permitting the labeled molecule to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of cells that contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope that is specifically immunoreactive with an antibody included in the kit. Preferably, the kits of the present invention further comprise a control antibody that does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope that is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening sera that may contain antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to, a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme that is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

Epitopes And Antibodies

Introduction

The polypeptides, their fragments or other derivatives, or analogs thereof, or cells expressing them can be used as an immunogen to produce antibodies thereto. These antibodies can be, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

The antibodies may be employed, for example, to target diseased tissue in inflammatory bowel disease, for example, in a method of directing anti-inflammatory agents which, when contacting inflamed bowel tissue, reduce inflammation and/or assist in tissue healing and regeneration. This is true since the antibodies are specific for the TNF-gamma-β, DR3, and/or TR6 polypeptides of the present invention. A linking of the antiinflammatory agent to the antibody would cause the interaction agent to be carried directly to the colon.

The antibodies of the present invention may also be employed, for example, to treat diseased tissue in inflammatory bowel disease, for example, in a method of acting as an antagonist of a TNF-gamma-β, DR3, and/or TR6 polypeptide, which when contacting inflamed bowel tissue, acts to reduce inflammation and/or assist in tissue healing and regeneration. This is true since the antibodies are specific for, and act as antagonists of the TNF-gamma-β polypeptides and receptors of the present invention. The specificity of the antibody would target it directly to the bowel while the agonistic nature of the antibody would act to reduce inflammation and promote healing of diseased bowel tissue.

Antibodies of this type may also be used in in vivo imaging, for example, by labeling the antibodies to facilitate scanning of the pelvic area and the colon. One method for imaging inflammatory bowel disease comprises contacting any diseased tissue of the bowel to be imaged with anti-TNF-gamma-β, DR3, and/or TR6 protein-antibodies labeled with a detectable marker. The method is performed under conditions such that a labeled antibody binds to a TNF-gamma-β, DR3, and/or TR6 polypeptide. In a specific example, the antibodies interact with the colon, for example, colon mucosal cells, and fluoresce upon contact such that imaging and visibility of the colon are enhanced to allow a determination of the diseased or non-diseased state of the colon.

Antibodies of this type may also be used in in vitro imaging, for example, by labeling the antibodies to facilitate immunocytological examination of biopy tissue samples from inflammatory bowel disease patients. One method for immunocytological imaging of inflammatory bowel disease comprises contacting any diseased tissue of the bowel with an anti-TNF-gamma-β, anti-DR3, and/or anti-TR6 protein-antibody labeled with a detectable marker. The method is performed under conditions such that a labeled antibody binds to a TNF-gamma-β, DR3, and/or TR6 polypeptide. In a specific example, the antibodies interact with the colon, for example, colon mucosal cells, and fluoresce upon contact such that imaging and visibility of the colon are enhanced to allow a determination of the diseased or non-diseased state of the colon.

Antibodies of this type may also be used in in vitro diagnostic tests, for example, by labeling antibodies to facilitate determination of altered TNF-gamma-β, DR3, and/or TR6 expression in biological samples from a patient suspected of having inflammatory bowel disease. One method for diagnosing inflammatory bowel disease comprises contacting a biologicdal sample to be tested with an anti-TNF-gamma-β, an anti-DR3, and/or an anti-TR6 protein-antibody labeled with a detectable marker. The method is performed under conditions such that a labeled antibody binds to a TNF-gamma-β, a DR3, and/or a TR6 polypeptide. In a specific example, the antibodies interact with the sample, for example, colon mucosal cells, and said binding may be measured to allow a determination of the diseased or non-diseased state of the colon.

Epitopes and Antibodies—Detailed Description

The present invention encompasses polypeptides comprising, or alternatively consisting of, an epitope of a polypeptide having an amino acid sequence of SEQ ID NOs:2, 4, or 6, or an epitope of a polypeptide sequence encoded by a polynucleotide sequence contained in ATCC deposit Nos. 203055, 97757, or 97810, or encoded by a polynucleotide that hybridizes to a complement of a sequence of SEQ ID NOs:1, 3, or 5, or contained in ATCC deposit Nos. 203055, 97757, or 97810, under stringent hybridization conditions or lower stringency hybridization conditions as defined supra. The present invention further encompasses polynucleotide sequences encoding an epitope of a polypeptide sequence of the invention (such as, for example, the sequence disclosed in SEQ ID NO:1), polynucleotide sequences of the complementary strand of a polynucleotide sequence encoding an epitope of the invention, and polynucleotide sequences which hybridize to the complementary strand under stringent hybridization conditions or lower stringency hybridization conditions defined supra.

The term “epitopes,” as used herein, refers to portions of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof. Antigenic epitopes are useful, for example, to raise antibodies, including monoclonal antibodies that specifically bind the epitope. Preferred antigenic epitopes include the antigenic epitopes disclosed herein, as well as any combination of two, three, four, five or more of these antigenic epitopes. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody that can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and as discussed above, the polypeptides of the present invention (e.g., those comprising an immunogenic or antigenic epitope) can be fused to heterologous polypeptide sequences. For example, polypeptides of the present invention (including fragments or variants thereof), may be fused with the constant domain of immunoglobulins (IgA, IgE, IgG, IgM), or portions thereof (CH1, CH2, CH3, or any combination thereof and portions thereof, resulting in chimeric polypeptides. By way of another non-limiting example, polypeptides and/or antibodies of the present invention (including fragments or variants thereof) may be fused with albumin (including but not limited to recombinant human serum albumin or fragments or variants thereof (see, e.g., U.S. Pat. No. 5,876,969, issued Mar. 2, 1999, EP Patent 0 413 622, and U.S. Pat. No. 5,766,883, issued Jun. 16, 1998, herein incorporated by reference in their entirety)). In a preferred embodiment, polypeptides and/or antibodies of the present invention (including fragments or variants thereof) are fused with the mature form of human serum albumin (i.e., amino acids 1-585 of human serum albumin as shown in FIGS. 1 and 2 of EP Patent 0 322 094) which is herein incorporated by reference in its entirety. In another preferred embodiment, polypeptides and/or antibodies of the present invention (including fragments or variants thereof) are fused with polypeptide fragments comprising, or alternatively consisting of, amino acid residues 1-z of human serum albumin, where z is an integer from 369 to 419, as described in U.S. Pat. No. 5,766,883 herein incorporated by reference in its entirety. Polypeptides and/or antibodies of the present invention (including fragments or variants thereof) may be fused to either the N- or C-terminal end of the heterologous protein (e.g., immunoglobulin Fc polypeptide or human serum albumin polypeptide). Polynucleotides encoding fusion proteins of the invention are also encompassed by the invention.

Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion desulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix-binding domain for the fusion-protein. Extracts from cells infected with the recombinant vaccinia virus are loaded onto Ni²⁺ nitriloacetic acid-agarose column and histidine-tagged proteins can be selectively eluted with imidazole-containing buffers.

Additional fusion proteins of the invention may be generated through the techniques of gene shuffling, motif shuffling, exon shuffling, and/or codon shuffling (collectively referred to as “DNA shuffling”). DNA shuffling may be employed to modulate the activities of polypeptides of the invention and such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, alteration of polynucleotides corresponding to SEQ ID NO:1 and the polypeptides encoded by these polynucleotides may be achieved by DNA shuffling. DNA shuffling involves the assembly of two or more DNA segments by homologous or site-specific recombination to generate variation in the polynucleotide sequence. In another embodiment, polynucleotides of the invention or the polypeptides encoded thereby, may be altered by being subjected to random mutagenesis by error-prone PCR, random nucleotide insertion or other methods prior to recombination. In another embodiment, one or more components, motifs, sections, parts, domains, fragments, etc., of a polynucleotide encoding a polypeptide of the invention may be recombined with one or more components, motifs, sections, parts, domains, fragments, etc. of one or more heterologous molecules.

Antibodies—Definition and Preparation

Further polypeptides of the invention relate to antibodies and T-cell antigen receptors (TCR) which immunospecifically bind a polypeptide, polypeptide fragment, or variant of one or more of SEQ ID NOs:2, 4, and/or 6, and/or an epitope, of the present invention (as determined by immunoassays well known in the art for assaying specific antibody-antigen binding). Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. In preferred embodiments, the immunoglobulin molecules of the invention are IgG1. In other preferred embodiments, the immunoglobulin molecules of the invention are IgG4.

Most preferably the antibodies are human antigen-binding antibody fragments of the present invention and include, but are not limited to, Fab, Fab′ and F(ab′)₂, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention, which they recognize or specifically bind. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies, which bind polypeptides encoded by polynucleotides, which hybridize to a polynucleotide of the present invention under stringent hybridization conditions (as described herein). Antibodies of the present invention may also be described or specified in terms of their binding affinity to a polypeptide of the invention.

In specific embodiments, antibodies of the invention bind polypeptides of the invention or fragments or variants thereof, with a dissociation constant or K_{D} of less than or equal to 5×10⁻² M, 10⁻² M, 5×10⁻³ M, 10⁻³ M, 5×10⁻⁴ M, 10⁻⁴M, 5×10⁻⁵ M, or 10⁻⁵ M. More preferably, antibodies of the invention bind polypeptides of the invention or fragments or variants thereof with a dissociation constant or K_{D} less than or equal to 5×10⁻⁶ M, 10⁻⁶ M, 5×10⁻⁷ M, 10⁻⁷ M, 5×10⁻⁸ M, or 10⁻⁸ M. Even more preferably, antibodies of the invention bind polypeptides of the invention or fragments or variants thereof with a dissociation constant or K_{D} less than or equal to 5×10⁻⁹ M, 10⁻⁹ M, 5×10⁻¹⁰ M, 10⁻¹¹ M, 5×10⁻¹¹ M, 10⁻¹¹ M, 5×10¹² M, 10⁻¹² M, 5×10⁻¹³ M, 10⁻³ M, 5×10⁻¹⁴ M, 10⁻¹⁴ M, 5×10⁻¹⁵ M, or 10⁻¹⁵ M. The invention encompasses antibodies that bind polypeptides of the invention with a dissociation constant or K_{D} that is within any one of the ranges that are between each of the individual recited values.

In specific embodiments, antibodies of the invention bind polypeptides of the invention or fragments or variants thereof with an off rate (k_{off}) of less than or equal to 5×10⁻² sec⁻¹, 10⁻² sec⁻¹, 5×10⁻³ sec⁻¹ or 10⁻³ sec⁻¹. More preferably, antibodies of the invention bind polypeptides of the invention or fragments or variants thereof with an off rate (k_{off}) less than or equal to 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹. The invention encompasses antibodies that bind polypeptides of the invention with an off rate (k_{off}) that is within any one of the ranges that are between each of the individual recited values.

In other embodiments, antibodies of the invention bind polypeptides of the invention or fragments or variants thereof with an on rate (n) of greater than or equal to 10³ M⁻¹sec⁻¹, 5×10³ M⁻¹sec⁻¹, 10⁴ M⁻¹sec⁻¹ or 5×10⁴ M⁻¹sec⁻¹. More preferably, antibodies of the invention bind polypeptides of the invention or fragments or variants thereof with an on rate (kₒₙ) greater than or equal to 10⁵ M⁻¹ sec⁻¹, 5×10⁵ M⁻¹sec⁻¹, 10⁶ M⁻¹sec⁻¹, or 5×10⁶ M⁻¹sec⁻¹ or 10⁷ M⁻¹sec⁻¹. The invention encompasses antibodies that bind polypeptides of the invention with on rate (kₒₙ) that is within any one of the ranges that are between each of the individual recited values.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, at least 50%, or at least 40%.

Antibodies of the present invention may act as agonists or antagonists of the polypeptides of the present invention. For example, the present invention includes antibodies that disrupt the receptor/ligand interactions with the polypeptides of the invention either partially or fully. Preferrably, antibodies of the present invention bind an antigenic epitope disclosed herein, or a portion thereof. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 111(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996) (which are all incorporated by reference herein in their entireties).

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are also described in the Examples (below). In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable mycloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments, which recognize specific epitopes, may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles that carry the polynucleotide sequences encoding them. In a particular embodiment, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 1879-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397, which are incorporated herein by reference in their entirety. Humanized antibodies are antibody molecules from non-human species antibody that binds the desired antigen having one or more complementarity determining regions (CDRs) from the non-human species and a framework region from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having an amino acid sequence of one of SEQ ID NOs:2, 4, or 6.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site-directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence aredescribed herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452(1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fe portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341(1992) (said references incorporated by reference in their entireties).

As discussed, supra, a polypeptide corresponding to a polypeptide, polypeptide fragment, or a variant of one of SEQ ID NOs:2, 4, or 6, may be fused or conjugated to the above antibody portions to increase the in vivo half life of the polypeptides or for use in immunoassays using methods known in the art. Further, a polypeptide corresponding to one of SEQ ID NOs:2, 4, or 6, may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide-linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, Bennett et al., J. Molecular Recognition 8:52-58 (1995); Johanson et al., J. Biol. Chem. 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detectioncan be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, ²¹³Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, β-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1 ”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Assays For Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 40 C, washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

Therapeutic Uses ofAntibodies

The present invention also encompasses antibody-based therapies which involve administering antibodies of the invention to an animal, preferably a mammal, and most preferably a human, patient for treating a disorder requiring a reduction of TNF-gamma-β polypeptide function and/or expression. Treatment of a disorder requiring reduction in TNF-gamma-β polypeptide function and/or expression may also be carried out using certain other embodiments of the present invention including, but not limited to, polypeptides, polypeptide fragments, and antagonists. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy, anti-tumor agents, and surgical treatments). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof.

In specific embodiments, antibodies of the invention bind polypeptides of the invention or fragments or variants thereof, with a dissociation constant or K_{D} of less than or equal to 5×10⁻² M, 10⁻² M, 5×10⁻³ M, 10⁻³ M, 5×10⁻⁴ M, 10⁻⁴ M, 5×10⁻⁵ M, or 10⁻⁵ M. More preferably, antibodies of the invention bind polypeptides of the invention or fragments or variants thereof with a dissociation constant or K_{D} less than or equal to 5×10⁻⁶ M, 10⁻⁶ M, 5×10⁻⁷ M, 10⁻⁷ M, 5×10⁻⁸ M, or 10⁻⁸ M. Even more preferably, antibodies of the invention bind polypeptides of the invention or fragments or variants thereof with a dissociation constant or K_{D} less than or equal to 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻⁴ M, 10⁻¹⁴ M, 5×10⁻¹⁵ M, or 10⁻¹⁵ M. The invention encompasses antibodies that bind polypeptides of the invention with a dissociation constant or K_{D} that is within any one of the ranges that are between each of the individual recited values.

In specific embodiments, antibodies of the invention bind polypeptides of the invention or fragments or variants thereof with an off rate (k_{off}) of less than or equal to 5×10⁻² sec⁻¹, 10⁻² sec⁻¹, 5×10⁻³ sec⁻¹ or 10⁻³ sec⁻¹. More preferably, antibodies of the invention bind polypeptides of the invention or fragments or variants thereof with an off rate (k_{off}) less than or equal to 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹. The invention encompasses antibodies that bind polypeptides of the invention with an off rate (k_{off}) that is within any one of the ranges that are between each of the individual recited values.

In other embodiments, antibodies of the invention bind polypeptides of the invention or fragments or variants thereof with an on rate (kₒₙ) of greater than or equal to 10³ M⁻¹sec⁻¹, 5×10³ M⁻¹sec⁻¹, 10⁴ M⁻¹sec⁻¹ or 5×10⁴ M⁻¹sec⁻¹. More preferably, antibodies of the invention bind polypeptides of the invention or fragments or variants thereof with an on rate (kₒₙ) greater than or equal to 10⁵ M⁻¹sec⁻¹, 5×10⁵ M⁻¹sec⁻¹, 10⁶ M⁻¹sec⁻¹, or 5×10⁶ M⁻¹sec⁻¹ or 10⁷ M⁻¹sec⁻¹. The invention encompasses antibodies that bind polypeptides of the invention with on rate (kₒₙ) that is within any one of the ranges that are between each of the individual recited values.

Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contain nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitate delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoictic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PA0815 (all available from Invitrogen, Carlbad, Calif.). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In one embodiment, the yeast Pichia pastoris is used to express the polypeptide of the present invention in a eukaryotic system. Pichia pastoris is a methylotrophic yeast which can metabolize methanol as its sole carbon source. A main step in the methanol metabolization pathway is the oxidation of methanol to formaldehyde using 2. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for 2. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al., Mol. Cell. Biol. 5:1111-21 (1985); Koutz, P. J, et al., Yeast 5:167-77 (1989); Tschopp, J. F., et al., Nucl. Acids Res. 15:3859-76 (1987). Thus, a heterologous coding sequence, such as, for example, a polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a polypeptide of the invention, as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PA0815, as one skilled in the art would readily appreciate, as long as the proposed expression construct provides appropriately located signals for transcription, translation, secretion (if desired), and the like, including an in-frame AUG as required.

In another embodiment, high-level expression of a heterologous coding sequence, such as, for example, a polynucleotide of the present invention, may be achieved by cloning the heterologous polynucleotide of the invention into an expression vector such as, for example, pGAPZ or pGAPZalpha, and growing the yeast culture in the absence of methanol.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to-include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination, resulting in the formation of a new transcription unit (see, e.g., U.S. Pat. No. 5,641,670, issued June 24, 1997; U.S. Pat. No. 5,733,761, issued March 31, 1998; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

In addition, polypeptides of the invention can be chemically synthesized using techniques known in the art (e.g., see Creighton, 1983, Proteins: Structures and Molecular Principles, W. H. Freeman & Co., N.Y., and Hunkapiller et al., Nature, 310:105-111 (1984). For example, a polypeptide corresponding to a fragment of a polypeptide sequence of the invention can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, omithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic or affinity label to allow for detection and isolation of the protein.

Also provided by the invention are chemically modified derivatives of the polypeptides of the invention which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to a protein via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J of Hematol. 68:1-18 (1998); U.S. Pat. No. 4,002,531; U.S. Pat. No. 5,349,052; WO 95/06058; and WO 98/32466, the disclosures of each of which are incorporated herein by reference.

One system for attaching polyethylene glycol directly to amino acid residues of proteins without an intervening linker employs tresylated MPEG, which is produced by the modification of monmethoxy polyethylene glycol (MPEG) using tresylchloride (ClSO₂CH₂CF₃). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoreothane sulphonyl group.

Polyethylene glycol can also be attached to proteins using a number of different intervening linkers. For example, U.S. Pat. No. 5,612,460, the entire disclosure of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number additional polyethylene glycol derivatives and reaction chemistries for attaching polyethylene glycol to proteins are described in WO 98/32466, the entire disclosure of which is incorporated herein by reference. Pegylated protein products produced using the reaction chemistries set out herein are included within the scope of the invention.

The number of polyethylene glycol moieties attached to each protein of the invention (i.e., the degree of substitution) may also vary. For example, the pegylated proteins of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3,2-4, 3-5,4-6, 5-7,6-8, 7-9,8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

The polypeptides of the invention may be in monomers or multimers (i.e., dimers, trimers, tetramers and higher multimers). Accordingly, the present invention relates to monomers and multimers of the polypeptides of the invention, their preparation, and compositions (preferably, Therapeutics) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only polypeptides corresponding to a single amino acid sequence of SEQ ID NOs:2, 4, or 6, or encoded by a cDNA contained in a single deposited clone (including fragments, variants, splice variants, and fusion proteins, corresponding to these polypeptides as described herein). These homomers may contain polypeptides having identical or different amino acid sequences. In a specific embodiment, a homomer of the invention is a multimer containing only polypeptides having an identical amino acid sequence. In another specific embodiment, a homomer of the invention is a multimer containing polypeptides having different amino acid sequences. In specific embodiments, the multimer of the invention is a homodimer (e.g., containing polypeptides having identical or different amino acid sequences) or a homotrimer (e.g., containing polypeptides having identical and/or different amino acid sequences). In additional embodiments, the homomeric multimer of the invention is at least a homodimer, at least a homotrimer, or at least a homotetramer.

As used herein, the term heteromer refers to a multimer containing one or more heterologous polypeptides (i.e., polypeptides of different proteins) in addition to the polypeptides of the invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the polypeptides of the invention. Such covalent associations may involve one or more amino acid residues contained in the polypeptide sequence (e.g., that recited in the sequence listing, or contained in the polypeptide encoded by a deposited clone). In one instance, the covalent associations are cross-linking between cysteine residues located within the polypeptide sequences which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a fusion protein of the invention.

In one example, covalent associations are between the heterologous sequence contained in a fusion protein of the invention (see, e.g., U.S. Pat. No. 5,478,925). In a specific example, the covalent associations are between the heterologous sequence contained in an Fc fusion protein of the invention (as described herein). In another specific example, covalent associations of fusion proteins of the invention are between heterologous polypeptide sequence from another protein that is capable of forming covalently associated multimers, such as for example, oseteoprotegerin (see, e.g., International Publication NO: WO 98/49305, the contents of which are herein incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Another method for preparing multimer polypeptides of the invention involves use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Trimeric polypeptides of the invention may offer the advantage of enhanced biological activity. Preferred leucine zipper moieties and isoleucine moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. Other peptides derived from naturally occurring trimeric proteins may be employed in preparing trimeric polypeptides of the invention.

In another example, proteins of the invention are associated by interactions between Flag® polypeptide sequence contained in fusion proteins of the invention containing Flag® polypeptide seuqence. In a further embodiment, associations proteins of the invention are associated by interactions between heterologous polypeptide sequence contained in Flag® fusion proteins of the invention and anti-Flagg antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hyrophobic or signal peptide) and which can be incorporated by membrane reconstitution techniques into liposomes (see, e.g., U.S. Pat. No. 5,478,925, which is herein incorporated by reference in its entirety).

Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents, based on actual sequence data (repeat polymorphisms), are presently available. Each polynucleotide of the present invention can be used as a chromosome marker.

Furthermore, increased or decreased expression of the gene in affected individuals as compared to unaffected individuals can be assessed using polynucleotides of the present invention. Any of these alterations (altered expression, chromosomal rearrangement, or mutation) can be used as a diagnostic or prognostic marker.

Thus, the invention also provides a diagnostic method useful during diagnosis of a disorder, involving measuring the expression level of polynucleotides of the present invention in cells or body fluid from an individual and comparing the measured gene expression level with a standard level of polynucleotide expression level, whereby an increase or decrease in the gene expression level compared to the standard is indicative of a disorder.

In still another embodiment, the invention includes a kit for analyzing samples for the presence of polynucleotides derived from a test subject. In a general embodiment, the kit includes at least one polynucleotide probe containing a nucleotide sequence that will specifically hybridize with a polynucleotide of the present invention and a suitable container. In a specific embodiment, the kit includes two polynucleotide probes defining an internal region of the polynucleotide of the present invention, where each probe has one strand containing a 31′mer-end internal to the region. In a further embodiment, the probes may be useful as primers for polymerase chain reaction amplification.

Where a diagnosis of a disorder, has already been made according to conventional methods, the present invention is useful as a prognostic indicator, whereby patients exhibiting enhanced or depressed polynucleotide of the present invention expression will experience a worse clinical outcome relative to patients expressing the gene at a level nearer the standard level.

By “measuring the expression level of polynucleotide of the present invention” is intended qualitatively or quantitatively measuring or estimating the level of the polypeptide of the present invention or the level of the mRNA encoding the polypeptide in a first biological sample either directly (e.g., by determining or estimating absolute protein level or mRNA level) or relatively (e.g., by comparing to the polypeptide level or mRNA level in a second biological sample). Preferably, the polypeptide level or mRNA level in the first biological sample is measured or estimated and compared to a standard polypeptide level or mRNA level, the standard being taken from a second biological sample obtained from an individual not having the disorder or being determined by averaging levels from a population of individuals not having a disorder. As will be appreciated in the art, once a standard polypeptide level or mRNA level is known, it can be used repeatedly as a standard for comparison.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

The method(s) provided above may preferrably be applied in a diagnostic method and/or kits in which polynucleotides and/or polypeptides are attached to a solid support. In one exemplary method, the support may be a “gene chip” or a “biological chip” as described in U.S. Pat. Nos. 5,837,832, 5,874,219, and 5,856,174. Further, such a gene chip with polynucleotides of the present invention attached may be used to identify polymorphisms between the polynucleotide sequences, with polynucleotides isolated from a test subject. The knowledge of such polymorphisms (i.e. their location, as well as, their existence) would be beneficial in identifying disease loci for many disorders, including cancerous diseases and conditions. Such a method is described in U.S. Pat. Nos. 5,858,659 and 5,856,104. The US Patents referenced supra are hereby incorporated by reference in their entirety herein.

The present invention encompasses polynucleotides of the present invention that are chemically synthesized, or reproduced as peptide nucleic acids (PNA), or according to other methods known in the art. The use of PNAs would serve as the preferred form if the polynucleotides are incorporated onto a solid support, or gene chip. For the purposes of the present invention, a peptide nucleic acid (PNA) is a polyamide type of DNA analog and the monomeric units for adenine, guanine, thymine and cytosine are available commercially (Perceptive Biosystems). Certain components of DNA, such as phosphorus, phosphorus oxides, or deoxyribose derivatives, are not present in PNAs. As disclosed by P. E. Nielsen, M. Egholm, R. H. Berg and O. Buchardt, Science 254, 1497 (1991); and M. Egholm, O. Buchardt, L. Christensen, C. Behrens, S. M. Freier, D. A. Driver, R. H. Berg, S. K. Kim, B. Norden, and P. E. Nielsen, Nature 365, 666 (1993), PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the strong binding. In addition, it is more likely that single base mismatches can be determined with PNA/DNA hybridization because a single mismatch in a PNA/DNA 15-mer lowers the melting point (T.sub.m) by 8°-20° C., vs. 4°-16° C. for the DNA/DNA 15-mer duplex. Also, the absence of charge groups in PNA means that hybridization can be done at low ionic strengths and reduce possible interference by salt during the analysis.

The present invention is useful for detecting diseases of the gastrointestinal tract in mammals. In particular the invention is useful during diagnosis of inflammatory bowel diseases that include, but are not limited to: Crohn's disease and ulcerative colitis. Preferred mammals include monkeys, apes, cats, dogs, cows, pigs, horses, rabbits and humans. Particularly preferred are humans.

In addition to the foregoing, a polynucleotide can be used to control gene expression through triple helix formation or antisense DNA or RNA. Antisense techniques are discussed, for example, in Okano, J. Neurochem. 56: 560 (1991); “Oligodeoxynucleotides as Antisense Inhibitors of Gene Expression, CRCPress, Boca Raton, Fla. (1988). Triple helix formation is discussed in, for instance Lee et al., Nucleic Acids Research 6: 3073 (1979); Cooney et al., Science 241: 456 (1988); and Dervan et al., Science 251: 1360 (1991). Both methods rely on binding of the polynucleotide to a complementary DNA or RNA. For these techniques, preferred polynucleotides are usually oligonucleotides 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxynucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat or prevent disease.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell.

Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell. Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment, which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

Thus, the invention provides a diagnostic method of a gastrointestinal disorder, which involves (a) assaying the expression of a polypeptide of the present invention in cells, body fluid, and/or stool of an individual; (b) comparing the level of gene expression with a standard gene expression level, whereby an increase or decrease in the assayed polypeptide gene expression level compared to the standard expression level is indicative of a gastrointestinal disorder. With respect to inflammatory bowel disease, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby reducing the severity of the symptoms of the disease.

Moreover, polypeptides of the present invention can be used to treat, prevent, and/or diagnose inflammatory bowel disease (IBD). For example, IBD patients can be administered a polypeptide of the present invention in an effort to reduce excess or increased levels of the polypeptide, or to bring about a desired response (e.g., reduced IFNγ secretion).

Similarly, antibodies binding to a polypeptide of the present invention can also be used to treat, prevent, and/or diagnose IBD. For example, administration of an antibody that binds a polypeptide of the present invention can bind and reduce overproduction of the polypeptide. Similarly, administration of an antibody can inhibit the polypeptide, such as by binding to a polypeptide bound to a membrane (receptor).

At the very least, the polypeptides of the present invention can be used as molecular weight markers on SDS-PAGE gels or on molecular sieve gel filtration columns using methods well known to those of skill in the art. Polypeptides can also be used to raise antibodies, which in turn are used to measure protein expression from a recombinant cell, as a way of assessing transformation of the host cell. Moreover, the polypeptides of the present invention can be used to test the following biological activities.

Formulations

The invention also provides methods of treatment and/or prevention of gastrointestinal diseases or disorders (such as, for example, any one or more of the gastrointestinal diseases or disorders disclosed herein) by administration to a subject of an effective amount of a Therapeutic. By therapeutic is meant polynucleotides or polypeptides of the invention (including fragments and variants), agonists or antagonists thereof, and/or antibodies thereto, in combination with a pharmaceutically acceptable carrier type (e.g., a sterile carrier).

The invention provides methods of treatment and/or prevention of inflammatory bowel diseases, such as Crohn's disease and ulcerative colitis, which result in destruction of the mucosal surface, and/or underlying layers, of the small and/or large intestine. Thus, TNF-gamma-β, DR3 and/or TR6 polynucleotides or polypeptides, as well as antagonists or antibodies thereto, could be used to inhibit and/or reduce mucosal inflammation, and thereby to promote the resurfacing of the mucosal surface to aid more rapid healing and to prevent or attenuate progression of inflammatory bowel disease. Treatment with TNF-gamma-β, DR3 and/or TR6 polynucleotides or polypeptides, as well as antagonists or antibodies thereto, is expected to have a significant effect on the production of mucus throughout the gastrointestinal tract and could be used to protect the intestinal mucosa from injurious substances that are ingested or following surgery. TNF-gamma-β, DR3 and/or TR6 polynucleotides or polypeptides, as well as antagonists or antibodies thereto, can also be used to promote healing of intestinal or colonic anastomosis and to treat diseases associate with the over expression of TNF-gamma-P.

The Therapeutic will be formulated and dosed in a fashion consistent with good medical practice, taking into account the clinical condition of the individual patient (especially the side effects of treatment with the Therapeutic alone), the site of delivery, the method of administration, the scheduling of administration, and other factors known to practitioners. The “effective amount” for purposes herein is thus determined by such considerations.

As a general proposition, the total pharmaceutically effective amount of the Therapeutic administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the Therapeutic is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Therapeutics can be are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

Therapeutics of the invention are also suitably administered by sustained-release systems. Suitable examples of sustained-release Therapeutics are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Therapeutics of the invention are also suitably administered by sustained-release systems. Suitable examples of sustained-release Therapeutics include suitable polymeric materials (such as, for example, semi-permeable polymer matrices in the form of shaped articles, e.g., films, or mirocapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2-hydroxyethyl methacrylate) (Langer et al., J. Biomed. Mater. Res. 15:167-277 (1981), and Langer, Chem. Tech. 12:98-105 (1982)), ethylene vinyl acetate (Langer et al., Id.) or poly-D-(−)-3-hydroxybutyric acid (EP 133,988).

In a preferred embodiment polypeptide, polynucleotide and/or antibody compositions of the invention are formulated in a biodegradable, polymeric drug delivery system, for example as described in U.S. Pat. Nos. 4,938,763; 5,278,201; 5,278,202; 5,324,519; 5,340,849; and 5,487,897 and in International Publication Numbers WO01/35929, WO00/24374, and WO00/06117 which are hereby incorporated by reference in their entirety. In specific preferred embodiments the polypeptide, polynucleotide and/or antibody compositions of the invention are formulated using the ATRIGEL® Biodegradable System of Atrix Laboratories, Inc. (Fort Collins, Colo.).

Examples of biodegradable polymers which can be used in the formulation of polypeptide, polynucleotide and/or antibody compositions, include but are not limited to, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), poly(methyl vinyl ether), poly(maleic anhydride), polyvinylpyrrolidone, polyethylene glycol, polyhydroxycellulose, chitin, chitosan, and copolymers, terpolymers, or combinations or mixtures of the above materials. The preferred polymers are those that have a lower degree of crystallization and are more hydrophobic. These polymers and copolymers are more soluble in the biocompatible solvents than the highly crystalline polymers such as polyglycolide and chitin which also have a high degree of hydrogen-bonding. Preferred materials with the desired solubility parameters are the polylactides, polycaprolactones, and copolymers of these with glycolide in which there are more amorphous regions to enhance solubility. In specific preferred embodiments, the biodegradable polymers which can be used in the formulation of polypeptide, polynucleotide and/or antibody compositions are poly(lactide-co-glycolides). Polymer properties such as molecular weight, hydrophobicity, and lactide/glycolide ratio may be modified to obtain the desired drug polypeptide, polynucleotide and/or antibody release profile (See, e.g., Ravivarapu et al., Journal of Pharmaceutical Sciences 89:732-741 (2000), which is hereby incorporated by reference in its entirety).

It is also preferred that the solvent for the biodegradable polymer be non-toxic, water miscible, and otherwise biocompatible. Examples of such solvents include, but are not limited to, N-methyl-2-pyrrolidone, 2-pyrrolidone, C2 to C6 alkanols, C1 to C₁₋₅ alchohols, dils, triols, and tetraols such as ethanol, glycerine propylene glycol, butanol; C3 to C₁₋₅ alkyl ketones such as acetone, diethyl ketone and methyl ethyl ketone; C3 to C15 esters such as methyl acetate, ethyl acetate, ethyl lactate; alkyl ketones such as methyl ethyl ketone, C1 to C15 amides such as dimethylformamide, dimethylacetamide and caprolactam; C3 to C20 ethers such as tetrahydrofuran, or solketal; tweens, triacetin, propylene carbonate, decylmethylsulfoxide, dimethyl sulfoxide, oleic acid, 1-dodecylazacycloheptan-2-one, Other preferred solvents are benzyl alchohol, benzyl benzoate, dipropylene glycol, tributyrin, ethyl oleate, glycerin, glycofural, isopropyl myristate, isopropyl palmitate, oleic acid, polyethylene glycol, propylene carbonate, and triethyl citrate. The most preferred solvents are N-methyl-2-pyrrolidone, 2-pyrrolidone, dimethyl sulfoxide, triacetin, and propylene carbonate because of the solvating ability and their compatibility.

Additionally, formulations comprising polypeptide, polynucleotide and/or antibody compositions and a biodegradable polymer may also include release-rate modification agents and/or pore-forming agents. Examples of release-rate modification agents include, but are not limited to, fatty acids, triglycerides, other like hydrophobic compounds, organic solvents, plasticizing compounds and hydrophilic compounds. Suitable release rate modification agents include, for example, esters of mono-, di-, and tricarboxylic acids, such as 2-ethoxyethyl acetate, methyl acetate, ethyl acetate, diethyl phthalate, dimethyl phthalate, dibutyl phthalate, dimethyl adipate, dimethyl succinate, dimethyl oxalate, dimethyl citrate, triethyl citrate, acetyl tributyl citrate, acetyl triethyl citrate, glycerol triacetate, di(n-butyl) sebecate, and the like; polyhydroxy alcohols, such as propylene glycol, polyethylene glycol, glycerin, sorbitol, and the like; fatty acids; triesters of glycerol, such as triglycerides, epoxidized soybean oil, and other epoxidized vegetable oils; sterols, such as cholesterol; alcohols, such as C.sub.6-C.sub.12 alkanols, 2-ethoxyethanol. The release rate modification agent may be used singly or in combination with other such agents. Suitable combinations of release rate modification agents include, but are not limited to, glycerin/propylene glycol, sorbitol/glycerine, ethylene oxide/propylene oxide, butylene glycol/adipic acid, and the like. Preferred release rate modification agents include, but are not limited to, dimethyl citrate, triethyl citrate, ethyl heptanoate, glycerin, and hexanediol. Suitable pore-forming agents that may be used in the polymer composition include, but are not limited to, sugars such as sucrose and dextrose, salts such as sodium chloride and sodium carbonate, polymers such as hydroxylpropylcellulose, carboxymethylcellulose, polyethylene glycol, and polyvinylpyrrolidone. Solid crystals that will provide a defined pore size, such as salt or sugar, are preferred.

In specific preferred embodiments the polypeptide, polynucleotide and/or antibody compositions of the invention are formulated using the BEMA™ BioErodible Mucoadhesive System, MCA™ MucoCutaneous Absorption System, SMP™ Solvent MicroParticle System, or BCP™ BioCompatible Polymer System of Atrix Laboratories, Inc. (Fort Collins, Colo.).

Sustained-release Therapeutics also include liposomally entrapped Therapeutics of the invention (see generally, Langer, Science 249:1527-1533 (1990); Treat et al., in Liposomes in the Therapy of Infectious Disease and Cancer, Lopez-Berestein and Fidler (eds.), Liss, New York, pp. 317-327 and 353-365 (1989)). Liposomes containing the Therapeutic are prepared by methods known per se: DE 3,218,121; Epstein et al., Proc. Natl. Acad. Sci. (USA) 82:3688-3692 (1985); Hwang et al., Proc. Natl. Acad. Sci.(USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal Therapeutic.

In yet an additional embodiment, the Therapeutics of the invention are delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)).

Other controlled release systems are discussed in the review by Langer (Science 249:1527-1533 (1990)).

For parenteral administration, in one embodiment, the Therapeutic is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to the Therapeutic.

Generally, the formulations are prepared by contacting the Therapeutic uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The Therapeutic is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any pharmaceutical used for therapeutic administration can be sterile. Sterility is readily accomplished by filtration through sterile filtration membranes (e.g., 0.2 micron membranes). Therapeutics generally are placed into a container having a sterile access port, for example, an intravenous solution bag or vial having a stopper pierceable by a hypodermic injection needle.

Therapeutics ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous Therapeutic solution, and the resulting mixture is lyophilized. The infusion solution is prepared by reconstituting the lyophilized Therapeutic using bacteriostatic Water-for-Injection.

The invention also provides a pharmaceutical pack or kit comprising one or more containers filled with one or more of the ingredients of the Therapeutics of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the Therapeutics may be employed in conjunction with other therapeutic compounds.

The Therapeutics of the invention may be administered alone or in combination with adjuvants. Adjuvants that may be administered with the Therapeutics of the invention include, but are not limited to, alum, alum plus deoxycholate (ImmunoAg), MTP-PE (Biocine Corp.), QS21 (Genentech, Inc.), BCG (e.g., THERACYS(O), MPL and nonviable prepartions of Corynebacterium parvum. In a specific embodiment, Therapeutics of the invention are administered in combination with alum. In another specific embodiment, Therapeutics of the invention are administered in combination with QS-21. Further adjuvants that may be administered with the Therapeutics of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the Therapeutics of the invention include, but are not limited to, vaccines directed toward protection against MMR (measles, mumps, rubella), polio, varicella, tetanus/diptheria, hepatitis A, hepatitis B, haemophilus influenzae B, whooping cough, pneumonia, influenza, Lyme's Disease, rotavirus, cholera, yellow fever, Japanese encephalitis, poliomyelitis, rabies, typhoid fever, and pertussis. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The Therapeutics of the invention may be administered alone or in combination with other therapeutic agents. Therapeutic agents, which may be administered in combination with one or more Therapeutics of the invention, include but are not limited to, chemotherapeutic agents, antibiotics, steroidal and non-steroidal anti-inflammatories, conventional immunotherapeutic agents, and/or therapeutic treatments described below. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

One or more therapeutic agent of the invention may be administered in combination with one or more agent used in the treatment and/or amelioration of inflammatory bowel disease such as, for example, Crohn's disease and ulcerative colitis. Agents which are used in the treatment and/or amelioration of inflammatory bowel disease and which may be administered in combination with one or more therapeutics of the present invention, include, but are not limited to, 5-aminosalicylates (5-ASA agents), for example, mesalamine (Asacol, Pentasa, or Rowasa), olsalazine sodium (olsalazine, or Dipentum), balsalazide sodium (balsalazide, Colazide, or Colazal), and sulfalazine (Azulfidine); antibiotics, for example, ciprofloxacin (Cipro), clarithromycin (Biaxin), and metronidazole (Flagyl); anti-TNF alpha monoclonal antibody (Infliximab or Remicade); corticosteroids, for example, budesonide (Entocort, or Budecol), cortisone (Cortone), dexamethasone (Decadron), hydrocortisone, and prednisone (Deltasone, or Orasone); and immunomodulators, for example; azathioprine, 6-mercaptopurine (6-MP, or Purinethol), methotrexate (Folex), and cyclosporine A (cyclosporine, Neoral, Sandimmune, or CSA). Agents which may be used in the treatment of inflammatory bowel disease and which may be administered in combination with one or more therapeutics of the present invention, include, but are not limited to, 4-aminosalicylic acid (4-ASA, or Quadrasa); anticoagulants, for example, heparin, and ridogrel (R-68070, or R-70416); antioxidants, for example, LY-213829 sulfoxide (Tazofelone), and BXT-51072; anti-TNFalpha monoclonal antibody (CDP571, or Humicade); interleukins, for example, interleukin 10 (IL-10), and interleukin 11 (IL-11); anti-interleukin antibody (anti-IL-12 antibody); interferon-beta la; ISIS-2302; anti-a4 integrin antibody (LDP-02, or anti-4-7 integrin monoclonal antibody); Etanercept (Enbrel); MAP kinase inhibitor (CNI-1493); corticosteroids, for example, prednisolone (ATL-2502, or AZM-110); fish oils, for example, purepa: hormones, for example, human growth hormone, medroxy-progesterone acetate (MPA), and DHEAS (dehydroepiandrosterone sulfate); immunomodulators, for example, tacrolimus (FK-506), and mycophenolate mofetil (MMF, or CellCept); tryptase inhibitors, for example, APC-2059; nicotine; thalidomide (CC-1088, or SelCID) and thalidomide analogues (CG-1088); and probiotic compositions, which alter the intestinal bacterial flora.

In a further embodiment one or more compositions, comprising therapeutic agents of the invention together with agents used in the treatment of inflammatory bowel disease, may be administered together with one or more agents useful in the prevention or reduction of side effects associated with administration of said compositions. Agents useful in the prevention or reduction of side effects associated with administration of compositions for the treatment of inflammatory bowel disease include, but are not limited to, antiemetic compounds, anti-inflammatory compounds, and folic acid.

In a further embodiment one or more compositions, comprising therapeutic agents of the invention together with or without agents used in the treatment of inflammatory bowel disease, may be administered together with other forms of treatment used inb the treatment and/or amelioration of inflammatory bowel disease. Treatments useful in the treatment and/or amelioration of inflammatory bowel disease include, but are not limited to, surgical procedures, for example, colostomy.

In a further embodiment, the Therapeutics of the invention are administered in combination with an antiviral agent. Antiviral agents that may be administered with the Therapeutics of the invention include, but are not limited to, acyclovir, ribavirin, amantadine, and remantidine.

In other embodiments, Therapeutics of the invention may be administered in combination with anti-opportunistic infection agents. Anti-opportunistic agents that may be administered in combination with the Therapeutics of the invention, include, but are not limited to, TRIMETHOPRIM-SULFAMETHOXAZOLE™, DAPSONE™, PENTAMIDINE™, ATOVAQUONE™, ISONIAZID™, RIFAMPIN™, PYRAZINAMIDE™, ETHAMBUTOL™, RIFABUTIN™, CLARITHROMYCIN™, AZITHROMYCIN™, GANCICLOVIR™, FOSCARNET™, CIDOFOVIR™, FLUCONAZOLE™, ITRACONAZOLE™, KETOCONAZOLE™, ACYCLOVIR™, FAMCICOLVIR™, PYRIMETHAMINE™, LEUCOVOR™, NEUPOGEN™ (filgrastim/G-CSF), and LEUKINE™ (sargramostim/GM-CSF). In a specific embodiment, Therapeutics of the invention are used in any combination with TRIMETHOPRIM-SULFAMETHOXAZOLE™, DAPSONE™, PENTAMIDINE™, and/or ATOVAQUONE™ to prophylactically treat or prevent an opportunistic Pneumocystis carinii pneumonia infection. In another specific embodiment, Therapeutics of the invention are used in any combination with ISONIAZID™, RIFAMPIN™, PYRAZINAMIDE™, and/or ETHAMBUTOL™ to prophylactically treat or prevent an opportunistic Mycobacterium avium complex infection. In another specific embodiment, Therapeutics of the invention are used in any combination with RIFABUTIN™, CLARITHROMYCIN™, and/or AZITHROMYCIN™ to prophylactically treat or prevent an opportunistic Mycobacterium tuberculosis infection. In another specific embodiment, Therapeutics of the invention are used in any combination with GANCICLOVIR™, FOSCARNET™, and/or CIDOFOVIR™ to prophylactically treat or prevent an opportunistic cytomegalovirus infection. In another specific embodiment, Therapeutics of the invention are used in any combination with FLUCONAZOLE™, ITRACONAZOLE™, and/or KETOCONAZOLE™ to prophylactically treat or prevent an opportunistic fungal infection. In another specific embodiment, Therapeutics of the invention are used in any combination with ACYCLOVIR™ and/or FAMCICOLVIR™ to prophylactically treat or prevent an opportunistic herpes simplex virus type I and/or type II infection. In another specific embodiment, Therapeutics of the invention are used in any combination with PYRIMETHAMINE™ and/or LEUCOVORIN™ to prophylactically treat or prevent an opportunistic Toxoplasma gondii infection. In another specific embodiment, Therapeutics of the invention are used in any combination with LEUCOVORIN™ and/or NEUPOGEN™ to prophylactically treat or prevent an opportunistic bacterial infection.

In a further embodiment, the Therapeutics of the invention are administered in combination with an antibiotic agent. Antibiotic agents that may be administered with the Therapeutics of the invention include, but are not limited to, amoxicillin, aminoglycosides, beta-lactam (glycopeptide), beta-lactamases, Clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamethoxazole, and vancomycin.

In particular embodiments, the use of compositions of the invention in combination with antiviral, anti-opportunistic infection, and antibiotic agents is contemplated for the treatment, prevention, and/or amelioration of an inflammatory bowel disease, such as for example, ulcerative colitis or Crohn's disease, as described herein.

In a particular embodiment, the use of compositions of the invention in combination with antiviral, anti-opportunistic infection, and antibiotic agents is contemplated for the treatment, prevention, and/or amelioration of ulcerative colitis. In a further particular embodiment, the use of compositions of the invention in combination with antiviral, anti-opportunistic infection, and antibiotic agents is contemplated for the treatment, prevention, and/or amelioration of Crohn's disease.

In other embodiments, Therapeutics of the invention are administered in combination with immunosuppressive agents. Immunosuppressive agents that may be administered in combination with the Therapeutics of the invention include, but are not limited to, steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells. Other immunosuppressive agents that may be administered in combination with the Therapeutics of the invention include, but are not limited to, prednisolone, methotrexate, thalidomide, methoxsalen, rapamycin, leflunomide, mizoribine (BREDININ™), brequinar, deoxyspergualin, and azaspirane (SKF 105685), ORTHOCLONE OKT® 3 (muromonab-CD3), SANDIMMUNE™, NEORAL™, SANGDYA™ (cyclosporine), PROGRAF® (FK506, tacrolimus), CELLCEPT® (mycophenolate motefil, of which the active metabolite is mycophenolic acid), IMURAN™ (azathioprine), glucocorticosteroids, adrenocortical steroids such as DELTASONE™ (prednisone) and HYDELTRASOL™ (prednisolone), FOLEX™ and MEXATE™ (methotrxate), OXSORALEN-ULTRA™ (methoxsalen) and RAPAMUNE™ (sirolimus). In a specific embodiment, immunosuppressants may be used to prevent rejection of organ or bone marrow transplantation.

In an additional embodiment, Therapeutics of the invention are administered alone or in combination with one or more intravenous immune globulin preparations. Intravenous immune globulin preparations that may be administered with the Therapeutics of the invention include, but not limited to, GAMMAR™, IVEEGAM™, SANDOGLOBULIN™, GAMMAGARD S/D™, ATGAM™ (antithymocyte glubulin), and GAMIMUNE™. In a specific embodiment, Therapeutics of the invention are administered in combination with intravenous immune globulin preparations in transplantation therapy (e.g., bone marrow transplant).

In certain embodiments, the Therapeutics of the invention are administered alone or in combination with an anti-inflammatory agent. Anti-inflammatory agents that may be administered with the Therapeutics of the invention include, but are not limited to, corticosteroids (e.g. betamethasone, budesonide, cortisone, dexamethasone, hydrocortisone, methylprednisolone, prednisolone, prednisone, and triamcinolone), nonsteroidal anti-inflammatory drugs (e.g., diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic acid, and tolmetin.), as well as antihistamines, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In particular embodiments, the use of compositions of the invention in combination with immune globulin preparations, iummunopsuppressive and antiinflammatory agents is contemplated for the treatment, prevention, and/or amelioration of an inflammatory bowel disease, such as for example, ulcerative colitis or Crohn's disease, as described herein.

In a particular embodiment, the use of compositions of the invention in combination with immune globulin preparations, iummunopsuppressive and antiinflammatory agents is contemplated for the treatment, prevention, and/or amelioration of ulcerative colitis. In a further particular embodiment, the use of compositions of the invention in combination with immune globulin preparations, iummunopsuppressive and antiinflammatory agents is contemplated for the treatment, prevention, and/or amelioration of Crohn's disease.

In an additional embodiment, the Therapeutics of the invention are administered in combination with angiogenic proteins. Angiogenic proteins that may be administered with the Therapeutics of the invention include, but are not limited to, Glioma Derived Growth Factor (GDGF), as disclosed in European Patent Number EP-399816; Platelet Derived Growth Factor-A (PDGF-A), as disclosed in European Patent Number EP-6821 10; Platelet Derived Growth Factor-B (PDGF-B), as disclosed in European Patent Number EP-282317; Placental Growth Factor (PlGF), as disclosed in International Publication Number WO 92/06194; Placental Growth Factor-2 (PlGF-2), as disclosed in Hauser et al., Growth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above-mentioned references are herein incorporated by reference in their entireties. Further examples of angiogenic proteins that may be administered with the compositions of the invention include, but are not limited to, epidermal growth factor alpha, epidermal growth factor beta, platelet-derived endothelial cell growth factor, hepatocyte growth factor, insulin-like growth factor, colony stimulating factor, and nitric oxide synthase.

In an additional embodiment, the Therapeutics of the invention may be administered in combination with Fibroblast Growth Factors. Fibroblast Growth Factors that may be administered with the Therapeutics of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

In an additional embodiment, the Therapeutics of the invention may be administered in combination with cytokines. Cytokines that may be administered with the Therapeutics of the invention include, but are not limited to, IL2, IL3, IL4, IL5, IL6, IL7, IL10, IL12, IL13, IL15, anti-CD40, CD40L, IFN-gamma and TNF-alpha. In another embodiment, Therapeutics of the invention may be administered with any interleukin, including, but not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, and IL-21.

In one embodiment, the Therapeutics of the invention are administered in combination with members of the TNF family. TNF, TNF-related or TNF-like molecules that may be administered with the Therapeutics of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TRANK, TR9 (International Publication No. WO 98/56892),TR10 (International Publication No. WO 98/54202), 312C2 (International Publication No. WO 98/06842), and TR12, and soluble forms CD154, CD70, and CD153.

In particular embodiments, the use of compositions of the invention in combination with angiogenic proteins, fibroblast growth factors, cytokines and TNF family members is contemplated for the treatment, prevention, and/or amelioration of an inflammatory bowel disease, such as for example, ulcerative colitis or Crohn's disease, as described herein.

In a particular embodiment, the use of compositions of the invention in combination with angiogenic proteins, fibroblast growth factors, cytokines and TNF family members is contemplated for the treatment, prevention, and/or amelioration of ulcerative colitis. In a further particular embodiment, the use of compositions of the invention in combination with angiogenic proteins, fibroblast growth factors, cytokines and TNF family members is contemplated for the treatment, prevention, and/or amelioration of Crohn's disease.

In an additional embodiment, the Therapeutics of the invention are administered in combination with hematopoietic growth factors. Hematopoietic growth factors that may be administered with the Therapeutics of the invention include, but are not limited to, granulocyte macrophage colony stimulating factor (GM-CSF) (sargramostim, LEUKINE™, PROKINE™), granulocyte colony stimulating factor (G-CSF) (filgrastim, NEUPOGEN™), macrophage colony stimulating factor (M-CSF, CSF-1) erythropoietin (epoetin alfa, EPOGEN™, PROCRIT™), stem cell factor (SCF, c-kit ligand, steel factor), megakaryocyte colony stimulating factor, PIXY321 (a GMCSF/IL-3 fusion protein), interleukins, especially any one or more of IL-1 through IL-12, interferon-gamma, or thrombopoietin.

In another embodiment, the Therapeutics of the invention are administered in combination with diuretic agents, such as carbonic anhydrase-inhibiting agents (e.g., acetazolamide, dichlorphenamide, and methazolamide), osmotic diuretics (e.g., glycerin, isosorbide, mannitol, and urea), diuretics that inhibit Na⁺-K⁺-2Cl⁻ symport (e.g., furosemide, bumetamide, azosemide, piretamide, tripamide, ethacrynic acid, muzolimine, and torsemide), thiazide and thiazide-like diuretics (e.g., bendroflumethiazide, benzthiazide, chlorothiazide, hydrochlorothiazide, hydroflumethiazide, methyclothiazide, polythiazide, trichormethiazide, chlorthalidone, indapamide, metolazone, and quinethazone), potassium sparing diuretics (e.g., amiloride and triamterene), and mineralcorticoid receptor antagonists (e.g., spironolactone, canrenone, and potassium canrenoate).

In one embodiment, the Therapeutics of the invention are administered in combination with treatments for endocrine and/or hormone imbalance disorders. Treatments for endocrine and/or hormone imbalance disorders include, but are not limited to, 271, radioactive isotopes of iodine such as and I; recombinant growth hormone, such as HUMATROPE™ (recombinant somatropin); growth hormone analogs such as PROTROPIN™ (somatrem); dopamine agonists such as PARLODEL™ (bromocriptine); somatostatin analogs such as SANDOSTATIN™ (octreotide); gonadotropin preparations such as PREGNYL™, A.P.L.™ and PROFASI™ (chorionic gonadotropin (CG)), PERGONAL™ (menotropins), and METRODIN™ (urofollitropin (uFSH)); synthetic human gonadotropin releasing hormone preparations such as FACTREL™ and LUTREPULSE™ (gonadorelin hydrochloride); synthetic gonadotropin agonists such as LUPRON™ (leuprolide acetate), SUPPRELN™ (histrelin acetate), SYNAREL™ (nafarelin acetate), and ZOLADEX™ (goserelin acetate); synthetic preparations of thyrotropin-releasing hormone such as RELEFACT TRH™ and THYPINONE™ (protirelin); recombinant human TSH such as THYROGEN™; synthetic preparations of the sodium salts of the natural isomers of thyroid hormones such as L-T₄™, SYNTHROID™ and LEVOTHROID™ (levothyroxine sodium), L-T₃™, CYTOMEL™ and TRIOSTAT™ (liothyroine sodium), and THYROLAR™ (liotrix); antithyroid compounds such as 6-n-propylthiouracil (propylthiouracil), 1-methyl-2-mercaptoimidazole and TAPAZOLE™ (methimazole), NEO-MERCAZOLE™ (carbimazole); beta-adrenergic receptor antagonists such as propranolol and esmolol; Ca²⁺ channel blockers; dexamethasone and iodinated radiological contrast agents such as TELEPAQUE™ (iopanoic acid) and ORAGRAFIN™ (sodium ipodate).

Additional treatments for endocrine and/or hormone imbalance disorders include, but are not limited to, estrogens or congugated estrogens such as ESTRACE™ (estradiol), ESTINYL™ (ethinyl estradiol), PREMARIN™, ESTRATAB™, ORTHO-EST™, OGEN™ and estropipate (estrone), ESTROVIS™ (quinestrol), ESTRADERM™ (estradiol), DELESTROGEN™ and VALERGEN™ (estradiol valerate), DEPO-ESTRADIOL CYPIONATE™ and ESTROJECT LA™ (estradiol cypionate); antiestrogens such as NOLVADEX™ (tamoxifen), SEROPHENE™ and CLOMID™ (clomiphene); progestins such as DURALUTIN™ (hydroxyprogesterone caproate), MPA™ and DEPO-PROVERA™ (medroxyprogesterone acetate), PROVERA™ and CYCRIN™ (MPA), MEGACE™ (megestrol acetate), NORLUTIN™ (norethindrone), and NORLUTATE™ and AYGESTIN™ (norethindrone acetate); progesterone implants such as NORPLANT SYSTEM™ (subdermal implants of norgestrel); antiprogestins such as RU 486™ (mifepristone); hormonal contraceptives such as ENOVID™ (norethynodrel plus mestranol), PROGESTASERT™ (intrauterine device that releases progesterone), LOESTRIN™, BREVICON™, MODICON™, GENORA™, NELONA™, NORINYL™, OVACON-35 ™ and OVACON-50™ (ethinyl estradiol/norethindrone), LEVLEN™, NORDETTE™, TRI-LEVLEN™ and TRIPHASIL-21™ (ethinyl estradiol/levonorgestrel) LO/OVRAL™ and OVRAL™ (ethinyl estradiol/norgestrel), DEMULEN™ (ethinyl estradiol/ethynodiol diacetate), NORINYL™, ORTHO-NOVUM™, NORETHN™, GENORA™, and NELOVA™ (norethindrone/mestranol), DESOGEN™ and ORTHO-CEPT™ (ethinyl estradiol/desogestrel), ORTHO-CYCLEN™ and ORTHO-TRICYCLEN™ (ethinyl estradiol/norgestimate), MICRONOR™ and NOR-QD™ (norethindrone), and OVRETTE™ (norgestrel).

Additional treatments for endocrine and/or hormone imbalance disorders include, but are not limited to, testosterone esters such as methenolone acetate and testosterone undecanoate; parenteral and oral androgens such as TESTOJECT-50™ (testosterone), TESTEX™ (testosterone propionate), DELATESTRYL™ (testosterone enanthate), DEPO-TESTOSTERONE™ (testosterone cypionate), DANOCRINE™ (danazol), HALOTESTIN™ (fluoxymesterone), ORETON METHYL™, TESTRED™ and VIRILON™ (methyltestosterone), and OXANDRIN™ (oxandrolone); testosterone transdermal systems such as TESTODERM™; androgen receptor antagonist and 5-alpha-reductase inhibitors such as ANDROCUR™ (cyproterone acetate), EULEXIN™ (flutamide), and PROSCAR™ (finasteride); adrenocorticotropic hormone preparations such as CORTROSYN™ (cosyntropin); adrenocortical steroids and their synthetic analogs such as ACLOVATE™ (alclometasone dipropionate), CYCLOCORT™ (amcinonide), BECLOVENT™ and VANCERIL™ (beclomethasone dipropionate), CELESTONE™ (betamethasone), BENISONE™ and UTICORT™ (betamethasone benzoate), DIPROSONE™ (betamethasone dipropionate), CELESTONE PHOSPHATE™ (betamethasone sodium phosphate), CELESTONE SOLUSPAN™ (betamethasone sodium phosphate and acetate), BETA-VAL™ and VALISONE™ (betamethasone valerate), TEMOVATE™ (clobetasol propionate), CLODERM™ (clocortolone pivalate), CORTEF™ and HYDROCORTONE™ (cortisol (hydrocortisone)), HYDROCORTONE ACETATE™ (cortisol (hydrocortisone) acetate), LOCOID™ (cortisol (hydrocortisone) butyrate), HYDROCORTONE PHOSPHATE™ (cortisol (hydrocortisone) sodium phosphate), A-HYDROCORT™ and SOLU CORTEF™ (cortisol (hydrocortisone) sodium succinate), WESTCORT™ (cortisol (hydrocortisone) valerate), CORTISONE ACETATE™ (cortisone acetate), DESOWEN™ and TRIDESILON™ (desonide), TOPICORT™ (desoximetasone), DECADRON™ (dexamethasone), DECADRON LA™ (dexamethasone acetate), DECADRON PHOSPHATE™ and HEXADROL PHOSPHATE™ (dexamethasone sodium phosphate), FLORONE™ and MAXIFLOR™ (diflorasone diacetate), FLORINEF ACETATE™ (fludrocortisone acetate), AEROBID™ and NASALIDE™ (flunisolide), FLUONID™ and SYNALAR™ (fluocinolone acetonide), LIDEX™ (fluocinonide), FLUOR-OP™ and FML™ (fluorometholone), CORDRAN™ (flurandrenolide), HALOG™ (halcinonide), HMS LIZUIFILM™ (medrysone), MEDROL™ (methylprednisolone), DEPO-MEDROL™ and MEDROL ACETATE™ (methylprednisone acetate), A-METHAPRED™ and SOLUMEDROL™ (methylprednisolone sodium succinate), ELOCON™ (mometasone furoate), HALDRONE™ (paramethasone acetate), DELTA-CORTEF™ (prednisolone), ECONOPRED™ (prednisolone acetate), HYDELTRASOL™ (prednisolone sodium phosphate), HYDELTRA-T.B.A.™ (prednisolone tebutate), DELTASONE™ (prednisone), ARISTOCORT™ and KENACORT™ (triamcinolone), KENALOG™ (triamcinolone acetonide), ARISTOCORT™ and KENACORT DIACETATE™ (triamcinolone diacetate), and ARISTOSPAN™ (triamcinolone hexacetonide); inhibitors of biosynthesis and action of adrenocortical steroids such as CYTADREN™ (aminoglutethimide), NIZORAL™ (ketoconazole), MODRASTANE™ (trilostane), and METOPIRONE™ (metyrapone).

Additional treatments for endocrine and/or hormone imbalance disorders include, but are not limited to bovine, porcine or human insulin or mixtures thereof, insulin analogs; recombinant human insulin such as HUMULIN™ and NOVOLIN™; oral hypoglycemic agents such as ORAMIDE™ and ORINASE™ (tolbutamide), DLABINESE™ (chlorpropamide), TOLAMIDE™ and TOLINASE™ (tolazamide), DYMELOR™ (acetohexamide), glibenclamide, MICRONASE™, DIBETA™ and GLYNASE™ (glyburide), GLUCOTROL™ (glipizide), and DIAMICRON™ (gliclazide), GLUCOPHAGE™ (metformin), PRECOSE™ (acarbose), AMARYL™ (glimepiride), and ciglitazone; thiazolidinediones (TZDs) such as rosiglitazone, AVANDIA™ (rosiglitazone maleate) ACTOS™ (piogliatazone), and troglitazone; alpha-glucosidase inhibitors; bovine or porcine glucagon; somatostatins such as SANDOSTATIN™ (octreotide); and diazoxides such as PROGLYCEM™ (diazoxide). In still other embodiments, Therapeutics of the invention are administered in combination with one or more of the following: a biguamide antidiabetic agent, a glitazone antidiabetic agent, and a sulfonylurea antidiabetic agent.

In additional embodiments, the Therapeutics of the invention are administered in combination with other therapeutic or prophylactic regimens, such as, for example, radiation therapy.

Gene Therapy Methods

Another aspect of the present invention is to gene therapy methods for treating or preventing disorders, diseases and conditions of the gastrointestinal tract, preferably inflammatory bowel disease. The gene therapy methods relate to the introduction of nucleic acid (DNA, RNA and antisense DNA or RNA) sequences into an animal to achieve expression of a polypeptide of the present invention. This method requires a polynucleotide which codes for a polypeptide of the invention that operatively linked to a promoter and any other genetic elements necessary for the expression of the polypeptide by the target tissue. Such gene therapy and delivery techniques are known in the art, see, for example, WO90/11092, which is herein incorporated by reference.

Thus, for example, cells from a patient may be engineered with a polynucleotide (DNA or RNA) comprising a promoter operably linked to a polynucleotide of the invention ex vivo, with the engineered cells then being provided to a patient to be treated with the polypeptide. Such methods are well known in the art. For example, see Belldegrun et al., J. Natl. Cancer Inst., 85:207-216 (1993); Ferrantini et al., Cancer Research, 53:107-1112 (1993); Ferrantini et al., J. Immunology 153: 4604-4615 (1994); Kaido, T., et al., Int. J. Cancer 60: 221-229 (1995); Ogura et al., Cancer Research 50: 5102-5106 (1990); Santodonato, et al., Human Gene Therapy 7:1-10 (1996); Santodonato, et al., Gene Therapy 4:1246-1255 (1997); and Zhang, et al., Cancer Gene Therapy 3: 31-38 (1996)), which are herein incorporated by reference. In one embodiment, the engineered cells are arterial cells. The arterial cells may be reintroduced into the patient through direct injection to the artery, the tissues surrounding the artery, or through catheter injection.

As discussed in more detail below, the polynucleotide constructs can be delivered by any method that delivers injectable materials to the cells of an animal, such as, injection into the interstitial space of tissues (preferably mucosal tissue of the mouth, esophagus, stomach, small intestine, large intestine, or rectum, or alternatively heart, muscle, skin, lung, liver, and the like). The polynucleotide constructs may be delivered in a pharmaceutically acceptable liquid or aqueous carrier.

In one embodiment, the polynucleotide of the invention is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, the polynucleotides of the invention can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The polynucleotide vector constructs of the invention used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF11V5, pcDNA3.1, and pRc/CMV2 available from Invitrogen. Other suitable vectors will be readily apparent to the skilled artisan.

Any strong promoter known to those skilled in the art can be used for driving the expression of polynucleotide sequence of the invention. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the beta-actin promoter; and human growth hormone promoters. The promoter also may be the native promoter for the polynucleotides of the invention.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct of the invention can be delivered to the interstitial space of tissues within an animal, including mucosal tissue of the mouth, esophagus, stomach, small intestine, large intestine or rectum, or alternatively muscle, skin, brain, lung, liver, spleen, bone marrow, thymus, heart, lymph, blood, bone, cartilage, pancreas, kidney, gall bladder, testis, ovary, uterus, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

The constructs may also be delivered with delivery vehicles such as viral sequences, viral particles, liposome formulations, lipofectin, precipitating agents, etc. Such methods of delivery are known in the art.

In certain embodiments, the polynucleotide constructs of the invention are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA, 84:7413-7416 (1987), which is herein incorporated by reference); mRNA (Malone et al., Proc. Natl. Acad. Sci. USA, 86:6077-6081 (1989), which is herein incorporated by reference); and purified transcription factors (Debs et al., J. Biol. Chem., 265:10189-10192 (1990), which is herein incorporated by reference), in functional form.

Cationic liposomes are readily available. For example, N[11-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl. Acad. Sci. USA, 84:7413-7416 (1987), which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication NO: WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., Felgner et al., Proc. Natl. Acad. Sci. USA, 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology, 101:512-527 (1983), which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca 2+-EDTA chelation (Papahadjopoulos et al., Biochim. Biophys. Acta, 394:483 (1975); Wilson et al., Cell, 17:77 (1979)); ether injection (Deamer et al., Biochim. Biophys. Acta, 443:629 (1976); Ostro et al., Biochem. Biophys. Res. Commun., 76:836 (1977); Fraley et al., Proc. Natl. Acad. Sci. USA, 76:3348 (1979)); detergent dialysis (Enoch et al., Proc. Natl. Acad. Sci. USA, 76:145 (1979)); and reverse-phase evaporation (REV) (Fraley et al., J. Biol. Chem., 255:10431 (1980); Szoka et al., Proc. Natl. Acad. Sci. USA, 75:145 (1978); Schaefer-Ridder et al., Science, 215:166 (1982)), which are herein incorporated by reference.

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,589, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals.

In certain embodiments, cells are engineered, ex vivo or in vivo, using a retroviral particle containing RNA that comprises a sequence encoding polypeptides of the invention. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14×, VT-19-17-H2, RCRE, RCRIP, GP+E-86, GP+envAm12, and DAN cell lines as described in Miller, Human Gene Therapy, 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include polynucleotide encoding polypeptides of the invention. Such retroviral vector particles then may be employed, to transduce eukaryotic cells, either in vitro or in vivo. The transduced eukaryotic cells will express polypeptides of the invention.

In certain other embodiments, cells are engineered, ex vivo or in vivo, with polynucleotides of the invention contained in an adenovirus vector. Adenovirus can be manipulated such that it encodes and expresses polypeptides of the invention, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, et al., Am. Rev. Respir. Dis., 109:233-238 (1974)). Finally, adenovirus mediated gene transfer has been demonstrated in a number of instances including transfer of alpha-1-antitrypsin and CFTR to the lungs of cotton rats (Rosenfeld et al., Science, 252:431-434 (1991); Rosenfeld et al., Cell, 68:143-155 (1992)). Furthermore, extensive studies to attempt to establish adenovirus as a causative agent in human cancer were uniformly negative (Green et al. Proc. Natl. Acad. Sci. USA, 76:6606 (1979)).

Suitable adenoviral vectors useful in the present invention are described, for example, in Kozarsky and Wilson, Curr. Opin. Genet. Devel., 3:499-503 (1993); Rosenfeld et al., Cell, 68:143-155 (1992); Engelhardt et al., Human Genet. Ther., 4:759-769 (1993); Yang et al., Nature Genet., 7:362-369 (1994); Wilson et al., Nature, 365:691-692 (1993); and U.S. Pat. No. 5,652,224, which are herein incorporated by reference. For example, the adenovirus vector Ad2 is useful and can be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, Curr. Topics in Microbiol. Immunol., 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The polynucleotide construct containing polynucleotides of the invention is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct of the invention. These viral particles are then used to transduce eukaryotic cells, either ex vivo or in vivo. The transduced cells will contain the polynucleotide construct integrated into its genome, and will express the desired gene product.

Another method of gene therapy involves operably associating heterologous control regions and endogenous polynucleotide sequences (e.g. encoding the polypeptide sequence of interest) via homologous recombination (see, e.g., U.S. Pat. No. 5,641,670, issued Jun. 24, 1997; International Publication NO: WO 96/29411, published Sep. 26, 1996; International Publication NO: WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous sequence is placed under the control of the promoter. The promoter then drives the expression of the endogenous sequence.

The polynucleotides encoding polypeptides of the present invention may be administered along with other polynucleotides encoding other angiongenic proteins. Angiogenic proteins include, but are not limited to, acidic and basic fibroblast growth factors, VEGF-1, VEGF-2 (VEGF-C), VEGF-3 (VEGF-B), epidermal growth factor alpha and beta, platelet-derived endothelial cell growth factor, platelet-derived growth factor, tumor necrosis factor alpha, hepatocyte growth factor, insulin like growth factor, colony stimulating factor, macrophage colony stimulating factor, granulocyte/macrophage colony stimulating factor, and nitric oxide synthase.

Preferably, the polynucleotide encoding a polypeptide of the invention contains a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers. (Kaneda et al., Science, 243:375 (1989)).

A preferred method of local administration is by direct injection. Preferably, a recombinant molecule of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189:11277-11281 (1992), which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian. Therapeutic compositions of the present invention can be administered to any animal, preferably to mammals and birds. Preferred mammals include humans, dogs, cats, mice, rats, rabbits sheep, cattle, horses and pigs, with humans being particularly

Targeted Delivery

In another embodiment, the invention provides a method of delivering compositions to targeted cells expressing a receptor for a polypeptide of the invention, or cells expressing a cell bound form of a polypeptide of the invention.

As discussed herein, polypeptides or antibodies of the invention may be associated with heterologous polypeptides, heterologous nucleic acids, toxins, or prodrugs' via hydrophobic, hydrophilic, ionic and/or covalent interactions. In one embodiment, the invention provides a method for the specific delivery of compositions of the invention to cells by administering polypeptides of the invention (including antibodies) that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides a method for the specific destruction of cells (e.g., the destruction of tumor cells) by administering polypeptides of the invention (e.g., polypeptides of the invention or antibodies of the invention) in association with toxins or cytotoxic prodrugs.

By “toxin” is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. By “cytotoxic prodrug” is meant a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

Drug Screening

Further contemplated is the use of the polypeptides of the present invention, or the polynucleotides encoding these polypeptides, to screen for molecules which modify the activities of the polypeptides of the present invention. Such a method would include contacting the polypeptide of the present invention with a selected compound(s) suspected of having antagonist or agonist activity, and assaying the activity of these polypeptides following binding.

This invention is particularly useful for screening therapeutic compounds by using the polypeptides of the present invention, or binding fragments thereof, in any of a variety of drug screening techniques. The polypeptide or fragment employed in such a test may be affixed to a solid support, expressed on a cell surface, free in solution, or located intracellularly. One method of drug screening utilizes eukaryotic or prokaryotic host cells which are stably transformed with recombinant nucleic acids expressing the polypeptide or fragment. Drugs are screened against such transformed cells in competitive binding assays. One may measure, for example, the formulation of complexes between the agent being tested and a polypeptide of the present invention.

Thus, the present invention provides methods of screening for drugs or any other agents which affect activities mediated by the polypeptides of the present invention. These methods comprise contacting such an agent with a polypeptide of the present invention or a fragment thereof and assaying for the presence of a complex between the agent and the polypeptide or a fragment thereof by methods well known in the art. In such a competitive binding assay, the agents to screen are typically labeled. Following incubation, free agent is separated from that present in bound form, and the amount of free or uncomplexed label is a measure of the ability of a particular agent to bind to the polypeptides of the present invention.

Another technique for drug screening provides high throughput screening for compounds having suitable binding affinity to the polypeptides of the present invention, and is described in great detail in European Patent Application 84/03564, published on Sep. 13, 1984, which is incorporated herein by reference herein. Briefly stated, large numbers of different small peptide test compounds are synthesized on a solid substrate, such as plastic pins or some other surface. The peptide test compounds are reacted with polypeptides of the present invention and washed. Bound polypeptides are then detected by methods well known in the art. Purified polypeptides are coated directly onto plates for use in the aforementioned drug screening techniques. In addition, non-neutralizing antibodies may be used to capture the peptide and immobilize it on the solid support.

This invention also contemplates the use of competitive drug screening assays in which neutralizing antibodies capable of binding polypeptides of the present invention specifically compete with a test compound for binding to the polypeptides or fragments thereof. In this manner, the antibodies are used to detect the presence of any peptide which shares one or more antigenic epitopes with a polypeptide of the invention.

Polypeptides of the Invention Binding Peptides and Other Molecules

The invention also encompasses screening methods for identifying polypeptides and nonpolypeptides that bind polypeptides of the invention, and the polypeptide of the invention binding molecules identified thereby. These binding molecules are useful, for example, as agonists and antagonists of the polypeptides of the invention. Such agonists and antagonists can be used, in accordance with the invention, in the therapeutic embodiments described in detail, below.

This method comprises the steps of: contacting a polypeptide of the invention with a plurality of molecules; and identifying a molecule that binds the polypeptide of the invention.

The step of contacting the polypeptide of the invention with the plurality of molecules may be effected in a number of ways. For example, one may contemplate immobilizing the polypeptide of the invention on a solid support and bringing a solution of the plurality of molecules in contact with the immobilized polypeptide of the invention. Such a procedure would be akin to an affinity chromatographic process, with the affinity matrix being comprised of the immobilized polypeptide of the invention. The molecules having a selective affinity for the polypeptide of the invention can then be purified by affinity selection. The nature of the solid support, process for attachment of the polypeptide of the invention to the solid support, solvent, and conditions of the affinity isolation or selection are largely conventional and well known to those of ordinary skill in the art.

Alternatively, one may also separate a plurality of polypeptides into substantially separate fractions comprising a subset of or individual polypeptides. For instance, one can separate the plurality of polypeptides by gel electrophoresis, column chromatography, or like method known to those of ordinary skill for the separation of polypeptides. The individual polypeptides can also be produced by a transformed host cell in such a way as to be expressed on or about its outer surface (e.g., a recombinant phage). Individual isolates can then be “probed” by the polypeptide of the invention, optionally in the presence of an inducer should one be required for expression, to determine if any selective affinity interaction takes place between the polypeptide of the invention and the individual clone. Prior to contacting the polypeptide of the invention with each fraction comprising individual polypeptides, the polypeptides could first be transferred to a solid support for additional convenience. Such a solid support may simply be a piece of filter membrane, such as one made of nitrocellulose or nylon. In this manner, positive clones could be identified from a collection of transformed host cells of an expression library, which harbor a DNA construct encoding a polypeptide having a selective affinity for a polypeptide of the invention. Furthermore, the amino acid sequence of the polypeptide having a selective affinity for the polypeptide of the invention can be determined directly by conventional means or the coding sequence of the DNA encoding the polypeptide can frequently be determined more conveniently. The primary sequence can then be deduced from the corresponding DNA sequence. If the amino acid sequence is to be determined from the polypeptide itself, one may use microsequencing techniques. The sequencing technique may include mass spectroscopy.

In certain situations, it may be desirable to wash away any unbound polypeptide of the invention, or alternatively, unbound polypeptides, from a mixture of the polypeptide of the invention and the plurality of polypeptides prior to attempting to determine or to detect the presence of a selective affinity interaction. Such a wash step may be particularly desirable when the polypeptide of the invention or the plurality of polypeptides is bound to a solid support.

The plurality of molecules provided according to this method may be provided by way of diversity libraries, such as random or combinatorial peptide or nonpeptide libraries which can be screened for molecules that specifically bind to a polypeptide of the invention. Many libraries are known in the art that can be used, e.g., chemically synthesized libraries, recombinant (e.g., phage display libraries), and in vitro translation-based libraries. Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710;Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, R. B., et al., 1992, J. Mol. Biol. 227:711-718); Lenstra, 1992, J. Immunol. Meth. 152:149-157; Kay et al., 1993, Gene 128:59-65; and PCT Publication No. WO 94/18318 dated Aug. 18, 1994.

In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

The variety of non-peptide libraries that are useful in the present invention is great. For example, Ecker and Crooke, 1995, Bio/Technology 13:351-360 list benzodiazepines, hydantoins, piperazinediones, biphenyls, sugar analogs, beta-mercaptoketones, arylacetic acids, acylpiperidines, benzopyrans, cubanes, xanthines, aminimides, and oxazolones as among the chemical species that form the basis of various libraries.

Non-peptide libraries can be classified broadly into two types: decorated monomers and oligomers. Decorated monomer libraries employ a relatively simple scaffold structure upon which a variety functional groups is added. Often the scaffold will be a molecule with a known useful pharmacological activity. For example, the scaffold might be the benzodiazepine structure.

Non-peptide oligomer libraries utilize a large number of monomers that are assembled together in ways that create new shapes that depend on the order of the monomers. Among the monomer units that have been used are carbamates, pyrrolinones, and morpholinos. Peptoids, peptide-like oligomers in which the side chain is attached to the alpha amino group rather than the alpha carbon, form the basis of another version of non-peptide oligomer libraries. The first non-peptide oligomer libraries utilized a single type of monomer and thus contained a repeating backbone. Recent libraries have utilized more than one monomer, giving the libraries added flexibility.

Screening the libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992; BioTechniques 13:422-427; Oldenburg et-al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and CT Publication No. WO 94/18318.

In a specific embodiment, screening to identify a molecule that binds a polypeptide of the invention can be carried out by contacting the library members with a polypeptide of the invention immobilized on a solid phase and harvesting those library members that bind to the polypeptide of the invention. Examples of such screening methods, termed “panning” techniques are described by way of example in Parnley and Smith, 1988, Gene 73:305-318; Fowlkes et al., 1992, BioTechniques 13:422-427; PCT Publication No. WO 94/18318; and in references cited herein.

In another embodiment, the two-hybrid system for selecting interacting proteins in yeast (Fields and Song, 1989, Nature 340:245-246; Chien et al., 1991, Proc. Natl. Acad. Sci. USA 88:9578-9582) can be used to identify molecules that specifically bind to a polypeptide of the invention.

Where the polypeptide of the invention binding molecule is a polypeptide, the polypeptide can be conveniently selected from any peptide library, including random peptide libraries, combinatorial peptide libraries, or biased peptide libraries. The term “biased” is used herein to mean that the method of generating the library is manipulated so as to restrict one or more parameters that govern the diversity of the resulting collection of molecules, in this case peptides.

Thus, a truly random peptide library would generate a collection of peptides in which the probability of finding a particular amino acid at a given position of the peptide is the same for all 20 amino acids. A bias can be introduced into the library, however, by specifying, for example, that a lysine occur every fifth amino acid or that positions 4, 8, and 9 of a decapeptide library be fixed to include only arginine. Clearly, many types of biases can be contemplated, and the present invention is not restricted to any particular bias. Furthermore, the present invention contemplates specific types of peptide libraries, such as phage displayed peptide libraries and those that utilize a DNA construct comprising a lambda phage vector with a DNA insert.

As mentioned above, in the case of a polypeptide of the invention binding molecule that is a polypeptide, the polypeptide may have about 6 to less than about 60 amino acid residues, preferably about 6 to about 10 amino acid residues, and most preferably, about 6 to about 22 amino acids. In another embodiment, a polypeptide of the invention binding polypeptide has in the range of 15-100 amino acids, or 20-50 amino acids.

The selected polypeptide of the invention binding polypeptide can be obtained by chemical synthesis or recombinant expression.

### EXAMPLES

### Example 1

### Bacterial Expression and Purification of TNF-gamma-β

The DNA sequence encoding the full-length TNF-gamma-β ORF, ATCC Deposit No. 203055, was initially amplified using PCR oligonucleotide primers corresponding to the 5′ and 3′ sequences of the TNF-gamma-β protein. Additional nucleotides corresponding to TNF-gamma-β were added to the 5′ and 3′ sequences respectively. The 5′ oligonucleotide primer is shown as SEQ ID NO:47 and has the sequence 5′-GCG CGG ATC CAC CAT GAG ACG CTT TTT AAG CAA AGT C-3′ which contains a Bam HI restriction enzyme site followed by the first 24 nucleotides of TNF-gamma-β coding sequence starting from the initiating methionine codon. The 3′ sequence 5′-CGC GTC TAG ACT ATA GTA AGA AGG CTC CAA AGA AGG-3′ (SEQ ID NO:48) contains sequences complementary to an Xba I site and 22 nucleotides of TNF-gamma-β. The restriction enzyme sites correspond to the restriction enzyme sites in the bacterial expression vector pQE-9 (Qiagen). pQE-9 was then digested with Bam HI and Xba I. The amplified sequences were ligated into pQE-9 and were inserted in frame with the sequence encoding for the histidine tag and the RBS. The ligation mixture was then used to transform an E. coli strain available from Qiagen under the trademark M15/rep 4 by the procedure described in Sambrook, J. et al., Molecular Cloning: A Laboratory Manual, Cold Spring Laboratory Press, (1989). M15/rep4 contains multiple copies of the plasmid pREP4, which expresses the lacI repressor and also confers kanamycin resistance (Kan^{r}). Transformants were identified by their ability to grow on LB plates and ampicillin/kanamycin resistant colonies were selected. Plasmid DNA was isolated and confirmed by restriction analysis. Clones containing the desired constructs were grown overnight (O/N) in liquid culture in LB media supplemented with both Amp (100 ug/ml) and Kan (25 ug/ml). The O/N culture was used to inoculate a large culture at a ratio of 1:100 to 1:250. The cells were grown to an optical density 600 (O.D.₆₀₀) of between 0.4 and 0.6. IPTG (“Isopropyl-B-D-thiogalactopyranoside”) was then added to a final concentration of 1 mM. IPTG induces by inactivating the lacI repressor, clearing the P/O leading to increased gene expression. Cells were grown an extra 3 to 4 hours. Cells were then harvested by centrifugation. The cell pellet was solubilized in the chaotropic agent 6 M Guanidine HCl (Guanidine HCl concentrations of greater than or equal to 2.5 M were empirically found to result in a higher level of purity of recovered recombinant protein). After clarification, solubilized TNF-gamma-β was purified from this solution by chromatography on a Nickel-Chelate column under conditions that allow for tight binding by proteins containing the 6-His tag (Hochuli, E. et al., J. Chromatography 411:177-184 (1984)). TNF-gamma-β was further purified by a second run on the Nickel-chelate column. TNF-gamma-β (90% pure) was eluted from the column in 6 M guanidine HCl pH 5.0 and for the purpose of renaturation was dialyzed in PBS buffer. The expression product was electrophoresed by SDS-PAGE, and the results may be seen in FIG. 5 where lanes labeled “M” contain molecular weight markers; lane 1 is induced cell lysate; lane 2 is uninduced call lysate; lane 3 is the TNF-gamma protein after two Nickel-chelate column purifications; lane 4 is the TNF-gamma protein after 1 column purification.

One of ordinary skill in the art will recognize that bacterial expression vectors other than pQE-9 may also be used to express TNF-gamma. One such preferred bacterial expression vector is pHE4-5. pHE4-5 may be obtained as pHE4-5/MPIFD23 plasmid DNA (this construct contains an unrelated insert which encodes an unrelated ORF). The pHE4-5/MPIFΔ23 plasmid was deposited with the American Type Culture Collection on Sep. 30, 1997 (Accession No. 209311). The ATCC is located at 10801 University Boulevard, Manassas, Va. 20110-2209, USA. Using the Nde I and Asp 718 restriction sites flanking the unrelated MPIF ORF insert, one of ordinary skill in the art could easily use current molecular biological techniques to replace the unrelated ORF in the pHE4-5/MPIFD23 plasmid with the TNF-gamma-β ORF, or variations thereof, of the present invention.

In a specific embodiment, a bacterial expression construct was generated using the pHE-4 vector to express amino acid residues L-72 through L-172 of SEQ ID NO:2.

In a specific embodiment, a bacterial expression construct was generated using the pHE-4 vector to express amino acid residues L-72 through L-251 of SEQ ID NO:2 fused to a 5′ histidine tag.

In a specific embodiment, a bacterial expression construct was generated using the pHE-4 vector to express amino acid residues L-72 through L-251 of SEQ ID NO:2 fused to a 3′ histidine tag.

In a specific embodiment, a bacterial expression construct was generated using the pHE-4 vector to express amino acid residues L-172 through L-251 of SEQ ID NO:2 fused to a 5′ lacZ tag.

In a preferred embodiment, a polynucleotide encoding amino acid residues Leu-72 through Leu-251 of a TNF-gamma-β polypeptide (e.g., as shown in SEQ ID NO:2) is cloned into a bacterial expression vector (e.g., pHE-4, pHE4-0 or pHE4b-0) and expressed in SG13009, W3110 (ton A-) or M15/REP4 E. coli cells.

Also in a preferred embodiment, TNF-gamma-β of the invention is produced and isolated from SG13009, W3110 or M15/REP4 E. coli cultures using the following protocol.

Stage I: (SI)−Shake Flasks

Media contains Phytone, Yeast Extract, L-Methionine, and NaCl is prepared in shake flasks. The gene for aminoglycoside 3′ phosphotransferase (kanR) is encoded on the expression plasmid so kanamycin is typically added to the seed medium to provide selective pressure for cells maintaining the plasmid. MCB or WCB vials are thawed and used to inoculate shake flasks. The shake flasks are bottom-baffled and covered with a permeable top to maximize the transfer of gases (oxygen, carbon dioxide, etc.). The shake flasks are incubated in a temperature-controlled shaker/incubator. Growth in the flasks is monitored using a spectrophotometer set in the visible wave-length. One or more 100, 150, 350, and/or 650 liter fermenters may be used for the production of TNF-gamma-P. All product contact parts are constructed of Type 1 Borosilicate glass, 316 L stainless steel, medical grade Silicone, Teflon or other FDA approved materials. When a sufficient optical density (e.g., A₆₀₀=1-4) is attained in the seed vessel, the culture is used to inoculate either a production fermenter or a seed fermenter (SII). Typically, shake-flasks are used to inoculate small production fermenters (<100L). A seed fermenter (SII) is used to prepare the larger volume of inoculum required by larger production fermenters.

Stage II (S2)—Seed Fermenter

Fermenters are engineered to provide a controlled environment for the growth of bacteria. Many of the fermenter's functions are preprogrammed and automated. They have agitators for mixing and have the capability of controlling many conditions including temperature, pH and dissolved oxygen. All gasses enter and exit through a hydrophobic 0.2 um filter to maintain sterility. Typically, the SII fermentation uses the same medium as SI including kanamycin. Dissolved oxygen is controlled using aeration, agitation, oxygen supplementation and back-pressure. pH is typically controlled using acid (e.g., phosphoric acid) and base (e.g., ammonium hydroxide) addition. Antifoam (e.g. Sigma Antifoam A) is used to neutralize foam. After inoculation with shake flasks, the SII fermenter is grown until the desired optical density is reached (e.g., A₆₀₀=1-4). The S2 fermenter is used to inoculate the production fermenter.

Stage III (S3):—Production Fermenter.

The production fermenter is batched with production medium (see table 3 below) and heat sterilized. A defined, high cell density fermentation medium is under development. After the fermenter has equilibrated to process temperature, batch nutrients (see table 3 below) are added. Dissolved oxygen is controlled using aeration, agitation, oxygen supplementation and back-pressure. pH is typically controlled using acid (e.g., phosphoric acid) and base (e.g., ammonium hydroxide) addition. S3 is inoculated by the culture from either a shake flasks or a seed fermenter. The cells are grown to a predetermined induction optical density (e.g., A₆₀₀=1-4). pHE4 plasmid is designed to suppress the transcription of recombinant TNF-gamma-β until desired. IPTG is added to the fermentation to stop the suppression (induce) of transcription of TNF-gamma-β. At a specified time after induction, the fermentation is concluded. Time limits for S3 are under development. All operations involving open handling of cultures, medium, or product are conducted using aseptic techniques in laminar flow hoods. Liquids are transferred in closed systems by overpressure using compressed air or a peristaltic pump to minimize the risk of introducing contaminants.

TABLE 3
Fermentation Media and Supplements.

| Batch Medium | Batch Supplements |
|---|---|
| currently contains: | currently contains: |
| KH₂PO₄ | Glucose |
| Na₂HPO₄ | Zinc Sulfate 7-hydrate |
| NaCl | Ferric Chloride 6-hydrate |
| NH₄Cl | Manganese Chloride 4-hydrate |
| Casamino Acids | Cupric Sulfate 5-hydrate |
| Tryptone | Cobalt Chloride 6-hydrate |
| Yeast Extract | Boric Acid |
| L-Cysteine | Hydrochloric Acid |
| Tryptophan | Magnesium Sulfate 7-hydrate |
| L-Histidine | Molybdic Acid Sodium Salt Dihydrate |
| Uridine-HCl Monohydrate | CaCl₂ |
| | Thiamine-HCL |

In specific embodiments, the concentrations of Batch Supplements are varied. In one embodiment, the concentration of zinc sulfate 7-hydrate is varied. In a specific embodiment, the concentration of zinc sulfate is increased by 0.25-fold, 0.75-fold, 1-fold, 1.25-fold, 1.5-fold, 1.75-fold, 2-fold, 2.5-fold, 3-fold, 4-fold, 5-fold, 7.5-fold, 10-fold, 15-fold, 20-fold, 50-fold, 100-fold, 250-fold, 500-fold, 750-fold, or 1000-fold.

TNF-gamma-β is produced in the cytosol and maintained inside the cell membrane. Cells are typically collected using centrifugation or filtration. Cell paste is either processed immediately or is stored at or below −20 C. Stability studies of cell paste will be conducted to establish expiration dating.

Recovery of TNF-gamma-β protein

Step I Cell Harvest

The induced cell suspension is harvested between 4 and 8 hours post IPTG induction. The TNF-gamma-β containing cell paste is obtained with continuous flow centrifugation. Following centrifugation, the cell paste is used immediately or stored at −80 C.

Step 2 Cell Supernatant Production

The cell paste is suspended in 50 mM Tris-HCl buffer pH 8.0 in a 10-fold volume of the cell paste. The cell suspension is homogenized and the supernatant is produced by removal of cell debris with continuous flow centrifugation.

Purification of TNF-gamma-β protein

Unless stated otherwise, the process is conducted at 4-8 C.

Step 1 Chromatography on QAE 550C column

The supernatant is loaded onto a QAE 550C column (weak anion exchanger, TosoHaas) which is equilibrated with 50 mM Tris-HCl, pH 8.0 containing 2 mM CaCl₂. The column is washed with the same buffer and then the TNF-gamma-β is eluted with 125 mM NaCl and 2 mM CaCl₂ in 50 mM Tris-HCl, pH 8.0. The elution is monitored by ultraviolet (UV) absorbance at 280 nm. Fractions are collected across the eluate peak, analyzed by SDS-PAGE, and appropriate fractions are pooled.

Step 2 Chromatography on Q-Sepharose Fast Flow (Q/FF)

The QAE pool is loaded onto a Q/FF (strong anion exchanger, Pharmacia) column equilibrated with 50 mM Tris-HCl containing 125 mM NaCl and 2 mM CaCl₂, pH 8.0. The column then is washed with the same buffer. The TNF-gamma-β is in the fraction of flow through. The loading and wash are monitored by ultraviolet (UV) absorbance at 280 nm.

Step 3 Chromatography on Toyopearl Butyl 650S column

The HQ50 pool is mixed with ammonium sulfate to produce a final concentration of 0.8 M and is loaded onto Toyopearl Butyl 650C (Hydrophobic interaction resin, TosoHaas) column equilibrated in 0.8 M ammonium sulfate in 100 mM Tris-HCl pH 7.3. The column is then washed with a linear gradient elution of TNF-gamma-β with 100 mM Tris-HCl pH 7.3 followed by a 20% ethanol wash. The elution is monitored by ultraviolet (UV) absorbance at 280 nm and conductivity. Fractions are collected across the eluate peak, analyzed by SDS-PAGE. Appropriate fractions are pooled.

Step 4 Concentration on Toyopearl Butyl 650S

The Butyl purified TNF-gamma-β is mixed with ammonium sulfate to produce a final concentration 0.8 M and is loaded onto a smaller Toyopearl Butyl 650C (Hydrophobic interaction resin, TosoHaas) column equilibrated in 0.8 M ammonium sulfate in 100 mM Tris-HCl pH 7.3. TNF-gamma-β is eluted by stepwise with 100 mM Tris-HCl, pH 7.3.

Step 5 Chromatography on Superdex 200 column

The Butyl concentrated TNF-gamma-β is loaded onto a Superdex 200 (Sizing Exclusive Chromatography, Pharmacia) column equilibrated in 10 mM sodium citrate, 150 mM sodium chloride, pH 6.0. Fractions are collected across the eluate peak and are analyzed by SDS-PAGE. Appropriate fractions (>90% purity) are pooled.

Step 6 Ultrafiltration, Filtration and Fill

The purified TNF-gamma-β is placed into a 5 KD MW cutoff membrane device to concentrate a target concentration. Then the protein concentration of purified TNF-gamma-beta is determined by absorbance at 280 nm using TNF-gamma-β extinction coefficient value (1 UV unit for 1 mg/ml). TNF-gamma-β formulation is adjusted to its final protein concentration with the appropriate buffer and filtered via 0.22 micrometer filter under controlled conditions. The filtrate (bulk substance) is stored in suitable sterilized container at 2-8 C (short-term storage) or at or below 20 C (long-term storage).

### Example 2

### Cloning and Expression of TNF-gamma Using the Baculovirus Expression System

The DNA sequence encoding the full length TNF-gamma-β protein, ATCC No. 203055, was amplified using PCR oligonucleotide primers corresponding to the 5′ and 3′ sequences of the gene: The 5′ primer has the sequence 5′-GCG CGG ATC CAC CAT GAG ACG CTT TTT AAG CAA AGT C-3′ (SEQ ID NO:47) and contains a Bam HI restriction enzyme site followed by 24 nucleotides of the TNF-gamma-β coding sequence. The 3′ primer has the sequence 5′-CGC GTC TAG ACT ATA GTA AGA AGG CTC CAA AGA AGG-3′ (SEQ ID NO:48) and contains the cleavage site for the restriction endonuclease Xba 1 and 22 nucleotides complementary to the 3′ non-translated sequence of the TNF-gamma gene. The amplified sequences were isolated from a 1% agarose gel using a commercially available kit (“Geneclean,” BIO 101 Inc., La Jolla, Calif.). The fragment was then digested with the endonucleases Bam HI and Xba I and then purified again on a 1% agarose gel. This fragment was designated F2.

The vector pA2 (modification of pVL941 vector, discussed below) was used for the expression of the TNF-gamma protein using the baculovirus expression system (for review see: Summers, M. D. and Smith, G. E. 1987, A manual of methods for baculovirus vectors and insect cell culture procedures, Texas Agricultural Experimental Station Bulletin No. 1555). This expression vector contains the strong polyhedrin promoter of the Autographa californica nuclear polyhedrosis virus (AcMNPV) followed by the recognition sites for the restriction endonucleases Bam HI and Xba I. The polyadenylation site of the simian virus SV40 is used for efficient polyadenylation. For an easy selection of recombinant virus the beta-galactosidase gene from E. coli was inserted in the same orientation as the polyhedrin promoter followed by the polyadenylation signal of the polyhedrin gene. The polyhedrin sequences were flanked at both sides by viral sequences for the cell-mediated homologous recombination of cotransfected wild-type viral DNA. Many other baculovirus vectors could have been used in place of pA2, such as pRG1, pAc373, pVL941 and pAcIM1 (Luckow, V. A. and Summers, M. D., Virology, 170:31-39).

The plasmid was digested with the restriction enzymes Bam HI and Xba I and then dephosphorylated using calf intestinal phosphatase by procedures known in the art. The DNA was then isolated from a 1% agarose gel using the commercially available kit (“Geneclean” BIO 101 Inc., La Jolla, Calif.). This vector DNA was designated V2.

Fragment F2 and the dephosphorylated plasmid V2 were ligated with T4 DNA ligase. E. coli XL1 blue cells were then transformed. The sequence of the cloned fragment was confirmed by DNA sequencing.

5 μg of the plasmid pBac TNF-gamma was cotransfected with 1.0 μg of a commercially available linearized baculovirus (“BaculoGold baculovirus DNA”, Pharmingen, San Diego, Calif.) using the lipofection method (Felgner et al. Proc. Natl. Acad. Sci. USA, 84:7413-7417 (1987)).

1 μg of BaculoGold virus DNA and 5 μg of the plasmid pBac TNF-gamma were mixed in a sterile well of a microtiter plate containing 50 μl of serum free Grace's medium (Life Technologies Inc., Gaithersburg, Md.). Afterwards, 10 μl Lipofectin plus 90 μl Grace's medium were added, mixed and incubated for 15 minutes at room temperature. Then the transfection mixture was added dropwise to the Sf9 insect cells (ATCC CRL 1711) seeded in a 35 mm tissue culture plate with 1 ml Grace' medium without serum. The plate was rocked back and forth to mix the newly added solution. The plate was then incubated for 5 hours at 27° C. After 5 hours, the transfection solution was removed from the plate and 1 ml of Grace's insect medium supplemented with 10% fetal calf serum was added. The plate was put back into an incubator and cultivation continued at 27° C. for four days.

After four days, the supernatant was collected and a plaque assay performed essentially as described by Summers and Smith (supra). As a modification, an agarose gel with “Blue Gal” (Life Technologies Inc., Gaithersburg) was used which allows an easy isolation of blue stained plaques. (A detailed description of a “plaque assay” can also be found in the user's guide for insect cell culture and baculovirology distributed by Life Technologies Inc., Gaithersburg, page 9-10).

Four days after the serial dilution, the virus was added to the cells, blue stained plaques were picked with the tip of an Eppendorf pipette. The agar containing the recombinant viruses was then resuspended in an Eppendorf tube containing 200 μl of Grace's medium. The agar was removed by a brief centrifugation and the supernatant containing the recombinant baculovirus was used to infect Sf9 cells seeded in 35 mm dishes. Four days later the supernatants of these culture dishes were harvested and then stored at 4° C.

Sf9 cells were grown in Grace's medium supplemented with 10% heat-inactivated FBS. The cells were infected with the recombinant baculovirus V-TNF-gamma at a multiplicity of infection (MOI) of 2. Six hours later the medium was removed and replaced with SF900 II medium minus methionine and cysteine (Life Technologies Inc., Gaithersburg). 42 hours later 5 μCi of [³⁵S]-methionine and 5 μCi [³⁵S]-cysteine (Amersham) were added. The cells were further incubated for 16 hours before they were harvested by centrifugation and the labeled proteins visualized by SDS-PAGE and autoradiography. FIG. 6 illustrates a gel where lanes 1 and 3 are the medium of the TNF-gamma and control cultures and lanes 2 and 4 are the cell lysates of the TNF-gamma and the control cultures.

In a specific embodiment, a baculoviral expression construct was generated using the pA2SPst vector to express amino acid residues A-61 through L-251 of SEQ ID NO:2.

In a specific embodiment, a baculoviral expression construct was generated using the pA2GP vector to express amino acid residues L-71 through L-251 of SEQ ID NO:2.

In a specific embodiment, a baculoviral expression construct was generated using the pA2GP vector to express amino acid residues L-71 through L-251 of SEQ ID NO:2 fused to a 5′ lacZ tag.

In a specific embodiment, a baculoviral expression construct was generated using the pA2 vector to express amino acid residues M-1 through L-251 of SEQ ID NO:2.

### Example 3

### Expression of Recombinant TNF-Gamma in COS Cells

The expression of plasmid, TNF-gamma-β-HA is derived from a vector pcDNAI/Amp (Invitrogen) containing: 1) SV40 origin of replication, 2) ampicillin resistance gene, 3) E. coli replication origin, 4) CMV promoter followed by a polylinker region, an SV40 intron, and a polyadenylation site. A DNA fragment encoding the entire TNF-gamma-β precursor and a hemagglutinin antigen (HA) tag fused in frame to its 3′ end was cloned into the polylinker region of the vector. Therefore, the recombinant protein expression is under the direction of the CMV promoter. The HA tag corresponds to an epitope derived from the influenza hemagglutinin protein as previously described (I. Wilson, H. Niman, R. Heighten, A Cherenson, M. Connolly, and R. Lerner, 1984, Cell 37, 767). The fusion of HA tag to our target protein allows easy detection of the recombinant protein with an antibody that recognizes the HA epitope.

The plasmid construction strategy is described as follows: The DNA sequence encoding TNF-gamma-β, ATCC # 203055 constructed by PCR on the original EST cloned using two primers: the 5′ primer (SEQ ID NO:47) contains a Bam HI site followed by 24 nucleotides of TNF-gamma-β coding sequence starting from the initiation codon; the 3′ sequence 5′-CGC TCT AGA TCA AGC GTA GTC TGG GAC GTC GTA TGG ATA GTA AGA AGG CTC CAA AG-3′ (SEQ ID NO:49) contains complementary sequences to Xba I site, translation stop codon, HA tag and the last 18 nucleotides of the TNF-gamma coding sequence (not including the stop codon). Therefore, the PCR product contained a Bam HI site, TNF-gamma-β coding sequence followed by HA tag fused in frame, a translation termination stop codon next to the HA tag, and an Xba I site. The PCR amplified DNA fragment and the vector, pcDNAI/Amp, were digested with Bam HI and Xba I restriction enzymes and ligated together. The ligation mixture was transformed into E. coli strain SURE (available from Stratagene Cloning Systems, 11099 North Torrey Pines Road, La Jolla, Calif. 92037) the transformed culture was plated on ampicillin media plates and resistant colonies were selected. Plasmid DNA was isolated from transformants and examined by restriction analysis for the presence of the correct fragment. For expression of the recombinant TNF-gamma-β, COS cells were transfected with the expression vector by DEAE-DEXTRAN method. (J. Sambrook, E. Fritsch, T. Maniatis, Molecular Cloning: A Laboratory Manual, Cold Spring Laboratory Press, (1989)). The expression of the TNF-gamma HA protein was detected by radiolabelling and immunoprecipitation method. (E. Harlow, D. Lane, Antibodies: A Laboratory Manual, Cold Spring Harbor Laboratory Press, (1988)). Cells were labeled for 8 hours with [³⁵S]-S-cysteine two days post transfection. Culture media were then collected and cells were lysed with detergent (RIPA buffer (150 mM NaCl, 1% NP-40, 0.1% SDS, 1% NP-40, 0.5% DOC, 50 mM Tris, pH 7.5; Wilson, I. et al., Id. 37:767 (1984)). Both cell lysate and culture media were precipitated with an HA-specific monoclonal antibody. Precipitated proteins were then analyzed on 15% SDS-PAGE gels.

In a specific embodiment, a mammalian expression construct was generated using the pC4 vector to express amino acid residues M-1 through L-251 of SEQ ID NO:2.

In a specific embodiment, a mammalian expression construct was generated using the pC4SPst vector to express amino acid residues A-61 through L-251 of SEQ ID NO:2.

In a specific embodiment, a mammalian expression construct was generated using the pC4 vector to express amino acid residues L-72 through L-251 of SEQ ID NO:2 fused to the Fc region of human immunoglobulin, as described supra.

In a specific embodiment, a mammalian expression construct was generated using the pC4SP vector to express amino acid residues L-72 through L-251 of SEQ ID NO:2 fused to lacZ at the amino terminus.

### Example 4

### Expression Pattern of TNF-Gamma in Human Tissue

RNA blot analysis was carried out to examine the levels of expression of TNF-gamma in human tissues. Total cellular RNA samples were isolated with RNAzol™ B system (Biotecx Laboratories, Inc. 6023 South Loop East, Houston, Tex. 77033). About 2 μg (for the RNA blot of FIG. 3A) of total RNA isolated from each human tissue specified was separated on 1% agarose-formaldehyde gel and blotted onto a nylon filter (Sambrook, Fritsch, and Maniatis, Molecular Cloning, Cold Spring Harbor Press, (1989)). The labeling reaction was done according to the Stratagene Prime-It kit with 50 ng TNF-gamma cDNA, to produce [³²P]-labeled TNF-gamma cDNA. The labeled DNA was purified with a Select-G-50 column (5 Prime-3 Prime, Inc. 5603 Arapahoe Road, Boulder, Colo. 80303). The filter was then hybridized with radioactive labeled full-length TNF-gamma gene at 1,000,000 cpm/ml in 0.5 M NaPO4, pH 7.4 and 7% SDS overnight at 65 C. After being washed twice at room temperature and twice at 60 C with 0.5×SSC, 0.1% SDS, the X-ray film was then exposed to the blot at −70 C overnight with an intensifying screen. The message RNA for TNF-gamma is abundant in kidney.

The same reaction was done, with the exception that 10 μg poly-A RNA isolated from the indicated tissues was used. In this experiment, the messenger RNA encoding TNF-gamma is expressed predominantly in HUVEC cells, but not in other cell lines examined; for example; CAMA1 (breast cancer); AN3CA (uterine cancer); SK.UT.1 (uterine cancer); MG63 (osteoblastoma); HOS (osteoblastoma); MCF7 (breast cancer); OVCAR-3 (ovarian cancer); CAOV-3 (ovarian cancer); AOSMIC (smooth muscle); and foreskin fibroblast.

Northern blot analyses were also performed to determine the relative expression level of the TNF-gamma RNA with respect to the proliferation state of HUVEC cell cultures. In these experiments, identical amounts of total RNA isolated from HUVEC cells (15 μg) were electrophoretically separated and blotted as described above. RNA was isolated from cultures 1, 2, 3, 4, 6, and 7 days post-seeding. As illustrated in FIG. 4, TNF-gamma RNA (labeled “VEGI”) was only seen at low levels in newly seeded cultures (days 1, 2, and 3). However, expression of TNF-gamma RNA was apparent as the HUVEC cells in the cultures began to reach confluence (days 4, 6, and 7). These experiments indicate that TNF-gamma expression increases as cells in a culture or tissue begin to reach the quiescent state of non- or reduced-proliferation.

In other experiments performed essentially as described above, the TNF-gamma-alpha transcript has been detected in many different human tissues, e.g., placenta, lung, kidney, skeletal muscle, pancreas, spleen, prostate, small intestine, and colon. Further experiments have shown that expression of the TNF-gamma-alpha molecule was greatest in a subset of endothelial cells, such as human umbilical vein endothelial cells (HUVECs) and human uterine myometrial microvascular endothelial cells (HMMVECs), but not in human pulmonary artery endothelial cells (HPAEC), human iliac artery endothelial cells (HIAEC), or human coronary artery endothelial cells (HCAEC). The transcript for TNF-gamma-beta has also been detected in placenta, lung, kidney, prostate, small intestine, stomach, liver, kidney, and pancreas, HUVECs, HMMVECs, human aortic endothelial cells (HAECs), and human microvascular endothelial cells (HUMECs).

### Example 5

### Expression via Gene Therapy

Fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask. At this time, fresh media is added (e.g., Ham's F12 media, supplemented with 10% FBS, penicillin, and streptomycin). The culture is then incubated at 37° C. for approximately one week. At this time, fresh media is added and subsequently changed every 2-3 days. After an additional two weeks in culture, a monolayer of fibroblasts will have emerged. The monolayer is trypsinized and scaled into larger flasks.

pMV-7 (Kirschmeier, P. T. et al, DNA, 7:219-25 (1988)), which is flanked by the long terminal repeats of the Moloney murine sarcoma virus, is digested with Eco RI and Hind III, and, subsequently, treated with calf intestinal phosphatase. The linear vector is fractionated on agarose gel and purified using glass beads.

The cDNA encoding a polypeptide of the present invention is amplified using PCR primers which correspond to the 5′ and 3′ end sequences respectively. The 5′ primer containing an Eco RI site and the 3′ primer includes a Hind III site. Equal quantities of the Moloney murine sarcoma virus linear backbone and the amplified Eco RI and Hind III fragment are added together, in the presence of T4 DNA ligase. The resulting mixture is maintained under conditions appropriate for ligation of the two fragments. The ligation mixture is used to transform bacteria HB101, which are then plated onto agar-containing kanamycin for the purpose of confirming that the vector had the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells are transduced with the vector. The packaging cells now produce infectious viral particles containing the gene (the packaging cells are now referred to as producer cells).

Fresh media is added to the transduced producer cells, and, subsequently, the media is harvested from a 10 cm plate of confluent producer cells. The spent media, containing the infectious viral particles, is filtered through a millipore filter to remove detached producer cells. This media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it may be necessary to use a retroviral vector that has a selectable marker, such as neo or his.

The engineered fibroblasts are then injected into the host, either alone or after having been grown to confluence on cytodex 3 microcarrier beads. The fibroblasts now produce the protein product.

### Example 6

### Protein Fusions of TNF-Gamma-β

TNF-gamma-β polypeptides of the invention are optionally fused to other proteins. These fusion proteins can be used for a variety of applications. For example, fusion of TNF-gamma-β polypeptides to His-tag, HA-tag, protein A, IgG domains, FLAG, and maltose binding protein facilitates purification. (See EP A 394,827; Traunecker, et al., Nature 331:84-86 (1988).) Similarly, fusion to IgG-1, IgG-3, and albumin increases the halflife time in vivo. Nuclear localization signals fused to TNF-gamma-β polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein.

In one embodiment, TNF-gamma-polynucleotides of the invention are fused to a polynucleotide encoding a “FLAG” polypeptide. Thus, a TNF-gamma- -FLAG fusion protein is encompassed by the present invention. The FLAG antigenic polypeptide may be fused to a TNF-gamma- polypeptide of the invention at either or both the amino or the carboxy terminus. In preferred embodiments, a TNF-gamma- -FLAG fusion protein is expressed from a pFLAG-CMV-5a or a pFLAG-CMV-1 expression vector (available from Sigma, St. Louis, Mo., USA). See, Andersson, S., et al., J. Biol. Chem. 264:8222-29 (1989); Thomsen, D. R., et al., Proc. Natl. Acad. Sci. USA, 81:659-63 (1984); and Kozak, M., Nature 308:241 (1984) (each of which is hereby incorporated by reference). In further preferred embodiments, a TNF-gamma- -FLAG fusion protein is detectable by anti-FLAG monoclonal antibodies (also available from Sigma).

In a specific embodiment, a TNF-gamma- -FLAG fusion protein expression construct was generated using the pFLAG-CMV-1 vector to express amino acid residues L-72 through L-251 of SEQ ID NO:2 fused to FLAG at the amino terminus.

In another specific embodiment, a TNF-gamma- -lacZ-FLAG fusion protein expression construct was generated using the pFLAG-CMV-1 vector to express amino acid residues L-72 through L-251 of SEQ ID NO:2 fused to FLAG and lacZ at the amino terminus.

All of the types of fusion proteins described above can be made using techniques known in the art or by using or routinely modifying the following protocol, which outlines the fusion of a polypeptide to an IgG molecule.

Briefly, the human Fc portion of the IgG molecule can be PCR amplified, using primers that span the 5′ and 3′ ends of the sequence described below. These primers also preferably contain convenient restriction enzyme sites that will facilitate cloning into an expression vector, preferably a mammalian expression vector.

For example, if the pC4 (Accession No. 209646) expression vector is used, the human Fc portion can be ligated into the Bam HI cloning site. Note that the 3′ BamHI site should be destroyed. Next, the vector containing the human Fe portion is re-restricted with BamHI, linearizing the vector, and TNF-gamma-β polynucleotide, isolated by the PCR protocol described in Example 1, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

Human IgG Fe region:

| | |
|---|---|
| GGGATCCGGAGCCCAAATCTTCTGACAAAACTCACACATGCCCACCGTGCCCAGCACCTG | (SEQ ID NO:50) |
| AATTCGAGGGTGCACCGTCAGTCTTCCTCTTCCCCCCAAAACCCAAGGACACCCTCATGA | |
| TCTCCCGGACTCCTGAGGTCACATGCGTGGTGGTGGACGTAAGCCACGAAGACCCTGAGG | |
| TCAAGTTCAACTGGTACGTGGACGGCGTGGAGGTGCATAATGCCAAGACAAAGCCGCGGG | |
| AGGAGCAGTACAACAGCACGTACCGTGTGGTCAGCGTCCTCACCGTCCTGCACCAGGACT | |
| GGCTGAATGGCAAGGAGTACAAGTGCAAGGTCTCCAACAAAGCCCTCCCAACCCCCATCG | |
| AGAAAACCATCTCCAAAGCCAAAGGGCAGCCCCGAGAACCACAGGTGTACACCCTGCCCC | |
| CATCCCGGGATGAGCTGACCAAGAACCAGGTCAGCCTGACCTGCCTGGTCAAAGGCTTCT | |
| ATCCAAGCGACATCGCCGTGGAGTGGGAGAGCAATGGGCAGCCGGAGAACAACTACAAGA | |
| CCACGCCTCCCGTGCTGGACTCCGACGGCTCCTTCTTCCTCTACAGCAAGCTCACCGTGG | |
| ACAAGAGCAGGTGGCAGCAGGGGAACGTCTTCTCATGCTCCGTGATGCATGAGGCTCTGC | |
| ACAACCACTACACGCAGAAGAGCCTCTCCCTGTCTCCGGGTAAATGAGTGCGACGGCCGC | |
| GACTCTAGAGGAT | |

### Example 7

### T Cell Proliferation Assay

A CD3-induced proliferation assay is performed on PBMCs and is measured by the uptake of ³H-thymidine. The assay is performed as follows. Ninety-six well plates are coated with 100 microliters/well of mAb to CD3 (HIT3a, Pharmingen) or isotype-matched control mAb (B33.1) overnight at 4 C (1 micrograms/ml in 0.05M bicarbonate buffer, pH 9.5), then washed three times with PBS. PBMC are isolated by F/H gradient centrifugation from human peripheral blood and added to quadruplicate wells (5×10⁴/well) of mAb coated plates in RPMI containing 10% FCS and P/S in the presence of varying concentrations of TNF-gamma-β protein (total volume 200 microliters). Relevant protein buffer and medium alone are controls. After 48 hours at 37 C, plates are spun for 2 min. at 1000 rpm and 100 microliters of supernatant is removed and stored at −20 C for measurement of IL-2 (or other cytokines) if an effect on proliferation is observed. Wells are supplemented with 100 microliters of medium containing 0.5 microcuries of 3H-thymidine and cultured at 37 C for 18-24 hr. Wells are harvested and incorporation of ³H-thymidine used as a measure of proliferation. Anti-CD3 alone is the positive control for proliferation. IL-2 (100 U/ml) is also used as a control which enhances proliferation. Control antibody which does not induce proliferation of T cells is used as the negative controls for the effects of TNF-gamma-β proteins.

The studies described in this example tested activity in TNF-gamma-β protein. However, one skilled in the art could easily modify the exemplified studies to test the activity of TNF-gamma-β polynucleotides (e.g., gene therapy), agonists, and/or antagonists of TNF-gamma-β.

### Example 8

### Effect of TNF-Gamma-β on the Expression of MHC Class II, Costimulatory and Adhesion Molecules and Cell Differentiation of Monocytes and Monocyte-Derived Human Dendritic Cells

Dendritic cells are generated by the expansion of proliferating precursors found in the peripheral blood: adherent PBMC or elutriated monocytic fractions are cultured for 7-10 days with GM-CSF (50 ng/ml) and IL-4 (20 ng/ml). These dendritic cells have the characteristic phenotype of immature cells (expression of CD1, CD8O, CD86, CD40 and MHC class II antigens). Treatment with activating factors, such as TNF-alpha, causes a rapid change in surface phenotype (increased expression of MHC class I and II, costimulatory and adhesion molecules, downregulation of FCgammaRII, upregulation of CD83). These changes correlate with increased antigen-presenting capacity and with functional maturation of the dendritic cells.

FACS analysis of surface antigens is performed as follows. Cells are treated 1-3 days with increasing concentrations of TNF-gamma-β or LPS (positive control), washed with PBS containing 1% BSA and 0.02 mM sodium azide, and then incubated with 1:20 dilution of appropriate FITC- or PE-labeled monoclonal antibodies for 30 minutes at 4 C. After an additional wash, the labeled cells are analyzed by flow cytometry on a FACScan (Becton Dickinson).

Effect on the Production of Cytokines.

Cytokines generated by dendritic cells, in particular IL-12, are important in the initiation of T-cell dependent immune responses. IL-12 strongly influences the development of Th1 helper T-cell immune response, and induces cytotoxic T and NK cell function. An ELISA is used to measure the IL-12 release as follows. Dendritic cells (10⁶/ml) are treated with increasing concentrations of TNF-gamma-β for 24 hours. LPS (100 ng/ml) is added to the cell culture as positive control. Supernatants from the cell cultures are then collected and analyzed for IL-12 content using commercial ELISA kit (e.g., R & D Systems (Minneapolis, Minn.)). The standard protocols provided with the kits are used.

Effect on the Expression of MHC Class II, Costimulatory and Adhesion Molecules.

Three major families of cell surface antigens can be identified on monocytes: adhesion molecules, molecules involved in antigen presentation, and Fc receptor. Modulation of the expression of MHC class II antigens and other costimulatory molecules, such as B7 and ICAM-1, may result in changes in the antigen presenting capacity of monocytes and ability to induce T cell activation. Increase expression of Fc receptors may correlate with improved monocyte cytotoxic activity, cytokine release and phagocytosis.

FACS analysis is used to examine the surface antigens as follows. Monocytes are treated 1-5 days with increasing concentrations of TNF-gamma alpha or TNF-gamma-b or LPS (positive control), washed with PBS containing 1% BSA and 0.02 mM sodium azide, and then incubated with 1:20 dilution of appropriate FITC- or PE-labeled monoclonal antibodies for 30 minutes at 4 C. After an additional wash, the labeled cells are analyzed by flow cytometry on a FACScan (Becton Dickinson).

Monocyte Activation and/or Increased Survival.

Assays for molecules that activate (or alternatively, inactivate) monocytes and/or increase monocyte survival (or alternatively, decrease, monocyte survival) are known in the art and may routinely be applied to determine whether a molecule of the invention functions as an inhibitor or activator of monocytes. TNF-gamma-β, agonists, or antagonists of TNF-gamma-β can be screened using the three assays described below. For each of these assays, Peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

1. Monocyte Survival Assay. Human peripheral blood monocytes progressively lose viability when cultured in absence of serum or other stimuli. Their death results from internally regulated process (apoptosis). Addition to the culture of activating factors, such as TNF-alpha dramatically improves cell survival and prevents DNA fragmentation. Propidium iodide (PI) staining is used to measure apoptosis as follows. Monocytes are cultured for 48 hours in polypropylene tubes in serum-free medium (positive control), in the presence of 100 ng/ml TNF-alpha (negative control), and in the presence of varying concentrations of the compound to be tested. Cells are suspended at a concentration of 2×10⁶/ml in PBS containing PI at a final concentration of 5 micrograms/ml, and then incubated at room temperature for 5 minutes before FACScan analysis. PI uptake has been demonstrated to correlate with DNA fragmentation in this experimental paradigm.

2. Effect on cytokine release. An important function of monocytes/macrophages is their regulatory activity on other cellular populations of the immune system through the release of cytokines after stimulation. An ELISA to measure cytokine release is performed as follows. Human monocytes are incubated at a density of 5×10⁵ cells/ml with increasing concentrations of TNF-gamma-β and under the same conditions, but in the absence of TNF-gamma-P. For IL-12 production, the cells are primed overnight with IFN-gamma (100 U/ml) in presence of TNF-gamma-P. LPS (10 ng/ml) is then added. Conditioned media are collected after 24 h and kept frozen until use. Measurement of TNF-alpha, IL-10, MCP-1 and IL-8 is then performed using a commercially available ELISA kit (e.g., R & D Systems (Minneapolis, Minn.)) and applying the standard protocols provided with the kit.

3. Oxidative burst. Purified monocytes are plated in 96-well plates at 2-1×10⁵ cell/well. Increasing concentrations of TNF-gamma-β are added to the wells in a total volume of 0.2 ml culture medium (RPMI 1640+10% FCS, glutamine and antibiotics). After 3 days incubation, the plates are centrifuged and the medium is removed from the wells. To the macrophage monolayers, 0.2 ml per well of phenol red solution (140 mM NaCl, 10 mM potassium phosphate buffer pH 7.0, 5.5 mM dextrose, 0.56 mM phenol red and 19 U/ml of HRPO) is added, together with the stimulant (200 mM PMA). The plates are incubated at 37 C for 2 hours and the reaction is stopped by adding 20 μl 1N NaOH per well. The absorbance is read at 610 nm. To calculate the amount of H₂O₂ produced by the macrophages, a standard curve of a H₂O₂ solution of known molarity is performed for each experiment.

The studies described in this example tested activity in TNF-gamma-β protein. However, one skilled in the art could easily modify the exemplified studies to test the activity of TNF-gamma-β polynucleotides (e.g., gene therapy), agonists, and/or antagonists of TNF-gamma-β.

### Example 9

### Method of Determining Alterations in the TNF-gamma-β Gene

RNA is isolated from entire families or individual patients presenting with a phenotype of interest (such as a disease). cDNA is then generated from these RNA samples using protocols known in the art. (See, Sambrook.) The cDNA is then used as a template for PCR, employing primers surrounding regions of interest in SEQ ID NO:1. Suggested PCR conditions consist of 35 cycles at 95° C. for 30 seconds; 60-120 seconds at 52-58° C.; and 60-120 seconds at 70° C., using buffer solutions described in Sidransky, D., et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons of TNF-gamma alpha or TNF-gamma-b are also determined and genomic PCR products analyzed to confirm the results. PCR products harboring suspected mutations in TNF-gamma-β are then cloned and sequenced to validate the results of the direct sequencing.

PCR products of TNF-gamma-β are cloned into T-tailed vectors as described in Holton, T. A. and Graham, M. W., Nucleic Acids Research, 19:1156 (1991) and sequenced with T7 polymerase (United States Biochemical). Affected individuals are identified by mutations in TNF-gamma-β not present in unaffected individuals.

Genomic rearrangements are also observed as a method of determining alterations in the TNF-gamma-β gene. Genomic clones isolated using techniques known in the art are nick-translated with digoxigenindeoxy-uridine 5′-triphosphate (Boehringer Manheim), and FISH performed as described in Johnson, Cg. et al., Methods Cell Biol. 35:73-99 (1991). Hybridization with the labeled probe is carried out using a vast excess of human cot-1 DNA for specific hybridization to the TNF-gamma-β genomic locus.

Chromosomes are counterstained with 4,6-diamino-2-phenylidole and propidium iodide, producing a combination of C— and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region of TNF-gamma-β (hybridized by the probe) are identified as insertions, deletions, and translocations. These TNF-gamma-β alterations are used as a diagnostic marker for an associated disease.

### Example 10

### Method of Detecting Abnormal Levels of TNF-gamma-β in a Biological Sample

TNF-gamma-β polypeptides can be detected in a biological sample, and if an increased or decreased level of TNF-gamma-β is detected, this polypeptide is a marker for a particular phenotype. Methods of detection are numerous, and thus, it is understood that one skilled in the art can modify the following assay to fit their particular needs.

For example, antibody-sandwich ELISAs are used to detect TNF-gamma-β in a sample, preferably a biological sample. Wells of a microtiter plate are coated with specific antibodies to TNF-gamma-β, at a final concentration of 0.2 to 10 ug/ml. The antibodies are either monoclonal or polyclonal and are produced using technique known in the art. The wells are blocked so that non-specific binding of TNF-gamma-β to the well is reduced.

The coated wells are then incubated for >2 hours at RT with a sample containing TNF-gamma-β. Preferably, serial dilutions of the sample should be used to validate results. The plates are then washed three times with deionized or distilled water to remove unbounded TNF-gamma-β.

Next, 50 microliters of specific antibody-alkaline phosphatase conjugate, at a concentration of 25-400 ng, is added and incubated for 2 hours at room temperature. The plates are again washed three times with deionized or distilled water to remove unbounded conjugate.

75 ul of 4-methylumbelliferyl phosphate (MUP) or p-nitrophenyl phosphate (NPP) substrate solution is then added to each well and incubated 1 hour at room temperature to allow cleavage of the substrate and flourescence. The flourescence is measured by a microtiter plate reader. A standard curve is prepared using the experimental results from serial dilutions of a control sample with the sample concentration plotted on the X-axis (log scale) and fluorescence or absorbance on the Y-axis (linear scale). The TNF-gamma-β polypeptide concentration in a sample is then interpolated using the standard curve based on the measured flourescence of that sample.

### Example 11

### TNF-Gamma-β Binding Assays

Preparation of Radiolabeled TNF-Gamma-β

Radio-iodination of TNF-gamma-β was performed using the Iodobead method. Briefly, one Iodobead (Pierce) per reaction was pre-washed with PBS and added to 1 mCi of NaI¹²⁵ in 80 microliters of PBS pH 6.5. The reaction was allowed to proceed for 5 minutes and then 10 micrograms of TNF-gamma-β was added and incubated for 5 minutes at room temperature. Iodinated protein was separated from unbound radioactivity using a G-25 Sephadex quick spin column previously equilibrated with PBS containing 0.1% BSA. Protein concentration and specific radioactivity of I¹²⁵-Vasolysin were determined by TCA precipitation of pre-column and post-column samples. The specific activity of I¹²⁵-Vasolysin used in the experiment was 15.2 microcuries per microgram.

Competitive Binding Assay to Determine Specific Binding

BAEC, HAEC and NHDF cells were plated (2×10⁵ cells/well) in 24 well plate overnight. The binding assay was performed in 500 microliters of binding buffer (Ham's F containing 0.5% BSA and 0.1% sodium azide) containing 0.3 nM 11²⁵-TNF-gamma-β in the absence or presence of 100-fold excess of unlabeled TNF-gamma-β. Binding to cells was performed in triplicates in a 96 well plate using 1×10⁶ cells in 100 microliters of binding buffer under similar conditions used for other cell types with 0.3 nM ¹²⁵I— TNF-gamma-β. The binding reaction was carried out at room temperature for 2 hr. Cell bound I¹²⁵-TNF-gamma-β was separated from unbound material by centrifugation through 200 microliters of 1.5 dibutylphthlate/1.0 bis (2-ethyl-hexyl) phthalate oil mixture in a polyethylene microfuge tubes (Bio-Rad) for 20 sec at 12,000 RPM. The microfuge tubes were then frozen quickly in liquid nitrogen and the bottom tip of the tubes was cut off using a tube cutter. Radioactivity in the bottom containing the cell pellet (bound fraction) and the top (unbound fraction) of the tubes were counted by using a gamma counter.

The cells were then washed three times with PBS containing 0.1% BSA and lysed with 1% NP40 solution and counted using a gamma counter.

To determine affinity (Kd) of TNF-gamma-β binding to cells, binding assay was performed with 0.3 nM I¹²⁵-TNF-gamma-β in presence of increasing concentrations of unlabeled TNF-gamma-β (0.01 to 639 nM). The data was analyzed by Prizm software (GraphPad Software, San Diego, Calif.) to determine dissociation constant (Kd) and number of binding sites.

### Example 12

### Generation and Characterization of Anti-TNF-Gamma-β Antibodies

Balb/C mice were immunized with TNF-gamma-β polypeptide (amino acid residues 72-251 of SEQ ID NO:2 according to the following schedule:

| Day | Dose/mouse | Route | Vehicle |
|---|---|---|---|
| 1 | 50 micrograms | Sub-cutaneous | Complete Freund's Adjuvant |
| 13 | 50 micrograms | Sub-cutaneous | Incomplete Freund's Adjuvant |
| 27 | 50 micrograms | Sub-cutaneous | Incomplete Freund's Adjuvant |
| 38 | 10 micrograms | Intra-peritoneal | PBS |
| 59 | 10 micrograms | Intra-peritoneal | PBS |
| 97 | 10 micrograms | Intra-peritoneal | PBS |

After the final immunization, hybridomas were generated according to standard protocols. Hybridomas were initially screened by ELISA for their ability to bind TNF-gamma-β (amino acid residues 72-251 of SEQ ID NO:2) by ELISA which identified eighteen positive hybridomas: 03CO₆, 04H08, 06CO₃, 06D09, 06F03, 08D06, 12D08, 12F11, 14A03, 15B03, 15E09, 16B05, 16H02, 17A03, 17D07, 18G08,20B01 and 20CO_{5.}

Characterization of Murine Monoclonal Anti-TNF-Gamma-β Antibodies:

TNF-gamma-b treatment induces production of secreted alkaline phosphatase in TF-1/SRE reporter cells. Additionally TNF-gamma-β treatment results in caspase activation in TF-1 cells. The ability of the murine monoclonal antibodies to neutralize the TNF-gamma-β mediated activities were investigated.

SEAP Assay

The ability of TNF-gamma-β to generate a signal that activates genes under the regulation of Signal Response Elements (SREs) was examined using TF-1 cell line transfected with an SRE/Secreted Alkaline Phosphatase (SEAP) reporter plasmid. Briefly, a poly-D-lysine coated 96-well plate is seeded with TF-1/SRE-SEAP cells (in RPMI+0.2% Fetal bovine serum) at 75,000 cells per well. Cells were incubated overnight and the media was aspirated the next morning and replaced with media (RPMI+0.2% fetal bovine serum) containing TNF-gamma-β. Again cells were incubated overnight. After overnight incubation, conditioned media were collected and SEAP activity was determined using the SEAP Reporter Gene Assay available from Roche Molecular Biochemicals (Indianapolis, Ind.) according to the manufacturer's directions. Briefly, Conditioned media were diluted 1:4 into dilution buffer. Samples were incubated at 65C for 30 minutes to eliminate contaminating AP activity usually present in culture medium. 25 microliters of the heat-inactivated samples were mixed with equal volume of inactivation buffer (containing a mixture of differential alkaline phosphatase inhibitors). Following a 5-minute incubation at room temperature, 50 uL of alkaline phosphatase substrate (CSPD) was added to each well. Chemiluminescence signal was read 10-15 minutes later using a luminometer. TNF-gamma-β induces SEAP production in a dose dependent fashion.

Antibodies generated against TNF-gamma-β were tested for the ability to inhibit the TNF-gamma-β induced SEAP production in TF-1/SRE SEAP reporter cells. Briefly, 24 micrograms/mL of each antibody (50×molar excess) was mixed with either 200 ng/mL of TNF-gamma-β in medium (RPMI+0.2% FBS) or in medium alone (RPMI+0.2% FBS) in a total volume of 150 microliters. These solutions were then incubated for 1 hour at room temperature. Fifty microliters of the media containing TNF-gamma-P+antibody or antibody alone solution was added to the TF-1 cells which were then incubated overnight. After the overnight incubation, the SEAP assay was performed as described above. Using this assay, monoclonal antibodies 12D08, 14A03, 15E09, and 16H02 were identified as potent TNF-gamma-β neutralizing antibodies.

Caspase Assay

The ability of TNF-gamma-β to induce caspase activity in TF-1 cells was analyzed using a Homogeneous Fluorimetric Caspases Assay available from Roche Molecular Biochemicals (Indianapolis, Ind.) according to the manufacturer's directions. Briefly, cells growing in microtiter plates are induced to undergo apoptosis, causing an activation of caspase activities. Equal volume of a caspase substrate (Asp-Glu-Val-Asp-Rhodamine 110, or DEVD-R110) solution is then added and incubated for at least 1 hour. During this incubation, cells are being lysed and free R110 is released from the substrate. The level of free R110 is determined fluorimetrically, using a fluorescence reader with excitation filter 470-500 nm and emission filter 500-560 nm.

A black 96-well plate with a clear bottom is seeded with 75,000 TF-1 cells in RPMI containing 1% fetal bovine serum and micrograms/milliliter cyclohexamide. An equal volume of 2×TNF-gamma-β is then added to the wells and incubated for 5 hours prior to performing the caspase assay. Following the manufacturer's directions, an equal volume of 1×substrate solution containing 50 micromolar DEVD-R110 diluted in incubation buffer is added to each well. The 96-well plates are then incubated for 2 hours after which the plate is read in a fluorescence reader with an excitation filter at 485 nm and an emission filter at 535 nm. TNF-gamma-β induces caspase production in a dose dependent fashion.

Antibodies generated against TNF-gamma-β were tested for the ability to inhibit the TNF-gamma-β induced caspase activation. Briefly, 24 micrograms/mL of each antibody (100×molar excess) was mixed with either 100 ng/mL of TNF-gamma-β in medium (RPMI+1% FBS+20 micrograms/mL cyclohexamide) or in medium alone (RPMI+1% FBS+20 micrograms/mL cyclohexamide) in a total volume of 150 microliters. These solutions were then incubated for 1 hour at room temperature. The media containing TNF-gamma-β+antibody or antibody alone solution were then added to the TF-1 cells and the caspase assay was performed as described above. Using this assay, monoclonal antibodies 12D08, 14A03, 15E09, and 16H02 were identified as potent TNF-gamma-β neutralizing antibodies.

### Example 13

### TR6 and DR3 Interact with TNF-Gamma-β

The premyeloid cell line TF-1 was stably transfected with SRE/SEAP (Signal Response Element/Secreted Alkaline Phosphatase) reporter plasmid that responds to the SRE signal transduction pathway. The TF1/SRE reporter cells were treated with TNF-gamma-β at 200 ng/mL and showed activation response as recorded by the SEAP activity. This activity can be neutralized by TR6.fc fusion protein in a dose dependent manner. The TR6.Fc by itself, in contrast, showed no activity on the TF1/SRE reporter cells. The results demonstrate that 1) TF-1 is a target cell for TNF-gamma-β ligand activity. 2) TR6 (International Publication Numbers WO98/30694 and WO00/52028) interacts with TNF-gamma-β and inhibits its activity on TF-1 cells. TR6 has two splice forms, alpha and beta; both splice forms have been shown to interact with TNF-gamma-β.

Similarly, the interaction of DR3 (International Publication Numbers WO97/33904 and WO/0064465) and TNF-gamma-β can be demonstrated using TF-1/SRE reporter cells. The results indicate that DR3.fc interacts with TNF-gamma-β, either by competing naturally expressed DR3 on TF-1 cells or forming inactive TNF-gamma-β/DR3.fc complex, or both.

At least three additional pieces of evidence demonstrate an interaction between TNF-gamma-β and DR3 and TR6. First, both TR6.Fc and DR3.Fc are able to inhibit TNF-gamma-β activation of NFkB in 293T cells, whereas in the same experiment, TNFR1.Fc was not able to inhibit TNF-gamma-β activation of NFkB in 293T cells. Secondly, both TR6.Fc and DR3.Fc can be used to immunoprecipitate TNF-gamma-β. Thirdly, TR6.Fc proteins can be detected by FACS analysis to specifically bind cells transfected with TNF-gamma-β.

### Example 14

### T Cell Proliferation and IFNγ ELISA

T Cell Proliferation Assay

The assay is performed as follows. PBMCs are purified from single donor whole blood by centrifugation through a histopaque gradient. PBMCs are cultured overnight in 10% RPMI and the following day non-adherent cells are collected and used for the proliferation assay. 96-well plates are pre-coated with either anti-CD3 or anti-CD3 and anti-CD28 and incubated overnight at 4 C. Plates are washed twice with PBS before use. TNF-gamma-β protein at desired concentrations in 10% RPMI is added to the 2×10⁴ cells/well in a final volume of 200 ul. 10 ng/ml recombinant human IL2 was used as a positive control. After 24 hours culture, samples are pulsed with 1 uCi/well 3H-thymidine. 26 hours after pulsing, cells are harvested and counted for 3H-thymidine.

IFNγ ELISA

The assay is performed as follows. Twenty-four well plates are coated with either 300 ng/ml or 600 ng/ml anti-CD3 and 5 ug/ml anti-CD28 (Pharmingen, San Diego, Calif.) in a final volume of 500 ul and incubated overnight at 4C. Plates are washed twice with PBS before use. PBMC are isolated by Ficoll (LSM, ICN Biotechnologies, Aurora, Ohio) gradient centrifugation from human peripheral blood, and are cultured overnight in 10% FCS(Fetal Calf Serum, Biofluids, Rockville, Md.)/RPMI (Gibco BRL, Gaithersburg, Md.). The following day, the non adherent cells are collected, washed and used in the costimulation assay. The assay is performed in the pre-coated twenty-four well plate using 1×10⁵ cells/well in a final volume of 900 ul. TNF-gamma-β protein is added to the cultures. Recombinant human IL-2 (purchased from R & D Systems, Minneapolis, Minn.) at a final concentration of 10 ng/ml was used as a positive control. Controls and unknown samples are tested in duplicate. Supernatant samples (250 ul) are collected 2 days and 5 days after the beginning of the assay. The level of IFNγ and 1L-2 in culture supernatants is then measured by ELISA.

Results

TNF-gamma-β treatment of PBMCs results in proliferation of T cells and a significant increase in IFNγ production compared to controls.

### Example 15

### Expression and Purification of TNFR-6 alpha and TNFR-6 beta in E. coli

The bacterial expression vector pQE60 is used for bacterial expression in this example (QIAGEN, Inc., 9259 Eton Avenue, Chatsworth, Calif., 91311). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., supra, and suitable single restriction enzyme cleavage sites. These elements are arranged such that a DNA fragment encoding a polypeptide may be inserted in such as way as to produce that polypeptide with the six His residues (i.e., a “6×His tag”) covalently linked to the carboxyl terminus of that polypeptide. However, in this example, the polypeptide coding sequence is inserted such that translation of the six His codons is prevented and, therefore, the polypeptide is produced with no 6×His tag.

The DNA sequences encoding the desired portions of TNFR-6 alpha and TNFR-6 beta proteins comprising the mature forms of the TNFR-6 alpha and TNFR-6 beta amino acid sequences are amplified from the deposited cDNA clones using PCR oligonucleotide primers which anneal to the amino terminal sequences of the desired portions of the TNFR-6α or -6β proteins and to sequences in the deposited constructs 3′ to the cDNA coding sequence. Additional nucleotides containing restriction sites to facilitate cloning in the pQE60 vector are added to the 5′ and 3′ sequences, respectively.

For cloning the mature form of the TNFR-6α protein, the 5′ primer has the sequence 5′CGCCCATGGCAGAAACACCCACCTAC 3′ (SEQ ID NO:51) containing the underlined NcoI restriction site. One of ordinary skill in the art would appreciate, of course, that the point in the protein coding sequence where the 5′ primer begins may be varied to amplify a desired portion of the complete protein shorter or longer than the mature form. The 3′ primer has the sequence 5′CGCAAGCTTCTCTTTCAGTGCAAGTG 3′ (SEQ ID NO:52) containing the underlined HindIII restriction site. For cloning the mature form of the TNFR-6β protein, the 5′ primer has the sequence of SEQ ID NO:19 above, and the 3′ primer has the sequence 5′CGCAAGCTTCTCCTCAGCTCCTGCAGTG 3′ (SEQ ID NO:53) containing the underlined HindIII restriction site.

The amplified TNFR-6 alpha and TNFR-6 beta DNA fragments and the vector pQE60 are digested with NcoI and HindIII and the digested DNAs are then ligated together. Insertion of the TNFR-6 alpha and TNFR-6 beta DNA into the restricted pQE60 vector places the TNFR-6 alpha and TNFR-6 beta protein coding region including its associated stop codon downstream from the IPTG-inducible promoter and in-frame with an initiating AUG. The associated stop codon prevents translation of the six histidine codons downstream of the insertion point.

The ligation mixture is transformed into competent E. coli cells using standard procedures such as those described in Sambrook et al., Molecular Cloning: a Laboratory Manual, 2nd Ed.; Cold Spring Harbor Laboratory Press, Cold Spring Harbor, N.Y. (1989). E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing TNFR-6α or -6β protein, is available commercially from QIAGEN, Inc., supra. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. isopropyl-13-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

To purify the TNFR-6 alpha and TNFR-6 beta polypeptide, the cells are then stirred for 3-4 hours at 4° C. in 6M guanidine-HCl, pH 8. The cell debris is removed by centrifugation, and the supernatant containing the TNFR-6 alpha and TNFR-6 beta is dialyzed against 50 mM Na-acetate buffer pH 6, supplemented with 200 mM NaCl. Alternatively, the protein can be successfully refolded by dialyzing it against 500 mM NaCl, 20% glycerol, 25 mM Tris/HCl pH 7.4, containing protease inhibitors. After renaturation the protein can be purified by ion exchange, hydrophobic interaction and size exclusion chromatography. Alternatively, an affinity chromatography step such as an antibody column can be used to obtain pure TNFR-6 alpha and TNFR-6 beta protein. The purified protein is stored at 40 C or frozen at −80° C.

The following alternative method may be used to purify TNFR-6α or -6β expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells are harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells ware then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GnHCl) for 2-4 hours. After 7000×g centrifugation for 15 min., the pellet is discarded and the TNFR-6α or -6β polypeptide-containing supernatant is incubated at 4° C. overnight to allow further GnHCl extraction.

Following high speed centrifugation (30,000×g) to remove insoluble particles, the GnHCl solubilized protein is refolded by quickly mixing the GnHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded TNF receptor polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 mm of the effluent is continuously monitored. Fractions are collected and further analyzed by SDS-PAGE.

Fractions containing the TNF receptor polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the TNFR-6α or -6β polypeptide (determined, for instance, by 16% SDS-PAGE) are then pooled.

The resultant TNF receptor polypeptide exhibits greater than 95% purity after the above refolding and purification steps. No major contaminant bands are observed from Commassie blue stained 16% SDS-PAGE gel when 5 μg of purified protein is loaded. The purified protein is also tested for endotoxin/LPS contamination, and typically the LPS content is less than 0.1 ng/ml according to LAL assays.

### Example 16

### Cloning and Expression of TNFR-6 alpha and TNFR-6 beta Proteins in a Baculovirus Expression System

In this illustrative example, the plasmid shuttle vector pA2 is used to insert the cloned DNA encoding complete protein, including its naturally associated secretory signal (leader) sequence, into a baculovirus to express the mature TNFR-6α or -6β protein, using standard methods as described in Summers et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

The cDNA sequence encoding the full length TNFR-6α or -6β protein in a deposited clone, including the AUG initiation codon and the naturally associated leader sequence shown in SEQ ID NO:2 or 4 is amplified using PCR oligonucleotide primers corresponding to the 5′ and 3′ sequences of the gene. The 5′ primer for TNFR-6 alpha and TNFR-6 beta has the sequence 5′CGCGGATCCGCCATCATGAGGGCTGGAGG GCCAG 3′ (SEQ ID NO:54) containing the underlined BamHI restriction enzyme site. All of the previously described primers encode an efficient signal for initiation of translation in eukaryotic cells, as described by Kozak, M., J. Mol. Biol. 196:947-950 (1987). The 3′ primer for TNFR-6α has the sequence 5′CGCGGTACCCTCTTTCAGT GCAAGTG 3′ (SEQ ID NO:55) containing the underlined Asp718 restriction site. The 3′ primer for TNFR-6p has the sequence 5′CGCGGTACCCTCCTCAGCTCCTGCAGTG 3′ (SEQ ID NO:56) containing the underlined Asp718 restriction site.

The amplified fragment is isolated from a 1% agarose gel using a commercially available kit (“Geneclean,” BIO 101 Inc., La Jolla, Calif.). The fragment then is digested with the appropriate restriction enzyme for each of the primers used, as specified above, and again is purified on a 1% agarose gel.

The plasmid is digested with the same restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and dephosphorylated plasmid are ligated together with T4 DNA ligase. E. coli HB101 or other suitable E. coli hosts such as XL-1 Blue (Statagene Cloning Systems, La Jolla, Calif.) cells are transformed with the ligation mixture and spread on culture plates. Bacteria are identified that contain the plasmid with the human TNF receptor gene by digesting DNA from individual colonies using the enzymes used immediately above and then analyzing the digestion product by gel electrophoresis. The sequence of the cloned fragment is confirmed by DNA sequencing. This plasmid is designated herein pA2-TNFR-6α or pA2TNFR-6β (collectively pA2-TNFR).

Five μg of the plasmid pA2-TNFR is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84: 7413-7417 (1987). One μg of BaculoGold™ virus DNA and 5 μg of the plasmid pA2-TNFR are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C.

To verify the expression of the TNF receptor gene Sf9 cells are grown in Grace's medium supplemented with 10% heat-inactivated FBS. The cells are infected with the recombinant baculovirus at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the mature form of the TNF receptor protein.

### Example 17

### Cloning and Expression of TNFR-6 alpha and TNFR-6 beta in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) marker is useful to develop cell lines that carry several hundred or even several thousand copies of the gene of interest. Another useful selection marker is the enzyme glutamine synthase (GS) (Murphy et al., Biochem J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### Example 17(a)

### Cloning and Expression in COS Cells

The expression plasmid, pTNFR-α-HA and -6β-HA, is made by cloning a portion of the cDNA encoding the mature form of the TNF receptor protein into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson et al., Cell 37: 767 (1984). The fusion of the HA tag to the target protein allows easy detection and recovery of the recombinant protein with an antibody that recognizes the HA epitope. pcDNAIII contains, in addition, the selectable neomycin marker.

A DNA fragment encoding the complete TNF receptor polypeptide is cloned into the polylinker region of the vector so that recombinant protein expression is directed by the CMV promoter. The plasmid construction strategy is as follows. The TNF receptor cDNA of a deposited clone is amplified using primers that contain convenient restriction sites, much as described above for construction of vectors for expression of a TNF receptor in E. coli. Suitable primers can easily be designed by those of ordinary skill in the art.

The PCR amplified DNA fragment and the vector, pcDNAI/Amp, are digested with XbaI and EcoRI and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the fragment encoding the TNFR-α and -6β polypeptides.

For expression of recombinant TNFR-α and -6β, COS cells are transfected with an expression vector, as described above, using DEAE-DEXTRAN, as described, for instance, in Sambrook et al., Molecular Cloning: a Laboratory Manual, Cold Spring Laboratory Press, Cold Spring Harbor, N.Y. (1989). Cells are incubated under conditions for expression of TNFR by the vector.

Expression of the pTNFR-α-HA and -6β-HA fusion protein is detected by radiolabeling and immunoprecipitation, using methods described in, for example Harlow et al., Antibodies: A Laboratory Manual, 2nd Ed.; Cold Spring Harbor Laboratory Press, Cold Spring Harbor, N.Y. (1988). To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### Example 17(b)

### Cloning and Expression in CHO Cells

The vector pC4 is used for the expression of TNFR-6 alpha and TNFR-6 beta polypeptides. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J. Biol. Chem. 253:1357-1370, Hamlin, J. L. and Ma, C. 1990, Biochem. et Biophys. Acta, 1097:107-143, Page, M. J. and Sydenham, M. A. 1991, Biotechnology 9:64-68). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rouse Sarcoma Virus (Cullen, et al., Molecular and Cellular Biology, March 1985:438-447) plus a fragment isolated from the enhancer of the immediate early gene of human cytomegalovirus (CMV) (Boshart et al., Cell 41:521-530 (1985)). Downstream of the promoter are the following single restriction enzyme cleavage sites that allow the integration of the genes: BamHI, Xba I, and Asp718. Behind these cloning sites the plasmid contains the 3′ intron and polyadenylation site of the rat preproinsulin gene. Other high efficiency promoters can also be used for the expression, e.g., the human β-actin promoter, the SV40 early or late promoters or the long terminal repeats from other retroviruses, e.g., HIV and HTLVI. Clontech's Tet-Off and Tet-On gene expression systems and similar systems can be used to express the TNF receptor polypeptide in a regulated way in mammalian cells (Gossen, M., & Bujard, H., Proc. Natl. Acad. Sci. USA 89:5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418, or hygromycin. It is advantageous to use more than one selectable marker in the beginning, e.g., G418 plus methotrexate.

The plasmid pC4 is digested with the restriction enzymes appropriate for the specific primers used to amplify the TNF receptor of choice as outlined below and then dephosphorylated using calf intestinal phosphates by procedures known in the art. The vector is then isolated from a 1% agarose gel.

The DNA sequence encoding the TNF receptor polypeptide is amplified using PCR oligonucleotide primers corresponding to the 5′ and 3′ sequences of the desired portion of the gene. The 5′ primer for TNFR-6 alpha and TNFR-6 beta containing the underlined BamHI site, has the following sequence: 5′CGCGGATCCGCCATCATGAG GGCGTGGAGGGGCCAG 3′ (SEQ ID NO:54). The 3′ primer for TNFR-6α has the sequence 5′CGCGGTACCCTCTTTCAGTGCA AGTG 3′ (SEQ ID NO:55) containing the underlined Asp718 restriction site. The 3′ primer for TNFR-6β has the sequence 5′ CGCGGTACCCTCCTCAGCTCCTGCAGTG 3′ (SEQ ID NO:56) containing the underlined Asp718 restriction site.

The amplified fragment is digested with the endonucleases which will cut at the engineered restriction site(s) and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. Five μg of the expression plasmid pC4 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 18

### Tissue Distribution of TNF Receptor mRNA Expression

Northern blot analysis is carried out to examine TNFR-6α or -6β gene expression in human tissues, using methods described by, among others, Sambrook et al., cited above. A cDNA probe containing the entire nucleotide sequence of a TNF receptor protein (i.e., TNFR-6 as shown in SEQ ID NO:5) is labeled with ³²P using the rediprime™ DNA labeling system (Amersham Life Science), according to manufacturer's instructions. After labeling, the probe is purified using a CHROMA SPIN-100™ column (Clontech Laboratories, Inc.), according to manufacturer's protocol number PT1200-1. The purified labeled probe is then used to examine various human tissues for TNF receptor mRNA.

Multiple Tissue Northern (MTN) blots containing various human tissues (H) or human immune system tissues (IM) are obtained from Clontech and are examined with the labeled probe using ExpressHyb™ hybridization solution (Clontech) according to manufacturer's protocol number PT1190-1. Following hybridization and washing, the blots are mounted and exposed to film at −70° C. overnight, and films developed according to standard procedures.

It will be clear that the invention may be practiced otherwise than as particularly described in the foregoing description and examples. Numerous modifications and variations of the present invention are possible in light of the above teachings and, therefore, are within the scope of the appended claims.

### Example 19

### Gene Therapy Using Endogenous TNFR-6 Gene

Another method of gene therapy according to the present invention involves operably associating the endogenous TNFR (i.e., TNFR-6) sequence with a promoter via homologous recombination as described, for example, in U.S. Pat. No. 5,641,670, issued Jun. 24, 1997; International application publication number WO 96/29411, published Sep. 26, 1996; International application publication number WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not expressed in the cells, or is expressed at a lower level than desired. Polynucleotide constructs are made which contain a promoter and targeting sequences, which are homologous to the 5′ non-coding sequence of endogenous TNFR-6, flanking the promoter. The targeting sequence will be sufficiently near the 5′ end of TNFR-6 so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous TNFR-6 sequence. This results in the expression of TNFR-6 in the cell. Expression may be detected by immunological staining, or any other method known in the art.

Fibroblasts are obtained from a subject by skin biopsy. The resulting tissue is placed in DMEM+10% fetal calf serum. Exponentially growing or early stationary phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na2 HPO4, 6 mM dextrose). The cells are recentrifuged, the supernatant aspirated, and the cells resuspended in electroporation buffer containing 1 mg/ml acetylated bovine serum albumin. The final cell suspension contains approximately 3×10⁶ cells/ml. Electroporation should be performed immediately following resuspension.

Plasmid DNA is prepared according to standard techniques. For example, to construct a plasmid for targeting to the TNFR-6 locus, plasmid pUC18 (MBI Fermentas, Amherst, N.Y.) is digested with HindIII. The CMV promoter is amplified by PCR with an XbaI site on the 5′ end and a BamHI site on the 3′ end. Two TNFR-6 non-coding sequences are amplified via PCR: one TNFR-6 non-coding sequence (TNFR-6 fragment 1) is amplified with a HindIII site at the 5′ end and an Xba site at the 3′ end; the other TNFR-6 non-coding sequence (TNFR-6 fragment 2) is amplified with a BamHI site at the 5′end and a HindIII site at the 3′ end. The CMV promoter and TNFR-6 fragments are digested with the appropriate enzymes (CMV promoter—XbaI and BamHI; TNFR-6 fragment 1—XbaI; TNFR-6 fragment 2—BamHI) and ligated together. The resulting ligation product is digested with HindIII, and ligated with the HindIII-digested pUC18 plasmid.

Plasmid DNA is added to a sterile cuvette with a 0.4 cm electrode gap (Bio-Rad). The final DNA concentration is generally at least 120 μg/ml. 0.5 ml of the cell suspension (containing approximately 1.5×10⁶ cells) is then added to the cuvette, and the cell suspension and DNA solutions are gently mixed. Electroporation is performed with a Gene-Pulser apparatus (Bio-Rad). Capacitance and voltage are set at 960 μF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 min, and the contents of the cuvette are then gently removed with a sterile transfer pipette. The cells are added directly to 10 ml of prewarmed nutrient media (DMEM with 15% calf serum) in a 10 cm dish and incubated at 37 C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 20

### Protein Fusions of TNFR-6 Alpha and/or TNFR-6 Beta

TNFR-6 alpha and/or TNFR-6 beta polypeptides of the invention are optionally fused to other proteins. These fusion proteins can be used for a variety of applications. For example, fusion of TNFR-6 alpha and/or TNFR-6 beta polypeptides to His-tag, HA-tag, protein A, IgG domains, and maltose binding protein facilitates purification. (See EP A 394,827; Traunecker, et al., Nature 331:84-86 (1988).) Similarly, fusion to IgG-1, IgG-3, and albumin increases the halflife time in vivo. Nuclear localization signals fused to TNFR-6 alpha and/or TNFR-6 beta polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made using techniques known in the art or by using or routinely modifying the following protocol, which outlines the fusion of a polypeptide to an IgG molecule.

Briefly, the human Fc portion of the IgG molecule (SEQ ID NO:50) can be PCR amplified, using primers that span the 5′ and 3′ ends of the sequence described below. These primers also preferably contain convenient restriction enzyme sites that will facilitate cloning into an expression vector, preferably a mammalian expression vector.

For example, if the pC4 (Accession No. 209646) expression vector is used, the human Fc portion can be ligated into the BamHI cloning site. Note that the 3′ BamHI site should be destroyed. Next, the vector containing the human Fc portion is re-restricted with BamHI, linearizing the vector, and TNFR-6 alpha and/or TNFR-6 beta polynucleotide, isolated by the PCR protocol described in Example 16, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., International application publication number WO 96/34891.)

### Example 21

### Modulation of T Cell Responses By TNFR6: Soluble TNFR6 Inhibits Alloactivation and Heart Allograft Rejection

The ability of TNFR6 to interact with LIGHT and the role of TNFR6 in modulating T cell activities and immunological responses that may be associated with LIGHT were analyzed according to the experiments detailed below.

Mice

Twelve week-old female C57BL/6 (B6, H-2^{b}), BALB/c, and BALB/c x C57BL/6 Fl (H-2^{bXd}) mice were purchased from Jackson Laboratory (Bar Harbor, Me.) or Charles River (LaSalle, Quebec, Canada). 2C TCR transgenic mice were bred in an animal facility as described in Chen, H., et al., 1996. J. Immunol. 157:4297, which is hereby incorporated by reference in its entirety.

Expression and Purification of the Human TR6-Fc Fusion Protein

Full-length human TNFR-6 alpha cDNA (SEQ ID NO:6, aa 1-300; referred in this example hereafter as “TR6”) was PCR-amplified using gene specific primers, fused to the sequence coding for the Fc domain of human IgG₁ and subcloned into a baculovirus expression vector pA2. The construct was named pA2-Fc:TR6. Sf9 cells infected pA2-Fc:TR6 were grown in media (100L) containing 1% ultra low IgG serum (100L). Conditioned culture supernatant from a bioreactor was harvested by continuous flow centrifugation. The pH of the supernatant was adjusted to pH 7.0, filtered through 0.22 um filter and loaded on to a Protein A column (BioSepra Ceramic HyperD, Life Technologies, Rockville, Md. 30 ml bed volume) previously conditioned with 20 mM phosphate buffer, 0.5 M NaCl (pH 7.2). The column was washed with 15 column volumes (CV) of 20 mM phosphate buffer (pH 7.2) containing 0.5 M NaCl followed by 5 CV of 0.1 M sodium citrate (pH 5.0). TR6-Fc was eluted with 0.1 M citric acid (pH 2.4), and 2 mL fractions were collected into tubes containing 0.6 ml Tris-HCl (pH 9.2). The TR6-Fc positive fractions were determined by SDS-PAGE. The peak fractions were pooled and concentrated with a Protein A column (7 mL bed volume) as described above. The concentrated TR6-Fc was loaded onto a Superdex 200 column (Amersham Pharmacia, Piscataway, N.J. 90 ml bed volume) and eluted with PBS containing 0.5 M NaCl. TR6-Fc positive fractions were determined by non-reducing SDS-PAGE. The pooled positive fractions were dialyzed against 12.5 mM HEPES buffer, pH 5.75 containing 50 mM NaCl. The dialysate was then passed through a 0.2 m filter (Minisart, Sartorius A G, Goettingen, Germany) followed by a Q15X-anion exchange membrane (Sartobind membrane, Sartorius A G, Goettingen, Germany).

Expression and Purification of Full-Length Human TR6 (without Fc)

The full-length TR6 cDNA was PCR-amplified and cloned in to the baculovirus expression vector pA2 as describe above. Sf9 cells were infected with the viral construct, and the culture supernatant of the infected cells was loaded onto a Poros HS-50 column (Applied Biosystems, Foster City, Calif.) equilibrated in a buffer containing 50 mM Tris-HCl, pH 7, and 0.1M NaCl. The column was washed with 0.1 M NaCl and eluted stepwise with 0.3M, 0.5M, and 1.5M NaCl. The eluded fractions were analyzed by SDS-PAGE, and the 0.5 M NaCl fraction containing TR6 protein was diluted and loaded onto a set of Poros HQ-50/CM-20 columns in a tandem mode. TR6 was eluted from the CM column with a linear gradient from 0.2M to 1.0 M NaCl.

Expression and Purification of Human TR2-Fc, MCIF-Fc, and Fas-Fc, Fusion Proteins

The cDNA sequences coding for the extracellular domain of TR2 (aa 1-192), the extracellular domain of Fas (aa 1-169) and a beta chemokine MCIF (aa 1-92) were fused with the cDNA sequence coding for the Fe domain of human IgG₁, and cloned into a eukaryotic expression vector pC4. The construct was stablely transfected into CHO cells. The Fe fusion proteins from the CHO supernatant were purified with methods used for TR6-Fc.

Expression and Purification of the Human LIGHT Protein

The coding sequence of the natural secreted form of LIGHT (aa 83-240) was cloned into a prokaryotic expression vector pHE4 (ATCC Deposit Number 209645, described in U.S. Pat. No. 6,194,168), and expressed in E. coli. Inclusion bodies from the transformed bacteria were dissolved for 48-72 hours at 4 C in 3.5 M guanidine hydrochloride containing 100 mM Tris-HCl, pH 7.4 and 2 mM CaCl₂. The solution was quickly diluted with 20-30 volumes of a buffer containing 50 mM Tris-HCl, pH 8 and 150 mM NaCl, adjusted to pH 6.6 and chromatographed with a strong cation exchange column (Poros HS-50). The protein was eluted with 3-5 CV of a stepwise gradient of 300 mM, 700 mM, and 1500 mM NaCl in 50 mM MES at pH 6.6. The fraction eluted with 0.7 M NaCl was diluted 3-fold with water, and applied to a set of strong anion (Poros HQ-50) and cation (Poros CM-20) exchange columns in a tandem mode. The CM column was eluted with 10-20 CV of a linear gradient from 50 mM MES pH 6.6, 150 mM NaCl to 50 mM Tris-HCl pH 8, 500 mM NaCl. Fractions containing purified LIGHT as analyzed by SDS-PAGE were combined.

Quality Control of the Recombinant Proteins

The endotoxin levels in the purified recombinant proteins were determined by the LAL assay on a Limulus Amebocyte Lysate (LAL)-5000 Automatic Endotoxin Detection System (Associates of Cape Cod, Inc. Falmouth, MA), according to the standard procedure recommended by the manufacturer. All the recombinant proteins were subjected to N-terminal sequence using an ABI-494 sequencer (PE Biosystems, Inc. Foster City, Calif.) for their authenticity. The proteins was dialyzed against PBS containing 20% (v/v) glycerol for storage at −80° C. For applications such as CTL, cytokine secretion and heart transplantation, the proteins were subsequently dialyzed against PBS to remove the glycerol in the solution.

BIAcore Analysis

The binding of human LIGHT to human TR6-Fc was first assessed by BIAcore analysis (BIAcore Biosensor, Piscataway, N.J.). TR6-Fc or TR2-Fc fusion proteins were covalently immobilized to the BIAcore sensor chip (CM5 chip) via amine groups using N-ethyl-N′-(dimethylaminopropyl)carbodiimide/N-hydroxysuccinimide. Various dilutions of LIGHT were passed through the TR6-Fc- or TR2-Fc-conjugated flow cells at 15 microliters/min for a total volume of 50 microliters. The amount of bound protein was determined during washing of the flow cell with HBS buffer (10 mM HEPES, pH 7.4, 150 mM NaCl, 3.4 mM EDTA, 0.005% Surfactant P20). The flow cell surface was regenerated by washing off the bound proteins with 20 microliters of 10 mM glycine-HCl pH 2.3. For kinetic analysis the flow cells were tested at different flow rates and with different density of the conjugated TR6-Fc or TR2-Fc proteins. The on- and off-rates were determined according a kinetic evaluation program in the BiaEvaluation 3 software using a 1:1 binding model and the global analysis method.

Generation of Stable Cell Lines that Express Human LIGHT

The full-length human LIGHT genes were PCR amplified and subcloned into pcDNA3.1. The parental vector and the LIGHT expression vectors were then transfected into 293F cells (Life Technologies, Grand Island, N.Y.) using Lipofectamine (Life Technology) and stable clones resistant to 0.5 mg/ml geneticin were selected.

Flow Cytometry

Cells were incubated with Fc-fusion proteins in 100 ul FACS buffer (d-PBS with 0.1% sodium azide and 0.1% BSA) for 15-20 minutes at room temperature. The cells were washed once and reacted with goat F (ab)₂ anti-human IgG (Southern Biotechnology, Birmingham, Ala.) for 15 minutes at room temperature. After wash, the cells were resuspended in 0.5 ug/ml propidium iodide, and live cells were gated and analyzed on a FACScan (BD Biosciences, Mansfield, Mass.).

Stimulation of Human T Cells for LIGHT Expression

Briefly, T cells were purified from human peripheral blood and stimulated with anti-CD3 in the presence of rhuIL-2 for 5 days. The cells were restimulated with PMA (100 ng/ml) and ionomycin (1 mg/ml) for additional 4 hours. LIGHT expression on the cells was assessed by the binding of TR6-Fc (10 ng/sample), TR2-Fc (250 ng/sample) or Fas-Fc (250 ng/sample) to the cells using flow cytometry.

Three-Way MLR of Human PBMC

PBMC from human donors were purified by density gradient using Lymphocyte Separation Medium (LSM, density at 1.0770 g/ml, Organon Teknika Corporation, West Chester, Pa.). PBMC from three donors were mixed at a ratio of 2:2:0.2 for a final density of 4.2×10⁶ cells/ml in RPMI-1640 (Life Technologies) containing 10% FCS and 2 mM glutamine. The cells were cultured for 5-6 days in round-bottomed microtitre plates (200 microliters/well) in triplicate, pulsed with [³H] thymidine for the last 16 h of culture, and the thymidine uptake was measured as describe before (Chen, H., et al., 1996. J. Immunol. 157:4297, which is hereby incorporated by reference in its entirety).

One-Way ex vivo MLR after in vivo Stimulation in Mice

The F1 of C57BL/6×BALB/c mice (H-2^{bXd}) were transfused i.v. with 1.5×10⁸ spleen cells from C57BL/6 mice (H-2^{b}) on day 1. TR6-Fc or a control fusion protein was administered i.v. daily for 9 days at 3 mg/kg/day starting one day before the transfusion. The spleen cells of the recipient F1 mice were harvested on day 8 for in vitro proliferation and cytokine assays.

Ex vivo Mouse Splenocyte Proliferation

Single splenocyte suspensions from normal and transfused Fl mice were cultured in triplicate in 96-well flat-bottomed plates (4×10⁵ cells/200 microliters/well) for 2-5 days as with the human MLR. After removing 100 microliters of supernatants per well on the day of harvest, 10 microliters alamar Blue (Biosource, Camarillo, Calif.) was added to each well and the cells were cultured for additional 4 h. The cell number in each well was assessed according to OD₅₉₀ using a CytoFlu apparatus (PerSeptive Biosystems, Framingham, Mass.).

Mouse Cytokine Assays

Cytokines in the culture supernatants of mouse spleen cells were measured with commercial ELISA kits from Endogen (Cambridge, Mass.) or R & D Systems (Minneapolis, Minn.).

Mouse Cytotoxic T Lymphocyte (CTL) Assay

Transgenic mice carrying L^{d}-specific TCR (2C mice) were used in this experiment. In the 2C mice, the majority (about 75%) of their T cells are CD8⁺, and almost all the CD8⁺ cells carry clonotypic TCR recognized by mAb 1B2. The 2C mice in our colony are of an H-2^{b} background. 2C spleen cells were stimulated with an equal number of mitomycin C-treated BALB/c spleen cells in 24-well plates at a final density of 4×10⁶ cells/2 ml/well. After 5 days of culture in the presence of 10 U/ml recombinant human IL-2, the viable cells were counted and assayed for their H-2^{d}-specific cytotoxic activity using ⁵¹Cr-labeled P815 cells (H-2 d) as targets. A standard 4-h ⁵¹Cr release assay (Chen, H., et al., 1996. J. Immunol. 157:4297, which is hereby incorporated by reference in its entirety) was carried out in 96-well round-bottomed plates with 0.15×10⁶ target cells/well/200 microliters at different ratios of effector/target cells (10:1, 3:1, 1:1 and 0.3:1). After 4-h incubation, 100 microliters of supernatant was collected from each well and counted in a gamma-counter. The percentage lysis of the test sample is calculated as follows:

$$\% lysis = \frac{cpm of the test sample - cpm of spontaneous release}{cpm of maximal release - cpm of spontaneous release}$$

where the spontaneous release is derived from 100 microliters supernatant of the target cells cultured alone for 4 h, and the maximal release is derived from 100 microliters lysate of 0.15×10⁶ target cells which were lysed by SDS in a total volume of 200 microliters.

Mouse Heart Transplantation

Three- to four-month-old C57BL/6 mice (H-2^{b}) were used as recipients, and 2-to 3-month-old BALB/c mice (H-2^{d}) were used as donors. The procedure of heterotopic heart transplantation was detailed in Chen, H., et al., 1996. J. Immunol. 157:4297, which is hereby incorporated by reference in its entirety. The contraction of the transplanted heart was assessed daily by abdominal palpation. The duration between the day of the operation and the first day when a graft totally lost its palpable activity was defined as the graft survival time. Animals that lost palpable activity of the graft within three days after transplantation were classified as technical failures (<5%) and were omitted from the analysis.

Results

Preparation of Recombinant Proteins of Human TR6-Fc, TR6, LIGHT, TR2-Fc, Fas-Fc and MCIF-Fc

The purified TR6-Fc protein was analyzed with SDS-PAGE under reducing and nonreducing conditions. The result demonstrate that the protein is a disulfide-linked dimer under the non-reducing condition. Light scattering analysis also confirmed that the protein behaves as a dimer in solution. N-terminal sequencing revealed that the mature secreted TR6-Fc had the predicted sequence of VAETP starting at aa 30. The estimated purity of the protein preparation was more than 98% according to SDS-PAGE. Endotoxin levels in the purified proteins were below 10 EU/mg. Human TR6 without Fc, TR2-Fc, Fas-Fc and MCIF-FC were also prepared to a similar purity as TR6-Fc and their authenticity was verified with N-terminal sequencing.

The Kinetics of Binding Between of TR6 and LIGHT

TR6-Fc has been previously shown to bind both LIGHT and FasL. We determined the kinetics of binding of LIGHT to both the Fe and non-Fc versions of TR6 according to BIAcore analysis. The Kd for LIGHT binding to TR6-Fc and non-Fc forms was 5.46 nM and 14.3 nM, respectively. The off rate (kd) for TR6 (4.83E-03 1/s) was approximately 2-fold higher than that of TR6-Fc (2.30E-03 1/s). The on-rates, ka, were 4.22E05 and 3.38E05 1/Ms for TR6-Fc and TR6, respectively, with TR6-Fc having a slightly higher on rate. The exact reason for the apparent higher Kd value for TR6-Fc compared to TR6-Fc is not known, but a comparable difference in binding affinity was also observed with FasL. The binding of LIGHT to TR2-Fc was also determined. The Kd was 4.56 nM, which is essentially the same as that between LIGHT and TR6-Fc.

TR6-Fc Binds LIGHT Directly and can Compete with TR2 for the Binding of LIGHT Overexpressed on 293 Cell Surface

After it was shown that TR6-Fc could bind to LIGHT in BIAcore chips, the ability of TR6-Fc to bind to LIGHT expressed on cell surface was analyzed. This was tested on 293 cells overexpressing LIGHT according to flow cytometry. Fas-Fc was used as a control, and it did not bind to the transfected cells. TR6-Fc could bind to the LIGHT-transfectants, but not on untransfected cells. The specificity of the binding was further demonstrated by competition of TR6-Fc binding with soluble non-Fc form of TR6. Dose-dependent competition of TR6-Fc binding was attained using increasing concentrations of TR6 protein, and nearly complete inhibition was achieved with 10 micrograms of TR6.

It has been shown that TR2 can bind to LIGHT. Since TR6 also binds to LIGHT as shown above, its ability to interfere with the binding between TR2 and LIGHT was analyzed. This possibility was examined with flow cytometry. TR2-Fc could bind to the 293 cells overexpressing LIGHT as expected. TR6 could compete off the binding in a dose-dependent fashion. At 10 micrograms of TR6, the binding of TR2-Fc to the 293 cells was almost completely disappeared.

The results from this section indicate that TR-6 can bind to the cell membrane LIGHT, and it can also compete with TR2 for the binding of LIGHT.

TR6-Fc Reactivity with Activated T Cells

LIGHT expression is upregulated on T cells activated with anti-CD3 and IL-2 followed by PMA and ionomycin treatment (Mauri, D. N., et al., 1998, Immunity. 8:21). Using this activation regimen, we confirmed previous results according to flow cytometry that TR2-Fc bound to T cells thus activated. We then extended this observation by showing that as with TR2-Fc, TR6-Fc also bound to these activated T cells. The binding was specific because a control Fc fusion protein Fas-Fc did not bind to these cells, and the binding could be competed off with soluble TR6. The interaction between TR6 and the activated T cells was mediated via LIGHT expressed on these T cells, because the same soluble TR6 protein could also compete off the binding of TR2-Fc and LTbetaR-Fc with the T cells, TR2 and LTbetaR being receptors of LIGHT. These results demonstrate that soluble TR6 could associate with endogenous LIGHT expressed on the activated T cells, and it can interfere with the interaction between LIGHT and TR2 in immune cells.

TR6-Fc Inhibits Human MLR

It has been shown that soluble LIGHT can enhance a 3-way MLR, and soluble recombinant TR2-Fc can inhibit the 3-way MLR or dendritic cells-stimulated alloresponse of the T cells. These immune regulations are likely via the interaction between soluble LIGHT and its cell surface receptor TR2. Since TR6 could interfere with the interaction between LIGHT and TR2 as shown in our flow cytometry, we analzyed its ability to alter T cell alloresponses by testing the effect of TR6 in a three-way human MLR. The results show that TR6-Fc inhibited the T cell proliferation in this system. A control Fc fusion protein had no effect, whereas TR6-Fc at 1 microgram/ml caused nearly 50% inhibition. Further increase of the TR6-Fc concentrations had no additional suppressive effect.

TR6-Fc Inhibits Splenocyte Alloactivation ex vivo in Mice

It has been shown previously that T cells stimulated by alloantigen in vivo have increased spontaneous proliferation ex vivo, and alloreactive T cells depend on LIGHT for some costimulation in certain case. We tested whether TR6 had any immune regulatory effects in vivo on alloantigen-stimulated T cells. Parental splenocytes (H-2^{b}) were transfused i.v. into H-2^{bXd} F1 mice, and the recipient mice were given TR6-Fc i.v. at 3 mg/kg/day for 8 days starting on day-1 (the day of transfusion was designated as day 0). The F1 mice were sacrificed on day 8 and the spleen weight of the mice were registered. The splenocytes were then cultured without additional stimulation to measure their spontaneous proliferation and cytokine production. Treatment with TR6-Fc reduced splenomagaly considerably, decreased spontaneous splenocyte proliferation as measured on day 4 after the culture, and inhibited the IFN-gammaand GM-CSF production by the spleen cells as measured from day 2 to day 5 of the culture. In contrast, all mice treated with control Fc or buffer had significantly more severe splenomagaly, higher splenocyte proliferation and higher INF-gamma and GM-CSF productions. Thus, our results show that TR6-Fc is immunologically active and can indeed modulate T cell-mediated alloactivation in vivo.

TR6-Fc and TR6 Inhibits Mouse CTL Activity Developed Against Alloantigens

L^{d}-specific transgenic 2C T cells were then used as a model system to evaluate the effect of TR6 on the differentiation of alloantigen-specific CD8 cells into effector cells, since the CD8 cells are mainly responsible to the alloresponsiveness, and the high alloreactive CD8 CTL precursors in the 2C mice gives out elevated read-out signals for easy detection of possible changes exerted by TR6. In the presence of either TR6-Fc or TR6, the CTL activity was decreased significantly compared with the cultures containing no recombinant protein or containing normal human IgG. The detection of similar effect of TR6 and TR6-Fc in this experiment is of significant importance, because it excludes the possibility that the effect seen with TR6-Fc is Fc-mediated. The CTL assay presented in the figure was carried out on day 6 of the culture. When CTL were assayed on day 5 of the culture, there was no obvious difference between samples with or without TR6. This indicates that the repression of CTL seen on day 6 is not due to a kinetic shift.

TR6-Fc Modulates Lymphokine Production of 2C T Cells Stimulated with H-2^{d} Alloantigens in vitro

The CTL differentiation and maturation are modulated by a plethora of lymphokines, and we examined the production of battery of lymphokines produced by 2C spleen cells upon stimulation of mitomycin C-treated BALB/c spleen cells (H-2d) in the presence of TR6-Fc. There was a suppression of IL-2 production between 24-72 h after the stimulation, while the levels of IL-10 were upregulated.

TR6-Fc Prolongs Heart Allograft Survival of the Mice

Since TR6-Fc could repress ex vivo T cell proliferation after the alloantigen stimulation, and inhibit CTL development in in vitro assays, we speculated that it could also modulate a more complete immune response such as graft rejection. This was tested in a model of mouse heterotopic heart allografting, with C57BL/6 as recipients and BALB/c as donors. The recipients were administrated with TR6-Fc i.v. daily at 7.5 mg/kg/day for 7 days starting from one day before the operation. For this test group, the mean survival time (MST) of the grafts was 10.0+1.2 days, while the MST of the control group was 6.8+0.4 days. The difference between the two groups was highly significant (p=0.0001, non-paired Student's t test). This result shows that TR6-Fc could modulate an authentic immune response such as allograft rejection.

### Example 22

### TNFR-6 Alpha, TNFR-6 Beta and DR3 Interact with TNF-Gamma-β

The premyeloid cell line TF-1 was stably transfected with SRE/SEAP (Signal Response Element/Secreted Alkaline Phosphatase) reporter plasmid that responds to the SRE signal transduction pathway. The TF1/SRE reporter cells were treated with TNF-gamma-β (International Publication Numbers WO96/14328, WO00/66608, and WO00/08139) at 200 ng/mL and showed activation response as recorded by the SEAP activity. This activity can be neutralized by A TNFR-6 alpha Fc fusion protein (hereinafter TR6.Fc in this example) in a dose dependent manner. The TR6.Fc by itself, in contrast, showed no activity on the TF1/SRE reporter cells. The results demonstrate that 1) TF-1 is a target cell for TNF-gamma-β ligand activity; and 2) TR6 interacts with TNF-gamma-β and inhibits its activity on TF-1 cells. TR6 is known to have two splice forms, TR6-alpha and TR6-beta; both splice forms have been shown to interact with TNF-gamma-β.

Similarly, the interaction of DR3 (International Publication Numbers WO97/33904 and WO/0064465) and TNF-gamma-beta can be demonstrated using TF-1/SRE reporter cells. The results indicate that DR3.Fc interacts with TNF-gamma-beta, either by competing naturally expressed DR3 on TF-1 cells or forming inactive TNF-gamma-β/DR3.fc complex, or both.

At least three additional pieces of evidence demonstrate an interaction between TNF-gamma-β and DR3 and TR6. First, both TR6.Fc and DR3.Fc are able to inhibit TNF-gamma-β activation of NFkB in 293T cells, whereas in the same experiment, TNFR1.Fc was not able to inhibit TNF-gamma-β activation of NFkB in 293T cells. Secondly, both TR6.Fc and DR3.Fc can be used to immunoprecipitate TNF-gamma-β. Thirdly, TR6.Fc proteins can be detected by FACS analysis to specifically bind cells transfected with TNF-gamma-β.

### Example 23

### TNF-gamma-β is a Novel Ligand for DR3 and TR6-Alpha (DcR3) and Functions as a T cell Costimulator

Introduction

Members of the TNF and TNFR superfamilies of proteins are involved in the regulation of many important biological processes, including development, organogenesis, innate and adaptive immunity (Locksley et al., Cell 104:487-501 (2001)). Interaction of TNF ligands such as TNF, Fas, LIGHT and BLyS with their cognate receptor (or receptors) has been shown to affect the immune responses, as they are able to activate signaling pathways that link them to the regulation of inflammation, apoptosis, homeostasis, host defense, and autoimmunity. The TNFR superfamily can be divided into two groups based on the presence of different domains in the intracellular portion of the receptor. One group contains a TRAF binding domain that enables them to couple to TRAFs (TNFR-associated factor); these in turn activate a signaling cascade that results in the activation of NF-κB and initiation of transcription. The other group of receptors is characterized by a 60 amino acid globular structure named Death Domain (DD). Historically death domain-containing receptors have been described as inducers of apoptosis via the activation of caspases. These receptors include TNFR1, DR3, DR4, DR5, DR6 and Fas. More recent evidence (Siegel et al., Nature Immunology 1:469-474 (2000) and references within) has shown that some members of this subgroup of receptors, such as Fas, also have the ability to positively affect T cell activation. A third group of receptors has also been described. The members of this group, that include DcR1, DcR2, OPG, and TNFR-6 alpha (also called DcR3, and hereinafter in this example referred to as “TR6”), have been named decoy receptors, as they lack a cytoplasmic domain and may act as inhibitors by competing with the signal transducing receptor for the ligand (Ashkenazi et al., Curr. Opin. Cell Biol. 11:255-260 (1999)). TR6, which exhibits closest homology to OPG, associates with high affinity to FasL and LIGHT, and inhibits FasL-induced apoptosis both in vitro and in vivo (Pitti et al., Nature 396:699-703 (1998), Yu, et al., J. Biol. Chem. 274:13733-6 (1999); Connolly, et al., J. Pharmacol. Exp. Ther. 298:25-33 (2001)). Its role in down-regulating immune responses was strongly suggested by the observation that TR6 surpresses T-cell responses against alloantigen (Zhang et al., J. Clin. Invest. 107:1459-68 (2001)) and certain tumors overexpress TR6 (Pitti et al., Nature 396:699-703 (1998), Bai et al., Proc. natl. Acad. Sci. 97:1230-1235 (2000)).

DR3 (described in International Publication Numbers WO97/33904 and WO/0064465 which are herein incorporated by reference in their entireties) is a DD-containing receptor that shows highest homology to TNFR1 (Chinnaiyan et al., Science 274:990-2 (1996); Kitson et al., Nature 384:372-5 (1996), Marsters et al., Curr. Biol. 6:1669-76 (1996); Bodmer et al., Immunity 6:79-88 (1997); Screaton et al., Proc. Natl. Acad. Sci. 94:4615-19 (1997); Tan et al., Gene 204:35-46 (1997)). In contrast to TNFR1, which is ubiquitously expressed, DR3 appears to be mostly expressed by lymphocytes and is efficiently induced following T cell activation. TWEAK/Apo3L was previously shown to bind DR3 in vitro (Marsters et al., Curr. Biol. 8:525-528 (1998)). However, more recent work raised doubt about this interaction and showed that TWEAK was able to induce NF-κB and caspase activation in cells lacking DR3 (Schneider et al., Eur. J. Immunol. 29:1785-92 (1999); Kaptein et al., FEBS Letters 485:135-141 (2000)).

In this example, the characterization of the ligand, TNF-gamma-β (also known as TL1β; described in International Publication Numbers: WO00/08139 and WO00/66608 which are herein incorporated by reference in their entireties), for both DR3 and TR6/DcR3 is described. TNF-gamma-beta is a longer variant of TNF-gamma-alpha (also known as VEG1 and TL1; described in International Publication Numbers WO96/14328, WO99/23105, WO00/08139 and WO00/66608 which are herein incorporated by reference in their entireties), which was previously identified as an endothelial-derived factor that inhibited endothelial cell growth in vitro and tumor progression in vivo (Tan et al., Gene 204:35-46 (1997); Zhai et al., FASEB J. 13:181-9 (1999); Zhai et al., Int. J. Cancer 82:131-6 (1999); Yue et al., J. Biol. Chem. 274:1479-86 (1999)). It was found that TNF-gamma-beta is the more abundant form than TNF-gamma-alpha and is upregulated by TNFα and IL-1α. U.S. Pat. No. 5,876,969

As shown herein, the interaction between TNF-gamma-beta and DR3 in 293T cells and in the erythroleukemic line TF-1 results in activation of NF-KB and induction of caspase activity, respectively. TR6 is able to inhibit these activities by competing with DR3 for TNF-gamma-beta. More importantly, it was found that in vitro, TNF-gamma-beta functions specifically on activated T cells to promote survival and secretion of the proinflammatory cytokines IFNγ and GMCSF, and it markedly enhances acute graft-versus-host reactions in mice.

Results

### TNF-gamma-beta is a Longer Variant of TNF-gamma-alpha, a Member of the TNF Superfamily of Ligands

To identify novel TNF like molecules, a database of over three million human expressed sequence tag (EST) sequences was analyzed using the BLAST algorithm. Several EST clones with high homology to TNF like molecule 1, TNF-gamma-alpha (Tan et al., Gene 204:35-46 (1997); Zhai et al., FASEB J. 13:181-9 (1999); Yue et al., J. Biol. Chem 274:1479-86 (1999)) were identified from endothelial cell cDNA libraries. Sequence analysis of these cDNA clones revealed a 2080 base pair (bp) insert encoding an open reading frame of 251 amino acids (aa) with two upstream in-frame stop codons. The predicted protein lacks a leader sequence but contains a hydrophobic transmembrane domain near the N-terminus, and a carboxyl domain that shares 20-30% sequence similarity with other TNF family members. Interestingly, the C-terminal 151-aa of this protein (residues 101-251) is identical to residues 24 to 174 of TNF-gamma-alpha, whereas the amino-terminal region shares no sequence similarity. The predicted extracellular receptor-interaction domain of TNF-gamma-beta contains two potential N-linked glycosylation sites and shows highest amino acid sequence identity to TNF (24.6%), followed by FasL (22.9%) and LT (22.2%). A 337-bp stretch of the TNF-gamma-beta cDNA, containing most of the 5′ untranslated region and the sequences encoding the first 70 amino acids of the TNF-gamma-beta protein, matches a genomic clone on human chromosome 9 (Genbank Accession: AL390240, clone RP11-428F18). Further analysis of the human genomic sequences reveals that TNF-gamma-alpha and TNF-gamma-beta are likely derived from the same gene. While TNF-gamma-beta is encoded by four putative exons, similar to most TNF-like molecules, TNF-gamma-alpha is encoded by only the last exon and the extended N-terminal intron region, and therefore lacks a putative transmembrane domain and the first conserved-sheet

Mouse and rat TNF-gamma-beta cDNAs isolated from normal kidney cDNAs each encode a 252-aa protein. The overall amino acid sequence homology between human and mouse, and human and rat TNF-gamma-beta proteins is 63.7% and 66.1%, respectively. Higher sequence homology was found in the predicted extracellular receptor-interaction domains, of which human and mouse share 71.8% and human and rat share 75.1% sequence identity. An 84.2% sequence identity is seen between the mouse and rat TNF-gamma-beta proteins.

Like most TNF ligands, TNF-gamma-beta exists as a membrane-bound protein and can also be processed into a soluble form when ectopically expressed. The N-terminal sequence of soluble TNF-gamma-beta protein purified from full length TNF-gamma-beta transfected 293T cells was determined to be Leu 72.

TNF-gamma-beta is Predominantly Expressed by Endothelial Cells, a More Abundant Form than TNF-gamma-alpha, and is Inducible by TNF and IL-1α

To determine the expression pattern of TNF-gamma-beta, TNF-gamma-beta specific primer and fluorescent probe were used for quantitative real-time polymerase chain reaction (TaqMan) and reverse transcriptase polymerase chain reaction (RT-PCR) (see Experimental Procedures below). TNF-gamma-beta is expressed predominantly by human endothelial cells, including the umbilical vein endothelial cells (HUVEC), the adult dermal microvascular endothelial cells (HMVEC-Ad), and uterus myometrial endothelial cells (UtMEC-Myo), with highest expression seen in HUVEC. A 750 bp DNA fragment was readily amplified from these endothelial cells by RT-PCR, indicating the presence of full length TNF-gamma-beta transcripts. Very little expression was seen in human aortic endothelial cells (HAEC) or other human primary cells including adult dermal fibroblast (NHDF-Ad and HFL-1), aortic smooth muscle cells (AoSMC), skeletal muscle cells (SkMC), adult keratinocytes (NHEK-Ad), tonsillar B cells, T cells, NK cells, monocytes, or dendritic cells. Consistent with these results, TNF-gamma-beta RNA was detected in human kidney, prostate, stomach, and low levels were seen in intestine, lung, and thymus, but not in heart, brain, liver, spleen, or adrenal gland. No significant levels of TNF-gamma-beta mRNA in any of the cancer cell lines tested, including 293T, HeLa, Jurkat, Molt4, Raji, IM9, U937, Caco-2, SK-N-MC, HepG2, KS4-1, and GH4C were detected.

As the expression pattern of TNF-gamma-beta is very similar to that of TNF-gamma-alpha (Tan et al., Gene 204:35-46 (1997); Zhai et al., FASEB J. 13:181-9 (1999)), the relative abundance of the two RNA species was analyzed using TNF-gamma-alpha and TNF-gamma-beta specific primers and fluorescence probes for conventional and quantitative RT-PCR. More TNF-gamma-beta mRNA was detected than that of TNF-gamma-alpha using both methods. The amount of TNF-gamma-beta mRNA is at least 15-fold higher than that of TNF-gamma-alpha in the same RNA samples. To determine if TNF-gamma-beta mRNA levels were inducible, HUVEC cells were stimulated with either TNF, IL-1α, PMA, bFGF or IFNγ. PMA and IL-1α rapidly induced high levels of TNF-gamma-beta mRNA, with a peak in expression reached at 6 hours after treatment. TNF was also able to induce TNF-gamma-beta mRNA, but the time course of induction appeared to be delayed compared to PMA and IL-1α. In contrast, bFGF and IFNγ did not significantly affect the expression of TNF-gamma-beta. TNF-gamma-beta protein levels in the supernatants of activated HUVEC cells were analyzed by ELISA and a similar profile of induction was observed.

Identification of DR3 and TR6 as Receptors for TL1β

To identify the receptor for TNF-gamma-beta, we generated HEK293F stable transfectants expressing full length TNF-gamma-beta on the cell surface (confirmed by Taqman and flow cytometric analysis using TNF-gamma-beta monoclonal antibody). These cells were used to screen the Fc-fusion form of the extracellular domain of TNFR family members, including TNFR1, Fas, HveA, DR3, DR4, DR5, DR6, DcR1, DcR2, TR6, OPG, RANK, AITR, TAC1, CD40, and OX40. DR3-Fc and TR6-Fc bound efficiently to cells expressing TNF-gamma-beta but not to vector control transfected cells. In contrast, HveA-Fc and all the other receptors testeddid not bind to the TNF-gamma-beta expressing cells. TR6 has been previously described as a decoy receptor (Pitti et al., Nature 396:699-703 (1998); Yu et al., J. Biol. Chem. 274:13733-6 (1999)) capable of competing with Fas and HveA for binding of FasL and LIGHT, respectively. Whether TR6 could compete with DR3 for TNF-gamma-beta binding was tested. When a 2:1 molar ratio of a non-tagged form of TR6 and DR3-Fc were used, no binding of DR3-Fc was detected on TNF-gamma-beta expressing cells. These results demonstrated that both DR3 and TR6 can bind to membrane-bound form of the TNF-gamma-beta protein.

Whether TNF-gamma-beta protein could bind to membrane-bound form of the receptor, DR3 was tested. A FLAG-tagged soluble form of the TL1 (aa 72-251) protein was tested for binding of cells transiently transfected with different members of the TNFR family, including TNFR2, LT R, 4-1BB, CD27, CD30, BCMA, DR3, DR4, DR5, DR6, DcR1, DcR2, RANK, HveA, and AITR. Binding of FLAG-TL1β to cells expressing full length or DD-deleted DR3, but not to any of the other receptors tested, was consisitently detected, demonstrating that TNF-gamma-beta interacts with membrane-associated DR3. The small shift (˜30%) seen when full length DR3 was used is likely due to the presence of low DR3-expressing cells while DR3 overexpressed cells undergone apoptosis.

Coimmunoprecipitation studies were also performed to confirm that TNF-gamma-beta could specifically bind DR3 and TR6. Consistent with what we observed in FACS analysis, we found that DR3-Fc and TR6-Fc specifically interacted with FLAG-TNF-gamma-beta. In contrast, Fas-Fc or TACI-Fc could not immunoprecipitate FLAG-TNF-gamma-beta, but efficiently bound their known ligands, FLAG-FasL and FLAG-BlyS, respectively.

To verify that the TNF-gamma-beta binding to DR3 and TR6 was specific and exhibited characteristics that were similar to those observed with other TNF family members to their cognate receptors, a BIAcore analysis using a non-tagged TNF-gamma-beta (aa 72-251) protein purified from E. coli was perfomed. The kinetics of TNF-gamma-beta binding to DR3-Fc was determined using three different batches of the TNF-gamma-beta protein. The ka and kd values were found to be 6.39E+05 Ms⁻¹ and 4.13E-03M⁻¹ respectively. The average Kd value was 6.45±0.2 nM. TNF-gamma-beta was also examined for its ability to bind to several other TNF-related receptors (HveA, BCMA, TACI, and TR6). In addition to DR3, only TR6 was found to have significant and specific binding to TNF-gamma-beta. The ka and kd values were 1.04E+06 Ms⁻¹ and 1.9E-03 M⁻¹, respectively, which gives a Kd of 1.8 nM. The specificity of binding of TL1β to DR3-Fc and TR6-Fc were confirmed by the competition of TNF-gamma-beta binding in the presence of excess soluble receptor-Fc. These Kd values for binding of TNF-gamma-beta to DR3-Fc and TR6-Fc are comparable to those determined for other TNFR-ligand interactions.

Interaction of TL1β with DR3 Induces Activation of NF-κB

Previous reports have demonstrated that ectopic expression of DR3 results in the activation of the transcription factor NF-κB (Chinnaiyan et al., Science 274:990-2 (1996); Kitson et al., Nature 384:372-5 (1996), Marsters et al., Curr. Biol. 6:1669-76 (1996); Bodmer et al., Immunity 6:79-88 (1997)). TNF-gamma-beta induced signaling in a reconstituted system in 293T cells in which DR3 and a NF-κB-SEAP reporter were introduced by transient transfection was studied. To avoid spontaneous apoptosis or NF-κB activation accompanied with DR3 overexpression, a limited amount of DR3-expression DNA that by itself minimally activated these pathways was used. Under these conditions, cotransfection of cDNAs encoding full length or the soluble form of TNF-gamma-beta resulted in significant NF-B activation. This signaling event was dependent on the ectopic expression of DR3 and the intactness of the DR3 death domain, as TNF-gamma-beta alone or in combination with a DD-deleted DR3 did not induce NF-κB activation in these cells. Cotransfection of DR3 with cDNAs encoding TNF-gamma-alpha (full length or N-terminal 24-aa truncated) failed to induce NF-κB activation. A similar induction of NF-κB activity was observed when increasing amounts of recombinant TL1β protein (aa 72-251, with or without FLAG tag) were added to DR3 expressing cells. This induction of NF-κB was specifically inhibited by the addition of excess amount of DR3-Fc or TR6-Fc, but not by the addition of Fas-Fc or TNFR1-Fc. These results demonstrated that TNF-gamma-beta is a signaling ligand for DR3 that induces NF-κB activation, and TR6 can specifically inhibit this event.

TL1β Induces IL-2 Responsiveness and Cytokine Secretion from Activated T Cells

As DR3 expression is mostly restricted to the lymphocytes (Chinnaiyan et al., Science 274:990-2 (1996); Kitson et al., Nature 384:372-5 (1996); Marsters et al., Curr. Biol. 6:1669-76 (1996); Bodmer et al., Immunity 6:79-88 (1997); Screaton et al., Proc. Natl. Acad. Sci. 94:4615-19 (1997); Tan et al., Gene 204:35-46 (1997)) and is upregulated upon T cell activation, we examined the biological activity of TNF-gamma-beta on T cells. Recombinant TNF-gamma-beta (aa 72-251) protein was tested for its ability to induce proliferation of resting or costimulated T cells (treated with amounts of anti-CD3 and anti-CD28 that are not sufficient to induce proliferation). In resting or costimulated T cells, no significant increase in proliferation over background was observed. Interestingly, cells that were previously treated with TNF-gamma-beta for 72 hours were able to proliferate significantly in the presence of IL-2 than cells without TNF-gamma-beta preincubation, indicating that TNF-gamma-beta increases the IL-2 responsiveness of costimulated T cells.

As enhanced IL-2 responsiveness has been associated with increased IL-2 receptor expression and altered cytokine secretion, it was of interest to assess these responses on costimulated T cells treated with TNF-gamma-beta. TNF-gamma-beta treatment indeed upregulated IL-2Roc (CD25) and IL-2Rβ (CD122) expression from these cells. The extent of the increase in IL-2 receptor expression is consistent with the moderate increase in IL-2 responsiveness compared with 1L-2 itself. We next measured cytokine secretion from these cells and found that both IFNγ and GMCSF were significantly induced, whereas IL-2, IL-4, IL-10, or TNF were not. This effect was mostly dependent on the T cell coactivator CD28, as treatment of the cells with anti-CD3 and TNF-gamma-beta only minimally induced cytokine secretion. The effect that we observed on T cells was specifically mediated by TNF-gamma-beta, as addition of monoclonal neutralizing antibody to TL1β, or addition of DR3-Fc or TR6-Fc proteins was able to inhibit TNF-gamma-beta-mediated IFNγ secretion. TNF-gamma-beta was also tested on a variety of primary cells, including B cells, NK cells, and monocytes, but no significant activity was detected, suggesting a specific activity of TNF-gamma-beta on T cells.

TL1β Induces Caspase Activation in TF-1 Cells but not in T Cells

Overexpression of DR3 in cell lines induces capase activation (Chinnaiyan et al., Science 274:990-2 (1996); Kitson et al., Nature 384:372-5 (1996); Marsters et al., Curr. Biol. 6:1669-76 (1996); Bodmer et al., Immunity 6:79-88 (1997)). We tested whether TL1 could induce caspase activation in primary T cells. Purified T cells were activated with PHA and incubated with recombinant TNF-gamma-beta or FasL in the presence or absence of cycloheximide (CHX). No induction of caspase activity was detected in TNF-gamma-beta treated T cells, but was readily measured when cells were triggered with FasL, suggesting that under this experimental condition, TNF-gamma-beta does not activate caspases in T cells (the assay we used detects activation of caspases 2, 3, 6, 7, 8, 9, and 10). Various cell lines for the expression of DR3 and found that the erythroleukimic cell line TF-1 expressed high levels of DR3 were then analyzed. The effect of recombinant TNF-gamma-beta protein on caspase activation in TF-1 cells was then measured. In the absence of cycloheximide, no significant increase in caspase activity was detected following TNF-gamma-beta treatment, while TNF-gamma-beta was able to efficiently induce caspase activation in the presence of cycloheximide. This effect was inhibited by either DR3-Fc or TR6-Fc protein but not by LIGHT-Fc. An anti-TNF-gamma-beta monoclonal antibody was also shown to completely inhibit this activity, confirming that the caspase activation was mediated by TNF-gamma-beta.

TL1β Promotes Splenocyte Alloactivation in Mice

To determine if the in vitro activities of TNF-gamma-beta could be reproduced in vivo, a mouse model of acute graft-versus-host-response (GVHR) was developed in which parental C57BL/6 splenocytes were injected intravenously into (BALB/c X C57 BL/6) F1 mice (CB6F1), and the recipient's immune responses were measured. Typical alloactivation results in increased splenic weight of the recipient mice and enhanced proliferation and cytokine production of the splenocytes cultured ex-vivo (Via, J. Immunol. 146:2603-9 (1991); Zhang et al., J. Clin. Invest. 107:1459-68 (2001)). The large number of T cells in the spleen and their expected upregulation of DR3 in response to alloactivation makes this an ideal model to assess the effect of TNF-gamma-beta on a defined in vivo immune response. Five day administration of 3 mg/kg of the recombinant TNF-gamma-beta protein markedly enhanced the graft-versus-host responses. The mean (n=4) weight of normal spleens obtained from naive CB6F1 mice was 0.091 g. Alloactivation resulted in a 2.5 fold increase in splenic weight (˜0, 228 g). Treatment of allografted CB6F1 mice with recombinant TNF-gamma-beta protein (aa 72-251) further increased splenic weight about 50%, to a mean value of 0.349 g. TNF-gamma-beta treatment also significantly enhanced ex-vivo splenocyte expansion, and secretion of IFNγ and GMCSF. Thus, TNF-gamma-beta strongly enhances GVHR in vivo, and this effect is consistent with TNF-gamma-beta's in vitro activities.

Experimental Procedures

Cells, Constructs, and Other Reagents

All human cancer cell lines and normal lung fibroblast (HFL-1) were purchased from American Tissue Culture Collection. Human primary cells were purchased from Clonetics Corp. Cells were cultured as recommended. Human cDNA encoding the full length TNF-gamma-alpha, TNF-gamma-beta, DR3; the extracellular domain of TNF-gamma-alpha (aa 25-174), TNF-gamma-beta (aa 72-251), BlyS (aa 134-285), FasL (aa 130-281), and death domain truncated DR3 (DR3ADD, aa 1-345) were amplified by PCR and cloned into the mammalian expression vectors pC4 and/or pFLAGCMV1 (Sigma). The extracellular domain of human DR3 (aa 1-199), TACI (aa 1-159), HveA (aa 1-192), Fas (aa 1-169), and full length TR6 (aa 1-300), was each fused in-frame, at its C-terminus, to the Fe domain of human IgG1 and cloned into pC4. Rabbit polyclonal TNF-gamma-beta antibody was generated using recombinant TNF-gamma-beta (aa 72-251) protein and purified on a TNF-gamma-beta affinity column. Monoclonal antibodies were raised against recombinant TNF-gamma-beta as described (Kohler and Milstein, Nature 256:503-519 (1975)).

Cloning of Human, Mouse, and Rat TNF-gamma-beta cDNA

TNF-gamma-beta was identified by screening a human EST database for sequence homology with the extracellular domain of TNF, using the blastn and tblastn algorithms. The extracellular domain of the mouse and rat TNF-gamma-beta cDNA was isolated by PCR amplification from mouse or rat kidney Marathon-Ready cDNAs (Clontech) using human TNF-gamma-beta specific primers. The resulting sequences were then used to design mouse and rat TNF-gamma-beta specific primers to amplify the 5′ and 3′ ends of the cDNA using Marathon cDNA Amplification kit (Clontech). Each sequence was derived and confirmed from at least two independent PCR products.

Generation of TNF-gamma-beta Stable Cell Line

HEK293F cells were transiently transfected with pcDNA3.1(+) (vector control) or pcDNA3.1(+) containing full length TNF-gamma-beta. Cells resistant to 0.5 mg/ml Genticin (Invitrogen) were selected and expanded. Expression of TNF-gamma-beta mRNA was confirmed by quantitative RT-PCR analysis and surface expression of TNF-gamma-beta protein confirmed by FACS analyses using TNF-gamma-beta monoclonal antibodies.

Quantitative Real-Time PCR (TaqMan) and RT-PCR Analysis

Total RNA was isolated from human cell lines and primary cells using TriZOL (Invitrogen). TaqMan was carried out in a 25 microliter reaction containing 25 ng of total RNA, 0.6 μM each of gene-specific forward and reverse primers and 0.2 μM of gene-specific fluorescence probe. TNF-gamma-beta specific primers (forward: 5′-CACCTCTT AGAGCAGACGGAGATAA-3′ (SEQ ID NO:57), reverse: 5′-TTAAAGTGCTGTGTGG GAGTTTGT-3′ (SEQ ID NO:58), and probe: 5′-CCAAGGGCACACCTGACAGT TGTGA-3′ (SEQ ID NO:59)) amplify an amplicon span nucleotide 257 to 340 of the TNF-gamma-beta cDNA (aa 86-114 of the protein), while TNF-gamma-alpha specific primers (forward: 5′-CAAAGTCTACAGTTTCCCAATGAGAA-3′ (SEQ ID NO:60); reverse: 5′-GGGAACTGATTTTTAAAGTGCTGTGT-3′ (SEQ ID NO:61); probe: 5′-T CCTCTTTCTTGTCTTTCCAGTTGTGAGACAAAC-3′ (SEQ ID NO:62)) amplify nucleotide 17 to 113 of the TNF-gamma-alpha cDNA (aa 7-37 of the protein). Gene-specific PCR products were measured using an ABI PRISM 7700 Sequence Detection System following the manufacturer's instruction (PE Corp.). The relative mRNA level of TNF-gamma-beta was normalized to the 18S ribosomal RNA internal control in the same sample.

For RT-PCR analysis, 0.5 micrograms of total RNA was amplified with TNF-gamma-alpha (5′-GCAAAGTCTACAGTTTCCCAATGAGAAAATTAATCC-3′(SEQ ID NO:63)) or TNF-gamma-beta specific sense primer (5′-ATGGCCGAGGATCTGGG ACTGAGC-3′ (SEQ ID NO:64)) and an antisense primer (5′-CTATAGTAAG AAGGCTCCAAAGAAGGTTTTATCTTC-3′ (SEQ ID NO:65)) using SuperScript One-Step RT-PCR System (Invitrogen). β-actin was used as internal control.

Transfection and NF-κB Reporter Assay

293T cells were transiently transfected using LipofectAMINE and PLUS reagents according to the manufacturer's instruction (Invitrogen). For reporter assays, 293T cells, at 5×10⁵ cells/well, were seeded in 6-well plates and transfected with a total of 1 microgram of DNA. pC4 DNA was used as filler DNA. Conditioned supernatant was collected 24 hr post-transfection and assayed for secreted alkaline phosphatase (SEAP) activity using the Phospha-Light™ chemiluminescent reporter gene assay system (Tropix). pCMV-lacZ was used as internal control for transfection efficiency normalization.

Recombinant Protein Purification

FLAG fusion proteins were produced from 293T cells by transient transfection, and purified on anti-Flag M2 affinity columns (Sigma) according to manufacturer's instruction. Receptor proteins with or without Fc fusion were produced from Baculovirus or CHO stable cell lines as described (Zhang et al., J. Clin. Invest. 107:1459-68 (2001)). Recombinant, untagged TNF-gamma-beta protein (aa 72-251) was generated and purified from E. coli. Briefly, E. Coli cell extract was separated on a HQ-50 anion exchange column (Applied Biosystems) and eluted with a salt gradient. The 0.2 M NaCl elution was diluted and loaded on a HQ-50 column, and the flow through was collected, adjusted to 0.8 M ammonia sulfate and loaded on a Butyl-650s column (Toso Haus). The column was eluted with a 0.6M to 0 M ammonia sulfate gradient and the fractions containing TNF-gamma-beta protein were pooled and further purified by size exclusion on a Superdex-200 column (Pharmacia) in PBS. All recombinant proteins were confirmed by NH₂-terminal sequencing on a ABI-494 sequencer (Applied Biosystem). The endotoxin level of the purified protein was less than 10 EU/mg as measured on a LAL-5000E (Cape Cod Associates).

Flow Cytometry, Immunoprecipitation, and Western Blot Analysis

One million cells, in 0.1 ml of FACS buffer (PBS, 0.1% BSA, 0.1% NaN₃), were incubated with 0.1-1 microgram of protein or antibody at RT for 15 min. The cells were washed with 3 ml of FACS buffer, reacted with biotinylated primary antibody, and stained with PE-conjugated secondary antibody at RT for 15 min. Cells were then washed again, resuspended in 0.5 microgram/ml of propidium iodide, and live cells were gated and analyzed on a FACScan using the CellQuest software (BD Biosciences).

For coimmunoprecipiation studies, 2 micrograms each of purified TNFR-Fc proteins was incubated with 1 microgram of Flag-tagged TNF-gamma-beta, FasL or BlyS protein and 20 microliters of protein A-Sepharose beads in 0.5 ml of IP buffer (DMEM, 10% FCS, 0.1% Triton X-100) at 4° C. for 4 hr. The beads were then precipitated and washed extensively with PBST buffer (PBS, 0.5% Triton X-100) before boiled in SDS-sample buffer. Proteins were resolved on 4-20% Tris-Glycine gels (NOVEX), transferred to nitrocellulose membranes, and blotted with anti-Flag M2 monoclonal antibody (1 microgram/ml, Sigma) and horseradish peroxidase (HRP)-conjugated goat anti-mouse IgG antibody (0.5 microgram/ml).

BIAcore Analysis

Recombinant TNF-gamma-beta (from E. Coli) binding to various human TNF receptors was analyzed on a BIAcore 3000 instrument. TNFR-Fc were covalently immobilized to the BIAcore sensor chip (CM5 chip) via amine groups using N-ethyl-N′-(dimethylaminopropyl) carbodiimide/N-hydroxysuccinimide chemistry. A control receptor surface of identical density was prepared, BCMA-Fc, that was negative for TNF-gamma-beta binding and used for background subtraction. Eight different concentrations of TNF-gamma-beta (range: 3-370 nM) were flowed over the receptor-derivatized flow cells at 15 microliters/min for a total volume of 50 microliters. The amount of bound protein was determined during washing of the flow cell with HBS buffer (10 mM HEPES, pH 7.4, 150 mM NaCl, 3.4 mM EDTA, 0.005% Surfactant P20). The flow cell surface was regenerated by displacing bound protein by washing with 20 microliters of 10 mM glycine-HCl, pH 2.3. For kinetic analysis, the on and off rates were determined using the kinetic evaluation program in BIAevaluation 3 software using a 1:1 binding model and the global analysis method.

T cell Proliferation Assays

Whole blood from human donors was separated by Ficoll (ICN Biotechnologies) gradient centrifugation and cells were cultured overnight in RPMI containing 10% FCS (Biofluids). T cells were separated using the MACS PanT separation kit (Milteny Biotech), the T cell purity achieved was usually higher that 90%. The cells were seeded on anti-CD3 (0.3 microgram/ml, Pharmingen) and anti-CD28 (5.0 4 microgram/ml) coated 96-well plates at 2×10⁴/well, and were incubated with medium alone, 1 ng/ml of IL-2 (R & D Systems), or 100 ng/ml of TNF-gamma-beta (aa 72-251) at 37° C. After 72 hour in culture, the cells were either untreated or treated with 1 ng/ml of IL-2, and pulsed with 0.5 μCi of ³H-thymidine for another 24 hours and incorporation of ³H measured on a scintillation counter.

Cytokine ELISA Assays for Primary Cells

1×10⁵ cells/ml of purified T cells were seeded in a 24-well tissue culture plate that had been coated with anti-CD3 (0.3 microgram/ml) and anti-CD28 (5.0 microgram/ml) overnight at 4° C. Recombinant TNF-gamma-beta (aa72-251) protein (100 ng/ml) was added to cells and supernatants were collected 72 hours later. ELISA assay for IFNγ, GM-CSF, IL-2 IL-4, IL-10 and TNFα were performed using kits purchased from R & D Systems. Recombinant human IL-2 (5 ng/ml) was used as a positive control. All samples were tested in duplicate and results were expressed as an average of duplicate samples plus or minus error.

Caspase Assay

TF-1 cells or PHA-activated primary T cells were seeded at 75,000 cells/well in a black 96-well plate with clear bottom (Becton Dickinson) in RPMI Medium containing 1% fetal bovine serum (Biowhittaker). Cells were treated with TNF-gamma-beta (aa72-251, 100 ng/ml) in the presence or absence of cycloheximide (10 micrograms/ml). Caspase activity was measured directly in the wells by adding equal volume of a lysis buffer containing 25 μM DEVD-rodamine 110 (Roche Molecular Biochemicals), and allowed the reaction to proceed at 37C for 1 to 2 hours. Release of rodamine 110 was monitored with a Wallac Victor2 fluorescence plate reader with excitation filter 485 nm and emission filter 535 nm.

For the inhibition studies using Fc-proteins or antibodies, the indicated amount of each protein was mixed with either medium or 100 ng/ml of TNF-gamma-beta in the presence or absence of cycloheximide. The reagents were incubated for 1 hour at RT to allow the formation of protein-TNF-gamma-beta complexes and then added to the cells. Caspase activity was measured as described above.

Murine Graft-Versus-Host Reaction

The F1 (CB6F1) of C57BL/6×BALB/c mice (H-2^{bxd}) were transfused intravenously with 1.5×10⁸ spleen cells from C57BL/6 mice (H-2^{b}) on day 0. Recombinant TNF-gamma-beta (aa 72-251) protein or buffer alone was administered intravenously daily for 5 days at 3 mg/kg/day starting on the same day as the transfusion. The spleens of the recipient F1 mice were harvested on day 5, weighed and single cell suspensions prepared for in vitro assays.

Ex-vivo Mouse Splenocyte Alamar Blue and Cytokine Assays

Splenocytes from normal and the transfused F1 mice were cultured in triplicate in 96-well flat-bottomed plates (4×10⁵ cells/200 microliters/well) for 2-4 days. After removing 100 microliters of supernatant per well on the day of harvest, 10 microliters Alamar Blue (Biosource) was added to each well and the cells cultured for additional 4 h. The cell number in each well was assessed according to OD_{590 nm} minus OD530 nm background, using a CytoFluor apparatus (PerSeptive Biosystems). Cytokines in the culture supernatant were measured with commercial ELISA kits from Endogen or R & D Systems following manufacturer's instructions.

### Example 24

### Refolding of TNFR-6 Alpha from Inclusion Bodies

Materials and Methods:

Reagents were of analytical grade and, unless stated otherwise in the protocol, purchased from Merck Eurolab. L-arginine was obtained from Ajinimoto Inc, kanamycin from Sigma, lysozyme from Sigma, Alamar Blue from Biosource and FasL-FALG from Alexis. Water was filtrated with a Milli-Q system (Millipore).

Protein marker: LMW-marker (Pharmacia, 17-0615-01), stock solution:

| | Molecular | Concentration |
|---|---|---|
| Protein | Weight (kDa) | (ug/mL) |
| phosphorylase b | 97.0 | 67 |
| albumin | 66.0 | 83 |
| Ovalbumin | 45.0 | 147 |
| Carboanhydrase | 30.0 | 83 |
| Trypsin inhibitor | 20.1 | 80 |
| Alpha-lactalbumin | 14.4 | 116 |

SDS-PAGE

The method of Laemmli (1970) was used as the basis for SDS-PAGEs. Concentration of acrylamide was always 15%. Every protein sample was boiled at 95 C for 5 min after addition of SDS-sample buffer and subsequently centrifugated for 5 min at 13,000 rpm (Centrifuge: Biofuge Pico, Heraeus). SDS-PAGE gels ran 70 min at 150 V in a Mini-Protean system (BioRad). Silver staining of SDS gels was done according to the protocol of Nesterenko et al. J. Biochem. Biophys. Meth. 28 (1984) 239-242).

Buffer Systems:

DS-sample buffer: 250 mM Tris/HCl, pH 8.0; 40% (v/v) glycerine; 5% (w/v) SDS; 5% (v/v) mercaptoethanol

Running buffer: 50 mM Tris/HCl; 19 mM glycine; 0.2% (w/v) SDS

Lower gel buffer (end concentration): 600 mM Tris/HCl, pH 8.0; 0.8% (w/v) SDS

Upper gel buffer (end concentration): 100 mM Tris/HCl, pH 6.8; 0.8% (w/v) SDS

Methods for Determination of Protein Concentrations

Bio-RAD protein assay (Cat. No. 500-0006) with BSA as a standard.

UV-vis-spectra using the theoretical ε_{280 nm} (23390 M⁻¹ cm⁻¹; http://www.expasy.ch/cgi-bin/protparam) were carried out on a Cary 300 system (Varian Inc.). An OD₂₈₀ of 0.716 corresponds to a solution of TNFR-6 alpha amino acid residues 30-300 of SEQ ID NO:2, hereinafter in this example “TNFR-alpha”) with a concentration of 1 mg/ml.

Bacterial Strains and Growth Media

BL21 (DE3) purchased from Novagen

LB 0.5 g NaCl, 0.5 g yeast extract, 1 g tryptone in 1 L water

LB-Agar LB with 15 g Agar-Agar per L water

2xYT 17 g tryptone, 10 g yeast extract, 5 g NaCl in 1 L water

SOC 20 g tryptone, 5 g yeast extract, 10 mM NaCl, 2.5 mM KCl, 10 mM MgSO₄, 10 mM MgCl₂, 0.4 g glucose in 1 L water

Mammalian Cells

Jurkat E6-1 cells (ATCC: TIB-152) were used in the apoptosis assay

Experimental Protocol:

Transformation of E. coli BL2] (DE3) and Cultivation

E. coli BL21 (DE3) cells were transformed with the pHE4 vector (ATCC Dpeosit Number 209645, described in U.S. Pat. No. 6,194,168) containing a polynucleotide encoding amino acid residues 30-300 of SEQ ID NO:2) using a Bio-Rad GenePulser 11 system (2.5 kV, 200 Q, Cuvette with a 2 mm gap). Cells were immediately transferred to SOC medium and shaken for 40 min at 37degrees C. and 600 rpm (Eppendorf Thermomixer Compact). They were subsequently plated on LB-Agar petri dishes containing 50 micrograms/ml kanamycin and grown overnight at 37 degrees C. A single colony was used for an overnight culture and grown in 175 ml LB medium containing 50 micrograms/ml kanamycin at 37 degrees C. and 200 rpm for 14 h. 4×5 L erlenmeyer flasks containing 1.5 L 2xYT medium with 50 micrograms/ml kanamycin were inoculated with 30 ml overnight culture each and grown at 37 degrees C. and 200 rpm for 4 h (until the OD₆₀₀ reached 1). Afterwards, cells were induced by addition of 3 mM IPTG and cultivated as before for 3 h more. Harvest was done using a Beckman Avanti J-20 centrifuge and a JLA 8.1000 rotor at 5000 g and 4 degrees C. for 10 min. Cell pellets were frozen and stored at −20 degrees C.

Preparation of Inclusion Bodies

15 g cells were thawed and homogenized in 75 ml 0.1 M Tris-HCl, pH 7.0, 1 mM EDTA using an ultraturrax. After addition of 23 mg lysozyme, the cells were mixed shortly with an ultraturrax and incubated at 4 degrees C. for 30 min. Subsequently 15,000 U benzonase and 3 mM MgCl₂ were added and the mixture was incubated at 25 degrees C. for 10 min. Cells were disrupted using a Constant Systems Z-Plus high-pressure homogenizer at 1,800 bar (two passages). 0.5 vol. of 67 mM EDTA, 6% Triton X-100, 1.5 M NaCl pH 7.0 were added and the homogenate was incubated at 4 degrees C. for 30 min. Inclusion bodies were sedimented by centrifugation at 4 degrees C. and 32,000 g for 10 min (Beckman Avanti J-25 centrifuge; JLA 16.250 rotor). Inclusion bodies were resuspended in 120 ml 0.1 M Tris-HCl, pH 7.0, 20 mM EDTA using an ultraturrax. The centrifugation step and the resuspension were repeated 4 times and the resulting inclusion bodies were stored at −20 degrees C. For following analysis of inclusion bodies in SDS-PAGE a very diminutive amount is sufficient.

Solubilization of TNFR-6 Alpha Inclusion Bodies

TNFR-6 alpha inclusion bodies were solubilized by dilution of approximately 1 g IBs into 15 ml solubilization buffer (100 mM Tris, pH 8.0; 8 M guanidiniumhydrochloride; 100 mM dithiothreitol, 1 mM EDTA) and incubated on a roller shaker at room temperature for 3 h. After centrifugation at 75,000 g (4 degrees C.; 1 h; Beckman centrifuge Avanti J-25; JA 25.50 rotor) the pH of the supernatant was lowered to 3-4 by dropwise addition of 1 M HCl Two dialysis steps for 2 h at room temperature against 4 M guanidiniumhydrochloride, 10 mM HCl using Spectra/Por dialysis membranes (MWCO 6000-8000 Da; Reorder-No. 132 650) followed by a dialysis against 4 M guanidiniumhydrochloride at 7 degrees C. overnight were carried out to remove dithiothreitol. Protein concentration was determined by UV-vis spectroscopy using the theoretical extinction coefficient of TNFR-6 alpha (see materials and methods).

Refolding of TNFR-6 Alpha

16.7 mg solubilized TNFR-6 alpha (21.6 mg/ml concentration) were added dropwise (under stirring) to 200 ml refolding buffer (50 mM BICINE, pH 9.0, 1 M L-arginine, 0.5 M NaCl, 5 mM oxidized glutathione, 1 mM reduced glutathione) at a temperature of 7 degrees C. This addition was repeated twice after 2 and 4 h, respectively. The solution was stirred gently overnight (approximately 20 h). After centrifugation at 4 degrees C. and 75,000 g (Beckman Avanti J-25 centrifuge, JA 25.50 rotor) for 1 h, the supernatant was used for buffer exchange.

Buffer Exchange

Buffer exchange took place by applying 60 ml of the refolding samples on an XK 50/20 column packed with 300 ml sephadex G-25 fine (Amersham Pharmacia Biotech; Cat. No. 170032-01), equilibrated with elution buffer (50 mM Na₂HPO₄, pH 7.5; 50 mM NaCl). The flow rate was 5 (injection) or 10 ml/min (elution) using a Pharmacia FPLC system at 7 degrees C. At the elution peak of proteins (rise of extinction at 280 nm) 10 fractions of 10 ml each were collected and fractions 2-7 pooled. Buffer exchange was repeated twice and the fractions containing TNFR-6 alpha were pooled. The protein concentration of the supernatant was determined and samples were taken for SDS-PAGE and activity assay.

Further Purification of TNFR-6 Alpha Using Ion Exchange Chromatography

TNFR-6 alpha fractions from buffer exchange were applied on a 1 ml HiTrap column packed with SP sepharose XL (Amersham Pharmacia Cat.-No. 17-5160-01), equilibrated with 50 mM Na₂HPO₄, pH 7.5; 50 mM NaCl. The flow rate was 0.5 ml/min. Afterwards the column was washed with 20 column volumes 50 mM Na2HPO₄, pH 7.5; 50 mM NaCl. TNFR-6 alpha was eluted by a step-gradient to 50 mM Na₂HPO₄, pH 7.5; 390 mM NaCl and collected in fractions of 1 ml each. Samples of peak fractions were used for determination of protein concentration and SDS/PAGE. Fractions containing TNFR-6 alpha were pooled and tested in the activity assay.

Determination of Activity

The determination of refolded TNFR-6-alpha protein activity was assessed using the in vitro soluble human FasL mediated cytoxicity assay largely as described in Example 22. A few minor modifications to the assay were made: Jurkat-E6 cells were used rather than HT-29 cells; the cell number per well was 10,000 rather than 50,000; the incubation time in the presence of alamar blue was 56 hours rather than 4 hours; and absorption measurements were carried out ar 620 nm. Concentrations of TNFR-6 alpha tested in the assay were 100 ng/ml, 1 microgram/ml and 10 micrograms/ml.

Aliquoting of Samples

Because TNFR-6 alpha tends to aggregate at concentrations above 1 mg/ml the sample was diluted to 0.7 mg/ml with 50 mM Na₂HPO₄, pH 7.5; 390 mM NaCl. Samples of 1 ml were aliquoted into 1.5 ml eppendorf tubes, frozen in liquid nitrogen and stored at −80 degrees C.

Results:

Cultivation and Preparation of Inclusion Bodies

From 6 L shake flask culture, 24 grams cells (wet weight) were obtained. These cells yielded approximately 1.5 grams inclusion bodies. The inclusion body preparation contained about 70% TNFR-6-alpha (residues 30-300) (estimation from SDS-PAGE).

Solubilization of TNFR-6 Alpha

From 1 grams inclusion bodies approximately 400 mg solubilized protein could be prepared. In general it is preferrable to have protein solubilisate with a high protein content to prevent adding too much guanidiniumhydrochloride to the refolding reaction. With our procedure we were able to obtain a solubilisate with 22 mg/ml protein content (estimated with the theoretical extinction coefficient of TNFR-6 alpha).

Refolding of TNFR-6 Alpha and Buffer Exchange

Optimal time for refolding was one day. After two days of refolding the yield decreased by approximately 40%. To find the optimal protein concentration for the refolding of TNFR-6 alpha, we tested concentrations ranging from 50-400 micrograms/ml. Although the yield of soluble protein was slightly higher at lower concentrations we chose 250 micrograms/ml, a concentration that yielded at least 55% soluble protein after refolding and avoided working with high refolding volumes. Even though only a small aggregation pellet appeared, about 60% of the initial protein amount were detected by protein determination using Bio-Rad protein assay after centrifugation (refolding yield). The pooled fractions obtained after buffer exchange contained 95% of the applied protein amount.

Purification of Refolded TNFR-6 Alpha by Ion Exchange Chromatography

Because TNFR-6 alpha inclusion bodies and solubilisate contained a high content of other proteins that may interfere with the activity assay we attempted to purify the protein using liquid chromatography. Because of the high theoretical pI we have chosen cation exchange chromatography. TNFR-6 alpha bound to SP sepahroseXL in the presence of 50 mM NaCl and could be eluted with 390 mM NaCl. The main protein contaminants did not bind to this material or eluted at a higher concentration of NaCl (see FIG. 3). TNFR-6 alpha could be purified to at least 90% (estimated from a silver stained SDS-PAGE; FIG. 3). Typical fractions contained 0.5-1.5 mg/ml TNFR-6 alpha. At concentrations above 1 mg/ml, the solution became sometimes turbid indicating aggregation of TNFR-6 alpha. We therefore diluted the pooled samples to a concentration below 1 mg/ml. To avoid aggregation we recommend to use a linear gradient to keep the potein concentration during elution low. Fractions eluted by a 100% step of high salt contained TNFR-6 alpha only at approximately 50%.

Because of aggregation, the yield of this purification procedure was less than 50% of the applied TNFR-6 alpha. So the overall yield of this step-gradient procedure, referred to the TNFR-6 alpha content, is 20% (Table 1).

Calculation of yield of the used refolding and purification steps

| | | Estmated | | | | |
|---|---|---|---|---|---|---|
| | | purity of | | Yield | | Overall |
| | | TNFR-6 (from | | (referred to | | (referred |
| | Applied | SDS-PAGE) | | total | | to TNFR- |
| Step | protein | before | after | protein content) | | 6 alpha) |
| Refolding | 50 mg | 70% | 70% | 30 mg | 60% | 60% |
| Buffer | 28 mg | 70% | 70% | 27 mg | 95% | 57% |
| exchange | | | | | | |
| Ion exchange | 17 mg | 70% | >90% | 4.6 mg | 27% | 20% |
| chromatography | | | | | | |

Pooled fractions of purified TNFR-6 alpha were tested for activity immediately or frozen in liquid nitrogen, stored at −80degrees C. and tested after 5 days in the activity assay.

Activity of the Refolded and Purified Samples

We used the determination of the absorption at 620 nm for the activity assay. With viable cells (without FasL-FLAG) the absorption was around 0.4 and with apoptotic cells (with FasL FLAG without TNFR-6 alpha) the absorption rose to 0.7. The refolded samples of TNFR-6 alpha and the positive control showed activity in a range from 1-10 micrograms/ml, but not below (e.g., at 1100 ng/ml). The further purified material from refolding showed a higher activity than samples after buffer exchange without further purification. This may be due to the lower purity of the samples from buffer exchange, that only contain approximately 70% TNFR-6 alpha. But it clearly shows that active (and not just soluble) TNFR-6 alpha can be obtained by refolding even at this high refolding yoeld (approximately 60% refolding yield).

Storage of refolded TNFR-6 alpha at −80 degrees C. has only a slight influence on the activity in the apoptosis assay.

### CONCLUSIONS

This refolding protocol in connection with the purification of TNFR-6 alpha by cation exchange chromatography can be used to produce TNFR-6 alpha at a mg-scale. From 6 L shake flask culture (24 grams wet cell weight) approximately 70 mg active TNFR-6 alpha with a purity of at least 90% can be obtained. After refolding and buffer exchange, a yield of 60%, referred to the employed amount of solubilized protein at the beginning of refolding.

### Example 25

### Expression and Purification in E. coli

The DNA sequence encoding the mature DR3-V1 protein in the cDNA contained in ATCC No. 97456 is amplified using PCR oligonucleotide primers specific to the amino terminal sequences of the DR3-V1 protein and to vector sequences 3′ to the gene. Additional nucleotides containing restriction sites to facilitate cloning are added to the 5′ and 3′ sequences respectively.

The following primers are used for expression of DR3 extracellular domain in E. coli. 5′ primer: 5′-GCGCCATGGGGGCCCGGCGGCAG-3′ (SEQ ID NO:66) contains an NcoI site and 15 nucleotide, from nucleotide 290 to nucleotide 304 in SEQ ID NO:3. 3′ primer: 5′-GCGAAGCTTCTAGGACCCAGAACATCTGCC-3′ (SEQ ID NO:67) contains a HindIII site, a stop codon and 18 nucleotides complimentary to nucleotides from 822 to 840 in SEQ ID NO:3. Vector is pQE60. The protein is not tagged.

The restriction sites are convenient to restriction enzyme sites in the bacterial expression vector pQE60, which are used for bacterial expression in these examples. (Qiagen, Inc. 9259 Eton Avenue, Chatsworth, Calif., 91311). pQE60 encodes ampicillin antibiotic resistance (“Ampr”) and contains a bacterial origin of replication (“ori”), an IPTG inducible promoter, a ribosome binding site (“RBS”).

The amplified DR3-V1 DNA and the vector pQE60 both are digested with NcoI and HindIII and the digested DNAs are then ligated together. Insertion of the DDCR protein DNA into the restricted pQE60 vector places the DR3-VI protein coding region downstream of and operably linked to the vector's IPTG-inducible promoter and in-frame with an initiating AUG appropriately positioned for translation of DR3-V1 protein.

The ligation mixture is transformed into competent E. coli cells using standard procedures. Such procedures are described in Sambrook et al., Molecular Cloning: a Laboratory Manual, 2nd Ed.; Cold Spring Harbor Laboratory Press, Cold Spring Harbor, N.Y. (1989). E. coli strain M15/rep4, containing multiple copies of the plasmid pREP4, which expresses lac repressor and confers kanamycin resistance (“Kan^{r} ”), is used in carrying out the illustrative example described herein. This strain, which is only one of many that are suitable for expressing DR3-V1 protein, is available commercially from Qiagen.

Transformants are identified by their ability to grow on LB plates in the presence of ampicillin and kanamycin. Plasmid DNA is isolated from resistant colonies and the identity of the cloned DNA confirmed by restriction analysis.

Clones containing the desired constructs are grown overnight (“O/N”) in liquid culture in LB media supplemented with both ampicillin (100 μg/ml) and kanamycin (25 μg/ml).

The O/N culture is used to inoculate a large culture, at a dilution of approximately 1:100 to 1:250. The cells are grown to an optical density at 600 nm (“OD600”) of between 0.4 and 0.6. Isopropyl-B-D-thiogalactopyranoside (“IPTG”) is then added to a final concentration of 1 mM to induce transcription from lac repressor sensitive promoters, by inactivating the lacI repressor. Cells subsequently are incubated further for 3 to 4 hours. Cells then are harvested by centrifugation and disrupted, by standard methods. Inclusion bodies are purified from the disrupted cells using routine collection techniques, and protein is solublized from the inclusion bodies into 8M urea. The 8M urea solution containing the solublized protein is passed over a PD-10 column in 2×phosphate-buffered saline (“PBS”), thereby removing the urea, exchanging the buffer and refolding the protein. The protein is purified by a further step of chromatography to remove endotoxin. Then, it is sterile filtered. The sterile filtered protein preparation is stored in 2×PBS at a concentration of 95 μ/ml.

### Example 26

### Expression of Extracellular Soluble Domain of DR3-V1 and DR3 in COS Cells

The expression plasmid, pDR3-V1 HA, is made by cloning a cDNA encoding DR3-V1 (ATCC No. 97456) into the expression vector pcDNAI/Amp (which can be obtained from Invitrogen, Inc.). Expression plasmid, pDR3 HA, is made by cloning a cDNA encoding DR3 (ATCC No. 97757) into the expression vector pcDNAI/Amp.

The expression vector pcDNAI/amp contains: (1) an E. coli origin of replication effective for propagation in E. coli and other prokaryotic cell; (2) an ampicillin resistance gene for selection of plasmid-containing prokaryotic cells; (3) an SV40 origin of replication for propagation in eukaryotic cells; (4) a CMV promoter, a polylinker, an SV40 intron, and a polyadenylation signal arranged so that a cDNA conveniently can be placed under expression control of the CMV promoter and operably linked to the SV40 intron and the polyadenylation signal by means of restriction sites in the polylinker.

A DNA fragment encoding the entire DR3-V1 or Dr3 precursor and a HA tag fused in frame to its 3′ end is cloned into the polylinker region of the vector so that recombinant protein expression is directed by the CMV promoter. The HA tag corresponds to an epitope derived from the influenza hemagglutinin protein described by Wilson et al., Cell 37:767 (1984). The fusion of the HA tag to the target protein allows easy detection of the recombinant protein with an antibody that recognizes the HA epitope.

The plasmid construction strategy is as follows:

The DR3-V1 or DR3 cDNA of the deposit cDNA is amplified using primers that contained convenient restriction sites, much as described above regarding the construction of expression vectors for expression of DR3-V1 or DR3 in E. coli and S. fugiperda.

To facilitate detection, purification and characterization of the expressed DR3-V1 or DR3, one of the primers contains a hemagglutinin tag (“HA tag”) as described above.

Suitable primers for DR3 include the following, which are used in this example, the 5′ primer: 5′CGCGGATCCGCCATCATGGAGCAGCGGCCGCGG 3′ (SEQ ID NO:68) contains the underlined BamHI site, an ATG start codon and 5 codons thereafter.

The 3′ primer for DR3, containing the underlined XbaI site, stop codon, hemagglutinin tag and last 14 nucleotide of 3′ coding sequence (at the 3′ end) has the following sequence: 5′GCGTCTAGATCAAAGCGTAGTCTGGGACGTCGTATGGG TACGGGCCGCGCTGCA3′ (SEQ ID NO:69).

The PCR amplified DNA fragment and the vector, pcDNAI/Amp, are digested with BamHI and XbaI and then ligated. The ligation mixture is transformed into E. coli strain SURE (available from Stratagene Cloning Systems, 11099 North Torrey Pines Road, La Jolla, Calif. 92037) the transformed culture is plated on ampicillin media plates which then are incubated to allow growth of ampicillin resistant colonies. Plasmid DNA is isolated from resistant colonies and examined by restriction analysis and gel sizing for the presence of the DR3-V1 or DR3-encoding fragment.

For expression of recombinant DR3-V1 or DR3, COS cells are transfected with an expression vector, as described above, using DEAE-DEXTRAN, as described, for instance, in Sambrook et al., Molecular Cloning: a Laboratory Manual, Cold Spring Laboratory Press, Cold Spring Harbor, N.Y. (1989).

Cells are incubated under conditions for expression of DR3-V1 or DR3 by the vector.

Expression of the DR3-VI HA fusion protein or the DR3 HA fusion protein is detected by radiolabelling and immunoprecipitation, using methods described in, for example Harlow et al., Antibodies: a Laboratory Manual, 2nd Ed., Cold Spring Harbor Laboratory Press, Cold Spring Harbor, N.Y. (1988). To this end, two days after transfection, the cells are labeled by incubation in media containing ³⁵S-cysteine for 8 hours. The cells and the media are collected, and the cells are washed and then lysed with detergent-containing RIPA buffer: 150 mM NaCl, 1% NP-40, 0.1% SDS, 1% NP-40, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE gels and autoradiography. An expression product of the expected size is seen in the cell lysate, which is not seen in negative controls.

### Example 27

### Expression and Purification of Human DR3-V1 and DR3 Using the CHO Expression System

The vector pC1 is used for the expression of DR3-V1 or DR3 (ATCC No. 97456 or ATCC No. 97757, respectively) protein. Plasmid pC1 is a derivative of the plasmid pSV2-dhfr (ATCC Accession No. 37146). Both plasmids contain the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., F. W. Alt et al., J. Biol. Chem. 253:1357-1370 (1978); J. L. Hamlin and C. Ma, Biochem. et Biophys. Acta, 1097:107-143 (1990); M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene it is usually co-amplified and over-expressed. It is state of the art to develop cell lines carrying more than 1,000 copies of the genes. Subsequently, when the methotrexate is withdrawn, cell lines contain the amplified gene integrated into the chromosome(s).

Plasmid pC1 contains for the expression of the gene of interest a strong promoter of the long terminal repeat (LTR) of the Rous Sarcoma Virus (Cullen et al., Molecular and Cellular Biology 5:438-447 (March 1985)), plus a fragment isolated from the enhancer of the immediate early gene of human cytomegalovirus (CMV) (Boshart et al., Cell 41:521-530 (1985)). Downstream from the promoter are the following single restriction enzyme cleavage sites that allow the integration of the genes: BamHI followed by the 3′ intron and the polyadenylation site of the rat preproinsulin gene. Other high efficient promoters can also be used for the expression, e.g., the human P-actin promoter, the SV40 early or late promoters or the long terminal repeats from other retroviruses, e.g., HIV and HTLVI. For the polyadenylation of the mRNA other signals, e.g., from the human growth hormone or globin genes can be used as well.

Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC1 is digested with the restriction enzyme BamHI and then dephosphorylated using calf intestinal phosphates by procedures known in the art. The vector is then isolated from a 1% agarose gel.

The DNA sequence encoding DR3-V1 or DR3 in the deposited cDNA is amplified using PCR oligonucleotide primers specific to the amino acid carboxyl terminal sequence of the DR3-V1 or DR3 protein and to vector sequences 3′ to the gene. Additional nucleotides containing restriction sites to facilitate cloning are added to the 5′ and 3′ sequences respectively.

The 5′ oligonucleotide primer for DR3 has the sequence: 5′ CG CGGATCCGCCATCATGGAGCAGCGGCCGCGG 3′ (SEQ ID NO:68) containing the underlined BamHI restriction site, which encodes a start AUG, followed by the Kozak sequence and 18 nucleotides of the DR3 coding sequence set out in SEQ ID NO:3 beginning with the first base of the ATG codon.

The 3′ primer for both DR3 and DR3-V1 has the sequence: 5′CGC GGATCCTCACGGGCCGCGCTGCA 3′ (SEQ ID NO:70) containing the underlined BamHI restriction site followed by 17 nucleotides complementary to the last 14 nucleotides of the DR3 coding sequence set out in SEQ ID NO:3, plus the stop codon.

The restrictions sites are convenient to restriction enzyme sites in the CHO expression vectors pC1.

The amplified DR3 or DR3-V1 DNA and the vector pC1 both are digested with BamHI and the digested DNAs then ligated together. Insertion of the DR3-V1 or DR3 DNA into the BamHI restricted vector placed the DR3-V1 or DR3 coding region downstream of and operably linked to the vector's promoter. The sequence of the inserted gene is confirmed by DNA sequencing.

Transfection of CHO-DHFR-Cells

Chinese hamster ovary cells lacking an active DHFR enzyme are used for transfection. 5 μg of the expression plasmid C1 are cotransfected with 0.5 μg of the plasmid pSVneo using the lipofecting method (Felgner et al., supra). The plasmid pSV2-neo contains a dominant selectable marker, the gene neo from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) and cultivated from 10-14 days. After this period, single clones are trypsinized and then seeded in 6-well petri dishes using different concentrations of methotrexate (25 nM, 50 nM, 100 nM, 200 nM, 400 nM). Clones growing at the highest concentrations of methotrexate are then transferred to new 6-well plates containing even higher concentrations of methotrexate (500 nM, 1 μM, 2 μM, 5 μM). The same procedure is repeated until clones grow at a concentration of 100 μM.

The expression of the desired gene product is analyzed by Western blot analysis and SDS-PAGE.

### Example 28

### Cloning and Expression of the Soluble Extracellular Domain of DR3-V1 and DR3 in a Baculovirus Expression System

The cDNA sequence encoding the soluble extracellular domain of DR3-V1 or DR3 protein in the deposited clone (ATCC No. 97456 or ATCC No. 97757, respectively) is amplified using PCR oligonucleotide primers corresponding to the 5′ and 3′ sequences of the gene:

The 5′ primer for DR3 has the sequence: 5′CGCGGATCCGCCA TCATGGAGCAGCGGCCGCGG 3′ (SEQ ID NO:68) containing the underlined BamHI restriction enzyme site followed by a Kozak sequence and a number of bases of the sequence of DR3 of SEQ ID NO:3. Inserted into an expression vector, as described below, the 5′ end of the amplified fragment encoding DR3 provides an efficient signal peptide. An efficient signal for initiation of translation in eukaryotic cells, as described by M. Kozak, J. Mol. Biol. 196:947-950 (1987) is appropriately located in the vector portion of the construct.

The 3′ primer for both DR3 and DR3-V1 has the sequence: 5′ GCGA GATCTAGTCTGGACCCAGAACATCTGCCTCC 3′ (SEQ ID NO:71) containing the underlined XbaI restriction followed by nucleotides complementary to the DR3 nucleotide sequence set out in SEQ ID NO:3, followed by the stop codon.

The amplified fragment is isolated from a 1% agarose gel using a commercially available kit (“Geneclean,” BIO 101 Inc., La Jolla, Calif.) The fragment then is digested with BamHI and Asp718 and again is purified on a 1% agarose gel. This fragment is designated herein F2.

The vector pA2 is used to express the DR3-V1 or DR3 protein in the baculovirus expression system, using standard methods, such as those described in Summers et al., A Manual of Methods for Baculovirus Vectors and Insect Cell Culture Procedures, Texas Agricultural Experimental Station Bulletin No. 1555 (1987). This expression vector contains the strong polyhedron promoter of the Autograph californica nuclear polyhedrosis virus (ACMNPV) followed by convenient restriction sites. For an easy selection of recombinant virus the β-galactosidase gene from E. coli is inserted in the same orientation as the polyhedron promoter and is followed by the polyadenylation signal of the polyhedron gene. The polyhedron sequences are flanked at both sides by viral sequences for cell-mediated homologous recombination with wild-type viral DNA to generate viable virus that express the cloned polynucleotide.

Many other baculovirus vectors could be used in place of pA2, such as pAc373, pVL941 and pAcIM1 provided, as those of skill readily will appreciate, that construction provides appropriately located signals for transcription, translation, trafficking and the like, such as an in-frame AUG and a signal peptide, as required. Such vectors are described in Luckow et al., Virology 170:31-39 (1989), among others.

The plasmid is digested with the restriction enzymes BamHI and XbaI and then is dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V2”.

Fragment F2 and the dephosphorylated plasmid V2 are ligated together with T4 DNA ligase. E. Coli HB101 cells are transformed with ligation mix and spread on culture plates. Bacteria are identified that contain the plasmid with the human DDCR gene by digesting DNA from individual colonies using BamHI and XbaI and then analyzing the digestion product by gel electrophoresis. The sequence of the cloned fragment is confirmed by DNA sequencing. This plasmid is designated herein pBac DR3-V1 or pBac DR3.

5 μg of the plasmid pBac DR3-V1 or pBac DR3 is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). 1 μg of BaculoGold™ virus DNA and 5 μg of the plasmid pBac DR3-VI are mixed in a sterile well of a microliter plate containing 50 μl of serum free Grace's medium (Life Technologies Inc., Gaithersburg, Md.). Afterwards 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5, hours the transfection solution is removed from the plate and 1 ml of Grace's insect medium supplemented with 10% fetal calf serum is added. The plate is put back into an incubator and cultivation is continued at 27° C. for four days.

After four days the supernatant is collected and a plaque assay is performed, as described by Summers and Smith, cited above. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10).

Four days after serial dilution, the virus is added to the cells. After appropriate incubation, blue stained plaques are picked with the tip of an Eppendorf pipette. The agar containing the recombinant viruses is then resuspended in an Eppendorf tube containing 200 μl of Grace's medium. The agar is removed by a brief centrifugation and the supernatant containing the recombinant baculovirus is used to infect Sf9 cells seeded in 35 mm dishes. Four days later the supernatants of these culture dishes are harvested and then they are stored at 4° C. A clone containing properly inserted DR3-V1 or DR3 is identified by DNA analysis including restriction mapping and sequencing. This is designated herein as V-DR3-V1 or V-DR3.

Sf9 cells are grown in Grace's medium supplemented with 10% heat-inactivated FBS. The cells are infected with the recombinant baculovirus V-DR3-V1 at a multiplicity of infection (“MOI”) of about 2 (about 1 to about 3). Six hours later the medium is removed and is replaced with SF900 II medium minus methionine and cysteine (available from Life Technologies Inc., Gaithersburg). 42 hours later, 5 μCi of ³⁵S-methionine and 5 μCi ³⁵S cysteine (available from Amersham) are added. The cells are further incubated for 16 hours and then they are harvested by centrifugation, lysed and the labeled proteins are visualized by SDS-PAGE and autoradiography.

### Example 29

### Tissue Distribution of DR3 Gene Expression

Northern blot analysis is carried out to examine DR3 gene (ATCC No. 97757) expression in human tissues, using methods described by, among others, Sambrook et al., cited above. A cDNA probe containing the entire nucleotide sequence of the DR3 protein (SEQ ID NO:3) is labeled with ³²P using the rediprime™ DNA labeling system (Amersham Life Science), according to manufacturer's instructions. After labeling, the probe is purified using a CHROMA SPIN-100™ column (Clontech Laboratories, Inc.), according to manufacturer's protocol number PT1200-1. The purified labeled probe is then used to examine various human tissues for DR3 mRNA.

Multiple Tissue Northern (MTN) blots containing various human tissues (H) or human immune system tissues (IM) are obtained from Clontech and are examined with labeled probe using ExpressHyb™ hybridization solution (Clontech) according to manufacturer's protocol number PT1190-1. Following hybridization and washing, the blots are mounted and exposed to film at −70° C. overnight, and films developed according to standard procedures.

Expression of DR3 was detected in tissues enriched in lymphocytes including peripheral blood leukocytes (PBLs), thymus, spleen, colon, and small intestine. By contrast, TNFR-1 is ubiquitously expressed and Fas/APO-1 is expressed in lymphocytes, liver, heart, lung, kidney, and ovary (Watanabae-Fukunaga et al., J. Immunol 148:1274-9 (1992)).

DR3 expression appears to be restricted to lymphocyte compartments, it can be envisaged that DR3 plays a role in lymphocyte homeostasis.

Northern Blot Analysis of DR3 in Various Cell Lines

Cells

Unless stated otherwise, cell lines were obtained from the American Type Culture Collection (Manassas, Va.). The myeloid (Koeffler et al. (1980); Koeffler (1983); Harris and Ralph (1985); and Tucker et al. (1987)) and B-cell lines (Jonak et al. (1922)) studied represent cell types at different stages of the differentiation pathway. KG1a and PLB 985 cells (Tucker et al. (1987)) were obtained from H. P. Koeffler (UCLA School of Medicine). BJA-B was from Z. Jonak (SmithKline Beecham). TF274, a stromal cell line exhibiting osteoblastic features, was generated from the bone marrow of a healthy male donor (Z. Jonak and K. B. Tan, unpublished). Primary carotid artery endothelial cells were purchased from Clonetics Corp. (San Diego, Calif.) and monocytes were prepared by differential centrifugation of peripheral blood mononuclear cells and adhesion to tissue culture dish. CD19+, CD4+ and CD8+ cells (>90% pure) were isolated with cell type specific immunomagnetic beads (Drynal, Lake Success, N.Y.).

RNA Analysis

Total RNA of adult tissues were purchased from Clonetech (Palo Alto, Calif.). Total RNA was extracted from cell lines (in exponential growth phase) and primary cells with TriReagent (Molecular Research Center, Inc., Cincinnati, Ohio). 5 to 7.5 μg of total RNA was fractionated in a 1% agarose gel containing formaldehyde cast in a Wide Mini-Sub Cell gel tray (Bio-Rad, Hercules, Calif.) as described (Sambrook, et al.) with slight modifications. The formaldehyde concentration was reduced to 0.5M and the RNA was stained prior to electrophoresis with 100 μg/ml of ethidium bromide that was added to the loading buffer. After electrophoresis with continuous buffer recirculation (60 volts/90 min), the gel was photographed and the RNA was transferred quantitatively to Zeta-probe nylon membrane (Biorad, Hercules, Calif.) by vacuum-blotting with 25 mM NaOH for 90 min. After neutralization for 5-10 min, with 1M Tris-HCl, pH 7.5 containing 3M NaCl, the blots were prehybridized with 50% formamide, 8% dextran sulfate, 6×SSPE, 0.1% SDS and 100 μg/ml of sheared and denatured salmon sperm DNA for at least 30 min at 42° C. cDNA inserts labeled with ³²P-dCTP by random priming (Stratagene, La Jolla, Calif.), were denatured with 0.25M NaOH (10 min at 37° C.) and added to the prehybridization solution. After 24-65 hr at 42° C., the blots were washed under high stringency conditions (Sambrook, et al.) and exposed to X-ray films.

Results

Expression of DR3 was assessed by Northern blot in the following cell lines: TF274 (bone marrow stromal); MG63, TE85 (osteosarcoma); K562 (erythroid); KG1a, KG1, PLB985, HL60, U937, TNHP-1 (myeloid); REH, BJAB, Raji, IM-9 (B cell); Sup-Ti, Jurkat, H9, Molt-3 (T cell); RL95-2 (endometrial carcinoma); MCF-7 (breast cancer); BE, HT29 (colon cancer); IMR32 (neuroblastoma) and could only be detected in KG1a cells. DR3 expression was detected in several lymphoblast cell lines. In the purified human hematopoietic cell populations, DR3 was weakly expressed in CD19+ cells, and more highly expressed in monocytes. However the highest levels were observed in T cells (CD4+ or CD8+) upon stimulation with PMA and PHA, indicating that DR3 probably plays a role in the regulation of T cell activation.

### Example 30

### Intracellular Signaling Molecules Used by DR3 Protein

In vitro and in vivo binding studies were undertaken to investigate DR3 signaling pathways. Since DR3 contains a death domain, the inventors postulated that DR3, like TNFR-1 and Fas/APO-1, may transduce signals by recruiting death domain-containing adapter molecules (DAMs) such as FADD, TRADD, and RIP.

Experimental Design

In vitro binding experiments were performed as described previously (A. M. Chinnaiyan et al., Cell 81: 505-12 (1995); M. P. Boldin et al., J Biol Chem 270: 7795-8 (1995); F. C. Kischkel et al., EMBO 14: 5579-5588 (1995)). Briefly, the cytoplasmic domains of DR3 (amino acid residues 215-393 (SEQ ID NO:4)) and the death domain mutant ΔDR3 (amino acid residues 215-321 (SEQ ID NO:4) were amplified by PCR using appropriate templates and primers into pGSTag. pGSTag and pGSTag-TNFR-1 were described previously (A. M. Chinnaiyan et al., Cell 81: 505-12 (1995); M. P. Boldin et al., J Biol Chem 270: 7795-8 (1995); F. C. Kischkel et al., EMBO 14: 5579-5588 (1995)). GST and GST fusion proteins were prepared from E. coli strain BL21(DE3)pLysS using standard published procedures and the recombinant proteins immobilized onto glutathione-agarose beads. ³⁵S-Labeled FADD, RIP and TRADD were prepared by in vitro transcription-translation using the TNT or T7 or SP6-coupled reticulocyte lysate system from Promega according to manufacturer's instructions, using pcDNA3 AU1-FADD (A. M. Chinnaiyan et al., Cell 81: 505-12 (1995); M. P. Boldin et al, J Biol Chem 270: 7795-8 (1995); F. C. Kischkel et al., EMBO 14: 5579-5588 (1995)), pRK myc-TRADD (H. Hsu et al., Cell 81: 495-504 (1995)), or pRK myc-RIP (H. Hsu et al., Immunity 4: 387-396 (1996)) as template. Following translation, equal amounts of total ³⁵S-labeled reticulocyte lysate were diluted into 150 μl GST binding buffer (50 mM Tris, pH 7.6, 120 mM NaCl, 1% NP-40) and incubated for 2 hrs. at 4° C. with the various GST fusion proteins complexed to beads, following the beads were pelleted by plus centrifugation, washed three times in GST buffer, boiled in SDS-sample buffer and resolved on a 12.5% SDS-PAGE. Bound proteins were visualized following autoradioraphy at −80° C. In vitro translated ³⁵S-labeled RIP, TRADD and FADD were incubated with glutathione beads containing GST alone or GST fusions of the cytoplasmic domain of Fas, TNFR-1, DR3 (215-393), or DDR3 (215-321). After the beads were washed, retained proteins were analyzed by SDS-PAGE and autoradiography. The gel was Coomassie stained to monitor equivalency of loading.

To demonstrate the association of DR3 and TRADD in vivo, constructs encoding Flag-TNFR-1 and Flag-ΔTNFR-1 were used. The Flag-TNFR-1 and Flag-ΔTNFR-1 constructs were described elsewhere (A. M. Chinnaiyan et al., J Biol Chem 271: 4961-4965 (1996)). The constructs encoding Flag-TNFR-1 and Flag-ΔTNFR-1 were described elsewhere (A. M. Chinnaiyan et al., J Biol Chem 271: 4961-4965 (1996)). To facilitate epitope tagging, DR3 and ΔDR3 (1-321) were cloned into the IBI Kodak FLAG plasmid (pCMV1FLAG) utilizing the signal peptide provided by the vector. 293 cells (2×10⁶/100 mm plate) were grown in DMEM media containing 10% heat-inactivated fetal bovine serum containing penicillin G, streptomycin, glutamine, and non-essential amino acids. Cells were transfected using calcium phosphate precipitation with the constructs encoding the indicated proteins in combination with pcDNA3-CrmA (M. Tewari et al., J Biol Chem 270: 3255-60 (1995)) to prevent cell death and thus maintain protein expression. Cells were lysed in 1 ml lysis buffer (50 mM Hepes, 150 mM NaCl, 1 mM EDTA, 1% NP-40, and a protease inhibitor cocktail). Lysates were immunoprecipitated with a control monoclonal antibody or anti-Flag antibody for at least 4 hrs, at 4° C. as previously described (A. M. Chinnaiyan et al., J Biol Chem 271: 4961-4965 (1996)). The beads were washed with lysis buffer 3×, but in the case of TRADD binding, the NaCl concentration was adjusted to 1M. The: precipitates were fractioned on 12.5% SDS-PAGE and transferred to nitrocellulose. Subsequent Western blotting was performed as described elsewhere (H. Hsu et al., Cell 84: 299-308 (1996); A. M. Chinnaiyan et al., J Biol Chem 271, 4961-4965 (1996)). After 24-32 hours, extracts were prepared and immunoprecipitated with a control monoclonal antibody or anti-Flag monoclonal antibody (IBI Kodak). Western analysis indicated that myc-TRADD and death receptor expression levels were similar in all samples. Coprecipitating myc-TRADD was detected by immunoblotting using an anti-myc HRP conjugated antibody (Boehringer Mannheim).

Results

As an initial screen, in vitro translated radiolabeled DAMs were precipitated with various glutathione S-transferase (GST) fusion proteins immobilized on glutathione-Sepharose beads. As predicted from previous studies (A. M. Chinnaiyan et al., Cell 81: 505-12 (1995); M. P. Boldin et al., J Biol Chem 270: 7795-8 (1995); F. C. Kischkel et al., EMBO 14: 5579-5588 (1995); H. Hsu et al., Cell 81: 495-504 (1995)), FADD associated with the GST-Fas cytoplasmic domain while TRADD associated with the GST-TNFR-1 cytoplasmic domain. In addition, there was a direct, albeit weak, interaction between RIP and GST-TNFR-1. Interestingly, GST-DDCR associated specifically with TRADD, but not FADD or RIP. Furthermore, a truncated death domain mutant of DR3 (GST-DDR3) failed to interact with TRADD. To demonstrate the association of DR3 and TRADD in vivo, 293 cells were transiently transfected with plasmids that direct the synthesis of myc-epitope tagged TRADD (myc-TRADD) and Flag-epitope tagged DR3 (Flag-DR3), Flag-TNFR-1 or mutants. Consistent with the in vitro binding study, TRADD specifically coprecipitated with DR3 and TNFR-1, but not with the death domain mutants, DDR3 and DTNFR-1. Thus, it appears that DR3, like TNFR-1, may activate downstream signaling cascades by virtue of its ability to recruit the adapter molecule TRADD.

Overexpression of TRADD induces apoptosis and NF-kB activation-two of the most important activities signaled by TNFR-1 (H. Hsu et al., supra). Upon oligomerization of TNFR-1 by trimeric TNF, TRADD is recruited to the receptor signaling complex (H. Hsu et al., Cell 84:299-308 (1996)). TRADD can then recruit the following signal transducing molecules: 1) TRAF2, a TNFR-2- and CD40—associated molecule (M. Rothe et al., Cell 78: 681-92 (1994); M. Rothe et al., Science 269:1424-1427 (1995)), that mediates NF-kB activation, 2) RIP, originally identified as a Fas/APO-1-interacting protein by two-hybrid analysis (B. Z. Stanger et al., Cell 81: 513-23 (1995)), that mediates NF-kB activation and apoptosis (H. Hsu et al., Immunity 4: 387-396 (1996)), and 3) FADD, a Fas/APO-1-associated molecule, that mediates apoptosis (A. M. Chinnaiyan et al., Cell 81: 505-12 (1995); M. P. Boldin et al., J Biol Chem 270:7795-8 (1995); F. C. Kischkel et al., EMBO 14: 5579-5588 (1995)). Thus, the inventors demonstrate that RIP, TRAF2 and FADD could be co-immunoprecipitated with DR3. In 293 cells expressing DR3 and RIP, only a weak association could be detected between the two molecules. However, in the presence of TRADD, RIP association with DR3 was significantly enhanced. Likewise, very little TRAF2 directly co-precipitated with DR3 in 293 cells. However, when DR3 and TRAF2 were expressed in the presence of TRADD and RIP (both of which can bind TRAF2), an enhanced binding of TRAF2 to DR3 could be detected. A similar association between FADD and DR3 was also observed. In the presence of TRADD, FADD efficiently coprecipitated with DR3.

Previous studies demonstrated that FADD could recruit the ICE/CED-3-like protease FLICE to the Fas/APO-1 death inducing signaling complex (M. Muzio et al., Cell 85: 817-827 (1996); M. P. Boldin et al., Cell 85: 803-815 (1996)). To demonstrate that FLICE can associate with TNFR-1 and DR3, coprecipitation experiments in 293 cells were carried out. Interestingly, FLICE was found complexed to TNFR-1 and DR3. Co-transfection of TRADD and/or FADD failed to enhance the FLICE-TNFR-1/DR3 interaction, suggesting that endogenous amounts of these adapter molecules were sufficient to maintain this association.

### Example 31

### Gene Therapy Using Endogenous DR3 Gene

Another method of gene therapy according to the present invention involves operably associating the endogenous DR3 sequence with a promoter via homologous recombination as described, for example, in U.S. Pat. No. 5,641,670, issued Jun. 24, 1997; International Publication Number WO 96/29411, published Sep. 26, 1996; International Publication Number WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not expressed in the cells, or is expressed at a lower level than desired. Polynucleotide constructs are made which contain a promoter and targeting sequences, which are homologous to the 5′ non-coding sequence of endogenous DR3, flanking the promoter. The targeting sequence will be sufficiently near the 5′ end of DR3 so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous DR3 sequence. This results in the expression of DR3-V1 or DR3 in the cell. Expression may be detected by immunological staining, or any other method known in the art.

Fibroblasts are obtained from a subject by skin biopsy. The resulting tissue is placed in DMEM+10% fetal calf serum. Exponentially growing or early stationary phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na2 HPO4, 6 mM dextrose). The cells are recentrifuged, the supernatant aspirated, and the cells resuspended in electroporation buffer containing 1 mg/ml acetylated bovine serum albumin. The final cell suspension contains approximately 3×10⁶ cells/ml. Electroporation should be performed immediately following resuspension.

Plasmid DNA is prepared according to standard techniques. For example, to construct a plasmid for targeting to the DR3 locus, plasmid pUC18 (MBI Fermentas, Amherst, N.Y.) is digested with HindIII. The CMV promoter is amplified by PCR with an XbaI site on the 5′ end and a BamHI site on the 3′ end. Two DR3 non-coding sequences are amplified via PCR: one DR3 non-coding sequence (DR3 fragment 1) is amplified with a HindIII site at the 5′ end and an XbaI site at the 3′end; the other DR3 non-coding sequence (DR3 fragment 2) is amplified with a BamHI site at the 5′end and a HindIII site at the 3′end. The CMV promoter and DR3 fragments are digested with the appropriate enzymes (CMV promoter—XbaI and BamHI; DR3 fragment 1—XbaI; DR3 fragment 2—BamHI) and ligated together. The resulting ligation product is digested with HindIII, and ligated with the HindIII-digested pUC18 plasmid.

Plasmid DNA is added to a sterile cuvette with a 0.4 cm electrode gap (Bio-Rad). The final DNA concentration is generally at least 120 μg/ml. 0.5 ml of the cell suspension (containing approximately 1.5×10⁶ cells) is then added to the cuvette, and the cell suspension and DNA solutions are gently mixed. Electroporation is performed with a Gene-Pulser apparatus (Bio-Rad). Capacitance and voltage are set at 960 μF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 minutes, and the contents of the cuvette are then gently removed with a sterile transfer pipette. The cells are added directly to 10 ml of prewarmed nutrient media (DMEM with 15% calf serum) in a 10 cm dish and incubated at 37_C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 32

### Method of Determining Alterations in the DR3 Gene

RNA is isolated from entire families or individual patients presenting with a phenotype of interest (such as a disease). cDNA is then generated from these RNA samples using protocols known in the art. (See, Sambrook et al., 1990) The cDNA is then used as a template for PCR, employing primers surrounding regions of interest in SEQ ID NO:3. Suggested PCR conditions consist of 35 cycles at 95° C. for 30 seconds; 60-120 seconds at 52-58° C.; and 60-120 seconds at 70° C., using buffer solutions described in Sidransky, D., et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons of DR3 are also determined and genomic PCR products analyzed to confirm the results. PCR products harboring suspected mutations in DR3 are then cloned and sequenced to validate the results of the direct sequencing.

PCR products of DR3 are cloned into T-tailed vectors as described in Holton, T. A. and Graham, M. W., Nucleic Acids Research, 19:1156 (1991) and sequenced with T7 polymerase (United States Biochemical). Affected individuals are identified by mutations in DR3 not present in unaffected individuals.

Genomic rearrangements are also observed as a method of determining alterations in the DR3 gene. Genomic clones isolated using techniques known in the art are nick-translated with digoxigenindeoxy-uridine 5′-triphosphate (Boehringer Manheim), and FISH performed as described in Johnson, C. et al, Methods Cell Biol. 35:73-99 (1991). Hybridization with the labeled probe is carried out using a vast excess of human cot-1 DNA for specific hybridization to the DR3 genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, C. et al., Genet. Anal Tech. Appl., 8:75 (1991).) Image collection, analysis and chromosomal fractional length measurements are performed using the ISee Graphical Program System. (Inovision Corporation, Durham, N.C.) Chromosome alterations of the genomic region of DR3 (hybridized by the probe) are identified as insertions, deletions, and translocations. These DR3 alterations are used as a diagnostic marker for an associated disease.

### Example 33

### Determination of Transcription of the TNF-gamma-β, DR3, and/or TR6 Genes

To assess the presence or absence of active transcription of a TNF-gamma-β, DR3, and/or TR6 gene RNA, approximately 6 ml of venous blood is obtained with a standard venipuncture technique using heparinized tubes. Whole blood is mixed with an equal volume of phosphate buffered saline, which is then layered over 8 ml of Ficoll (Pharmacia, Uppsala, Sweden) in a 15-ml polystyrene tube. The gradient is centrifuged at 1800×g for 20 min at 5° C. The lymphocyte and granulocyte layer (approximately 5 ml) is carefully aspirated and rediluted up to 50 ml with phosphate-buffered saline in a 50-ml tube, which is centrifuged again at 1800×g for 20 min. at 5° C. The supernatant is discarded and the pellet containing nucleated cells is used for RNA extraction using the RNazole B method as described by the manufacturer (Tel-Test Inc., Friendswood, Tex.).

To determine the quantity of mRNA from a gene of interest, a probe is designed with an identity to a mRNA sequence transcribed from a human gene whose coding portion includes a DNA sequence of FIGS. 1A-1B, 3A-3C, and/or 5A-5B. This probe is mixed with the extracted RNA and the mixed DNA and RNA are precipitated with ethanol −70° C. for 15 minutes). The pellet is resuspended in hybridization buffer and dissolved. The tubes containing the mixture are incubated in a 72° C. water bath for 10-15 mins. to denature the DNA. The tubes are rapidly transferred to a water bath at the desired hybridization temperature. Hybridization temperature depends on the G+C content of the DNA. Hybridization is done for 3 hrs. 0.3 ml of nuclease-S 1 buffer is added and mixed well. 50 l of 4.0 M ammonium acetate and 0.1 M EDTA is added to stop the reaction. The mixture is extracted with phenol/chloroform and 20 g of carrier tRNA is added and precipitation is done with an equal volume of isopropanol. The precipitate is dissolved in 401 of TE (pH 7.4) and run on an alkaline agarose gel. Following electrophoresis, the RNA is microsequenced to confirm the nucleotide sequence. (See Favaloro, J. et al., Methods Enzymol., 65:718 (1980) for a more detailed review).

Two oligonucleotide primers are employed to amplify the sequence isolated by the above methods. The 5′ primer is 20 nucleotides long and the 3′ primer is a complimentary sequence for the 3′ end of the isolated mRNA. The primers are custom designed according to the isolated mRNA. The reverse transcriptase reaction and PCR amplification are performed sequentially without interruption in a Perkin Elmer 9600 PCR machine (Emeryville, Calif.). Four hundred ng total RNA in 20 ul diethylpyrocarbonate-treated water are placed in a 65° C. water bath for 5 min. and then quickly chilled on ice immediately prior to the addition of PCR reagents. The 50-ul total PCR volume consisted of 2.5 units Taq polymerase (Perkin-Elmer). 2 units avian myeloblastosis virus reverse transcriptase (Boehringer Mannheim, Indianapolis, Ind.); 200 uM each of dCTP, dATP, dGTP and dTTP (Perkin Elmer); 18 pM each primer, 10 mM Tris-HCl; 50 mM KCl; and 2 mM MgCl (Perkin Elmer). PCR conditions are as follows: cycle 1 is 42° C. for 15 min then 97° C. for 15 s (1 cycle); cycle 2 is 95° C. for 1 min. 60° C. for 1 min, and 72° C. for 30 s (15 cycles); cycle 3 is 95° C. for 1 min. 60° C. for 1 min., and 72° C. for 1 min. (10 cycles); cycle 4 is 95° C. for 1 min., 60° C. for 1 min., and 72° C. for 2 min. (8 cycles); cycle 5 is 72° C. for 15 min. (1 cycle); and the final cycle is a 4° C. hold until sample is taken out of the machine. The 50-ul PCR products are concentrated down to 10 ul with vacuum centrifugation, and a sample is then run on a thin 1.2% Tris-borate-EDTA agarose gel containing ethidium bromide. A band of expected size would indicate that this gene is present in the tissue assayed. The amount of RNA in the pellet may be quantified in numerous ways, for example, it may be weighed.

Verification of the nucleotide sequence of the PCR products is done by microsequencing. The PCR product is purified with a Qiagen PCR Product Purification Kit (Qiagen, Chatsworth, Calif.) as described by the manufacturer. One g of the PCR product undergoes PCR sequencing by using the Taq DyeDeoxy Terminator Cycle sequencing kit in a Perkin-Elmer 9600 PCR machine as described by Applied Biosystems (Foster, Calif.). The sequenced product is purified using Centri-Sep columns (Princeton Separations, Adelphia, N.J.) as described by the company. This product is then analyzed with an ABI model 373A DNA sequencing system (Applied Biosystems) integrated with a Macintosh IIci computer.

### Example 34

### Transgenic Animals.

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (11989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 35

### Knock-Out Animals.

Endogenous gene expression can also be reduced by inactivating or “knocking out” the gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly. suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, eg., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 36

### Production of an Antibody

The antibodies of the invention include, but are not limited to, antagonists of TNF-gamma-β and DR3 and are useful in diagnosing, preventing, ameliorating, and/or treating inflammatory bowel disease, including Crohn's or ulcerative colitis.

Hybridoma Technology

The antibodies of the present invention can be prepared by a variety of methods. (See, Current Protocols, Chapter 2.) As one example of such methods, cells expressing polypeptide(s) of the invention are administered to an animal to induce the production of sera containing polyclonal antibodies. In a preferred method, a preparation of polypeptide(s) of the invention is prepared and purified to render it substantially free of natural contaminants. Such a preparation is then introduced into an animal in order to produce polyclonal antisera of greater specific activity.

Monoclonal antibodies specific for polypeptide(s) of the invention (e.g., TNF-gamma-β and DR3) are prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)). In general, an animal (preferably a mouse) is immunized with polypeptide(s) of the invention, or, more preferably, with a secreted polypeptide-expressing cell. Such polypeptide-expressing cells are cultured in any suitable tissue culture medium, preferably in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the polypeptide(s) of the invention.

Alternatively, additional antibodies capable of binding polypeptide(s) of the invention can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the polypeptide(s) of the invention protein-specific antibody can be blocked by polypeptide(s) of the invention. Such antibodies comprise anti-idiotypic antibodies to the polypeptide(s) of the invention protein-specific antibody and are used to immunize an animal to induce formation of further polypeptide(s) of the invention protein-specific antibodies.

For in vivo use of antibodies in humans, an antibody is “humanized”. Such antibodies can be produced using genetic constructs derived from hybridoma cells producing the monoclonal antibodies described above. Methods for producing chimeric and humanized antibodies are known in the art and are discussed herein. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al.) Nature 312:643 (1984); Neuberger et al., Nature 314:268 (1985).)

Isolation of Antibody Fragments Directed Polypeptide(s) of the Invention from a Library of scFvs

Naturally occurring V-genes isolated from human PBLs are constructed into a library of antibody fragments which contain reactivities against polypeptide(s) of the invention to which the donor may or may not have been exposed (see e.g., U.S. Pat. No. 5,885,793 incorporated herein by reference in its entirety).

Rescue of the Library. A library of scFvs is constructed from the RNA of human PBLs as described in PCT publication WO 92/01047. To rescue phage displaying antibody fragments, approximately 109 E. coli harboring the phagemid are used to inoculate 50 ml of 2×TY containing 1% glucose and 100 μg/ml of ampicillin (2×TY-AMP-GLU) and grown to an O.D. of 0.8 with shaking. Five ml of this culture is used to innoculate 50 ml of 2×TY-AMP-GLU, 2×10⁸ TU of delta gene 3 helper (Ml 3 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2×TY containing 100 μg/ml ampicillin and 50 ug/ml kanamycin and grown overnight. Phages are prepared as described in PCT publication WO 92/01047.

M13 delta gene III is prepared as follows: M13 delta gene III helper phage does not encode gene III protein, hence the phage(mid) displaying antibody fragments have a greater avidity of binding to antigen. Infectious M13 delta gene m particles are made by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are spun down (IEC-Centra 8,400 r.p.m. for 10 min), resuspended in 300 ml 2×TY broth containing 100 μg ampicillin/ml and 25 μg kanamycin/ml (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 μm filter (Minisart NML; Sartorius) to give a final concentration of approximately 1013 transducing units/ml (ampicillin-resistant clones).

Panning of the Library. Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders

Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

### Example 37

### Increased Mucosal Expression of and Response to TNF-gamma-β Augments Production of the TH1 Effector Cytokine IFNγ, in Crohn's Disease

Background

Mucosal inflammation in Crohn's disease (CD) is provoked by excessive secretion of TH1-inducing cytokines, IL-12 and IL-18, as well as TNFα, which, in turn, stimulates increased IFNγ production by lamina propria T (LP-T) cells. Other factors, however, are likely to be present and active in exacerbating inflammation in Crohn's mucosa.

Aims

To test the effect of recombinant TNF-gamma-β on IFNγ production by stimulated Lamina Propria Mononuclear Cells, and its interaction with IL-12 and IL-18; to evaluate the role of endogenous TNF-gamma-β expression; and to quantify TNF-gamma-β receptor expression (TNF-gamma-β R) on LP-T cells.

Methods

Lamina Propria Mononuclear Cells from normal, ulcerative colitis and CD specimens were cultured overnight in IL-2 (10 U/ml), aliquots stained for FACS analysis of TNF-gamma-β receptor expression, then incubated for 72 hrs with a pair of activating anti-CD2 antibodies (Biogen, Inc.) and recombinant TNF-gamma-β, or antibody to TNF-gamma-β, alone or in combination with IL-12, IL-18 or with neutralizing antibody to IL-12 or IL-18 or to TNF-gamma-β. IFNγ in supernatants was detected by ELISA.

Results

In contrast to Peripheral Blood Lymphocytes, TNF-gamma-β was expressed on a substantial fraction of CD3+CD4+ and CD8+ Lamina Propria-T cells from all guts, independently of IL-2 culture or in vitro activation, but Crohn's Disease samples had higher DR3+fractions. TNF-gamma-β (1-100 ng/ml) dose-dependently increased IFNγ production, up to 2-fold or more, as did anti-TNF-gamma-β Mab (0.2-0.3 ug/ml). This was highest with Lamina Propria Mononuclear Cells from Crohn's Disease samples. In the presence of neutralizing antibody to IL-12, IL-18, or both, TNF-gamma-β still increased IFNγ above control. TNF-gamma-β synergized with high dose IL-12 and IL-18 and with both together, resulting in even more IFNγ secretion. Thus, TNF-gamma-β acts independently of IL-12 and IL-18. Anti-TLx antibody reduced IFNγ most markedly in Crohn's Disease samples.

### CONCLUSIONS

TNF-gamma-β acts on Lamina Propria Mononuclear Cells to substantially increase stimulated IFNγ secretion, independently of, but in synergy with, IL-12 and IL-18. A large fraction of Lamina Propria-T cells expresses TNF-gamma-β receptor, especially in Crohn's Disease. The increase in IFNγ production in response to exogenous TNF-gamma-β is greatest with Lamina Propria Mononuclear Cells from Crohn's Disease, as is the reduction in IFNγ in the presence of anti-TNF-gamma-β neutralizing antibody. TNF-gamma-β produced in the mucosa and acting on TNF-gamma-βR+ Lamina Propria-T cells could be an important novel factor exacerbating inflammation in Crohn's disease.

### Example 38

### TNF-gamma-β Augments IFNγ Production from Stimulated T Cells Independently of, but in Synergy with, IL-12 and IL-18

Background

Intestinal mucosal inflammation, especially in Crohn's disease, is characterized by a powerful TH1 response, well known to be dependent on IL-12 and IL-18, marked by increased activated T cell expression of the inflammatory mediators IFNγ and TNFα by activated T. cells. TNFα, in turn, potentiates IFNγ production from lamina propria T cells. TNF-gamma-β, like IL-12 and IL-18, augments IFNγ production from CD3 activated peripheral T cells from blood.

Aims

To analyze the effect of TNF-gamma-β on IFNγ production by stimulated T cells, in relation to that of IL-12 and IL-18, and to examine the regulation of TNF-gamma-β receptor (“TNF-gamma-βR”, i.e., DR3) expression.

Methods

Non-adherent blood cells were stimulated with PHA (1-2 μg/ml) and incubated with either recombinant TNF-gamma-β, IL-12, IL-18, or neutralizing antibodies to each. IFNγ level was measured from culture supernatants 72 hrs later by ELISA. TNF-gamma-βR expression on CD3/CD4 or CD8 cells was detected by indirect staining with anti-TNF-gamma-βR mAb followed by FACS analysis.

Results

PHA induced TNF-gamma-βR expression on receptor-negative resting T cells from normal donors: up to 33% of CD4+ cells and 33% of CD8+ cells. TNF-gamma-β (10-150 ng/ml) increased IFNγ expression dose dependently, up to 5-7-fold with low dose PHA. Two anti-TNF-gamma-βR monoclonal antibodies had the same effect when used in the same experiment, demonstrating they are agonistic antibodies. IFNγ increased markedly from 24 to 72 hrs, and the large augmentation by TNF-gamma-β was maintained. IFNγ production continued to be enhanced by TNF-gamma-β in the presence of neutralizing antibody to IL-12, IL-18, or both. TNF-gamma-β synergized with high concentrations of added recombinant IL-12 (1.0 ng/ml), IL-18 (50 ng/ml), and with both together, increasing IFNγ well above the enhanced levels produced by either IL-12, IL-18, or both. Anti-TNF-gamma-βR acted similarly with similar potency. Together, these results provide strong evidence that TNF-gamma-β acts independently of these well-studied potentiators of the TH1 response.

### CONCLUSION

TNF-gamma-β, a novel TNF homologue, potentiates IFNγ production from peripheral T cells in synergy with IL-12 and IL-18, but independently of them, and anti-TNF-gamma-βR antibodies act similarly. TNF-gamma-PR is upregulated on a large fraction of T cells by activation. Thus, local TNF-gamma-β production and its receptor expression on TH1 cells, could exacerbate pathological inflammation, for instance, in the colitides.

### CONCLUSION

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference.

Further, the hard copy of the sequence listing submitted herewith and the corresponding computer readable form are both incorporated herein by reference in their entireties.

Additionally, the disclosure and teaching contained in the specifications and sequence listings of U.S. Provisional Application No. 60/336,695, filed Dec. 7, 2001, U.S. patent application Ser. No. 10/226,294, filed Aug. 23, 2002, U.S. Provisional Application No. 60/314,381, filed Aug. 24, 2001, U.S. patent application Ser. No. 09/899,059, filed Jul. 6, 2001, U.S. Provisional Application No. 60/278,449, filed Mar. 26, 2001, U.S. Provisional Application No. 60/216,879, filed Jul. 7, 2000, U.S. patent application Ser. No. 09/559,290, filed Apr. 27, 2000, U.S. Provisional Application No. 60/180,908, filed Feb. 8, 2000, U.S. Provisional Application No. 60/134,067, filed May 13, 1999, U.S. Provisional Application No. 60/132,227, filed May 3, 1999, U.S. Provisional Application No. 60/131,963, filed Apr. 30, 1999, U.S. patent application Ser. No. 09/246,129, filed Feb. 8, 1999, U.S. Provisional Application No. 60/074,047, filed Feb. 9, 1998, U.S. patent application Ser. No. 09/131,237, filed Aug. 7, 1998, U.S. patent application Ser. No. 09/005,020, filed Jan. 9, 1998, U.S. patent application Ser. No. 08/461,246, filed Jun. 5, 1995, and PCT/US94/12880 filed Nov. 7, 1994, are herein incorporated by reference in their entireties.

What is claimed is:

1. A method for treating or ameliorating a disease or disorder of the gastrointestinal tract comprising administering a composition comprising an antagonist of TNF-gamma-β to a person with, or suspected of having, said disease or disorder.

2. The method of claim 1, wherein said antagonist is an antibody or fragment thereof that specifically binds to a TNF-gamma-β polypeptide consisting of amino acid residues 62-251 of SEQ ID NO:2.

3. The method of claim 2, wherein said antagonist is an antibody or fragment thereof that specifically binds to a complex selected from the group consisting of:
(a) a homotrimer comprising a TNF-gamma-β polypeptide consisting of amino acid residues 62-251 of SEQ ID NO:2;
(b) a heterotrimer comprising a TNF-gamma-β polypeptide consisting of amino acid residues 62-251 of SEQ ID NO:2; and
(c) both (a) and (b).

4. The method of claim 1, wherein said antagonist prevents increased secretion of IFN-γ by lamina propria mononuclear cells.

5. The method of claim 1, wherein said antagonist is an antibody or fragment thereof that specifically binds to a polypeptide consisting of amino acid residues 25-201 of SEQ ID NO:4.

6. The method of claim 5, wherein said antagonist is an antibody or fragment thereof that specifically binds to a complex selected from the group consisting of:
(a) a homotrimer comprising a polypeptide consisting of amino acid residues 25-201 of SEQ ID NO:4;
(b) a heterotrimer comprising a polypeptide consisting of amino acid residues 25-201 of SEQ ID NO:4; and
(c) both (a) and (b).

7. The method of claim 1, wherein said antagonist is a polypeptide comprising amino acid residues 25-201 of SEQ ID NO:4, or a TNF-gamma-β-binding fragment thereof.

8. The method of claim 7, wherein said antagonist is fused to a heterologous polypeptide.

9. The method of claim 8, wherein said heterologous polypeptide is human serum albumin.

10. The method of claim 8, wherein said heterologous polypeptide is an immunoglobulin Fc domain.

11. The method of claim 1, wherein said antagonist is a polypeptide comprising amino acid residues 31-300 of SEQ ID NO:6, or a TNF-gamma-β-binding fragment thereof.

12. The method of claim 11, wherein said antagonist is fused to a heterologous polypeptide.

13. The method of claim 12, wherein said heterologous polypeptide is human serum albumin.

14. The method of claim 12, wherein said heterologous polypeptide is an immunoglobulin Fc domain.

15. The method of claim 1, wherein said disease is inflammatory bowel disease.

16. The method of claim 15, wherein said inflammatory bowel disease is Crohn's disease.

17. The method of claim 15, wherein said inflammatory bowel disease is ulcerative colitis.

18. A method of diagnosing a disease or disorder of the gastrointestinal tract comprising detecting abnormal levels of a polypeptide selected from the group consisting of:
(a) a TNF-gamma-β polypeptide consisting of amino acid residues 62-251 of SEQ ID NO:2;
(b) a polypeptide consisting of amino acid residues 25-201 of SEQ ID NO:4 or a fragment thereof; and
(c) a polypeptide comprising amino acid residues 31-300 of SEQ ID NO:6 or a fragment thereof, in a biological sample.

19. The method of claim 18, wherein the polypeptide being detected is (a).

20. The method of claim 19, wherein the level of expression of said protein is abnormally elevated.

21. The method of claim 18, wherein the polypeptide being detected is (b).

22. The method of claim 21, wherein the level of expression of said protein is abnormally elevated.

23. The method of claim 18, wherein the polypeptide being detected is (c).

24. The method of claim 23, wherein the level of expression of said protein is abnormally elevated.

25. The method of claim 18, wherein the disease or disorder is inflammatory bowel disease.

26. The method of claim 25, wherein said inflammatory bowel disease is Crohn's disease.

27. The method of claim 25, wherein said inflammatory bowel disease is ulcerative colitis.


SEQUENCE LISTING (71 sequences)

SEQ ID NO: 1 - DNA, 1116 bp, human
atggccgaggatctgggactgagctttggggaaacagccagtgtggaaatgctgccagagcacggcagctgcaggcccaaggccaggagcagcagcgcacgctgggctctcacctgctgcctggtgttgctccccttccttgcaggactcaccacatacctgcttgtcagccagctccgggcccagggagaggcctgtgtgcagttccaggctctaaaaggacaggagtttgcaccttcacatcagcaagtttatgcacctcttagagcagacggagataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactataggaggagagcaaatatcattatatgaaagtcctctgccaccgagttcctaattttctttgttcaaatgtaattataaccaggggttttcttggggccgggagtaggggcattccacagggacaacggtttagctatgaaatttggggcccaaaatttcacacttcatgtgccttactgatgagagtactaactggaaaaaggctgaagagagcaaatatattattaagatgggttggaggattggcgagtttctaaatattaagacactgatcactaaatgaatggatgatctactcgggtcaggattgaaagagaaatatttcaacaccttcctgctatacaatggtcaccagtggtcca

SEQ ID NO: 2 - protein, 251 aa, human
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 3 - DNA, 1254 bp, human
atggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtaa

SEQ ID NO: 4 - protein, 417 aa, human
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 5 - DNA, 1077 bp, human
gctctccctgctccagcaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccccctcttatttattctacatccttggcaccccacttgcactgaaagaggcttttttttaaatagaagaaatgaggtttcttaaagcttatttttataaagctttttcataaaaaaaaaaaaaaaaaaaaaaaaaaaaaaaaa

SEQ ID NO: 6 - protein, 300 aa, human
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 7 - DNA, 1325 bp, human
gaggtttattgggcctcggtcctcctgcacctgctgcctggatccccggcctgcctgggcctgggccttggttctccccatgacaccacctgaacgtctcttcctcccaagggtgtgtggcaccaccctacacctcctccttctggggctgctgctggttctgctgcctggggcccaggggctccctggtgttggcctcacaccttcagctgcccagactgcccgtcagcaccccaagatgcatcttgcccacagcaccctcaaacctgctgctcacctcattggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtagaacttggaaaaatccagaaagaaaaaataattgatttcaagaccttctccccattctgcctccattctgaccatttcaggggtcgtcaccacctctcctttggccattccaacagctcaagtcttccctgatcaagtcaccggagctttcaaagaaggaattctaggcatcccaggggacccacactccctgaaccatccctgatgtctgtctggctgaggatttcaagcctgcctaggaattcccagcccaaagctgttggtcttgtccaccagctaggtggggcctagatccacacacagaggaagagcaggcacatggaggagcttgggggatgactagaggcagggaggggactatttatgaaggcaaaaaaattaaattatttatttatggaggatggagagagggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctga

SEQ ID NO: 8 - protein, 205 aa, human
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 9 - DNA, 1643 bp, human
gcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaattgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgctt

SEQ ID NO: 10 - protein, 233 aa, human
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 11 - DNA, 894 bp, human
cagtctcaatgggggcactggggctggagggcaggggtgggaggctccaggggaggggttccctcctgctagctgtggcaggagccacttctctggtgaccttgttgctggcggtgcctatcactgtcctggctgtgctggccttagtgccccaggatcagggaggactggtaacggagacggccgaccccggggcacaggcccagcaaggactggggtttcagaagctgccagaggaggagccagaaacagatctcagccccgggctcccagctgcccacctcataggcgctccgctgaaggggcaggggctaggctgggagacgacgaaggaacaggcgtttctgacgagcgggacgcagttctcggacgccgaggggctggcgctcccgcaggacggcctctattacctctactgtctcgtcggctaccggggccgggcgccccctggcggcggggacccccagggccgctcggtcacgctgcgcagctctctgtaccgggcggggggcgcctacgggccgggcactcccgagctgctgctcgagggcgccgagacggtgactccagtgctggacccggccaggagacaagggtacgggcctctctggtacacgagcgtggggttcggcggcctggtgcagctccggaggggcgagagggtgtacgtcaacatcagtcaccccgatatggtggacttcgcgagagggaagaccttctttggggccgtgatggtggggtgagggaatatgagtgcgtggtgcgagtgcgtgaatattgggggcccggacgcccaggaccccatggcagtgggaaaaatgtaggagactgtttggaaattgattttgaacctgatgaaaataaagaatggaaagcttcagtgctgccgataaa

SEQ ID NO: 12 - protein, 244 aa, human
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 13 - DNA, 3362 bp, human
ccatatcttcatcttccctctacccagattgtgaagatggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttgaggggctgatggcaatatctaaaaccaggcaccagcatgaacaccaagctgggggtggacagggcatggattcttcattgcaagtgaaggagcctcccagctcagccacgtgggatgtgacaagaagcagatcctggccctcccgcccccacccctcagggatatttaaaacttattttatataccagttaatcttatttatccttatattttctaaattgcctagccgtcacaccccaagattgccttgagcctactaggcacctttgtgagaaagaaaaaatagatgcctcttcttcaagatgcattgtttctattggtcaggcaattgtcataataaacttatgtcattgaaaacggtacctgactaccatttgctggaaatttgacatgtgtgtggcattatcaaaatgaagaggagcaaggagtgaaggagtggggttatgaatctgccaaaggtggtatgaaccaacccctggaagccaaagcggcctctccaaggttaaattgattgcagtttgcatattgcctaaatttaaactttctcatttggtgggggttcaaaagaagaatcagcttgtgaaaaatcaggacttgaagagagccgtctaagaaataccacgtgctttttttctttaccattttgctttcccagcctccaaacatagttaatagaaatttcccttcaaagaactgtctggggatgtgatgctttgaaaaatctaatcagtgacttaagagagattttcttgtatacagggagagtgagataacttattgtgaagggttagctttactgtacaggatagcagggaactggacatctcagggtaaaagtcagtacggattttaatagcctggggaggaaaacacattctttgccacagacaggcaaagcaacacatgctcatcctcctgcctatgctgagatacgcactcagctccatgtcttgtacacacagaaacattgctggtttcaagaaatgaggtgatcctattatcaaattcaatctgatgtcaaatagcactaagaagttattgtgccttatgaaaaataatgatctctgtctagaaataccatagaccatatatagtctcacattgataattgaaactagaagggtctatatcagcctatgccagggcttcaatggaatagtatccccttatgtttagttgaaatgtccccttaacttgatataatgtgttatgcttatggcgctgtgacaatctgatttttcatgtcaacttccagatgatttgtaacttctctgtgccaaaccttttataaacataaatttttgagatatgtattttaaaattgtagcacatgtttccctgacattttcaatagaggatacaacatcacagaatctttctggatgattctgtgttatcaaggaattgtactgtgctacaattatctctagaatctccagaaaggtggagggctgttcgcccttacactaaatggtctcagttggatttttttttcctgttttctatttcctcttaagtacaccttcaactatattcccatccctctattttaatctgttatgaaggaaggtaaataaaaatgctaaatagaagaaattgtaggtaaggtaagaggaatcaagttctgagtggctgccaaggcactcacagaatcataatcatggctaaatatttatggagggcctactgtggaccaggcactggctaaatacttacatttacaagaatcattctgagacagatattcaatgatatctggcttcactactcagaagattgtgtgtgtgtttgtgtgtgtgtgtgtgtgtgtatttcactttttgttattgaccatgttctgcaaaattgcagttactcagtgagtgatatccgaaaaagtaaacgtttatgactataggtaatatttaagaaaatgcatggttcatttttaagtttggaatttttatctatatttctcacagatgtgcagtgcacatgcaggcctaagtatatgttgtgtgtgtttgtctttgacgtcatggtcccctctcttaggtgctcactcgctttgggtgcacctggcctgctcttcccatgttggcctctgcaaccacacagggatatttctgctatgcaccagcctcactccaccttccttccatcaaaaatatgtgtgtgtgtctcagtccctgtaagtcatgtccttcacagggagaattaacccttcgatatacatggcagagttttgtgggaaaagaattgaatgaaaagtcaggagatcagaattttaaatttgacttagccactaactagccatgtaaccttgggaaagtcatttcccatttctgggtcttgcttttctttctgttaaatgagaggaatgttaaatatctaacagtttagaatcttatgcttacagtgttatctgtgaatgcacatattaaatgtctatgttcttgttgctatgagtcaaggagtgtacacttctcctttactatgttgaatgtatttttttctggacaagcttacatcttcctcagccatctttgtgagtccttcaagagcagttatcaattgttagttagatattttctatttagagaatgcttaagggattccaatcccgatccaaatcataatttgttcttaagtatactgggcaggtcccctattttaagtcataattttgtatttagtgctttcctggctctcagagagtattaatattgatattaataatatagttaatagtaatattgctatttacatggaaacaaataaaagatctcagaattc

SEQ ID NO: 14 - protein, 183 aa, human
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 15 - DNA, 1803 bp, human
tgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacatt

SEQ ID NO: 16 - protein, 261 aa, human
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 17 - DNA, 972 bp, human
tctagactcaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagatgttctaga

SEQ ID NO: 18 - protein, 281 aa, human
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 19 - DNA, 926 bp, human
ccagagaggggcaggcttgtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgtctcccagcgcctgacgcccctggcccgaggggacacactctgcaccaacctcactgggacacttttgccttcccgaaacactgatgagaccttctttggagtgcagtgggtgcgcccctgaccactgctgctgattagggttttttaaattttattttattttatttaagttcaagagaaaaagtgtacacacaggggccacccggggttggggtgggagtgtggtggggggtagtttgtggcaggacaagagaaggcattgagctttttctttcattttcctattaaaaaatacaaaaatcaaaacaaaaaaaa

SEQ ID NO: 20 - protein, 193 aa, human [sequence omitted]

SEQ ID NO: 21 - DNA, 1906 bp, human
ccaagtcacatgattcaggattcagggggagaatccttcttggaacagagatgggcccagaactgaatcagatgaagagagataaggtgtgatgtggggaagactatataaagaatggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagagctccattcaagaagtcatgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactgaacagtttctcttggccttcaggaagaaagcgcctctctaccatacagtatttcatccctccaaacacttgggcaaaaagaaaactttagaccaagacaaactacacagggtattaaatagtatacttctccttctgtctcttggaaagatacagctccagggttaaaaagagagtttttagtgaagtatctttcagatagcaggcagggaagcaatgtagtgtggtgggcagagccccacacagaatcagaagggatgaatggatgtcccagcccaaccactaattcactgtatggtcttgatctatttcttctgttttgagagcctccagttaaaatggggcttcagtaccagagcagctagcaactctgccctaatgggaaatgaaggggagctgggtgtgagtgtttacactgtgcccttcacgggatacttcttttatctgcagatggcctaatgcttagttgtccaagtcgcgatcaaggactctctcacacaggaaacttccctatactggcagatacacttgtgactgaaccatgcccagtttatgcctgtctgactgtcactctggcactaggaggctgatcttgtactccatatgaccccacccctaggaacccccagggaaaaccaggctcggacagccccctgttcctgagatggaaagcacaaatttaatacaccaccacaatggaaaacaagttcaaagacttttacttacagatcctggacagaaagggcataatgagtctgaagggcagtcctccttctccaggttacatgaggcaggaataagaagtcagacagagacagcaagacagttaacaacgtaggtaaagaaatagggtgtggtcactctcaattcactggcaaatgcctgaatggtctgtctgaaggaagcaacagagaagtggggaatccagtctgctaggcaggaaagatgcctctaagttcttgtctctggccagaggtgtggtatagaaccagaaacccatatcaagggtgactaagcccggcttccggtatgagaaattaaacttgtatacaaaatggttgccaaggcaacataaaattataagaattc

SEQ ID NO: 22 - protein, 234 aa, human
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 23 - DNA, 1619 bp, human
gtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataacgcccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttggggggggggtcttcgacattgccgaggctggtcttgaactcctggacttagacgatcctcctgcctcagcctcccaagcaactgggattcatcctttctattaattcattgtacttatttgcctatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcc

SEQ ID NO: 24 - protein, 254 aa, human
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 25 - DNA, 1769 bp, human
cctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtgcaaaaattagctgggtgtgttggcacatgcctgtagtcccagctacttgagaggctgaggcaggagaatcgtttgaacccgggaggcagaggttgcagtgtggtgagatcatgccactacactccagcctggcgacagagcgagacttggtttcaaaaaaaaaaaaaaaaaaaaacttcagtaagtacgtgttatttttttcaataaaattctattacagtatgtcaaaaaaaaaaaaaaaaaa

SEQ ID NO: 26 - protein, 281 aa, human [sequence omitted]

SEQ ID NO: 27 - DNA, 2271 bp, human
aagcttggtaccgagctcggatccactactcgacccacgcgtccgcgcgccccaggagccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacagccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatctgtcaagagtagttatataattgttgaacaggtgtttttccacaagtgccgcaaattgtacctttttttttttttcaaaatagaaaagttattagtggtttatcagcaaaaaagtccaattttaatttagtaaatgttatcttatactgtacaataaaaacattgcctttgaatgttaattttttggtacaaaaataaatttatatgaaaaaaaaaaaaaaagggcggccgctctagagggccctattctatag

SEQ ID NO: 28 - protein, 317 aa, human
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 29 - DNA, 1306 bp, human
cacagccccccgcccccatggccgcccgtcggagccagaggcggagggggcgccggggggagccgggcaccgccctgctggtcccgctcgcgctgggcctgggcctggcgctggcctgcctcggcctcctgctggccgtggtcagtttggggagccgggcatcgctgtccgcccaggagcctgcccaggaggagctggtggcagaggaggaccaggacccgtcggaactgaatccccagacagaagaaagccaggatcctgcgcctttcctgaaccgactagttcggcctcgcagaagtgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggccagttccctcgggccccagctccgcctctgccaggtgtctgggctgttggccctgcggccagggtcctccctgcggatccgcaccctcccctgggcccatctcaaggctgcccccttcctcacctacttcggactcttccaggttcactgaggggccctggtctccccacagtcgtcccaggctgccggctcccctcgacagctctctgggcacccggtcccctctgccccaccctcagccgctctttgctccagacctgcccctccctctagaggctgcctgggcctgttcacgtgttttccatcccacataaatacagtattcccactcttatcttacaactcccccaccgcccactctccacctcactagctccccaatccctgaccctttgaggcccccagtgatctcgactcccccctggccacagacccccagggcattgtgttcactgtactctgtgggcaaggatgggtccagaagaccccacttcaggcactaagaggggctggacctggcggcaggaagccaaagagactgggcctaggccaggagttcccaaatgtgaggggcgagaaacaagacaagctcctcccttgagaattccctgtggatttttaaaacagatattatttttattattattgtgacaaaatgttgataaatggatattaaatagaataagtcag

SEQ ID NO: 30 - protein, 249 aa, human
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 31 - DNA, 1348 bp, human
ggtacgaggcttcctagagggactggaacctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcatcttcctgggtttggctccccgttcctcacttttcccttttcattcccaccccctagactttgattttacggatatcttgcttctgttccccatggagctccgaattcttgcgtgtgtgtagatgaggggcgggggacgggcgccaggcattgttcagacctggtcggggcccactggaagcatccagaacagcaccaccatctta

SEQ ID NO: 32 - protein, 250 aa, human [sequence omitted]

SEQ ID NO: 33 - DNA, 1126 bp, human
cccacccgtccgcccacgcgtccgccactgcccgtacccttacccgccccgccacctacttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcagaggacaggaggcccctcccagaatggggaagggtatccctggcagagtctcccggagcagagttccgatgccctggaagcctgggagagtggggagagatcccggaaaaggagagcagtgctcacccaaaaacagaagaatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcgtcttcctgggtttggctccccgttcctcacttttcccttttcattcccaccccctagactttgattttacggatatcttgcttctgttccccatggagctccgaattcttgcgtgtgtgtagatgaggggcggggggacgggcgccaggcattgttcagacctggtcggggcccactggaagcatccagaacagcaccaccatcta

SEQ ID NO: 34 - protein, 234 aa, human
MPASSPFLLAPKGPPGNMGGPVREPALSVALWLSWGAALGAVACAMALLTQQTELQSLRREVSRLQRTGGPSQNGEGYPWQSLPEQSSDALEAWESGERSRKRRAVLTQKQKNDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 35 - DNA, 858 bp, human
atggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcttggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtga

SEQ ID NO: 36 - protein, 285 aa, human
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 37 - DNA, 798 bp, human
atggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctg

SEQ ID NO: 38 - protein, 266 aa, human
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 39 - DNA, 1169 bp, human
gaggttgaaggacccaggcgtgtcagccctgctccagagaccttgggcatggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggctgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtgtgaaggaaggagcgtggtgcattggacatgggtctgacacgtggagaactcagagggtgcctcaggggaaagaaaactcacgaagcagaggctgggcgtggtggctctcgcctgtaatcccagcactttgggaggccaaggcaggcggatcacctgaggtcaggagttcgagaccagcctggctaacatggcaaaaccccatctctactaaaaatacaaaaattagccggacgtggtggtgcctgcctgtaatccagctactcaggaggctgaggcaggataattttgcttaaacccgggaggcggaggttgcagtgagccgagatcacaccactgcactccaacctgggaaacgcagtgagactgtgcctcaaaaaaaaaaaaaaaaaaaaaaaaaaaaa

SEQ ID NO: 40 - protein, 240 aa, human
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 41 - DNA, 2785 bp, human
  misc_feature (49)..(49): n equals a, g, t, or c
agtgcagtatctcatggaggtgtttggatgtctcttcctgtggggggtnccaaagcccatgtctcttggcattttctttcagattctatcagccctctctctttctctcctgtctctctctttcattcatacactgagtcattcagagatggcttctctccaactcggagctgcaagtaattctggatctggtcacacacacaaagtccccagagttgccaatttatctagttcatctgtgcctgttcaagatgatgtaactaaacatttaccttcagggaggtgtttccaaagaattttcatcgatatatagaaatcaagagaaaatccatactatcaccaaatcaagagaaattccatactatcaccagttggccaactttccaagtctagtgcagaaatccaaggcacctcacacctagagttcctatacctctgagactccagaggaaagaacaagacagtgcagaaggatatgttagaacccactgaaaacctagaaggttaaaaaggaagcataccctcctgacctataagaaaattttcagtctgcagggggatatccttgtggcccaagacattggtgttatcatttgactaagaggaaattatttgtggtgagctctgagtgaggattaggaccagggagatgccaagtttctatcacttacctcatgcctgtaagacaagtgttttgttccaattgatgaatggggataaaacagttcagccaatcacttatggggcaaagaatgggaatttgaagggtctggtgcctggccttgtcatacgtaaacaagagaggcatcgatgagttttatctgagtcatttgggaaaggataattcttgcagcaagccattttcctaaacacagaagaatagggggattccttaaccttcattgttctccaggatcataggtctcaggtaaaattaaaaattttcaggtcagaccactcagtctcagaaaggcaaagtaatttgccccaggtcactagtccaagatgttattctctttgaacaaatgtgtatgtccagtcacatattcttcattcattcctccccaaagcagtttttagctgttaggtatattcgatcactttagtctattttgaaaatgatatgagacgctttttaagcaaagtctacagtttcccaatgagaaaattaatcctctttcttgtctttccagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactataggaggagagcaaatatcattatatgaaagtcctctgccaccgagttcctaattttctttgttcaaatgtaattataaccaggggttttcttggggccgggagtagggggcattccacagggacaacggtttagctatgaaatttggggccaaaatttcacacttcatgtgccttactgatgagagtactaactggaaaaaggctgaagagagcaaatatattattaagatgggttggaggattggcgagtttctaaatattaagacactgatcactaaatgaatggatgatctactcgggtcaggattgaaagagaaatatttcaacacctcctgctatacaatggtcaccagtggtccagttattgttcaatttgatcataaatttgcttcaattcaggagctttgaaggaagtccaaggaaagctctagaaaacagtataaactttcagaggcaaaatccttcaccaatttttccacatactttcatgccttgcctaaaaaaaatgaaaagagagttggtatgtctcatgaatgttcacacagaaggagttggttttcatgtcatctacagcatatgagaaaagctacctttcttttgattatgtacacagatatctaaataaggaagtatgagtttcacatgtatatcaaaaatacaacagttgcttgtattcagtagagttttcttgcccacctattttgtgctgggttctaccttaacccagaagacactatgaaaaacaagacagactccactcaaaatttatatgaacaccactagatacttcctgatcaaacatcagtcaacatactctaaagaataactccaagtcttggccaggcgcagtggctcacacctgtaatcccaacactttgggaggccaaggtgggtggatcatctaaggccgggagttcaagaccagcctgaccaacgtggagaaaccccatctctactaaaaatacaaaattagccgggcgtggtagcgcatggctgtaatcctggctactcaggaggccgaggcagaagaattgcttgaactggggaggcagaggttgcggtgagcccagatcgcgccattgcactccagcctgggtaacaagagcaaaactctgtccaaaaaaaaaaaaaaaaaaaa

SEQ ID NO: 42 - protein, 174 aa, human
MRRFLSKVYSFPMRKLILFLVFPVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 43 - DNA, 534 bp, human
atgtgtttgagccacttggaaaatatgcctttaagccattcaagaactcaaggagctcagagatcatcctggaagctgtggctcttttgctcaatagttatgttgctatttctttgctccttcagttggctaatctttatttttctccaattagagactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctag

SEQ ID NO: 44 - protein, 177 aa, human
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 45 - DNA, 5307 bp, human
  misc_feature (4242)..(4242): n equals a, g, t, or c
attccctcggcgggccgagcctcccctctctcccgcccctcctcctccctttcccacccctcggagtagagctgcacatgcggctgctccctgctccgtcccgcccagccactgtcgcgcaggaacgggtccctgcagcccccagccgatggcaggacagtagccgcctgtcagaggtcgtgaacggctgaggcagacgcagcggctcccgggcctcaagagagtggatgtctccggaggccatgggctacccggaggtggagcgcagggaactcctgcctgcagcagcgccgcgggagcgagggagccagggctgcgggtgtggcggggcccctgcccgggcgggcgaagggaacagctgcctgctcttcctgggtttctttggcctctcgctggccctccacctgctgacgttgtgctgctacctagagttgcgctcggagttgcggcgggaacgtggagccgagtcccgccttggcggctcgggcacccctggcacctctggcaccctaagcagcctcggtggcctcgaccctgacagccccatcaccagtcaccttgggcagccgtcacctaagcagcagccattggaaccgggagaagccgcactccactctgactcccaggacgggcaccagatggccctattgaatttcttcttccctgatgaaaagccatactctgaagaagaaagtaggcgtgttcgccgcaataaaagaagcaaaagcaatgaaggagcagatggcccagttaaaaacaagaaaaagggaaagaaagcaggacctcctggacccaatggccctccaggacccccaggacctccaggaccccagggacccccaggaattccagggattcctggaattccaggaacaactgttatgggaccacctggtcctccaggtcctcctggtcctcaaggaccccctggcctccagggaccttctggtgctgctgataaagctggaactcgagaaaaccagccagctgtggtgcatctacagggccaagggtcagcaattcaagtcaagaatgatctttcaggtggagtgctcaatgactggtctcgcatcactatgaaccccaaggtgtttaagctacatccccgcagcggggagctggaggtactggtggacggcacctacttcatctatagtcaggtagaagtatactacatcaacttcactgactttgccagctatgaggtggtggtggatgagaagcccttcctgcagtgcacacgcagcatcgagacgggcaagaccaactacaacacttgctataccgcaggcgtctgcctcctcaaggcccggcagaagatcgccgtcaagatggtgcacgctgacatctccatcaacatgagcaagcacaccacgttctttggggccatcaggctgggtgaagcccctgcatcctagattccccccattttgcctctgtccgtgccccttccctgggtttgggagccaggactcccagaacctctaagtgctgctgtggagtgaggtgtattggtgttgcagccgcagagaaatgccccagtgttatttattccccagtgactccagggtgacaaggcctgcttgactttccagaatgaccttgagttaacaggacagttgatggagccccagggtttacatgaagcagaaccttctttggttccatgttgactgacttatggcatgactcttcaaccccgaggtccctgttgtcagatctattgtttgttgcactaaaatgaggatccagggcagcaggccagagaaagcaaaggtgcactccagactctgggggtggacatctgaccccaagggggctgctgctcctctcttgggtagggtagtggctggggtggagtgggaagkgagcattgcagcctaagaagaaggccagagagggaaaaggcaggtgcttttggcagagaccataagagaaacctgccaaggagcatccttggcagtgggaatgttctttctgctctatactgtggcctgcaggagggttggagtgctcttcccactccagctgacagccacaccgtggcagcttgctgggctttgggaagtttgctgtgctttggaacaatcacagggaatggccacaaacctgcccgcctaagaccctgaatccgtacttgggtcacatgactctcattttatttacagctgtgctccacactcagaaaattccctggggtcaccttctagttgcccccattcccagcctgactagaactcctgtcttctttctccatggagcctacctctgtctgagacaggtgcctaacctgggacctgtggtcatgtgagtctgggatattctttagcttacctgggcacagacagaattttccatttattaagcagtacagatgtttttcatccattcctaatcaaattctgtctggggacgaagggttggacgggatgacctccagaagtcccttcaatttctagtacctgtgactcttagccctcaccacagccttctaaattcccaaatcctagactgctcctgggcattagcaaggcagagcctttttacctggcctagaaagggcaaggggtgaggataggacagagggattttgttcaagtttgctgcaacccaagtggacgttaggccaggccttatctgaaaggccagcagctgatgctgtactaacccagtctttcttcactctggcttcaaaaagccacagcagagcattgtcaccgcaggtccccatgctgctcccctaaagccaggctcaggagaagccagtgtctaggcactgagcagggatctgccccctagttcaggtccaaattcaccttcccctaaaccccaagcttcccaacagatcatatggtaggaccctcgagagccttacttcaaagtgcctgggctcagcctggtttctgggtgctagatccagcccaaacctgggaaggccagccttgtacagtctgctcctcttgttcctgaaatgtgtttccttttcaggagatggggaataatttccttcaggcagctgaaattcaccaagaacagcgggtacttatttctcaagctgtgccttccctttctaagcaaccacactgcttggcccttcaagggtcagggtgagacgtgatgggctaggcctccgttgtctggttgctaatgacagccttgcaacccaaggtgaggtgaactccaggcatgtgtctggccctaactcctataaagtgcctcggacagtccgcagttgtagcagaaaccaacaagaaccactccttcatgtttggaaaataatttctcttgtattatctcctttgaagaaggcaaggctgataatatgacaaacatcattgtttagatgaggctcagagaggtagcactctcagagtgttttgaccagtttaagccgcagacctggagcttcagccaggtctgactccaaagctgttccattacaccacagcattgtgtggaatttgaggtctagagagaaccaataaaagtggtaattgggaactgaaatccttgagagttccggggagaaacccagagatgcctgatttcattcctcgatggtaatacccgtcctctcggctgccaggggctctgtggcaaaaagagtcagacatttctttggaaaacagcgaacagccttagagctcttgtgttcagaagaatcttcctggcacaatgttggagcagcaggcctctgggacccacagaacttgtggcctttatgttctttcacccatcctaggaaccagccaaccatcatgtgtagagcccctactgtgggcaaagtcctcctttcattaccctacagacagcttacaggagccagcctgcttcccacaactactagtgtgactccttatctctttccaccataccttagagactttgatactaccagggtctctcagggatggagggaagacctgaaagagaggactggttctgaggccagaaaggtgtgaggagagaggaggaaaagtcttcctaattgtgcccctaaagagcatcctgataccattctattctccagacatggaggggatgataaaggaaataggatctccactggacccttgattcattctgaaccctccaaaggaactctaagagggcgagggatgatgagggaagcaataggtagctggggagccctattgctgctaagtcattggcaaagtgcaaagcaatttactgatgagagaatgtggaaatagatgtgcagtttggaattatgttggtgtnaatttgccagaggaccaatgcttgcatggagaatggacgaggacatttgtgggcaagcagatgacagaggtttgaaggagaatggcatggcaggagtctctgccagttacttgggcttcaacagccaagctggcacaaaagacagctggcggaggctgctcggctactggttacctggagaagtagtatttgcctatttcccccttcatccatcctgagccaaatttcntttgctgaacaggaaagagcyaggaaccctggaggtaaacaaagactttgancctgtntnagtgtatgtgtttntgtaacttcctgtggagtgcaaatagattcagagaaatttagagctaaaaaggcccttagagggaatctagcccaacctacattccaccctgttacttatgtagaaactgaggcccagagagggaagatgacctgccccaagtggtgagcaagcaccaacctccagactcagcagagtgagggggtaaagcagttcctgtcccacatggccatcttctttcttccacccacaaactccaggctggaagtacttggcccccttcaggagcctggccaggcagggagagagtagctgcagccttcatcagaactcttcctcctcccaaggcattctcccagctctagcctctggactggaaagcacaagactggcccagtgccagcaagtccttaggctactgtaatgctgcctcaggacccatccctgcctggaggctcctctaggccctgtgagcacaaagaagaaagctgatttttgtcttttaatccatttcaggactctctccaggagggctcggggtgtgtcatttctatattcctccagctgggattggggggtgggctttgttgtgagaatggcctggagcaggcccaatgctgcttttgggggtcagcatccagtgtgagatactgtgtatataaactatatataatgtatataaactgggatgtaagtttgtgtaaattaatgttttattctttgcaaataaaacgctttccccgtcaaaaaaaaaaaaaaaaaaaaaaaaaaaaaaaaaaaaa

SEQ ID NO: 46 - protein, 391 aa, human
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 47 - DNA, 37 bp, human
gcgcggatccaccatgagacgctttttaagcaaagtc

SEQ ID NO: 48 - DNA, 36 bp, human
cgcgtctagactatagtaagaaggctccaaagaagg

SEQ ID NO: 49 - DNA, 56 bp, human
cgctctagatcaagcgtagtctgggacgtcgtatggatagtaagaaggctccaaag

SEQ ID NO: 50 - DNA, 733 bp, human
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 51 - DNA, 26 bp, human
cgcccatggcagaaacacccacctac

SEQ ID NO: 52 - DNA, 26 bp, human
cgcaagcttctctttcagtgcaagtg

SEQ ID NO: 53 - DNA, 28 bp, human
cgcaagcttctcctcagctcctgcagtg

SEQ ID NO: 54 - DNA, 36 bp, human
cgcggatccgccatcatgagggcgtggaggggccag

SEQ ID NO: 55 - DNA, 26 bp, human
cgcggtaccctctttcagtgcaagtg

SEQ ID NO: 56 - DNA, 28 bp, human
cgcggtaccctcctcagctcctgcagtg

SEQ ID NO: 57 - DNA, 25 bp, human
cacctcttagagcagacggagataa

SEQ ID NO: 58 - DNA, 24 bp, human
ttaaagtgctgtgtgggagtttgt

SEQ ID NO: 59 - DNA, 25 bp, human
ccaagggcacacctgacagttgtga

SEQ ID NO: 60 - DNA, 26 bp, human
caaagtctacagtttcccaatgagaa

SEQ ID NO: 61 - DNA, 26 bp, human
gggaactgatttttaaagtgctgtgt

SEQ ID NO: 62 - DNA, 34 bp, human
tcctctttcttgtctttccagttgtgagacaaac

SEQ ID NO: 63 - DNA, 36 bp, human
gcaaagtctacagtttcccaatgagaaaattaatcc

SEQ ID NO: 64 - DNA, 24 bp, human
atggccgaggatctgggactgagc

SEQ ID NO: 65 - DNA, 36 bp, human
ctatagtaagaaggctccaaagaaggttttatcttc

SEQ ID NO: 66 - DNA, 23 bp, human
gcgccatgggggcccggcggcag

SEQ ID NO: 67 - DNA, 30 bp, human
gcgaagcttctaggacccagaacatctgcc

SEQ ID NO: 68 - DNA, 33 bp, human
cgcggatccgccatcatggagcagcggccgcgg

SEQ ID NO: 69 - DNA, 54 bp, human
gcgtctagatcaaagcgtagtctgggacgtcgtatgggtacgggccgcgctgca

SEQ ID NO: 70 - DNA, 26 bp, human
cgcggatcctcacgggccgcgctgca

SEQ ID NO: 71 - DNA, 35 bp, human
gcgagatctagtctggacccagaacatctgcctcc